(19) United States
(12) Patent Application Publication
(10) Pub. No.: US 2007/0248590 A1
(43) Pub. Date: Oct. 25, 2007

(54) Modulators of CDC2-like kinases (CLKS) and methods of use thereof

(75) Inventors: Jill Milne, Brookline, MA (US); Karl D. Normington, Acton, MA (US); Pere Puigserver, Baltimore, MD (US); Joseph Rodgers, Baltimore, MD (US)

Correspondence Address: FISH & NEAVE IP GROUP;ROPES & GRAY LLP, ONE INTERNATIONAL PLACE, BOSTON, MA, 02110-2624, US

(73) Assignee: Sirtris Pharmaceuticals, Inc., Cambridge, MA (US)

(21) Appl. No.: 11/607,783

(22) Filed: Dec. 1, 2006

Related U.S. Application Data
(60) Provisional application No. 60/741,782, filed on Dec. 2, 2005.

(51) Int. Cl. A61K 31/00; A61K 31/045; A61K 31/35; A61K 31/355; A61K 31/385; A61K 31/426; A61K 31/435; A61K 31/495; A61K 31/51; A61K 31/525; A61K 31/56; A61K 31/70; A61K 31/7052; A61K 38/43; A61K 39/395; A61P 21/00; A61P 3/00; A61P 3/04; A61P 35/00; A61P 43/00; A61P 5/50; A61P 7/00; A61P 7/12; C12N 5/00

(52) U.S. Cl. 424/130.1; 424/94.1; 435/325; 514/1; 514/177; 514/23; 514/249; 514/251; 514/276; 514/357; 514/367; 514/43; 514/44; 514/440; 514/456; 514/458; 514/733

(57) ABSTRACT

Provided herein are methods for using Cdc2-like kinase (Clk) modulators for treating and/or preventing a wide variety of diseases and disorders including, for example, diseases or disorders related to aging or stress, diabetes, obesity, neurodegenerative diseases, cardiovascular disease, blood clotting disorders, inflammation, cancer, ocular disorders, and/or flushing as well as diseases or disorders that would benefit from increased mitochondrial activity. Also provided are compositions comprising a Clk modulating compound in combination with another therapeutic agent.

### RELATED APPLICATIONS

This application claims the benefit of U.S. Provisional Application No. 60/741,782, filed Dec. 2, 2005, which application is hereby incorporated by reference in its entirety.

### GOVERNMENT SUPPORT

This invention was made with government support under Grant Numbers R01-DK069966 awarded by the National Institutes of Health. The government has certain rights in this invention.

### BACKGROUND

Cellular signal transduction is a fundamental mechanism whereby extracellular stimuli are relayed to the interior of cells and subsequently regulate diverse cellular processes. One of the key biochemical mechanisms of signal transduction involves the reversible phosphorylation of proteins. Phosphorylation of polypeptides regulates the activity of mature proteins by altering their structure and function. Phosphate most often resides on the hydroxyl moiety (—OH) of serine, threonine, or tyrosine amino acids in proteins. Enzymes that mediate phosphorylation of cellular effectors fall into two classes. While protein phosphatases hydrolyze phosphate moieties from phosphoryl protein substrates, protein kinases transfer a phosphate moiety from adenosine triphosphate to protein substrates. The converse functions of protein kinases and protein phosphatases balance and regulate the flow of signals in signal transduction processes.

Protein kinases and protein phosphatases are typically divided into two groups: receptor and non-receptor type proteins. Receptor protein kinases are comprised of an extracellular domain, a membrane spanning region, and a catalytic domain.

A class of non-receptor protein kinases are implicated in regulating RNA splicing (Fu, 1995 RNA 1:663-680; Staknis and Reed, 1994, Mol. Cell. Biol. 14:7670-7682). These protein kinases phosphorylate polypeptides rich in serine and arginine (SR proteins). SR proteins are characterized as containing at least one amino-terminal RNA recognition motif and a basic carboxyterminal domain rich in serine and arginine residues, often arranged in tandem repeats (Zahler et al., 1992, Genes Dev 6:837-847). Experimental evidence supports the idea that the SR domain is involved in protein-protein interactions (Kohtz et al., 1994, Nature 368:119-124) as well as protein-RNA interactions (Harada et al., 1996, Nature 380:175-179), and may contribute to a localization signal directing proteins to nuclear speckles (Hedley et al., 1995, Proc. Natl. Acad. Sci. USA 92:11524-11528).

The selection of splice site can be altered by numerous extracellular stimuli, including growth factors, cytokines, hormones, depolarization, osmotic shock, and UVC irradiation through synthesis, phosphorylation, and a change in localization of serine/arginine-rich (SR) proteins (Stamm (2002) Hum. Mol. Genet. 11: 2409).

SR proteins are a family of essential factors required for constitutive splicing of pre-mRNA (Krainer et al. (1991) Cell 66: 383) and play an important role in modulating alternative splicing (Blencowe (2000) Trends Biochem. Sci. 25: 106). They are highly conserved in eukaryotes and are characterized by having one or two RNA-recognition motifs at the amino terminus and an RS domain at the carboxyl terminus (Zahler et al. (1992) Genes Dev. 6: 837; Caceres et al. (1993) EMBO J. 12: 4715). RS domains consist of multiple consecutive RS/SR dipeptide repeats and differ in length among different SR proteins. Extensive phosphorylation of serines in the RS domain occurs in all SR proteins (Kohtz, et al (1994) Nature 368: 119; Gui et al. (1994) Nature 369: 678). Although its precise physiological role is still unknown, phosphorylation of SR proteins affects their protein-protein and protein-RNA interactions (Xiao et al. (1997) Genes Dev. 11: 334), intracellular localization and trafficking (Caceres et al. (1998) Genes Dev. 12: 55; Misteli et al. (1998) J. Cell Biol. 143: 297), and alternative splicing of pre-mRNA (Duncan et al. (1997) Mol. Cell. Biol. 17: 5996). Spliceosome assembly may be promoted by phosphorylation of SR proteins that facilitate specific protein interactions, while preventing SR proteins from binding randomly to RNA (Xiao et al. (1997) Genes Dev. 11: 334). Once a functional spliceosome has formed, dephosphorylation of SR proteins appears to be necessary to allow the transesterification reactions to occur (Cao et al. (1997) RNA (New York) 3: 1456). Therefore, the sequential phosphorylation and dephosphorylation of SR proteins may mark the transition between stages in each round of the splicing reaction. To date, several kinases have been reported to phosphorylate SR proteins, including SRPK family kinases (Gui et al. (1994) Proc. Natl. Acad. Sci. U.S.A. 91: 10824; Kuroyanagi et al. (1998) Biochem. Biophys. Res. Commun. 242: 357-64), hPRP4 (Kojima et al. (2001) J. Biol. Chem. 276: 32247), and Topoisomerase 1 (Rossi et al. (1996) Nature 381: 80), and a family of kinases termed CLK (Cdc2-like kinase), or LAMMER kinases from the consensus motif, consisting of four members (CLK1/Sty and CLK2, CLK3 and CLK4) (Colwill et al. (1996) EMBO J. 15: 265; Nayler et al. (1997) Biochem. J. 326: 693).

Mammalian CLK family kinases contain an SR domain and are demonstrated to phosphorylate SR proteins in vitro and SF2/ASF in vivo (Nayler et al. (1997) Biochem. J. 326: 693). Clks are shown to be dual-specificity kinases that autophosphorylate on tyrosine, serine, and threonine residues in overexpression systems and in vitro (Nayler et al. (1997) Biochem. J. 326: 693; Ben-David et al. (1991) EMBO J. 10: 317; Howell et al. (1991) Mol. Cell. Biol. 11: 568). When overexpressed, the catalytically inactive mutant kinases localize to nuclear speckles where splicing factors are concentrated, whereas the wild-type enzymes distribute throughout the nucleus and cause speckles to dissolve (Colwill et al. (1996) EMBO J. 15: 265). The overexpression of CLKs also affects splicing site selection of pre-mRNA of both its own transcript and adenovirus E1A transcripts in vivo (Duncan et al. (1997) Mol. Cell. Biol. 17: 5996).

CLK's are well conserved in many organisms. mCLK1 is a dual specificity protein kinase originally isolated in mouse expression libraries (Ben-David et al., 1991, EMBO J. 10:317-325; Howell et al., 1991, Mol. Cell. Biol. 11:568-572) and human (hCLK1, hCLK2, hCLK3, hCLK4), plant (AFC1, AFC2, AFC3) and fly (DOA) CLK protein kinases have since been identified (Johnson and Smith, 1991, J. Biol. Chem. 266:3402-3407; Hanes et al., 1994, J. Mol. Biol. 244:665-672; Bender and Fink, 1994, Proc. Natl. Acad. Sci. USA 91:12105-12109; Yun et al., 1994, Genes. Dev. 8:1160-1173). Three of the genes for human CLKs have been mapped to unique chromosomal locations; specifically hCLK1-2q33, hCLK2-1q21 and hCLK3-15q24 (Talmadge et al., Hum Genet. 1998 103 (4):523-4). The amino terminal domain of these proteins is rich in serine and arginine, whereas the catalytic domain can be most similar to CDC2, a serine/threonine protein kinase (Ben-David et al., 1991, EMBO J. 10:317-325). CLKs are also known as STY or LAMMER kinases (the latter based on a signature motif ‘EHLAMMERILG’ conserved between the CLK family members).

U.S. Pat. No. 6,797,513 (“Nucleic acid encoding CLK2 protein kinases”) describes nucleic acid molecules encoding mCLK2, mCLK3, and mCLK4 polypeptides, nucleic acid molecules-encoding portions of their amino acid sequences, nucleic acid vectors harboring such nucleic acid molecules, cells containing such nucleic acid vectors, purified polypeptides encoded by such nucleic acid molecules, and antibodies to such polypeptides. Also included are assays that contain at least one CLK protein kinase related molecule. Diagnosis and treatment of an abnormal condition related to RNA splicing or cell proliferation in an organism by using a CLK protein kinase related molecule or compound are disclosed. A method of using a CLK protein kinase related molecule or compound as a contraceptive to reproduction in male organisms is also disclosed.

Both mCLK1 and the Drosophila homologue, DOA (Dead On Arrival), regulate RNA splicing events. Each of these have two alternatively spliced products coding for either the full-length catalytically active protein or a truncated protein lacking the catalytic domain (Yun et al., 1994, Genes. Dev. 8:1160-1173; Duncan et al., 1995, J. Biol. Chem. 270:21524-21531). Identical splice forms were also found in human CLK protein kinases (Hanes et al., 1994, J. Mol. Biol. 244:665-672). The ratio of these splice products appears to be developmentally regulated in Drosophila (Yun et al., 1994, Genes. Dev. 8:1160-1173), and in a tissue and cell type specific manner in mammals (Hanes et al., 1994, J. Mol. Biol. 244:665-672; Duncan et al., 1995, J. Biol. Chem. 270:21524-21531). In addition, the expression of several other, larger transcripts, are observed to be differentially regulated and are shown to represent partially spliced products (Duncan et al., 1995, J. Biol. Chem. 270:21524-21531).

To date, a number of diseases caused by mis-splicing have been reported; in some cases, mutation(s) found around splice sites appear to be responsible for changing the splicing pattern of a transcript by unusual exon inclusion or exclusion and/or alteration of 5′ or 3′ sites (reviewed in Stoss et al. (2000) Gene Ther. Mol. Biol. 5: 9; Philips et al. (2000) Cell. Mol. Life. Sci. 57: 235; Faustino et al. (2003) Genes Dev. 17: 419). A typical example is beta-thalassemia, an autosomal recessive disease, which is often associated with mutations in intron 2 of the alpha-globin gene. The generation of aberrant 5′ splice sites activates a common 3′ cryptic site upstream of the mutations and induces inclusion of a fragment of the intron-containing stop codon. As a result, the amount of functional alpha-globin protein is reduced. For therapeutic modulation of alternative splicing, several trials with antisense oligonucleotide (Sazani et al. (2003) J. Clin. Investig. 112: 481), peptide nucleic acid oligonucleotide, and RNAi (Epstein (1998) Methods 14: 21; Celotto et al. (2002) RNA (New York) 8: 718) have been reported. These approaches could be useful for manipulating a specific splice site selection of a known target sequence like beta-globin (Sazani et al. (2003) J. Clin. Investig. 112: 481). However, the aberrant splicing, found in the patients of breast cancer, Wilm's tumor, and amyotrophic lateral sclerosis (ALS), are not always accompanied with mutations around splice sites. In sporadic ALS patients, EAAT2 (excitatory amino acid transporters 2) RNA processing is often aberrant in motor cortex and in spinal cord, the regions specifically affected by the disease. As exon 9 is aberrantly skipped in some ALS patients without any mutation in the gene (Lin et al. (1998) Neuron 20: 589), the disorders could be attributed to abnormalities in regulatory factors of splicing. Actually the balance of alternative splicing products can be affected by changes in the ratio of heterogeneous nuclear ribonucleoprotein and SR proteins (Mayeda et al. (1992) Cell 68: 365; Caceres et al. (1994) Science 265: 1706) and in the phosphorylation state and localization of SR proteins (Duncan et al. (1997) Mol. Cell. Biol. 17: 5996).

U.S. patent publication 2005/0171026 (“Therapeutic composition of treating abnormal splicing caused by the excessive kinase induction”), provides a composition for treating or preventing abnormal splicing caused by the excessive kinase induction, which comprises compounds and a method for using the compounds for treating or preventing abnormal splicing caused by the excessive kinase induction. The compositions and methods so described would be useful for treatment of diseases that have as a cause excessive kinase activity leading to abnormal splicing, including some forms of cancer and neurodegeneration as described within the application.

Surpisingly, it has been discovered that in addition to the role CLKs play in splicing, CLKs directly phosphorylate proteins involved in, among other things, gene transcription; deacetylation of proteins that have been post-translationally modified by acetylation of specific lysine residues; and mitochondrial function, biogenesis, and/or activity. Specifically CLKs have been shown to phosphorylate sirtuins and PGC-1 alpha thereby modulating pathways involved in gene transcription and mitochondrial function, biogenesis, and/or activity. In this way, modulators of CLK activity have been shown to modulate these cellular processes and would therefore be useful in treating numerous diseases and disorders, as specified in the instant application.

### SUMMARY

In one aspect, the invention provides methods for using CLK-modulating compounds, or compostions comprising CLK-modulating compounds.

In certain embodiments, CLK-inhibiting compounds may be used for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, and/or flushing, etc. CLK-inhibiting compounds may also be used for treating a disease or disorder in a subject that would benefit from increased mitochondrial activity, for enhancing muscle performance, for increasing muscle ATP levels, or for treating or preventing muscle tissue damage associated with hypoxia or ischemia. In exemplary embodiments, the methods may comprise administering a CLK-inhibiting compound in combination with at least one other therapeutic agent, including, for example, a sirtuin-activating compound.

In other embodiments, CLK-activating compounds may be used for a variety of therapeutic applications including, for example, increasing cellular sensitivity to stress, increasing apoptosis, treatment of cancer, stimulation of appetite, and/or stimulation of weight gain, etc. In exemplary embodiments, the methods may comprise administering a CLK-activating compound in combination with at least one other therapeutic agent, including, for example, a sirtuin-inhibiting compound.

As described further below, the methods comprise administering to a subject in need thereof a pharmaceutically effective amount of a CLK-modulating compound.

In one aspect, the invention provides a method for promoting survival of a eukaryotic cell comprising contacting the cell with at least one CLK-inhibiting compound, or a pharmaceutically acceptable salt or prodrug thereof. The CLK-inhibiting compound may increase the lifespan of the cell. The CLK-inhibiting compound may increase the cell's ability to resist stress, such as, for example, stress due to heatshock, osmotic stress, DNA damage, inadequate salt level, inadequate nitrogen level, or inadequate nutrient level. The CLK-inhibiting compound may mimic the effect of nutrient restriction on the cell. In an exemplary embodiment, the eukaryotic cell is a mammalian cell.

In another aspect, the invention provides a method for treating or preventing a disease or disorder associated with cell death or aging in a subject, comprising administering to a subject in need thereof a therapeutically effective amount of at least one CLK-inhibiting compound, or a pharmaceutically acceptable salt or prodrug thereof. The aging-related disease may be, for example, stroke, a cardiovascular disease, arthritis, high blood pressure, or Alzheimer's disease.

In another aspect, the invention provides a method for treating or preventing insulin resistance, a metabolic syndrome, diabetes, or complications thereof, or for increasing insulin sensitivity in a subject, comprising administering to a subject in need thereof a therapeutically effective amount of at least one CLK-inhibiting compound, or a pharmaceutically acceptable salt or prodrug thereof.

In another aspect, the invention provides a method for reducing the weight of a subject, or preventing weight gain in a subject, comprising administering to a subject in need thereof a therapeutically effective amount of at least one CLK-inhibiting compound, or a pharmaceutically acceptable salt or prodrug thereof. In an exemplary embodiment, the subject does not reduce calorie consumption, increase activity or a combination thereof to an extent sufficient to cause weight loss in the absence of a CLK-inhibiting compound.

In another aspect, the invention provides a method for preventing the differentiation of a pre-adipocyte, comprising contacting the pre-adipocyte with at least one CLK-inhibiting compound, or a pharmaceutically acceptable salt or prodrug thereof.

In another aspect, the invention provides a method for prolonging the lifespan of a subject comprising administering to a subject a therapeutically effective amount of at least one CLK-inhibiting compound, or a pharmaceutically acceptable salt or prodrug thereof.

In another aspect, the invention provides a method for treating or preventing a neurodegenerative disorder in a subject, comprising administering to a subject in need thereof a therapeutically effective amount of at least one CLK-inhibiting compound, or a pharmaceutically acceptable salt or prodrug thereof. The neurodegenerative disorder may be, for example, Alzheimer's disease (AD), Parkinson's disease (PD), Huntington disease (HD), amyotrophic lateral sclerosis (ALS; Lou Gehrig's disease), diffuse Lewy body disease, chorea-acanthocytosis, primary lateral sclerosis, Multiple Sclerosis (MS) and Friedreich's ataxia.

In another aspect, the invention provides a method for treating or preventing a blood coagulation disorder in a subject, comprising administering to a subject in need thereof a therapeutically effective amount of at least one CLK-inhibiting compound, or a pharmaceutically acceptable salt or prodrug thereof. The blood coagulation disorder may be, for example, thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, disseminated intravascular coagulation, thrombotic thrombocytopenic purpura, cancer metastasis, sickle cell disease, glomerular nephritis, drug induced thrombocytopenia, and re-occlusion during or after therapeutic clot lysis or procedures such as angioplasty or surgery.

In another aspect, the invention provides a method for treating or preventing an ocular disease or disorder, comprising administering to a subject in need thereof a therapeutically effective amount of at least one CLK-inhibiting compound, or a pharmaceutically acceptable salt or prodrug thereof. An ocular disease or disorder may be, for example, vision impairment, glaucoma, optic neuritis, macular degeneration, or anterior ischemic optic neuropathy. The vision impairment may be cause, for example, by damage to the optic nerve or central nervous system (such as, for example, by high intraocular pressure, swelling of the optic nerve, or ischemia) or by retinal damage (such as, for example, by disturbances in blood flow to the retina or disruption of the macula).

In another aspect, the invention provides a method for treating or preventing chemotherapeutic induced neuropathy comprising administering to a subject in need thereof a therapeutically effective amount of at least one CLK-inhibiting compound, or a pharmaceutically acceptable salt or prodrug thereof. In an exemplary embodiment, the chemotherapeutic comprises a vinka alkaloid or cisplatin.

In another aspect, the invention provides a method for treating or preventing neuropathy associated with an ischemic event or disease comprising administering to a subject in need thereof a therapeutically effective amount of at least one CLK-inhibiting compound, or a pharmaceutically acceptable salt or prodrug thereof. The ischemic event may be, for example, a stroke, coronary heart disease (including congestive heart failure or myocardial infarction), stroke, emphysema, hemorrhagic shock, arrhythmia (e.g. atrial fibrillation), peripheral vascular disease, or transplant related injuries.

In another aspect, the invention provides a method for treating or preventing a polyglutamine disease comprising administering to a subject in need thereof a therapeutically effective amount of at least one CLK-inhibiting compound, or a pharmaceutically acceptable salt or prodrug thereof. The polyglutamine disease may be, for example, spinobulbar muscular atrophy (Kennedy disease), Huntington's disease, dentatorubralpallidoluysian atrophy (Haw River syndrome), spinocerebellar ataxia type 1, spinocerebellar ataxia type 2, spinocerebellar ataxia type 3 (Machado-Joseph disease), spinocerebellar ataxia type 6, spinocerebellar ataxia type 7, or spinocerebellar ataxia type 17. In certain embodiments, the method for treating or preventing a polyglutamine disease further comprises administering a therapeutically effective amount of an HDAC I/II inhibitor.

In another aspect, the invention provides a method for treating a disease or disorder in a subject that would benefit from increased mitochondrial activity, comprising administering to a subject in need thereof a therapeutically effective amount of at least one CLK-inhibiting compound, or a pharmaceutically acceptable salt or prodrug thereof. In certain embodiments, the method may further comprise administering to the subject one or more of the following: a vitamin, cofactor or antioxidant, including, for example, coenzyme Q₁₀, L-carnitine, thiamine, riboflavin, niacinamide, folate, vitamin E, selenium, lipoic acid, or prednisone. In certain embodiments, the method may further comprise administering to the subject one or more agents that alleviate a symptom of the disease or disorder, such as, for example, an agent that alleviates seizures, neuropathic pain or cardiac dysfunction. In certain embodiments, the disorder is associated with administration of a pharmaceutical agent that decreases mitochondrial activity, such as, for example, a reverse transcriptase inhibitor, a protease inhibitor, or an inhibitor or dihydroorotate dehydrogenase (DHOD).

In another aspect, the invention provides a method for enhancing motor performance or muscle endurance, decreasing fatigue, or increasing recovery from fatigue, comprising administering to a subject in need thereof a therapeutically effective amount of at least one CLK-inhibiting compound, or a pharmaceutically acceptable salt or prodrug thereof. In certain embodiments, the subject may be an athlete. Fatigue may be associated, for example, with administration of a chemotherapeutic.

In another aspect, the invention provides a method for treating or preventing a condition wherein motor performance or muscle endurance is reduced, comprising administering to a subject in need thereof a therapeutically effective amount of at least one CLK-inhibiting compound, or a pharmaceutically acceptable salt or prodrug thereof. The condition may be, for example, a muscle dystrophy, a neuromuscular disorder, McArdle's disease, myasthenia gravis, a muscle injury, multiple sclerosis, amyotrophic lateral sclerosis, or age-related sarcopenia.

In another aspect, the invention provides a method for treating or preventing muscle tissue damage associated with hypoxia or ischemia, comprising administering to a subject in need thereof a therapeutically effective amount of at least one CLK-inhibiting compound, or a pharmaceutically acceptable salt or prodrug thereof.

In another aspect, the invention provides a method for increasing muscle ATP levels in a subject, comprising administering to a subject in need thereof a therapeutically effective amount of at least one CLK-inhibiting compound, or a pharmaceutically acceptable salt or prodrug thereof.

In certain embodiments, the methods described herein do not involve treating or preventing a disease or disorder associated with alternate, abnormal, aberrant or undesired splicing.

In certain embodiments, the methods described herein do not involve treating or preventing one or more of the following diseases or disorders: beta-thalassemia, FTDP-17, NF2, FRASIER, Wilms tumor, breast cancer, ovarian cancer, renal cancer, lung cancer, urothellal cancer, gastric cancer, papillary thyroid cancer, HNSCC, invasive breast cancer, glant cell tumors of bone, prostate cancer, melanoma, lymphoma, oral cancer, pharyngeal cancer, progeria, neurodegenerative diseases such as Alzheimer's disease, Parkinson's disease, amyotrophic lateral sclerosis (ALS), Huntington disease, spinocerebellar ataxia, spinal and bulbar muscular atrophy (SBMA) and epilepsy, progressive supranuclear palsy, and Pick's disease.

In certain embodiments, the methods described herein comprise administering to a subject at least one CLK-inhibiting compound and at least one sirtuin-activating compound. Examples of sirtuin-activating compounds, include, for example, resveratrol, butein, fisetin, piceatannol, quercetin, and nicotinamide riboside. An exemplary CLK-inhibiting compound is TG003. Other examples of CLK-inhibiting compounds include, for example, an siRNA, an antisense oligonucleotide, a ribozyme, an aptamer, or an antibody

In certain embodiments, the CLK-inhibiting compound decreases CLK associated phosphorylation of a sirtuin protein and/or PGC-1 alpha.

In certain embodiments, the CLK-inhibiting compound is an inhibitor of at least one human CLK protein, such as, one or more of hCLK1, hCLK2, hCLK3, and/or hCLK4.

In another aspect, the invention provides a method for treating or preventing cancer in a subject, comprising administering to a subject in need thereof (i) a therapeutically effective amount of at least one CLK-activating compound, or a pharmaceutically acceptable salt or prodrug thereof, or (ii) a polynucleotide that promotes overexpression of a CLK protein. The method may further comprise administering to the subject a chemotherapeutic agent.

In another aspect, the invention provides a method for stimulating weight gain in a subject, comprising administering to a subject in need thereof (i) a therapeutically effective amount of at least one CLK-activating compound, or a pharmaceutically acceptable salt or prodrug thereof, or (ii) a polynucleotide that promotes overexpression of a CLK protein.

In another aspect, the invention provides a method for increasing the radiosensitivty or chemosensitivity of a cell comprising (i) contacting the cell with at least one CLK-activating compound, or a pharmaceutically acceptable salt or prodrug thereof, or (ii) introducing into the cell a polynucleotide that promotes overexpression of a CLK protein. The call may be, for example, a mammalian cell.

In certain embodiments, the methods described herein a CLK-activating compound promotes CLK associated phosphorylation of a sirtuin protein and/or PGC-1 alpha.

In certain embodiments, a CLK-activating compound is a polynucleotide such as, for example, an expression vector comprising a nucleic acid sequence encoding a CLK protein (such as, for example a mammalian CLK or human CLK) or a biologically active fragment thereof. Examples of human CLKs include hCLK1, hCLK2, hCLK3, and hCLK4.

In certain embodiments, the CLK-activating compound is an activator of at least one human CLK protein, such as, hCLK1, hCLK2, hCLK3, and/or hCLK4.

In certain embodiments, the methods described herein comprise administering to a subject at least one CLK-activating compound and at least one sirtuin-inhibiting compound. Examples of sirtuin-inhibiting compounds include, for example, nicotinamide, sirtinol, and splitomicin.

In another aspect, the invention provides a composition comprising at least one CLK-inhibiting compound and at least one sirtuin-activating compound. In an exemplary embodiment, the CLK-inhibiting compound is TG003. Examples of sirtuin-activating compounds include, for example, resveratrol, butein, fisetin, piceatannol, quercetin, and nicotinamide riboside.

In another aspect, the invention provides a composition comprising at least one CLK-activating compound and at least one sirtuin-inhibiting compound. Examples of sirtuin-inhibiting compounds include, for example, nicotinamide, sirtinol, and splitomicin.

In another aspect, the invention provides use of a CLK-inhibiting compound for the preparation of a medicament for increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, flushing, treating a disease or disorder in a subject that would benefit from increased mitochondrial activity, for enhancing muscle performance, for increasing muscle ATP levels, or for treating or preventing muscle tissue damage associated with hypoxia or ischemia.

In another aspect, the invention provides use of a CLK-activating compound for preparation of a medicament for increasing cellular sensitivity to stress, increasing apoptosis, treatment of cancer, stimulation of appetite, and/or stimulation of weight gain, etc.

In another aspect, the invention provides a CLK-inhibiting compound for use in increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, flushing, treating a disease or disorder in a subject that would benefit from increased mitochondrial activity, for enhancing muscle performance, for increasing muscle ATP levels, or for treating or preventing muscle tissue damage associated with hypoxia or ischemia.

In another aspect, the invention provides a CLK-activating compound for use in increasing cellular sensitivity to stress, increasing apoptosis, treatment of cancer, stimulation of appetite, and/or stimulation of weight gain, etc.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Sirt1 phosphorylation sites confirmed by mass spectroscopy. Conservation of phosphorylation sites between human, mouse, rat, C. elegans, and/or chicken as indicated were determined by alignment of Sirt1/Sir2 protein sequences. Phosphorylation of one or several serines (S164, S165, S166) is of particular interest because of their conservation between species. These sites were identified as a potential CLK2 kinase target by Scansite <http://scansite.mit.edu/>. The S164-166 phosphorylation sites illustrated in FIG. 1 correspond to the sequence of the Mouse SIRT1 protein (SEQ ID NO: 12). The equivalent residues in human SIRT1 are found at S172, S173 and S174 (SEQ ID NO: 10). Equivalent residues in other sirtuin proteins may be determined by one of skill in the art by aligning the sirtuin proteins using publicly available databases (see also, R. A. Frye, Biochem. Biophys. Res. Comm. 273: 793-7989 (2000)).

FIG. 2 demonstrates that Sirt1 is a target of CLK2 kinase. FIG. 2A shows that overexpression of CLK2 causes a shift in Sirt1 mobility. HEK 293 cells were transfected with empty vector (pcDNA) or an overexpression Flag-CLK2 construct. Overexpression of CLK2 caused a marked shift in Sirt1 mobility as determined by SDS-PAGE and western blot using anti-Sirt1 antibody (Upstate). FIG. 2B shows that CLK2 phosphorylates SIRT1 as determined by metabolic labeling. HEK 293 cells were transfected with either a dual tagged Flag-HA-Sirt1 wild type construct or a construct that contains three alanines substituted for serine at sites 164, 165, and 166 (Flag-HA-Sirt1 S164-6A). Flag-HA-Sirt1 WT showed incorporation of ³²phosphate that was noticeably decreased upon treatment with 40 uM TG003 (CLK kinase inhibitor). Overexpression of CLK2 caused a shift in Sirt1 mobility that was abrogated by treatment with 40 uM TG003. Overexpression of kinase dead CLK2 mutant (K192R) did not cause a shift in Sirt1 mobility. Flag-HA-Sirt1 S164-6A shows basal phosphorylation however CLK2 overexpression still causes a shift in mobility but total phosphorylation is dramatically lower, possibly indicating that S164, S165, and S166 may not be the only CLK2 phosphorylation sites on mouse Sirt1. Mouse SIRT1 (SEQ ID NO: 12), mouse CLK2, mouse PGC-1alpha (GenBank Accession Nos. AAH66868 or O70343), and mouse HNF4alpha were used for the experiments described in FIGS. 2-4, 6-9 and 11.

FIG. 3 demonstrates that CLK inhibition decreases total phosphorylation of PGC-1alpha and Sirt1 in hepatocytes. FAO cells, rat hepatoma hepatocytes, were infected with adenoviruses overexpression Flag-HA-PGC-1 alpha or Flag-Sirt1 and incubated with or without 40 μM TG003. As determined by incorporation of ³²phosphate relative to protein levels, total phosphorylation of PCG-1α and Sirt1 is decreased in cell treated with TG003.

FIG. 4 shows that CLK2 interacts with Sirt1 and PGC-1alpha. FIG. 4A shows that CLK2 interacts with SIRT1. HEK 293 cells were transfected with Flag-CLK2 and treated as indicated. Cells were harvested and subject to immunoprecipitation with M2 anti-Flag agarose (Sigma). Endogenous Sirt1 was able to co-immunoprecipitate with CLK2. Interestingly upon treatment with TG003 the co-immunoprecipitated Sirt1 shifted to a band with faster mobility. FIG. 4B shows that CLK2 interacts with Sirt1 and PGC-1alpha in hepatocytes. FAO hepatocytes were infected as indicated, harvested and subject to immunoprecipitation using anti-HA agarose (Roche). Immunoprecipitation of overexpressed Flag-HA-Sirt1 and Flag-HA-PGC-1alpha was able to co-immunoprecipitate CLK2.

FIG. 5 provides schematics and sequence alignments of the Cdc-2 Like Kinase (Clk) family of kinases. FIG. 5A provides a representation of mammalian CLK kinases 1-4 conserved CLK/LAMMER kinase domain and highly variable N-termini. The sensitivity of each CLK family to the CLK inhibitor TG003 is indicated to the right (see Muraki, M. et al., J. Biol. Chem. (2004) 279 (23):24246-54). Potential phosphorylation sites AKT/pKB or pKA on Mouse CLKs (with P<0.01) were determined using Scansite (see world wide web at scansite.mit.edu). FIG. 5B shows an alignment of human CLK amino acid sequences. seqCLK1, seqCLK2, seqCLK3 and seqCLK4 correspond to SEQ ID NO: 1, SEQ ID NO: 2, SEQ ID NO: 3 and SEQ ID NO: 4, respectively.

FIG. 6 shows that CLK2 represses PGC-1alpha coactivation of nuclear receptors. HEK 293 cells were transfected with the gAF1-luciferase (HNF4alpha response element) and HNF4alpha (hepatocyte nuclear factor 4 alpha). Overexpression of PGC-1alpha shows an ˜1000-fold activation in luciferase activity which is markedly decreased by overexpression of CLK2. Treatment of the cells with 20 μM TG003 partially rescues the CLK2 repression of PGC-L alpha. Similar repression by CLK2 of PGC-1 alpha coactivation of nuclear receptors was also seen with PPAR-alpha (Peroxisome Proliferator-Activated Receptor alpha), ERR-alpha (Estrogen-related Receptor alpha), and Glucocorticoid receptor (data not shown).

FIG. 7 shows that CLK inhibitor TG003 induces gluconeogenic genes in hepatocytes. FAO hepatocytes were infected with adenovirus overexpressing PGC-1alpha and treated with increasing amounts of TG003. Induction of the gluconeogenic genes (PGC-1alpha targets), Pepck (phosphoenolpyruvate carboxykinase) and G6Pase (glucose-6-phosphatase), were seen in a dose dependent manner with increasing TG003. The fatty acid oxidation gene, MCAD (medium-chain acyl-CoA dehydrogenase), another PCG-1alpha target also showed similar induction.

FIG. 8 shows that CLK2 siRNA significantly reduces CLK2 expression in HEK 293 cells. Adenovirus constructs comprising Flag-CLK2 and/or CLK2 siRNA were introduced into HEK 293 cells. Lane 2 (Ad-Flag-CLK2) shows that Flag-CLK2 is overexpressed in HEK 293 cells upon introduction of the Flag-Clk2 adenovirus construct. An adenovirus construct designed to express a short 21 nucleotide hairpin siRNA corresponding to mouse, rat, and human CLK2 was capable of largely reducing the protein expression of the adenovirus CLK2 overexpression (lane 4; Ad-Flag-CLK2+Ad-CLK2 siRNA) compared to an adenovirus control siRNA containing a single basepair mutation of the CLK2 sequence in HEK 293 cells (lane 3; Ad-Flag-CLK2+Ad-Cntrl SiRNA).

FIG. 9 shows CLK2 repression of PGC-1alpha induction of gluconeogenic genes and rescue by the CLK2 inhibitor TG003. FIG. 9A shows that CLK2 represses PGC-1 alpha induction of gluconeogenic genes. FAO hepatocytes were infected with the indicated adenovirusese (Ad-GFP, Ad-PGC-1a+Ad-GFP, and Ad-PGC-1a+Ad-CLK2) overnight in RPMI+0.5% BSA. 48 hours after infection cells were either not treated (RPMI+BSA), treated with Forskolin and Dexamethasome (Fsk+Dex) for 3.5 hours or treated with Forskolin and Dex for 2 hours followed by Insulin (Fsk+Dex then Ins) for 1.5 hours. The top panel shows a Northern blot from total RNA isolation. The bottom panel is a quantitation of G6 Pase and Pepck expression corrected by 36b4 performed by phospho-imager analysis. FIG. 9B shows that CLK2 repression of PGC-1alpha induction of gluconeogenic genes is rescued by the CLK inhibitor TG003. FAO Hepatocytes were infected with the indicated adenoviruses (Ad-PGC-1a+Ad-GFP and Ad-PGC-1a+Ad-CLK2) overnight in RPMI+0.5% BSA. 48 Hours after infection cells were incubated with or without 40 μM TG003 for 1 hour followed by treatment with Forskolin and Dexamethasome (Fsk+Dex) as indicated. The top panel shows a Northern blot from total RNA isolation. The bottom panel is a quantitation of G6Pase and Pepck expression corrected by 36b4 performed by phospho-imager analysis.

FIG. 10 is a schematic of CLK2 transcription structure and splicing. CLKs regulate alternate splicing of their own transcript. Inclusion of Exon 4 in CLK2 mRNA results in a full length, catalytically active CLK2 protein. Exclusion of Exon 4 results in a truncated, catalytically inactive peptide. Active CLKs promote ‘exon skipping’ resulting in exon 4 exclusion (see e.g., Duncan et al. Mol. Cell. Biol. 17 (10): 5996).

FIG. 11 shows that CLK2 is activated by insulin. FIG. 11A (top) provides the results of a CLK2 transcription assay. Primers flanking exon 4 will produce a 195 basepair product if exon 4 is included in the CLK transcript or a 110 basepair product if exon 4 is excluded. FIG. 11A (bottom) shows an RT-PCR of CLK2 transcripts from FAO cells treated with or without insulin either alone (NT=no treatment) or in the presence of 20 μM of TG003, LY94002 (LY) PI3 Kinase inhibitor, U0126 MEK kinase inhibitor, or Rapammycin mTOR inhibitor. Full length CLK2 transcript is the primary transcript product in FAO cells grown in RPMI+BSA, however, following treatment with insulin, the truncated CLK2 transcript dramatically increases in abundance. Pretreatment of the cells with TG003 blocks the insulin induction of alternate splicing, indicating that CLK kinase activity is required for induction of alternate splicing. Additionally, treatment with LY and Rapamycin but not U0126 blocks the insulin induction of CLK alternate splicing, hinting that CLK2 is in the insulin pathway. This regulation of CLK2 splicing appears to be specific as CLK1 does not show splicing regulation. FIG. 11B shows that CLK2 phosphorylation on AKT consensus sites is induced by insulin treatment. Mouse H2.35 SV40 transformed hepatocytes were infected with Flag-CLK2 adenovirus and treated with or without insulin. CLK2 was immunoprecipitated using anti-flag agarose and subjected to western blotting using anti-phospho-Akt substrate antibodies (Cell Signaling). CLK2 phosphorylation was dramatically induced on Akt consensus sites upon treatment with insulin. Phospho-Akt Substrate antibodies (Cell Signaling) recognizes the epitope RXRXX(S/T)* (where x indicates any amino acid and * denotes phosphor serine or threonine). CLK2 possesses at least three possible recognition sites for this antibody including S34, S125, and T127 (FIG. 5A).

FIG. 12 shows a schematic of a CLK2 assay

FIG. 13 provides structural information about CLK and CLK inhibitors.

FIG. 13A shows a representation of an hCLK1 crystal structure in complex with 10Z-2 hymenialdisine at 1.7 angstrom as reported in the pdb data base as 1Z57. FIG. 13B shows the structure of 10Z-2 hymenialdisine.

FIG. 14 provides examples of CLK inhibitors.

FIG. 15 is a schematic of the synthesis of TG003, a CLK inhibitor.

FIG. 16 shows the results of synthesized TG003 in a fat mobilization cell based assay.

FIG. 17 shows the following nucleotide and amino acid sequences: SEQ ID NO: 1 (GenBank Accession # P49759 and # AAH31549, CLK1 (CDC-like kinase 1), Human, 484 aa), SEQ ID NO: 2 (GenBank Accession # NP_{—}003984 and AAH53603, CLK2 (CDC-like kinase 2), human, 498 aa), SEQ ID NO: 3 (GenBank Accession # P49761 and AAH19881, CLK3 (CDC-like kinase 3), Human, 490 aa), SEQ ID NO: 4 (GenBank Accession # Q9HAZ1 or NP_{—}065717, CLK4 (CDC-like kinase 4), Human, 481 aa), SEQ ID NO: 5 (GenBank Accession # BC031549, CLK1 mRNA, human, 1773 bp), SEQ ID NO: 6 (GenBank Accession # BC053603, CLK2 mRNA, human, 2110 bp), SEQ ID NO: 7 (GenBank Accession # BC019881, CLK3 mRNA (CDC-like kinase 3), human, 1760 bp), SEQ ID NO: 8 (GenBank Accession # NM_{—}020666, CLK4 mRNA (CDC-like kinase 4), human, 2524 bp), SEQ ID NO: 9 (29 amino acid synthetic peptide of SF2/ASF RS domain), SEQ ID NO: 10 (Human SIRT1), SEQ ID NO: 11 (14 amino acid acetylated peptide derived from p53), SEQ ID NO: 12 (Mouse SIRT1), SEQ ID NO: 13 (a 20 amino acid acetylated and fluorescently tagged peptides derived from p53), SEQ ID NO: 14 (a 20 amino acid acetylated and fluorescently tagged peptides derived from p53), SEQ ID NO: 15 (an oligonucleotide corresponding to mouse, rat and human CLK2), and SEQ ID NO: 16 (a control siRNA).

FIG. 18. CLK2 Phosphorylation is Stimulated by Insulin.

FIG. 19. AKT Phosphorylates CLK2 in vitro.

FIGS. 20A and 20B. PGC-1alpha and SIRT1 Phosphorylation in vivo is stimulated by insulin and blocked by LY and TG003.

FIGS. 21A and 21B. CLK2 is involved in the induction of PEPCK mRNA expression (FIG. 21A) and CLK2 knock-down causes partial insulin resistance (FIG. 21B).

FIG. 22. Hepatic CLK2 knock-down causes partial insulin resistance in whole animals.

FIGS. 23A and 23B. Hepatic CLK2 knock-down affects serum and liver triglycerides (FIG. 23A) and hepatic CLK2 knock-down affects serum free fatty acids and glycemia (FIG. 23B).

FIG. 24. Hepatic CLK2 knock-down decreases liver lipids.

FIGS. 25A and 25B. Plasma levels of TG003 following oral (FIG. 25A) or IP (FIG. 25B) dosing at the indicated doses.

FIG. 26. Change in body weight of mice in various treatment groups with TG003 (100 mg/kg IP dosing) or vehicle in diet induced obesity (DIO) or chow animals.

FIGS. 27A, 27B and 27C. Blood insulin levels in mice following IP dosing at 100 mg/kg TG003 compared to vehicle in DIO or chow fed animals at 0 weeks (FIG. 27A), 2 weeks (FIG. 27B) or 4 weeks (FIG. 27C).

FIGS. 28A and 28B. Fed blood glucose levels in mice following IP dosing at 100 mg/kg TG003 compared to vehicle in DIO or chow fed animals at 0 weeks (FIG. 28A) or 2 weeks (FIG. 28B).

FIGS. 29A and 29B. Fasted blood glucose at 3 weeks (FIG. 29A) and IPGTT curves (FIG. 29B) following IP dosing at 100 mg/kg TG003 compared to vehicle in DIO or chow fed animals.

FIG. 30. Change in body weight of mice in various treatment groups with TG003 (30 mg/kg IP dosing) or vehicle in DIO or normal chow animals.

FIGS. 31A, 31B and 31C. Blood insulin levels in mice following IP dosing at 30 mg/kg TG003 compared to vehicle in DIO or chow fed animals at 0 weeks (FIG. 31A), 2 weeks (FIG. 31B) or 4 weeks (FIG. 31C).

FIGS. 32A, 32B and 32C. Fed blood glucose levels in mice following IP dosing at 30 mg/kg TG003 compared to vehicle in DIO or chow fed animals at 0 weeks (FIG. 32A), 2 weeks (FIG. 32B) or 4 weeks (FIG. 32C).

FIG. 33. Fasted blood glucose at 3 weeks following IP dosing at 30 mg/kg TG003 compared to vehicle in DIO or chow fed animals.

FIGS. 34A and 34B. Change in body weight of mice in various treatment groups with TG003 (100 mg/kg peroral (PO) dosing) or vehicle in DIO or chow animals (FIG. 34A). The change in body temperature of mice in various treatment groups with TG003 (100 mg/kg PO dosing) or vehicle in DIO or chow animals (FIG. 34B).

FIGS. 35A and 35B. Fed insulin levels at 2 weeks post dosing (FIG. 35A) and blood glucose levels at 4 weeks post dosing (FIG. 35B) of mice in various treatment groups with TG003 (100 mg/kg PO dosing) or vehicle in DIO or chow animals.

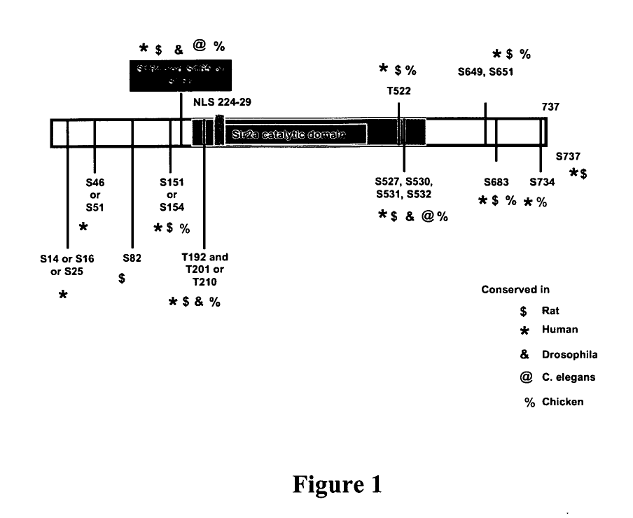

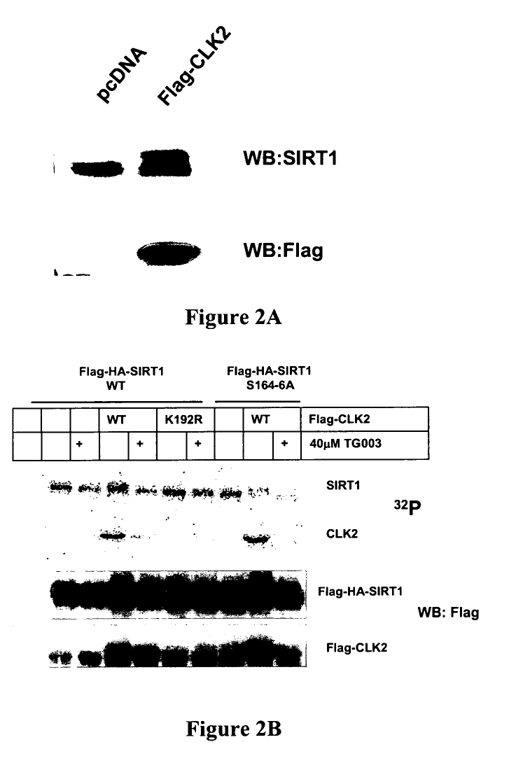

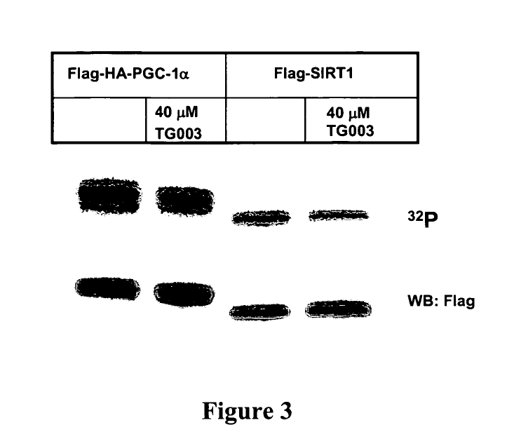

### DETAILED DESCRIPTION

1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The terms “CLK protein” or “CLK” refer to a member of the Cdc2-like kinase protein family. Exemplary members of the Cdc2-like kinase family include, for example, CLK proteins from human (hCLK1, hCLK2, hCLK3, and hCLK4), mouse (mCLK1, mCLK2, mCLK3, and mCLK4), plant (AFC1, AFC2, and AFC3) and fly (DOA) CLK protein kinases, as well as homologs (e.g., orthologs and paralogs), variants, or fragments thereof. In an exemplary embodiment, a CLK protein refers to hCLK1 (SEQ ID NO: 1), hCLK2 (SEQ ID NO: 2), hCLK3 (SEQ ID NO: 3), or hCLK4 (SEQ ID NO: 4). In other embodiments, a CLK protein refers to a polypeptide comprising a sequence consisting of, or consisting essentially of, the amino acid sequence set forth in SEQ ID NOs: 1, 2, 3 or 4; polypeptides comprising all or a portion of the amino acid sequence set forth in SEQ ID NOs: 1, 2, 3 or 4; the amino acid sequence set forth in SEQ ID NOs: 1, 2, 3 or 4 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to SEQ ID NOs: 1, 2, 3 or 4; and functional fragments of any of the foregoing. CLK proteins preferably have protein kinase activity. Fragments of the full length CLK proteins having kinase activity may be identified using techniques well known in the art, such as sequence comparisons and assays such as those described herein.

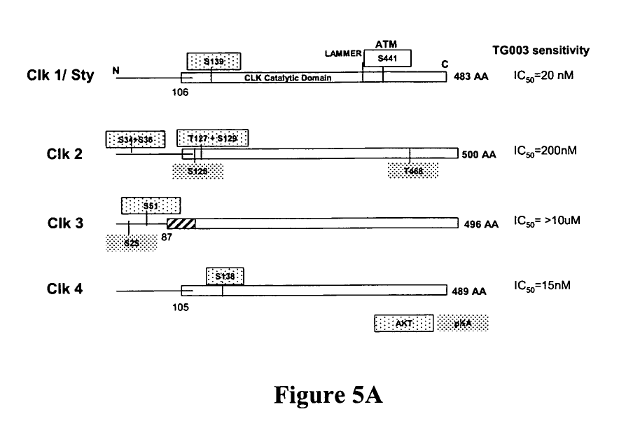

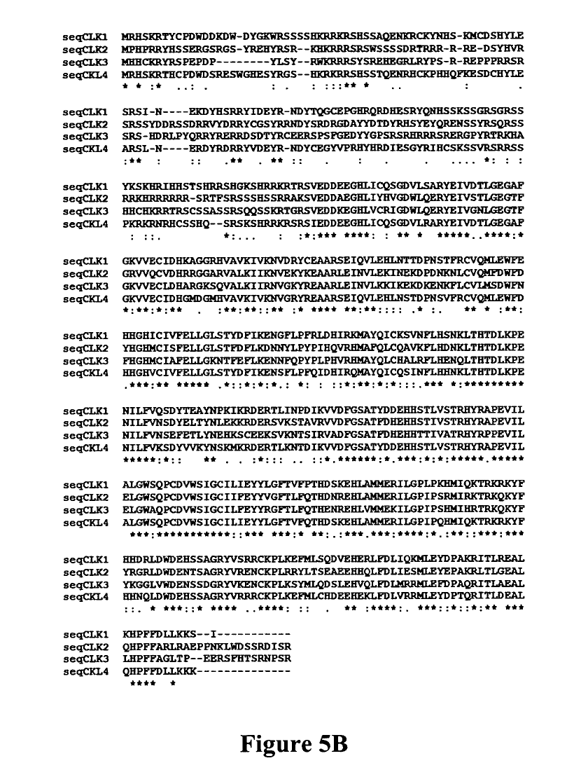

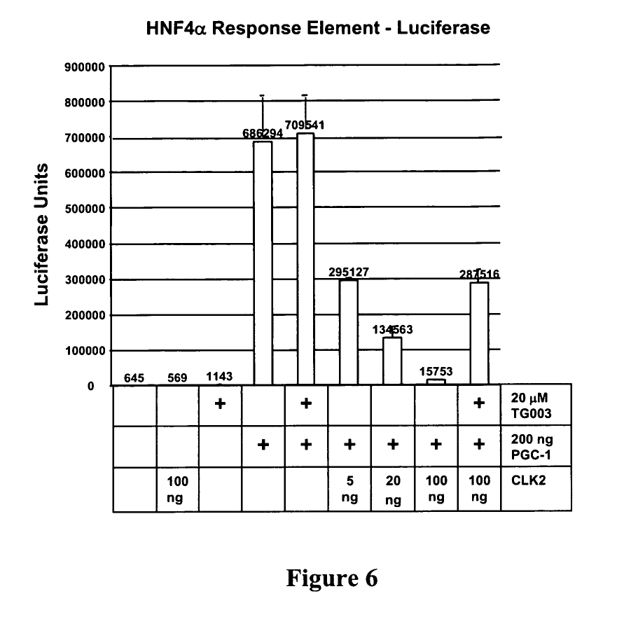

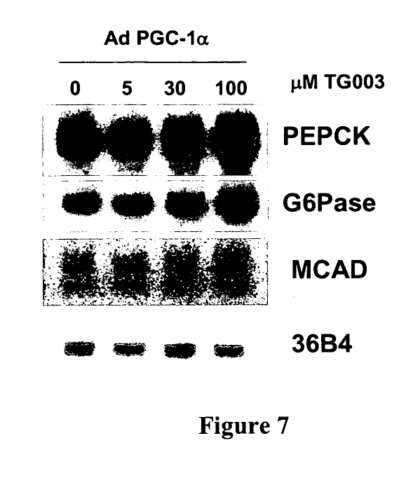

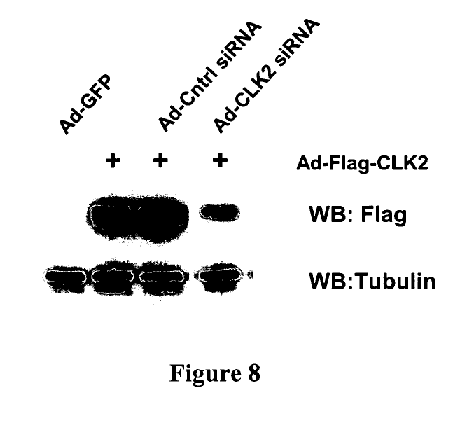

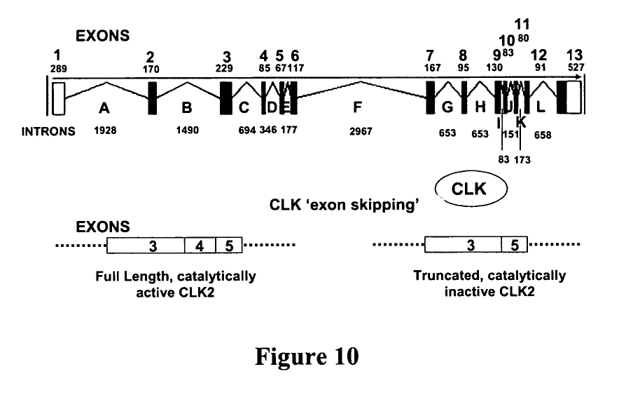

“Biologically active portion of a CLK” refers to a portion of a CLK protein having at least one biological activity of a CLK protein, such as kinase activity. Biologically active portions of CLKs may comprise the CLK catalytic domain (see e.g., FIG. 5A) or Exon 4 (see e.g., FIG. 10).

The term “companion animals” refers to cats and dogs. As used herein, the term “dog(s)” denotes any member of the species Canis familiaris, of which there are a large number of different breeds. The term “cat(s)” refers to a feline animal including domestic cats and other members of the family Felidae, genus Felis.

The terms “comprise” and “comprising” are used in the inclusive, open sense, meaning that additional elements may be included.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and H is, (ii) a positively-charged group, consisting of Lys, Arg and H is, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

“Diabetes” refers to high blood sugar or ketoacidosis, as well as chronic, general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

A “direct activator” of a polypeptide is a molecule that activates the polypeptide by binding to it.

A “direct inhibitor” of a polypeptide is a molecule that inhibits the polypeptide by binding to it.

The term “ED50” is art-recognized. In certain embodiments, ED50 means the dose of a drug which produces 50% of its maximum response or effect, or alternatively, the dose which produces a pre-determined response in 50% of test subjects or preparations. The term “LD50” is art-recognized. In certain embodiments, LD50 means the dose of a drug which is lethal in 50% of test subjects. The term “therapeutic index” is an art-recognized term which refers to the therapeutic index of a drug, defined as LD50/ED50.

The term “hyperinsulinemia” refers to a state in an individual in which the level of insulin in the blood is higher than normal.

The term “including” is used to mean “including but not limited to”. “Including” and “including but not limited to” are used interchangeably.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The term “livestock animals” refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus.

The term “mammal” is known in the art, and exemplary mammals include humans, primates, livestock animals (including bovines, porcines, etc.), companion animals (e.g., canines, felines, etc.) and rodents (e.g., mice and rats).

The term “naturally occurring form” when referring to a compound means a compound that is in a form, e.g., a composition, in which it can be found naturally. A compound is not in a form that is naturally occurring if, e.g., the compound has been purified and separated from at least some of the other molecules that are found with the compound in nature.

A “naturally occurring compound” refers to a compound that can be found in nature, i.e., a compound that has not been designed by man. A naturally occurring compound may have been made by man or by nature. A “non-naturally occurring compound” is a compound that is not known to exist in nature or that does not occur in nature.

“Obese” individuals or individuals suffering from obesity are generally individuals having a body mass index (BMI) of at least 25 or greater. Obesity may or may not be associated with insulin resistance.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject”, “individual” or “host” refers to either a human or a non-human animal.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, Vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. (See Meth. Mol. Biol. 70: 173-187, 1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “pyrogen-free”, with reference to a composition, refers to a composition that does not contain a pyrogen in an amount that would lead to an adverse effect (e.g., irritation, fever, inflammation, diarrhea, respiratory distress, endotoxic shock, etc.) in a subject to which the composition has been administered. For example, the term is meant to encompass compositions that are free of, or substantially free of, an endotoxin such as, for example, a lipopolysaccharide (LPS).

“Replicative lifespan” of a cell refers to the number of daughter cells produced by an individual “mother cell.” “Chronological aging” or “chronological lifespan,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress (for example, heatshock, osmotic stress, high energy radiation, chemically-induced stress, DNA damage, inadequate salt level, inadequate nitrogen level, or inadequate nutrient level). Lifespan can be increased by at least about 20%, 30%, 40%, 50%, 60% or between 20% and 70%, 30% and 60%, 40% and 60% or more using methods described herein.

“CLK-activating compound” refers to a compound that increases the level of a CLK protein and/or increases at least one activity of a CLK protein. In an exemplary embodiment, a CLK-activating compound may increase at least one biological activity of a CLK protein by at least about 10%, 25%, 50%, 75%, 100%, or more. Exemplary biological activities of CLK proteins include, for example, kinase activity, ability to phosphorylate a sirtuin protein, ability to phosphorylate a SIRT1 protein, ability to phosphorylate PGC-1 alpha, ability to phosphorylate a SR protein, ability to autophosphorylate, ability to phosphorylate a splicing factor, ability to regulate splicing, ability to bind to a sirtuin protein, ability to bind to a SIRT1 protein, or ability to bind to PGC-1alpha.

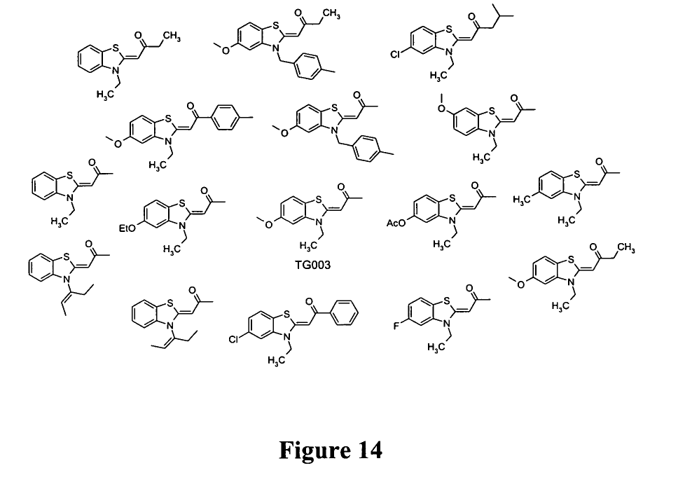

“CLK-inhibiting compound” refers to a compound that decreases the level of a CLK protein and/or decreases at least one activity of a CLK protein. Examples of CLK-inhibiting compounds are exemplified in US patent application 2005/0171026 (“Therapeutic composition of treating abnormal splicing caused by the excessive kinase induction”) or are illustrated in FIG. 14. In an exemplary embodiment, a CLK-inhibiting compound is TG003. In an exemplary embodiment, a CLK-inhibiting compound may decrease at least one biological activity of a CLK protein by at least about 10%, 25%, 50%, 75%, 100%, or more. Exemplary biological activities of CLK proteins include, for example, kinase activity, ability to phosphorylate a sirtuin protein, ability to phosphorylate a SIRT1 protein, ability to phosphorylate PGC-1 alpha, ability to phosphorylate a SR protein, ability to autophosphorylate, ability to phosphorylate a splicing factor, ability to regulate splicing, ability to bind to a sirtuin protein, ability to bind to a SIRT1 protein, or ability to bind to PGC-1alpha.

“CLK-modulating compound” refers to a compound that modulates the activity and/or level of a CLK protein. In exemplary embodiments, a CLK-modulating compound may either up regulate (e.g., activate or stimulate), down regulate (e.g., inhibit or suppress), or otherwise change a functional property or biological activity of a CLK protein. CLK-modulating compounds may act to modulate a CLK protein either directly or indirectly. In certain embodiments, a CLK-modulating compound may be a CLK-activating compound or a CLK-inhibiting compound.

“Sirtuin protein” refers to a member of the Sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 or NP_{—}036370 (or AF083106)), and human SIRT2 (GenBank Accession No. NM_{—}012237, NM_{—}030593, NP_{—}036369, NP_{—}085096, or AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, or P53685. SIRT1 proteins include polypeptides comprising all or a portion of the amino acid sequence set forth in GenBank Accession Nos. NP_{—}036370, NP_{—}501912, NP_{—}085096, NP_{—}036369, or P53685; the amino acid sequence set forth in GenBank Accession Nos. NP_{—}036370, NP_{—}501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, or P53685, and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, or P53685.

“SIRT3 protein” refers to a member of the sirtuin deacetylase protein family and/or to a homolog of a SIRT1 protein. In one embodiment, a SIRT3 protein includes human SIRT3 (GenBank Accession No. AAH01042, NP_{—}036371, or NP_{—}001017524) and mouse SIRT3 (GenBank Accession No. NP_{—}071878) proteins, and equivalents and fragments thereof. In another embodiment, a SIRT3 protein includes a polypeptide comprising a sequence consisting of, or consisting essentially of, the amino acid sequence set forth in GenBank Accession Nos. AAH01042, NP_{—}036371, NP_{—}001017524, or NP_{—}071878. SIRT3 proteins include polypeptides comprising all or a portion of the amino acid sequence set forth in GenBank Accession AAH01042, NP_{—}036371, NP_{—}001017524, or NP_{—}071878; the amino acid sequence set forth in GenBank Accession Nos. AAH01042, NP_{—}036371, NP_{—}001017524, or NP_{—}071878 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. AAH01042, NP_{—}036371, NP_{—}001017524, or NP_{—}071878, and functional fragments thereof. Polypeptides of the invention also include homologs (e.g., orthologs and paralogs), variants, or fragments, of GenBank Accession Nos. AAH01042, NP_{—}036371, NP_{—}001017524, or NP_{—}071878. In one embodiment, a SIRT3 protein includes a fragment of SIRT3 protein that is produced by cleavage with a mitochondrial matrix processing peptidase (MPP) and/or a mitochondrial intermediate peptidase (MIP). The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

“Sirtuin-activating compound” refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. Examples of sirtuin-activating compounds include, for example, resveratrol, butein, fisetin, piceatannol, quercetin, nicotinamide riboside, and derivatives of the foregoing, as well as the sirtuin-activating compounds described in U.S. Patent Publication No. 2005/0136537. In an exemplary embodiment, a sirtuin-activating compound has no substantial modulating activity for a CLK protein.

“Sirtuin-inhibiting compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. Examples of sirtuin-inhibiting compounds include, for example, sirtinol and splitomicin, and derivatives thereof, as well as the sirtuin-inhibiting compounds described in U.S. Patent Publication No. 2005/0136537. In an exemplary embodiment, a sirtuin-inhibiting compound has no substantial modulating activity for a CLK protein.

The terms “PGC-1alpha protein” or “PGC-1α protein” or “PGC1a protein” refers to a member of the PPARγ Coactivator 1 (“PGC-1”) family of proteins. Examples of PGC-1alpha proteins include the mouse PGC-1alpha and human PGC-1alpha proteins which are described in U.S. Pat. No. 6,908,987 as well as homologs (e.g., orthologs and paralogs), variants, or fragments thereof. Exemplary fragments of PGC-1alpha include fragments that maintain at least one biological activity of a PGC-1alpha protein, such as, for example: ability to interact with (e.g., bind to) PPARγ; ability to modulate PPARγ activity; ability to modulate UCP expression; ability to modulate thermogenesis in adipocytes (e.g., thermogenesis in brown adipocytes) or muscle; ability to modulate oxygen consumption in adipocytes or muscle; ability to modulate adipogenesis (e.g., differentiation of white adipocytes into brown adipocytes); ability to modulate insulin sensitivity of cells (e.g., insulin sensitivity of muscle cells, liver cells, adipocytes); ability to interact with (e.g., bind to) nuclear hormone receptors (e.g., the thyroid hormone receptor, the estrogen receptor, the retinoic acid receptor); ability to modulate the activity of nuclear hormone receptors; or ability to interact with (e.g., bind to) the transcription factor C/EBPα. GenBank Accession numbers for mouse PGC-1 alpha are AAH66868 or O7034; GenBank Accession numbers for human PGC-1alpha are NP_{—}037393 or Q9UBK2.

The term “synthetic” is art-recognized and refers to production by in vitro chemical or enzymatic synthesis.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of a protein coding sequence is under the control of a promoter sequence (or other transcriptional regulatory sequence) which controls the expression of the protein coding sequence in a cell-type in which expression is intended. It will also be understood that the protein coding sequence can be under the control of transcriptional regulatory sequences which are the same or which are different from those sequences which control transcription of the naturally-occurring form of thr protein coding sequence.

“Treating” a condition or disease refers to curing as well as ameliorating at least one symptom of the condition or disease.

A “vector” is a self-replicating nucleic acid molecule that transfers an inserted nucleic acid molecule into and/or between host cells. The term includes vectors that function primarily for insertion of a nucleic acid molecule into a cell, replication vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). In exemplary embodiments, an expression vector comprises one or more transcriptional regulatory sequences operably linked to a protein coding sequence. An “expression system” usually connotes a suitable host cell comprised of an expression vector that can function to yield a desired expression product.

The term “vision impairment” refers to diminished vision, which is often only partially reversible or irreversible upon treatment (e.g., surgery). Particularly severe vision impairment is termed “blindness” or “vision loss”, which refers to a complete loss of vision, vision worse than 20/200 that cannot be improved with corrective lenses, or a visual field of less than 20 degrees diameter (10 degrees radius).

An “indicator of mitochondrial function” is any parameter that is indicative of mitochondrial function that can be measured by one skilled in the art. In certain embodiments, the indicator of mitochondrial function is a mitochondrial electron transport chain enzyme, a Krebs cycle enzyme, a mitochondrial matrix component, a mitochondrial membrane component or an ATP biosynthesis factor. In other embodiments, the indicator of mitochondrial function is mitochondrial number per cell or mitochondrial mass per cell. In other embodiments, the indicator of mitochondrial function is an ATP biosynthesis factor. In other embodiments, the indicator of mitochondrial function is the amount of ATP per mitochondrion, the amount of ATP per unit mitochondrial mass, the amount of ATP per unit protein or the amount of ATP per unit mitochondrial protein. In other embodiments, the indicator of mitochondrial function comprises free radical production. In other embodiments, the indicator of mitochondrial function comprises a cellular response to elevated intracellular calcium. In other embodiments, the indicator of mitochondrial function is the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the relative or absolute amount of mitochondrial DNA per cell in the patient.

“Improving mitochondrial function” or “altering mitochondrial function” may refer to (a) substantially (e.g., in a statistically significant manner, and preferably in a manner that promotes a statistically significant improvement of a clinical parameter such as prognosis, clinical score or outcome) restoring to a normal level at least one indicator of glucose responsiveness in cells having reduced glucose responsiveness and reduced mitochondrial mass and/or impaired mitochondrial function; or (b) substantially (e.g., in a statistically significant manner, and preferably in a manner that promotes a statistically significant improvement of a clinical parameter such as prognosis, clinical score or outcome) restoring to a normal level, or increasing to a level above and beyond normal levels, at least one indicator of mitochondrial function in cells having impaired mitochondrial function, or in cells having normal mitochondrial function, respectively. Improved or altered mitochondrial function may result from changes in extramitochondrial structures or events, as well as from mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

“Impaired mitochondrial function” may include a full or partial decrease, inhibition, diminution, loss or other impairment in the level and/or rate of any respiratory, metabolic or other biochemical or biophysical activity in some or all cells of a biological source. As non-limiting examples, markedly impaired electron transport chain (ETC) activity may be related to impaired mitochondrial function, as may be generation of increased reactive oxygen species (ROS) or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of mitochondrial function. These and other non-limiting examples of impaired mitochondrial function are described in greater detail below.

Other technical terms used herein have their ordinary meaning in the art that they are used, as exemplified by a variety of technical dictionaries, such as the McGraw-Hill Dictionary of Chemical Terms and the Stedman's Medical Dictionary.

2. Exemplary Uses

In certain aspects, the invention provides methods for modulating the level and/or activity of a CLK protein and methods of use thereof.

In certain embodiments, the invention provides methods for using CLK-inhibiting compounds. CLK-inhibiting compounds may be useful for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. CLK-inhibiting compounds may also be used for treating a disease or disorder in a subject that would benefit from increased mitochondrial activity, for enhancing muscle performance, for increasing muscle ATP levels, or for treating or preventing muscle tissue damage associated with hypoxia or ischemia. The methods comprise administering to a subject in need thereof a pharmaceutically effective amount of a CLK-inhibiting compound.

In other embodiments, CLK-inhibiting compounds may be useful for a variety of therapeutic application including, for example, increasing cellular sensitivity to stress (including increasing radiosensitivity and/or chemosensitivity), increasing the amount and/or rate of apoptosis, treatment of cancer (optionally in combination another chemotherapeutic agent), stimulation of appetite, and/or stimulation of weight gain, etc. The methods comprise administering to a subject in need thereof a pharmaceutically effective amount of a CLK-inhibiting compound.

In certain embodiments, a CLK-modulating compounds described herein may be taken alone or in combination with other compounds. In one embodiment, a mixture of two or more CLK-modulating compounds may be administered to a subject in need thereof. In another embodiment, a CLK-inhibiting compound may be administered with one or more sirtuin-activating compounds. Exemplary sirtuin-activating compounds include, for example, resveratrol, butein, fisetin, piceatannol, quercetin, nicotinamide riboside, and derivatives or analogs of the foregoing as well as the sirtuin-activating compounds described in U.S. Patent Publication No. 2005/0136537. In an exemplary embodiment, a CLK-inhibiting compound may be administered in combination with nicotinic acid. In another embodiment, a CLK-activating compound may be administered with one or more sirtuin-inhibiting compounds. Exemplary sirtuin-inhibiting compounds include, for example, nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′, 5′); (−)-epigallocatechin gallate (Hydroxy sites 5,7,3′,4′,5′ and gallate ester on 3); cyanidin chloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone); sirtinol; splitomicin (see e.g., Howitz et al. (2003) Nature 425:191; Grozinger et al. (2001) J. Biol. Chem. 276:38837; Dedalov et al. (2001) PNAS 98:15113; and Hirao et al. (2003) J. Biol. Chem. 278:52773); and the sirtuin-inhibiting compounds described in U.S. Patent Publication No. 2005/0136537. In yet another embodiment, one or more CLK-modulating compounds may be administered with one or more therapeutic agents for the treatment or prevention of various diseases, including, for example, cancer, diabetes, neurodegenerative diseases, cardiovascular disease, blood clotting, inflammation, flushing, obesity, ageing, stress, ocular disorders, etc. In another embodiment, a CLK-inhibiting compound may be administered with one or more agents that promote mitochondrial biogenesis, mitochondrial activity, and/or muscle performance. In various embodiments, combination therapies comprising a CLK-modulating compound may refer to (1) pharmaceutical compositions that comprise one or more CLK-modulating compounds in combination with one or more therapeutic agents; and (2) co-administration of one or more CLK-modulating compounds with one or more therapeutic agents wherein the CLK-modulating compound and therapeutic agent have not been formulated in the same compositions. When using separate formulations, the CLK-modulating compound may be administered at the same time, intermittent, staggered, prior to, subsequent to, or combinations thereof, with the administration of another therapeutic agent.

In certain embodiments, methods for reducing, preventing or treating diseases or disorders that involve activating a CLK protein may comprise increasing the protein level of a CLK, such as human CLK1, CLK2, CLK3 and/or CLK4, or homologs thereof. Increasing protein levels can be achieved by introducing into a cell one or more copies of a nucleic acid that encodes a CLK protein. For example, the level of a CLK protein can be increased in a mammalian cell by introducing into the mammalian cell a nucleic acid encoding the CLK protein, e.g., increasing the level of CLK1 by introducing a nucleic acid encoding the amino acid sequence set forth as SEQ ID NO: 1 and/or increasing the level of CLK2 by introducing a nucleic acid encoding the amino acid sequence set forth as SEQ ID NO: 2 and/or increasing the level of CLK3 by introducing a nucleic acid encoding the amino acid sequence set forth as SEQ ID NO: 3 and/or increasing the level of CLK4 by introducing a nucleic acid encoding the amino acid sequence set forth as SEQ ID NO: 4. The nucleic acid may be under the control of a transcriptional regulatory sequence (e.g., a promoter) that regulates the expression of the CLK1, CLK2, CLK3 and/or CLK4 nucleic acid. Alternatively, the nucleic acid may be introduced into the genome of the cell at a location in the genome that is downstream from a promoter. Methods for increasing the level of a protein using these methods are well known in the art.

A nucleic acid that is introduced into a cell to increase the protein level of a CLK may encode a protein that is at least about 80%, 85%, 90%, 95%, 98%, or 99% identical to the sequence of a CLK, e.g., CLK1 (GenBank Accession # P49759 and # AAH31549), CLK2 (GenBank Accession # NP_{—}003984 and AAH53603), CLK3 (GenBank Accession # P49761 and AAH19881) and/or CLK4 (GenBank Accession # Q9HAZ1 or NP_{—}065717) protein. For example, the nucleic acid encoding the protein may be at least about 80%, 85%, 90%, 95%, 98%, or 99% identical to a nucleic acid encoding a CLK1 (e.g. GenBank Accession # BC031549), CLK2 (e.g., GenBank Accession # BC053603), CLK3 (GenBank Accession # BC019881) and/or CLK4 (GenBank Accession # NM_{—}020666) protein. The nucleic acid may also be a nucleic acid that hybridizes, preferably under stringent hybridization conditions, to a nucleic acid encoding a wild-type CLK, e.g., CLK1 (GenBank Accession # P49759 and # AAH31549), CLK2 (GenBank Accession # NP_{—}003984 and AAH53603), CLK3 (GenBank Accession # BC019881) and/or CLK4 (GenBank Accession # NM_{—}020666) protein. Stringent hybridization conditions may include hybridization and a wash in 0.2×SSC at 65° C. When using a nucleic acid that encodes a protein that is different from a wild-type CLK protein, such as a protein that is a fragment of a wild-type CLK, the protein is preferably biologically active, e.g., is capable of phosphorylating a substrate polypeptide. It is only necessary to express in a cell a portion of the CLK that is biologically active. Whether a protein retains a biological function, e.g., phosphorylation capabilities, can be determined according to methods known in the art.

In certain embodiments, methods for reducing, preventing or treating diseases or disorders that involve inhibiting CLK protein activity may comprise decreasing the protein level of one or more CLK proteins, such as human CLK1, CLK2, CLK3 and/or CLK4, or homologs thereof. Decreasing a CLK protein level can be achieved according to methods known in the art. For example, an siRNA, an antisense nucleic acid, or a ribozyme targeted to a nucleic acid encoding the CLK protein can be introduced into or expressed in the cell. A dominant negative CLK mutant, e.g., a mutant that does not have kinase activity may also be used. Alternatively, agents that inhibit transcription can be used to decreases CLK expression.

Methods for modulating CLK protein levels also include methods for modulating the transcription of genes encoding CLKs, methods for stabilizing/destabilizing the corresponding mRNAs, and other methods known in the art.

In certain embodiments, CLK-inhibiting compounds are not used for treating and/or preventing diseases and disorders associated with alternate, abnormal, aberrant or undesired splicing including abnormal splicing related to a mutation around a splice site, abnormal splicing not related to a splice site mutation, abnormal splicing associated with levels of splicing that are too high or too low at a particular splice site, and/or abnormal splicing associated with selection of a splice site. In exemplary embodiments, CLK-inhibiting compounds are not used for treating and/or preventing one or more of the following diseases or disorders: beta-thalassemia, FTDP-17, NF2, FRASIER, Wilms tumor, breast cancer, ovarian cancer, renal cancer, lung cancer, urothellal cancer, gastric cancer, papillary thyroid cancer, HNSCC, invasive breast cancer, glant cell tumors of bone, prostate cancer, melanoma, lymphoma, oral cancer, pharyngeal cancer, progeria, neurodegenerative diseases such as Alzheimer's disease, Parkinson's disease, amyotrophic lateral sclerosis (ALS), Huntington disease, spinocerebellar ataxia, spinal and bulbar muscular atrophy (SBMA) and epilepsy, progressive supranuclear palsy, and/or Pick's disease.

i. Aging/Stress

In one embodiment, the invention provides a method extending the lifespan of a cell, extending the proliferative capacity of a cell, slowing ageing of a cell, promoting the survival of a cell, delaying cellular senescence in a cell, mimicking the effects of calorie restriction, increasing the resistance of a cell to stress, or preventing apoptosis of a cell, by contacting the cell with a CLK-inhibiting compound.

The methods described herein may be used to increase the amount of time that cells, particularly primary cells (i.e., cells obtained from an organism, e.g., a human), may be kept alive in a cell culture. Embryonic stem (ES) cells and pluripotent cells, and cells differentiated therefrom, may also be treated with a CLK-inhibiting compound to keep the cells, or progeny thereof, in culture for longer periods of time. Such cells can also be used for transplantation into a subject, e.g., after ex vivo modification.

In one embodiment, cells that are intended to be preserved for long periods of time may be treated with a CLK-inhibiting compound. The cells may be in suspension (e.g., blood cells, serum, biological growth media, etc.) or in tissues or organs. For example, blood collected from an individual for purposes of transfusion may be treated with a CLK-inhibiting compound to preserve the blood cells for longer periods of time. Additionally, blood to be used for forensic purposes may also be preserved using a CLK-inhibiting compound. Other cells that may be treated to extend their lifespan or protect against apoptosis include cells for consumption, e.g., cells from non-human mammals (such as meat) or plant cells (such as vegetables).

CLK-inhibiting compound may also be applied during developmental and growth phases in mammals, plants, insects or microorganisms, in order to alter, retard or accelerate the developmental and/or growth process.

In another embodiment, a CLK-inhibiting compound may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the CLK-inhibiting compound prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a CLK-inhibiting compound or may have a subset of cells/tissue treated locally with a CLK-inhibiting compound. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, cells may be treated with a CLK-inhibiting compound in vivo, e.g., to increase their lifespan or prevent apoptosis. For example, skin can be protected from aging (e.g., developing wrinkles, loss of elasticity, etc.) by treating skin or epithelial cells with a CLK-inhibiting compound. In an exemplary embodiment, skin is contacted with a pharmaceutical or cosmetic composition comprising a CLK-inhibiting compound. Exemplary skin afflictions or skin conditions that may be treated in accordance with the methods described herein include disorders or diseases associated with or caused by inflammation, sun damage or natural aging. For example, compositions comprising a CLK-inhibiting compound find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, a CLK-inhibiting compound may be used for the treatment of wounds and/or burns to promote healing, including, for example, first-, second- or third-degree burns and/or thermal, chemical or electrical burns. The formulations may be administered topically, to the skin or mucosal tissue, as an ointment, lotion, cream, microemulsion, gel, solution or the like, as further described herein, within the context of a dosing regimen effective to bring about the desired result.

Topical formulations comprising one or more CLK-inhibiting compounds may also be used as preventive, e.g., chemopreventive, compositions. When used in a chemopreventive method, susceptible skin is treated prior to any visible condition in a particular individual.

CLK-inhibiting compounds may be delivered locally or systemically to a subject. In one embodiment, a CLK-inhibiting compound is delivered locally to a tissue or organ of a subject by injection, topical formulation, etc.

In another embodiment, a CLK-inhibiting compound may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a CLK-inhibiting compound may be administered to a subject in order to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan.

CLK-inhibiting compounds may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. CLK-inhibiting compounds can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

CLK-inhibiting compounds can also be administered to a subject suffering from an acute disease, e.g., damage to an organ or tissue, e.g., a subject suffering from stroke or myocardial infarction or a subject suffering from a spinal cord injury. CLK-inhibiting compound may also be used to repair an alcoholic's liver.

ii. Cardiovascular Disease

In another embodiment, the invention provides a method for treating and/or preventing a cardiovascular disease by administering to a subject in need thereof a CLK-inhibiting compound.

Cardiovascular diseases that can be treated or prevented using a CLK-inhibiting compound include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using the CLK-inhibiting compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The CLK-inhibiting compounds may also be used for increasing HDL levels in plasma of an individual.

Yet other disorders that may be treated with CLK-inhibiting compounds include restenosis, e.g., following coronary intervention, and disorders relating to an abnormal level of high density and low density cholesterol.

In one embodiment, a CLK-inhibiting compound may be administered as part of a combination therapeutic with another cardiovascular agent including, for example, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a natriuretic agent.

In one embodiment, a CLK-inhibiting compound may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocainide, procainamide, encainide, flecanide, tocainide, phenyloin, propafenone, quinidine, disopyramide, and flecainide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltaizem, digitalis, adenosine, nickel chloride, and magnesium ions.

In another embodiment, a CLK-inhibiting compound may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; anti-arrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorothalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetamide, triamterene, amiloride and spironolactone and salts of such compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex (APSAC, Eminase, Beecham Laboratories), or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this invention within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin. Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of the brain metabolic stimulants include amantadine, tiapride, and .gamma.-aminobutyric acid. Examples of the anticoagulant include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene. Examples of the thrombolytic drug include urokinase, tissue-type plasminogen activators (such as alteplase, tisokinase, nateplase, pamiteplase, monteplase, and rateplase), and nasaruplase. Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine. Examples of the antianginal drug include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, and xybenolol), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline) trimetazidine, dipyridamole, etafenone, dilazep, trapidil, nicorandil, enoxaparin, and aspirin. Examples of the diuretic include thiazide diuretics (such as hydrochlorothiazide, methyclothiazide, trichlormethiazide, benzylhydrochlorothiazide, and penflutizide), loop diuretics (such as furosemide, etacrynic acid, bumetamide, piretanide, azosemide, and torasemide), K⁺ sparing diuretics (spironolactone, triamterene, and potassium canrenoate), osmotic diuretics (such as isosorbide, D-mannitol, and glycerin), nonthiazide diuretics (such as meticrane, tripamide, chlorthalidone, and mefruside), and acetazolamide. Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as amrinone, olprinone, and milrinone), denopamine, ubidecarenone, pimobendan, levosimendan, aminoethylsulfonic acid, vesnarinone, carperitide, and colforsin daropate. Examples of the antiarrhythmic drug include ajmaline, pirmenol, procainamide, cibenzoline, disopyramide, quinidine, aprindine, mexiletine, lidocaine, phenyloin, pilsicainide, propafenone, flecainide, atenolol, acebutolol, sotalol, propranolol, metoprolol, pindolol, amiodarone, nifekalant, diltiazem, bepridil, and verapamil. Examples of the antihyperlipidemic drug include atorvastatin, simvastatin, pravastatin sodium, fluvastatin sodium, clinofibrate, clofibrate, simfibrate, fenofibrate, bezafibrate, colestimide, and colestyramine. Examples of the immunosuppressant include azathioprine, mizoribine, cyclosporine, tacrolimus, gusperimus, and methotrexate.

iii. Cell Death/Cancer

CLK-inhibiting compounds may be administered to subjects who have recently received or are likely to receive a dose of radiation or toxin. In one embodiment, the dose of radiation or toxin is received as part of a work-related or medical procedure, e.g., working in a nuclear power plant, flying an airplane, an X-ray, CAT scan, or the administration of a radioactive dye for medical imaging; in such an embodiment, the compound is administered as a prophylactic measure. In another embodiment, the radiation or toxin exposure is received unintentionally, e.g., as a result of an industrial accident, habitation in a location of natural radiation, terrorist act, or act of war involving radioactive or toxic material. In such a case, the compound is preferably administered as soon as possible after the exposure to inhibit apoptosis and the subsequent development of acute radiation syndrome.

CLK-modulating compounds may also be used for treating and/or preventing cancer. In certain embodiments, CLK-inhibiting compounds may be used for treating and/or preventing cancer. Accordingly, a decrease in the level and/or activity of a CLK protein may be useful for treating and/or preventing the incidence of age-related disorders, such as, for example, cancer. In other embodiments, CLK-activating compounds may be used for treating or preventing cancer. For example, CLK-activating compounds may be used to increase apoptosis, as well as to reduce the lifespan of cells and organisms, render them more sensitive to stress, and/or increase the radiosensitivity and/or chemosensitivity of a cell or organism. Thus, CLK-activating compounds may be used, e.g., for treating cancer. Exemplary cancers that may be treated using a CLK-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Benign cell growth can also be treated, e.g., warts. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of a CLK-activating compound. Alternatively, cells can be obtained from a subject, treated ex vivo to remove certain undesirable cells, e.g., cancer cells, and administered back to the same or a different subject.

Chemotherapeutic agents that may be co-administered with CLK-activating compounds (e.g., compounds that induce apoptosis, compounds that reduce lifespan or compounds that render cells sensitive to stress) include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These chemotherapeutic agents may be used by themselves with a CLK-activating compound (e.g., a compound that induces cell death or reduces lifespan or increases sensitivity to stress) and/or in combination with other chemotherapeutics agents. Many combinatorial therapies have been developed, including but not limited to those listed in Table 1.

TABLE 1
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, |
| | Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, |
| | Cyclophosphamide, Vincristine, Procarbazine, |
| | Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, |
| | with or without/Bleomycin |
| CEV | Cyclophosphamide, Etoposide, Vincristine |
| CF | Cisplatin, Fluorouracil or Carboplatin |
| | Fluorouracil |
| CHAP | Cyclophosphamide or Cyclophosphamide, |
| | Altretamine, Doxorubicin, Cisplatin |
| ChlVPP | Chlorambucil, Vinblastine, Procarbazine, |
| | Prednisone |
| CHOP | Cyclophosphamide, Doxorubicin, Vincristine, |
| | Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, |
| | Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, |
| | Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, |
| | Etoposide |
| COPP | Cyclophosphamide, Vincristine, Procarbazine, |
| | Prednisone |
| CP(Chronic | Chlorambucil, Prednisone |
| lymphocytic leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| ICE-T | Ifosfamide, Carboplatin, Etoposide, Paclitaxel, |
| | Mesna |
| IDMTX/6-MP | Methotrexate, Mercaptopurine, Leucovorin |
| JE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, |
| | Prednisone, Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, |
| | Prednisone |
| MAID | Mesna, Doxorubicin, Ifosfamide, Dacarbazine |
| m-BACOD | Bleomycin, Doxorubicin, Cyclophosphamide, |
| | Vincristine, Dexamethasone, |
| | Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, |
| | Prednisone |
| MOPP/ABV | Mechlorethamine, Vincristine, Procarbazine, |
| | Prednisone, Doxorubicin, |
| | Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, |
| | Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, |
| | Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, |
| | Doxorubicin, Cyclophosphamide, |
| | Etoposide |
| ProMACE/cytaBOM | Prednisone, Doxorubicin, Cyclophosphamide, |
| | Etoposide, Cytarabine, Bleomycin, Vincristine, |
| | Methotrexate, Leucovorin, Cotrimoxazole |
| PRoMACE/MOPP | Prednisone, Doxorubicin, Cyclophosphamide, |
| | Etoposide, Mechlorethamine, Vincristine, |
| | Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, |
| | Asparaginase |
| SMF | Streptozocin, Mitomycin, Fluorouracil |
| TAD | Mechlorethamine, Doxorubicin, Vinblastine, |
| | Vincristine, Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, |
| | Cisplatin, Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, |
| | Flouxymesterone |
| VBAP | Vincristine, Carmustine, Doxorubicin, Prednisone |
| VBCMP | Vincristine, Carmustine, Melphalan, |
| | Cyclophosphamide, Prednisone |
| VC | Vinorelbine, Cisplatin |
| VCAP | Vincristine, Cyclophosphamide, Doxorubicin, |
| | Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, |
| | Prednisone |
| VP | Etoposide, Cisplatin |
| V-TAD | Etoposide, Thioguanine, Daunorubicin, |
| | Cytarabine |
| 5 + 2 | Cytarabine, Daunorubicin, Mitoxantrone |
| 7 + 3 | Cytarabine with/, Daunorubicin or |
| | Idarubicin or Mitoxantrone |
| “8 in 1” | Methylprednisolone, Vincristine, Lomustine, |
| | Procarbazine, Hydroxyurea, Cisplatin, |
| | Cytarabine, Dacarbazine |

In addition to conventional chemotherapeutics, the CLK-activating compounds described herein as capable of inducing cell death or reducing lifespan can also be used with antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

Combination therapies comprising CLK-activating compounds and a conventional chemotherapeutic agent may be advantageous over combination therapies known in the art because the combination allows the conventional chemotherapeutic agent to exert greater effect at lower dosage. In a preferred embodiment, the effective dose (ED₅₀) for a chemotherapeutic agent, or combination of conventional chemotherapeutic agents, when used in combination with a CLK-activating compound is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a CLK-activating compound described herein can be at least 2 fold greater than the TI for conventional chemotherapeutic regimen alone, and even more preferably at 5 fold, 10 fold or even 25 fold greater.

iv. Neuronal Diseases/Disorders

In certain aspects, CLK-inhibiting compounds can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. CLK-inhibiting compounds can be used to treat these disorders and others as described below.

AD is a chronic, incurable, and unstoppable CNS disorder that occurs gradually, resulting in memory loss, unusual behavior, personality changes, and a decline in thinking abilities. These losses are related to the death of specific types of brain cells and the breakdown of connections and their supporting network (e.g. glial cells) between them. AD has been described as childhood development in reverse. In most people with AD, symptoms appear after the age 60. The earliest symptoms include loss of recent memory, faulty judgment, and changes in personality. Later in the disease, those with AD may forget how to do simple tasks like washing their hands. Eventually people with AD lose all reasoning abilities and become dependent on other people for their everyday care. Finally, the disease becomes so debilitating that patients are bedridden and typically develop coexisting illnesses.

PD is a chronic, incurable, and unstoppable CNS disorder that occurs gradually and results in uncontrolled body movements, rigidity, tremor, and dyskinesia. These motor system problems are related to the death of brain cells in an area of the brain that produces dopamine, a chemical that helps control muscle activity. In most people with PD, symptoms appear after age 50. The initial symptoms of PD are a pronounced tremor affecting the extremities, notably in the hands or lips. Subsequent characteristic symptoms of PD are stiffness or slowness of movement, a shuffling walk, stooped posture, and impaired balance. There are wide ranging secondary symptoms such as memory loss, dementia, depression, emotional changes, swallowing difficulties, abnormal speech, sexual dysfunction, and bladder and bowel problems. These symptoms will begin to interfere with routine activities, such as holding a fork or reading a newspaper. Finally, people with PD become so profoundly disabled that they are bedridden.

ALS (motor neuron disease) is a chronic, incurable, and unstoppable CNS disorder that attacks the motor neurons, components of the CNS that connect the brain to the skeletal muscles. In ALS, the motor neurons deteriorate and eventually die, and though a person's brain normally remains fully functioning and alert, the command to move never reaches the muscles. Most people who get ALS are between 40 and 70 years old. The first motor neurons that weaken are those controlling the arms or legs. Those with ALS may have trouble walking, they may drop things, fall, slur their speech, and laugh or cry uncontrollably. Eventually the muscles in the limbs begin to atrophy from disuse. This muscle weakness will become debilitating and a person will need a wheel chair or become unable to function out of bed.

The causes of these neurological diseases have remained largely unknown. They are conventionally defined as distinct diseases, yet clearly show extraordinary similarities in basic processes and commonly demonstrate overlapping symptoms far greater than would be expected by chance alone. Current disease definitions fail to properly deal with the issue of overlap and a new classification of the neurodegenerative disorders has been called for.

HD is another neurodegenerative disease resulting from genetically programmed degeneration of neurons in certain areas of the brain. This degeneration causes uncontrolled movements, loss of intellectual faculties, and emotional disturbance. HD is a familial disease, passed from parent to child through a dominant mutation in the wild-type gene. Some early symptoms of HD are mood swings, depression, irritability or trouble driving, learning new things, remembering a fact, or making a decision. As the disease progresses, concentration on intellectual tasks becomes increasingly difficult and the patient may have difficulty feeding himself or herself and swallowing.

Tay-Sachs disease and Sandhoff disease are glycolipid storage diseases caused by the lack of lysosomal β-hexosaminidase (Gravel et al., in The Metabolic Basis of Inherited Disease, eds. Scriver et al., McGraw-Hill, New York, pp. 2839-2879, 1995). In both disorders, GM2 ganglioside and related glycolipidssubstrates for β-hexosaminidase accumulate in the nervous system and trigger acute neurodegeneration. In the most severe forms, the onset of symptoms begins in early infancy. A precipitous neurodegenerative course then ensues, with affected infants exhibiting motor dysfunction, seizure, visual loss, and deafness. Death usually occurs by 2-5 years of age. Neuronal loss through an apoptotic mechanism has been demonstrated (Huang et al., Hum. Mol. Genet. 6: 1879-1885, 1997).

It is well-known that apoptosis plays a role in AIDS pathogenesis in the immune system. However, HIV-1 also induces neurological disease. Shi et al. (J. Clin. Invest. 98: 1979-1990, 1996) examined apoptosis induced by HIV-1 infection of the CNS in an in vitro model and in brain tissue from AIDS patients, and found that HIV-1 infection of primary brain cultures induced apoptosis in neurons and astrocytes in vitro. Apoptosis of neurons and astrocytes was also detected in brain tissue from 10/11 AIDS patients, including 5/5 patients with HIV-1 dementia and 4/5 nondemented patients.

There are four main peripheral neuropathies associated with HIV, namely sensory neuropathy, AIDP/CIPD, drug-induced neuropathy and CMV-related.

The most common type of neuropathy associated with AIDS is distal symmetrical polyneuropathy (DSPN). This syndrome is a result of nerve degeneration and is characterized by numbness and a sensation of pins and needles. DSPN causes few serious abnormalities and mostly results in numbness or tingling of the feet and slowed reflexes at the ankles. It generally occurs with more severe immunosuppression and is steadily progressive. Treatment with tricyclic antidepressants relieves symptoms but does not affect the underlying nerve damage.

A less frequent, but more severe type of neuropathy is known as acute or chronic inflammatory demyelinating polyneuropathy (AIDP/CIDP). In AIDP/CIDP there is damage to the fatty membrane covering the nerve impulses. This kind of neuropathy involves inflammation and resembles the muscle deterioration often identified with long-term use of AZT. It can be the first manifestation of HIV infection, where the patient may not complain of pain, but fails to respond to standard reflex tests. This kind of neuropathy may be associated with seroconversion, in which case it can sometimes resolve spontaneously. It can serve as a sign of HIV infection and indicate that it might be time to consider antiviral therapy. AIDP/CIDP may be auto-immune in origin.

Drug-induced, or toxic, neuropathies can be very painful. Antiviral drugs commonly cause peripheral neuropathy, as do other drugs e.g. vincristine, dilantin (an anti-seizure medication), high-dose vitamins, isoniazid, and folic acid antagonists. Peripheral neuropathy is often used in clinical trials for antivirals as a dose-limiting side effect, which means that more drugs should not be administered. Additionally, the use of such drugs can exacerbate otherwise minor neuropathies. Usually, these drug-induced neuropathies are reversible with the discontinuation of the drug.

CMV causes several neurological syndromes in AIDS, including encephalitis, myelitis, and polyradiculopathy.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. CLK-inhibiting compounds may be useful for treating or preventing neuronal loss due to these prior diseases.

In another embodiment, a CLK-inhibiting compound may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy. The most distal portions of axons are usually the first to degenerate, and axonal atrophy advances slowly towards the nerve's cell body. If the noxious stimulus is removed, regeneration is possible, though prognosis decreases depending on the duration and severity of the stimulus. Those with distal axonopathies usually present with symmetrical glove-stocking sensori-motor disturbances. Deep tendon reflexes and autonomic nervous system (ANS) functions are also lost or diminished in affected areas.

Diabetic neuropathies are neuropathic disorders that are associated with diabetes mellitus. These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy. Clinical manifestations of diabetic neuropathy include, for example, sensorimotor polyneuropathy such as numbness, sensory loss, dysesthesia and nighttime pain; autonomic neuropathy such as delayed gastric emptying or gastroparesis; and cranial neuropathy such as oculomotor (3rd) neuropathies or Mononeuropathies of the thoracic or lumbar spinal nerves.

Peripheral neuropathy is the medical term for damage to nerves of the peripheral nervous system, which may be caused either by diseases of the nerve or from the side-effects of systemic illness. Peripheral neuropathies vary in their presentation and origin, and may affect the nerve or the neuromuscular junction. Major causes of peripheral neuropathy include seizures, nutritional deficiencies, and HIV, though diabetes is the most likely cause. Mechanical pressure from staying in one position for too long, a tumor, intraneural hemorrhage, exposing the body to extreme conditions such as radiation, cold temperatures, or toxic substances can also cause peripheral neuropathy.

In an exemplary embodiment, a CLK-inhibiting compound may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

MS is a chronic, often disabling disease of the central nervous system. Various and converging lines of evidence point to the possibility that the disease is caused by a disturbance in the immune function, although the cause of this disturbance has not been established. This disturbance permits cells of the immune system to “attack” myelin, the fat containing insulating sheath that surrounds the nerve axons located in the central nervous system (“CNS”). When myelin is damaged, electrical pulses cannot travel quickly or normally along nerve fiber pathways in the brain and spinal cord. This results in disruption of normal electrical conductivity within the axons, fatigue and disturbances of vision, strength, coordination, balance, sensation, and bladder and bowel function.

As such, MS is now a common and well-known neurological disorder that is characterized by episodic patches of inflammation and demyelination which can occur anywhere in the CNS. However, almost always without any involvement of the peripheral nerves associated therewith. Demyelination produces a situation analogous to that resulting from cracks or tears in an insulator surrounding an electrical cord. That is, when the insulating sheath is disrupted, the circuit is “short circuited” and the electrical apparatus associated therewith will function intermittently or nor at all. Such loss of myelin surrounding nerve fibers results in short circuits in nerves traversing the brain and the spinal cord that thereby result in symptoms of MS. It is further found that such demyelination occurs in patches, as opposed to along the entire CNS. In addition, such demyelination may be intermittent. Therefore, such plaques are disseminated in both time and space.

It is believed that the pathogenesis involves a local disruption of the blood brain barrier which causes a localized immune and inflammatory response, with consequent damage to myelin and hence to neurons.

Clinically, MS exists in both sexes and can occur at any age. However, its most common presentation is in the relatively young adult, often with a single focal lesion such as a damage of the optic nerve, an area of anesthesia (loss of sensation), or paraesthesia (localize loss of feeling), or muscular weakness. In addition, vertigo, double vision, localized pain, incontinence, and pain in the arms and legs may occur upon flexing of the neck, as well as a large variety of less common symptoms.

An initial attack of MS is often transient, and it may be weeks, months, or years before a further attack occurs. Some individuals may enjoy a stable, relatively event free condition for a great number of years, while other less fortunate ones may experience a continual downhill course ending in complete paralysis. There is, most commonly, a series of remission and relapses, in which each relapse leaves a patient somewhat worse than before. Relapses may be triggered by stressful events, viral infections or toxins. Therein, elevated body temperature, i.e., a fever, will make the condition worse, or as a reduction of temperature by, for example, a cold bath, may make the condition better.

In yet another embodiment, a CLK-inhibiting compound may be used to treat trauma to the nerves, including, trauma due to disease, injury (including surgical intervention), or environmental trauma (e.g., neurotoxins, alcoholism, etc.).

CLK-inhibiting compounds may also be useful to prevent, treat, and alleviate symptoms of various PNS disorders, such as the ones described below. The PNS is composed of the nerves that lead to or branch off from the spinal cord and CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

Peripheral neuropathy is a widespread disorder, and there are many underlying causes. Some of these causes are common, such as diabetes, and others are extremely rare, such as acrylamide poisoning and certain inherited disorders. The most common worldwide cause of peripheral neuropathy is leprosy. Leprosy is caused by the bacterium Mycobacterium leprae, which attacks the peripheral nerves of affected people.

Leprosy is extremely rare in the United States, where diabetes is the most commonly known cause of peripheral neuropathy. It has been estimated that more than 17 million people in the United States and Europe have diabetes-related polyneuropathy. Many neuropathies are idiopathic; no known cause can be found. The most common of the inherited peripheral neuropathies in the United States is Charcot-Marie-Tooth disease, which affects approximately 125,000 persons.

Another of the better known peripheral neuropathies is Guillain-Barré syndrome, which arises from complications associated with viral illnesses, such as cytomegalovirus, Epstein-Barr virus, and human immunodeficiency virus (HIV), or bacterial infection, including Campylobacter jejuni and Lyme disease. The worldwide incidence rate is approximately 1.7 cases per 100,000 people annually. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the PNS and the CNS, as well as other body tissues.

Other PNS diseases treatable CLK-inhibiting compound include: Brachial Plexus Neuropathies (diseases of the cervical and first thoracic roots, nerve trunks, cords, and peripheral nerve components of the brachial plexus. Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp 1351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy (see Adams et al., Principles of Neurology, 6th ed, p 1325); mononeuropathies (disease or trauma involving a single peripheral nerve in isolation, or out of proportion to evidence of diffuse peripheral nerve dysfunction). Mononeuritis multiplex refers to a condition characterized by multiple isolated nerve injuries. Mononeuropathies may result from a wide variety of causes, including ischemia; traumatic injury; compression; connective tissue diseases; cumulative trauma disorders; and other conditions; Neuralgia (intense or aching pain that occurs along the course or distribution of a peripheral or cranial nerve); Peripheral Nervous System Neoplasms (neoplasms which arise from peripheral nerve tissue). This includes neurofibromas; Schwannomas; granular cell tumors; and malignant peripheral nerve sheath tumors. See DeVita Jr et al., Cancer: Principles and Practice of Oncology, 5th ed, pp 1750-1); and Nerve Compression Syndromes (mechanical compression of nerves or nerve roots from internal or external causes). These may result in a conduction block to nerve impulses, due to, for example, myelin sheath dysfunction, or axonal loss. The nerve and nerve sheath injuries may be caused by ischemia; inflammation; or a direct mechanical effect; Neuritis (a general term indicating inflammation of a peripheral or cranial nerve). Clinical manifestation may include pain; paresthesias; paresis; or hyperesthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

In another embodiment, a CLK-inhibiting compound may be used to treat or prevent chemotherapeutic induced neuropathy. The CLK-inhibiting compounds may be administered prior to administration of the chemotherapeutic agent, concurrently with administration of the chemotherapeutic drug, and/or after initiation of administration of the chemotherapeutic drug. If the CLK-inhibiting compound is administered after the initiation of administration of the chemotherapeutic drug, it is desirable that the CLK-inhibiting compound be administered prior to, or at the first signs, of chemotherapeutic induced neuropathy.

Chemotherapy drugs can damage any part of the nervous system. Encephalopathy and myelopathy are fortunately very rare. Damage to peripheral nerves is much more common and can be a side effect of treatment experienced by people with cancers, such as lymphoma. Most of the neuropathy affects sensory rather than motor nerves. Thus, the common symptoms are tingling, numbness or a loss of balance. The longest nerves in the body seem to be most sensitive hence the fact that most patients will report numbness or pins and needles in their hands and feet.

The chemotherapy drugs which are most commonly associated with neuropathy, are the Vinca alkaloids (anti-cancer drugs originally derived from a member of the periwinkle—the Vinca plant genus) and a platinum-containing drug called Cisplatin. The Vinca alkaloids include the drugs vinblastine, vincristine and vindesine. Many combination chemotherapy treatments for lymphoma for example CHOP and CVP contain vincristine, which is the drug known to cause this problem most frequently. Indeed, it is the risk of neuropathy that limits the dose of vincristine that can be administered.

Studies that have been performed have shown that most patients will lose some reflexes in their legs as a result of treatment with vincristine and many will experience some degree of tingling (paresthesia) in their fingers and toes. The neuropathy does not usually manifest itself right at the start of the treatment but generally comes on over a period of a few weeks. It is not essential to stop the drug at the first onset of symptoms, but if the neuropathy progresses this may be necessary. It is very important that patients should report such symptoms to their doctors, as the nerve damage is largely reversible if the drug is discontinued. Most doctors will often reduce the dose of vincristine or switch to another form of Vinca alkaloid such as vinblastine or vindesine if the symptoms are mild. Occasionally, the nerves supplying the bowel are affected causing abdominal pain and constipation.

In another embodiment, a CLK-inhibiting compound may be used to treat or prevent a polyglutamine disease. Huntington's Disease (HD) and Spinocerebellar ataxia type 1 (SCA1) are just two examples of a class of genetic diseases caused by dynamic mutations involving the expansion of triplet sequence repeats. In reference to this common mechanism, these disorders are called trinucleotide repeat diseases. At least 14 such diseases are known to affect human beings. Nine of them, including SCA1 and Huntington's disease, have CAG as the repeated sequence (see Table 1 below). Since CAG codes for an amino acid called glutamine, these nine trinucleotide repeat disorders are collectively known as polyglutamine diseases.

Although the genes involved in different polyglutamine diseases have little in common, the disorders they cause follow a strikingly similar course. Each disease is characterized by a progressive degeneration of a distinct group of nerve cells. The major symptoms of these diseases are similar, although not identical, and usually affect people in midlife. Given the similarities in symptoms, the polyglutamine diseases are hypothesized to progress via common cellular mechanisms. In recent years, scientists have made great strides in unraveling what the mechanisms are.

Above a certain threshold, the greater the number of glutamine repeats in a protein, the earlier the onset of disease and the more severe the symptoms. This suggests that abnormally long glutamine tracts render their host protein toxic to nerve cells.

To test this hypothesis, scientists have generated genetically engineered mice expressing proteins with long polyglutamine tracts. Regardless of whether the mice express full-length proteins or only those portions of the proteins containing the polyglutamine tracts, they develop symptoms of polyglutamine diseases. This suggests that a long polyglutamine tract by itself is damaging to cells and does not have to be part of a functional protein to cause its damage.

For example, it is thought that the symptoms of SCA1 are not directly caused by the loss of normal ataxin-1 function but rather by the interaction between ataxin-1 and another protein called LANP. LANP is needed for nerve cells to communicate with one another and thus for their survival. When the mutant ataxin-1 protein accumulates inside nerve cells, it “traps” the LANP protein, interfering with its normal function. After a while, the absence of LANP function appears to cause nerve cells to malfunction.

TABLE 1
Summary of Polyglutamine Diseases.

| | | | | | Normal | Disease |
|---|---|---|---|---|---|---|
| | Gene | Chromosomal | Pattern of | | repeat | repeat |
| Disease | name | location | inheritance | Protein | length | length |
| Spinobulbar | AR | Xq13-21 | X-linked | androgen | 9-36 | 38-62 |
| muscular | | | recessive | receptor | | |
| atrophy | | | | (AR) | | |
| (Kennedy | | | | | | |
| disease) | | | | | | |
| Huntington's | HD | 4p16.3 | autosomal | huntingtin | 6-35 | 36-121 |
| disease | | | dominant | | | |
| Dentatorubral- | DRPLA | 12p13.31 | autosomal | atrophin-1 | 6-35 | 49-88 |
| pallidoluysian | | | dominant | | | |
| atrophy (Haw | | | | | | |
| River | | | | | | |
| syndrome) | | | | | | |
| Spinocerebellar | SCA1 | 6p23 | autosomal | ataxin-1 | 6-44 | 39-82 |
| ataxia type 1 | | | dominant | | | |
| Spinocerebellar | SCA2 | 12q24.1 | autosomal | ataxin-2 | 15-31 | 36-63 |
| ataxia type 2 | | | dominant | | | |
| Spinocerebellar | SCA3 | 14q32.1 | autosomal | ataxin-3 | 12-40 | 55-84 |
| ataxia type 3 | | | dominant | | | |
| (Machado- | | | | | | |
| Joseph disease) | | | | | | |
| Spinocerebellar | SCA6 | 19p13 | autosomal | α1_{A}- | 4-18 | 21-33 |
| ataxia type 6 | | | dominant | voltage- | | |
| | | | | dependent | | |
| | | | | calcium | | |
| | | | | channel | | |
| | | | | subunit | | |
| Spinocerebellar | SCA7 | 3p12-13 | autosomal | ataxin-7 | 4-35 | 37-306 |
| ataxia type 7 | | | dominant | | | |
| Spinocerebellar | SCA17 | 6q27 | autosomal | TATA | 25-42 | 45-63 |
| ataxia type 17 | | | dominant | binding | | |
| | | | | protein | | |

Many transcription factors have also been found in neuronal inclusions in different diseases. It is possible that these transcription factors interact with the polyglutamine-containing proteins and then become trapped in the neuronal inclusions. This in turn might keep the transcription factors from turning genes on and off as needed by the cell. Another observation is hypoacetylation of histones in affected cells. This has led to the hypothesis that Class I/II Histone Deacetylase (HDAC I/II) inhibitors, which are known to increase histone acetylation, may be a novel therapy for polyglutamine diseases (US Patent Publication No. 2004/0142859; “Method of treating neurodegenerative, psychiatric, and other disorders with deacetylase inhibitors”).

In yet another embodiment, the invention provides a method for treating or preventing neuropathy related to ischemic injuries or diseases, such as, for example, coronary heart disease (including congestive heart failure and myocardial infarctions), stroke, emphysema, hemorrhagic shock, peripheral vascular disease (upper and lower extremities) and transplant related injuries.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. By way of example, the normal amount of perfusion to brain gray matter in humans is about 60 to 70 mL/100 g of brain tissue/min. Death of central nervous system cells typically occurs when the flow of blood falls below approximately 8-10 mL/100 g of brain tissue/min, while at slightly higher levels (i.e. 20-35 mL/100 g of brain tissue/min) the tissue remains alive but not able to function. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a CLK-inhibiting compound to a subject to treat a central nervous system ischemic condition. A number of central nervous system ischemic conditions may be treated by the CLK-inhibiting compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. Generally speaking, brain stem strokes strike the brain stem, which control involuntary life-support functions such as breathing, blood pressure, and heartbeat. In another alternative of this embodiment, the stroke is a cerebellar stroke. Typically, cerebellar strokes impact the cerebellum area of the brain, which controls balance and coordination. In still another embodiment, the stroke is an embolic stroke. In general terms, embolic strokes may impact any region of the brain and typically result from the blockage of an artery by a vaso-occlusion. In yet another alternative, the stroke may be a hemorrhagic stroke. Like ischemic strokes, hemorrhagic stroke may impact any region of the brain, and typically result from a ruptured blood vessel characterized by a hemorrhage (bleeding) within or surrounding the brain. In a further embodiment, the stroke is a thrombotic stroke. Typically, thrombotic strokes result from the blockage of a blood vessel by accumulated deposits.

In another embodiment, the ischemic condition may result from a disorder that occurs in a part of the subject's body outside of the central nervous system, but yet still causes a reduction in blood flow to the central nervous system. These disorders may include, but are not limited to a peripheral vascular disorder, a venous thrombosis, a pulmonary embolus, arrhythmia (e.g. atrial fibrillation), a myocardial infarction, a transient ischemic attack, unstable angina, or sickle cell anemia. Moreover, the central nervous system ischemic condition may occur as result of the subject undergoing a surgical procedure. By way of example, the subject may be undergoing heart surgery, lung surgery, spinal surgery, brain surgery, vascular surgery, abdominal surgery, or organ transplantation surgery. The organ transplantation surgery may include heart, lung, pancreas, kidney or liver transplantation surgery. Moreover, the central nervous system ischemic condition may occur as a result of a trauma or injury to a part of the subject's body outside the central nervous system. By way of example, the trauma or injury may cause a degree of bleeding that significantly reduces the total volume of blood in the subject's body. Because of this reduced total volume, the amount of blood flow to the central nervous system is concomitantly reduced. By way of further example, the trauma or injury may also result in the formation of a vaso-occlusion that restricts blood flow to the central nervous system.

Of course it is contemplated that the CLK-inhibiting compounds may be employed to treat the central nervous system ischemic condition irrespective of the cause of the condition. In one embodiment, the ischemic condition results from a vaso-occlusion. The vaso-occlusion may be any type of occlusion, but is typically a cerebral thrombosis or an embolism. In a further embodiment, the ischemic condition may result from a hemorrhage. The hemorrhage may be any type of hemorrhage, but is generally a cerebral hemorrhage or a subarachnoid hemorrhage. In still another embodiment, the ischemic condition may result from the narrowing of a vessel. Generally speaking, the vessel may narrow as a result of a vasoconstriction such as occurs during vasospasms, or due to arteriosclerosis. In yet another embodiment, the ischemic condition results from an injury to the brain or spinal cord.

In yet another aspect, a CLK-inhibiting compound may be administered to reduce infarct size of the ischemic core following a central nervous system ischemic condition. Moreover, a CLK-inhibiting compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include one or more CLK-inhibiting compounds and one or more anti-neurodegeneration agents. For example, one or more CLK-inhibiting compounds can be combined with an effective amount of one or more of: L-DOPA; a dopamine agonist; an adenosine A_{2A} receptor antagonist; a COMT inhibitor; a MAO inhibitor; an N—NOS inhibitor; a sodium channel antagonist; a selective N-methyl D-aspartate (NMDA) receptor antagonist; an AMPA/kainate receptor antagonist; a calcium channel antagonist; a GABA-A receptor agonist; an acetyl-choline esterase inhibitor; a matrix metalloprotease inhibitor; a PARP inhibitor; an inhibitor of p38 MAP kinase or c-jun-N-terminal kinases; TPA; NDA antagonists; beta-interferons; growth factors; glutamate inhibitors; and/or as part of a cell therapy.

Exemplary N—NOS inhibitors include 4-(6-amino-pyridin-2-yl)-3-methoxyphenol 6-[4-(2-dimethylamino-ethoxy)-2-methoxy-phenyl]-pyridin-2-yl-amine, 6-[4-(2-dimethylamino-ethoxy)-2, 3-dimethyl-phenyl]-pyridin-2-yl-amine, 6-[4-(2-pyrrolidinyl-ethoxy)-2, 3-dimethyl-phenyl]-pyridin-2-yl-amine, 6-[4-(4-(n-methyl)piperidinyloxy)-2, 3-dimethyl-phenyl]-pyridin-2-yl-amine, 6-[4-(2-dimethylamino-ethoxy)-3-methoxy-phenyl]-pyridin-2-yl-amine, 6-[4-(2-pyrrolidinyl-ethoxy)-3-methoxy-phenyl]-pyridin-2-yl-amine, 6-{4-[2-(6,7-dimethoxy-3,4-dihydro-1h-isoquinolin-2-yl)-ethoxy]-3-methoxy-phenyl}-pyridin-2-yl-amine, 6-{3-methoxy-4-[2-(4-phenethyl-piperazin-1-yl)-ethoxy]-phenyl}-pyridin-2-yl-amine, 6-{3-methoxy-4-[2-(4-methyl-piperazin-1-yl)-ethoxy]-phenyl}-pyridin-2-yl-amine, 6-{4-[2-(4-dimethylamino-piperidin-1-yl)-ethoxy]-3-methoxy-phenyl}-pyridin-2-yl-amine, 6-[4-(2-dimethylamino-ethoxy)-3-ethoxy-phenyl]-pyridin-2-yl-amine, 6-[4-(2-pyrrolidinyl-ethoxy)-3-ethoxy-phenyl]-pyridin-2-yl-amine, 6-[4-(2-dimethylamino-ethoxy)-2-isopropyl-phenyl]-pyridin-2-yl-amine, 4-(6-amino-pyridin-yl)-3-cyclopropyl-phenol 6-[2-cyclopropyl-4-(2-dimethylamino-ethoxy)-phenyl]-pyridin-2-yl-amine, 6-[2-cyclopropyl-4-(2-pyrrolidin-1-yl-ethoxy)-phenyl]-pyridin-2-yl-amine, 3-[3-(6-amino-pyridin-2-yl)-4-cyclopropyl-phenoxy]-pyrrolidine-1-carboxylic acid tert-butyl ester 6-[2-cyclopropyl-4-(1-methyl-pyrrolidin-3-yl-oxy)-phenyl]-pyridin-2-yl-amine, 4-(6-amino-pyridin-2-yl)-3-cyclobutyl-phenol 6-[2-cyclobutyl-4-(2-dimethylamino-ethoxy)-phenyl]-pyridin-2-yl-amine, 6-[2-cyclobutyl-4-(2-pyrrolidin-1-yl-ethoxy)-phenyl]-pyridin-2-yl-amine, 6-[2-cyclobutyl-4-(1-methyl-pyrrolidin-3-yl-oxy)-phenyl]-pyridin-2-yl-amine, 4-(6-amino-pyridin-2-yl)-3-cyclopentyl-phenol 6-[2-cyclopentyl-4-(2-dimethylamino-ethoxy)-phenyl]-pyrid-in-2-yl-amine, 6-[2-cyclopentyl-4-(2-pyrrolidin-1yl-ethoxy)-phenyl]-pyridin-2-yl-amine, 3-[4-(6-amino-pyridin-2-yl)-3-methoxy-phenoxy]-pyrrolidine-1-carboxylic acid tert butyl ester 6-[4-(1-methyl-pyrrolidin-3-yl-oxy)-2-methoxy-phenyl]-pyridin-2-yl-amine, 4-[4-(6-amino-pyridin-2-yl)-3-methoxy-phenoxy-]-piperidine-1-carboxylic acid tert butyl ester 6-[2-methoxy-4-(1-methyl-piperidin-4-yl-oxy)-phenyl]-pyridin-2-yl-amine, 6-[4-(allyloxy)-2-methoxy-phenyl]-pyridin-2-yl-amine, 4-(6-amino-pyridin-2-yl)-3-methoxy-6-allyl-phenol 12 and 4-(6-amino-pyridin-2-yl)-3-methoxy-2-allyl-phenol 13 4-(6-amino-pyridin-2-yl)-3-methoxy-6-propyl-phenol 6-[4-(2-dimethylamino-ethoxy)-2-methoxy-5-propyl-phenyl]-pyridin-yl-amine, 6-[2-isopropyl-4-(pyrrolidin-3-yl-oxy)-phenyl]-pyridin-2-yl-amine, 6-[2-isopropyl-4-(piperidin-3-yl-oxy)-phenyl]-pyridin-2-yl-amine, 6-[2-isopropyl-4-(1-methyl-azetidin-3-yl-oxy)-phenyl]-pyridin-2-yl-amine, 6-[2-isopropyl-4-(1-methyl-piperidin-4-yl-oxy)-phenyl]-pyridin-2-yl-amine, 6-[2-isopropyl-4-(1-methyl-pyrrolidin-3-yl-oxy)-phenyl]-pyridin-2-yl-amine 6-[2-isopropyl-4-(1-methyl-pyrrolidin-3-yl-oxy)-phenyl]-pyridin-2-yl-amine, 6-[2-isopropyl-4-(2-methyl-2-aza-bicyclo[2.2.1]hept-5-yl-oxy)-phenyl]-pyridin-2-yl-amine, 6-[4-(2-dimethylamino-ethoxy)-2-methoxy-phenyl]-pyridin-2-yl-amine, 6-{4-[2-(benzyl-methyl-amino)-ethoxy]-2-methoxy-phenyl}-pyridin-2-yl-amine, 6-[2-methoxy-4-(2-pyrrolidin-1-yl-ethoxy)-phenyl]-pyridin-2-yl-amine, 2-(6-amino-pyridin-2-yl)-5-(2-dimethylamino-ethoxy)-phenol 2-[4-(6-amino-pyridin-2-yl)-3-methoxy-phenoxy]-acetamide 6-[4-(2-amino-ethoxy)-2-methoxy-phenyl]-pyridin-2-yl-amine, 6-{4-[2-(3,4-dihydro-1h-isoquinolin-2-yl)-ethoxy]-2-methoxy-phenyl}-pyrid-in-2-yl-amine, 2-[4-(6-amino-pyridin-2-yl)-3-methoxy-phenoxy]-ethanol 6-{2-methoxy-4-[2-(2,2,6,6-tetramethyl-piperidin-1-yl)-ethoxy]-phenyl}-pyridin-2-yl-amine, 6-{4-[2-(2,5-dimethyl-pyrrolidin-1-yl)-ethoxy]-2-methoxy-phenyl}-pyridin-2-yl-amine, 6-{4-[2-(2,5-dimethyl-pyrrolidin-1-yl)-ethoxy]-2-methoxy-phenyl}-pyridin-2-yl-amine, 2-[4-(6-amino-pyridin-2-yl)-3-methoxy-phenoxy]-1-(2,2,6,6-tetramethyl-piperidin-1-yl)-ethanone 6-[2-methoxy-4-(1-methyl-pyrrolidin-2-yl-methoxy)-phenyl]-pyridin-2-yl-amine, 6-[4-(2-dimethylamino-ethoxy)-2-propoxy-phenyl]-pyridin-2-yl-amine, 6-{4-[2-(benzyl-methyl-amino)-ethoxy]-2-propoxy-phenyl}-pyridin-2-yl-amine 6-[4-(2-ethoxy-ethoxy)-2-methoxy-phenyl]-pyridin-2-yl-amine, 6-[4-(2-dimethylamino-ethoxy)-2-isopropoxy-phenyl]-pyridin-2-yl-amine, 6-[4-(2-ethoxy-ethoxy)-2-isopropoxy-phenyl]-pyridin-2-yl-amine, 6-[2-methoxy-4-(3-methyl-butoxy)-phenyl]-pyridin-2-yl-amine, 6-[4-(2-dimethylamino-ethoxy)-2-ethoxy-phenyl]-pyridin-2-yl-amine, 6-{4-[2-(benzyl-methyl-amino)-ethoxy]-2-ethoxy-phenyl}-pyridin-2-yl-amine, 6-[2-ethoxy-4-(3-methyl-butoxy)-phenyl]-pyridin-2-yl-amine, 1-(6-amino-3-aza-bicyclo[3.1.0]hex-3-yl)-2-[4-(6-amino-pyridin-2-yl)-3-ethoxy-phenoxy]-ethanone 6-[2-ethoxy-4-(2-pyrrolidin-1-yl-ethoxy)-phenyl]-pyridin-2-yl-amine, 3-{2-[4-(6-amino-pyridin-2-yl)-3-ethoxy-phenoxy]-ethyl}-3-aza-bicyclo[3.1.0]hex-6-yl-amine, 1-(6-amino-3-aza-bicyclo[3.1.0]hex-3-yl)-2-[4-(6-amino-pyridin-2-yl)-3-methoxy-phenoxy]-ethanone 3-{2-[4-(6-amino-pyridin-2-yl)-3-methoxy-phenoxy]-ethyl}-3-aza-bicyclo[3.-1.0]hex-6-yl-amine, 6-[2-isopropoxy-4-(2-pyrrolidin-1-yl-ethoxy)-phenyl]-pyridin-2-yl-amine, 6-{4-[2-(benzyl-methyl-amino)-ethoxy]-2-isopropoxy-phenyl-}-pyridin-2-yl-amine, 6-[4-(2-dimethylamino-ethoxy)-2-methoxy-5-propyl-phenyl]-pyridin-2-yl-amine, 6-[5-allyl-4-(2-dimethylamino-ethoxy)-2-methoxy-phenyl]-pyridin-2-yl-amine, 6-[5-allyl-2-methoxy-4-(2-pyrrolidin-1-yl-ethoxy)-phenyl]-pyridin-2-yl-amine, 6-[3-allyl-4-(2-dimethylamino-ethoxy)-2-methoxy-phenyl]-pyridin-2-yl-amine, 6-[2-methoxy-4-(pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-ethoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(piperidin-4-yl-oxy)-phenyl]-pyridin-2-yl-amine, 6-[2-methoxy-4-(2,2, 6,6-tetramethyl-piperidin-4-yl-oxy)-phenyl]-pyridin-2-yl-amine, 6-[2-isopropoxy-4-(pyrrolidin-3-yl-oxy)-phenyl]-pyridin-2-yl-amine, 3-[4-(6-amino-pyridin-2-yl)-3-methoxy-phenoxy]-azetidine-1-carboxylic acid tert-butyl ester 6-[4-(azetidin-3-yl-oxy)-2-methoxy-phenyl]-pyridin-2-yl-amine, 6-[2-methoxy-4-(1-methyl-azetidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-methoxy-4-(2-methyl-2-aza-bicyclo[2.2.1]hept-5-yl-oxy)-phenyl]-pyrid-in-2-yl-amine, 6-[2-methoxy-4-(1-methyl-piperidin-4-yl-oxy)-phenyl]-pyridin-2-yl-amine, 6-[4-(1-ethyl-piperidin-4-yl-oxy)-2-methoxy-phenyl]-pyridin-2-yl-amine, 6-[5-allyl-2-methoxy-4-(1-methyl-pyrrolidin-3-yl-oxy)-phenyl]-pyridin-2-yl-amine, 6-[4-(2-dimethylamino-ethoxy)-2,6-dimethyl-phenyl]-pyridin-2-yl-amine, 6-[2,6-dimethyl-4-(3-piperidin-1-yl-propoxy)-phenyl]-pyridin-2-yl-amine, 6-[2,6-dimethyl-4-(2-pyrrolidin-1-yl-ethoxy)-phenyl]-pyridin-2-yl-amine, 6-{2,6-dimethyl-4-[3-(4-methyl-piperazin-1-yl)-propoxy]-phenyl}-pyridin-2-yl-amine, 6-[2,6-dimethyl-4-(2-morpholin-4-yl-ethoxy)-phenyl]-pyrid-in-2-yl-amine, 6-{4-[2-(benzyl-methyl-amino)-ethoxy]-2,6-dimethyl-phenyl}-pyridin-2-yl-amine, 2-[4-(6-amino-pyridin-2-yl)-3,5-dimethyl-phenoxy]-acetamide 6-[4-(2-amino-ethoxy)-2,6-dimethyl-phenyl]-pyridin-2-yl-amine, 6-[2-isopropyl-4-(2-pyrrolidin-1-yl-ethoxy)-phenyl]-pyridin-2-yl-amine, 2-(2,5-dimethyl-pyrrolidin-1-yl)-6-[2-isopropyl-4-(2-pyrrolidin-1-yl-ethoxy)-phenyl]-pyridine 6-{4-[2-(3,5-dimethyl-piperidin-1-yl)-ethoxy]-2-isopropyl-phenyl}-pyridin-2-yl-amine, 6-[4-(2-dimethylamino-ethoxy)-2-isopropyl-phenyl]-pyridin-2-yl-amine, 6-[2-tert-butyl-4-(2-dimethylamino-ethoxy)-phenyl]-pyridin-2-yl-amine, 6-[2-tert-butyl-4-(2-pyrrolidin-1-yl-ethoxy)-phenyl-]-pyridin-2-yl-amine, 6-[4-(2-pyrrolidinyl-ethoxy)-2, 5-dimethyl-phenyl]-pyridin-2-yl-amine, 6-[4-(2-dimethylamino-ethoxy)-2, 5-dimethyl-phenyl]-pyridin-2-yl-amine, 6-[4-(2-(4-phenethylpiperazin-1-yl)-ethoxy)-2, 5-dimethyl-phenyl]-pyridin-2-yl-amine, 6-[2-cyclopropyl-4-(2-dimethylamino-1-methyl-ethoxy)-phenyl]-pyridin-2-yl-amine, 6-[cyclobutyl-4-(2-dimethylamino-1-methyl-ethoxy)-phenyl]-pyridin-2-yl-amine, 6-[4-(allyloxy)-2-cyclobutyl-phenyl]-pyridin-2-ylamine, 2-allyl-4-(6-amino-pyridin-2-yl)-3-cyclobutyl-phenol and 2-allyl-4-(6-amino-pyridin-2-yl)-5-cyclobutyl-phenol 4-(6-amino-pyridin-2-yl)-5-cyclobutyl-2-propyl-phenol 4-(6-amino-pyridin-2-yl)-3-cyclobutyl-2-propyl-phenol 6-[2-cyclobutyl-4-(2-dimethylamino-1-methyl-ethoxy)-5-propyl-phenyl]-pyridin-2-yl-amine, 6-[2-cyclobutyl-4-(2-dimethylamino-1-methyl-ethoxy)-3-propyl-phenyl]-pyridin-2-yl-amine, 6-[2-cyclobutyl-4-(2-dimethylamino-ethoxy)-5-propyl-phenyl]-pyridin-2-yl-amine, 6-[2-cyclobutyl-4-(2-dimethylamino-ethoxy)-3-propyl-phenyl]-pyridin-2-yl-amine, 6-[2-cyclobutyl-4-(1-methyl-pyrrolidin-3-yl-oxy)-5-propyl-phenyl]-pyridin-2-yl-amine, 6-[cyclobutyl-4-(1-methyl-1-pyrrolidin-3-yl-oxy)-3-propyl-phenyl]-pyridin-2-yl-amine, 2-(4-benzyloxy-5-hydroxy-2-methoxy-phenyl)-6-(2,5-dimethyl-pyrrol-1-yl)-pyridine 6-[4-(2-dimethylamino-ethoxy)-5-ethoxy-2-methoxy-phenyl]-pyridin-2-yl-amine, 6-[5-ethyl-2-methoxy-4-(1-methyl-piperidin-4-yl-oxy)-phenyl]-pyridin-2-yl-amine, 6-[5-ethyl-2-methoxy-4-(piperidin-4-yl-oxy)-phenyl]-pyridin-2-yl-amine, 6-[2,5-dimethoxy-4-(1-methyl-pyrrolidin-3-yl-oxy)-phenyl]-pyridin-2-yl-amine, 6-[4-(2-dimethylamino-ethoxy)-5-ethyl-2-methoxy-phenyl]-pyridin-2-yl-amine.

Exemplary NMDA receptor antagonist include (+)-(1S,2S)-1-(4-hydroxy-phenyl)-2-(4-hydroxy-4-phenylpiperidino)-1-propanol, (1S,2S)-1-(4-hydroxy-3-methoxyphenyl)-2-(4-hydroxy-4-phenylpiperidino)-1-propanol, (3R,4S)-3-(4-(4-fluorophenyl)-4-hydroxypiperidin-1-yl-)-chroman-4,7-diol, (1R*, 2R*)-1-(4-hydroxy-3-methylphenyl)-2-(4-(4-fluoro-phenyl)-4-hydroxypiperidin-1-yl)-propan-1-ol-mesylate or a pharmaceutically acceptable acid addition salt thereof.

Exemplary dopamine agonist include ropininole; L-dopa decarboxylase inhibitors such as carbidopa or benserazide, bromocriptine, dihydroergocryptine, etisulergine, AF-14, alaptide, pergolide, piribedil; dopamine D1 receptor agonists such as A-68939, A-77636, dihydrexine, and SKF-38393; dopamine D2 receptor agonists such as carbergoline, lisuride, N-0434, naxagolide, PD-118440, pramipexole, quinpirole and ropinirole; dopamine/β-adrenegeric receptor agonists such as DPDMS and dopexamine; dopamine/5-HT uptake inhibitor/5-HT-1A agonists such as roxindole; dopamine/opiate receptor agonists such as NIH-10494; α2-adrenergic antagonist/dopamine agonists such as terguride; α2-adrenergic antagonist/dopamine D2 agonists such as ergolines and talipexole; dopamine uptake inhibitors such as GBR-12909, GBR-13069, GYKI-52895, and NS-2141; monoamine oxidase-B inhibitors such as selegiline, N-(2-butyl)-N-methylpropargylamine, N-methyl-N-(2-pentyl)propargylamine, AGN-1133, ergot derivatives, lazabemide, LU-53439, MD-280040 and mofegiline; and COMT inhibitors such as CGP-28014.

Exemplary acetyl cholinesterase inhibitors include donepizil, 1-(2-methyl-1H-benzimidazol-5-yl)-3-[1-(phenylmethyl)-4-piperidinyl]-1-propanone; 1-(2-phenyl-1H-benzimidazol-5-yl)-3-[1-(phenylmethyl)-4-piperidinyl]-1-propanone; 1-(1-ethyl-2-methyl-1H -benzimidazol-5-yl)-3-[1-(phenylmethyl)-4-piperidinyl]-1-propanone; 1-(2-methyl-6-benzothiazolyl)-3-[1-(phenylmethyl)-4-piperidinyl]-1-propanone; 1-(2-methyl-6-benzothiazolyl)-3-[1-[(2-methyl-4-thiazolyl)methyl]-4-piperidinyl]-1-propanone; 1-(5-methyl-benzo[b]thien-2-yl)-3-[1-(phenylmethyl)-4-piperidinyl]-1-propanone; 1-(6-methyl-benzo[b]thien-2-yl)-3-[1-(phenylmethyl)-4-piperidinyl]-1-propanone; 1-(3,5-dimethyl-benzo[b]thien-2-yl)-3-[1-(phenylmethyl)-4-piperidinyl]-1-propanone; 1-(benzo[b]thien-2-yl)-3-[1-(phenylmethyl)-4-piperidinyl]-1-propanone; 1-(benzofuran-2-yl)-3-[1-(phenylmethyl)-4-piperidinyl]-1-propanone; 1-(1-phenylsulfonyl-6-methyl-indol-2-yl)-3-[1-(phenylmethyl)-4-piperidinyl]-1-propanone; 1-(6-methyl-indol-2-yl)-3-[1-(phenylmethyl)-4-piperidinyl]-1-propanone; 1-(1-phenylsulfonyl-5-amino-indol-2-yl)-3-[1-(phenylmethyl)-4-piperidinyl]-1-propanone; 1-(5-amino-indol-2-yl)-3-[1-(phenylmethyl)-4-piperidinyl]-1-propanone; and 1-(5-acetylamino-indol-2-yl)-3-[1-(phenylmethyl)-4-piperidinyl]-1-propanone. 1-(6-quinolyl)-3-[1-(phenylmethyl)-4-piperidinyl]-1-propanone; 1-(5-indolyl)-3-[1-(phenylmethyl)-4-piperidinyl]-1-propanone; 1-(5-benzthienyl)-3-[1-(phenylmethyl)-4-piperidinyl]-1-propanone; 1-(6-quinazolyl)-3-[1-(phenylmethyl)-4-piperidinyl]-1-propanone; 1-(6-benzoxazolyl)-3-[1-(phenylmethyl)-4-piperidinyl]-1-propanone; 1-(5-benzofuranyl)-3-[1-(phenylmethyl)-4-piperidinyl]-1-propanone; 1-(5-methyl-benzimidazol-2-yl)-3-[1-(phenylmethyl)-4-piperidinyl]-1-propanone; 1-(6-methyl-benzimidazol-2-yl)-3-[1-(phenylmethyl)-4-piperidinyl]-1-propanone; 1-(5-chloro-benzo[b]thien-2-yl)-3-[1-(phenylmethyl)-4-piperidinyl]-1-propanone; 1-(5-azaindol-2-yl)-3-[1-(phenylmethyl)-4-piperidinyl]-1-propanone; 1-(6-azabenzo[b]thien-2-yl)-3-[1-(phenylmethyl)-4-piperidinyl]-1-propanone; 1-(1H-2-oxo-pyrrolo[2′,3′,5,6]benzo[b]thieno-2-yl)-3-[1-(phenylmethyl)-4-piperidinyl]-1-propanone; 1-(6-methyl-benzothiazol-2-yl)-3-[1-(phenylmethyl)-4-piperidinyl]-1-propanone; 1-(6-methoxy-indol-2-yl)-3-[1-(phenylmethyl)-4-piperidinyl]-1-propanone; 1-(6-methoxy-benzo[b]thien-2-yl)-3-[1-(phenylmethyl)-4-piperidinyl]-1-propanone; 1-(6-acetylamino-benzo[b]thien-2-yl)-3-[1-(phenylmethyl)-4-piperidinyl]-1-propanone; 1-(5-acetylamino-benzo[b]thien-2-yl)-3-[1-(phenylmethyl-)-4-piperidinyl]-1-propanone; 6-hydroxy-3-[2-[1-(phenylmethyl)-4-piperidinyl]ethyl]-1,2-benzisoxazole; 5-methyl-3-[2-[1-(phenylmethyl)-4-piperidinyl-]ethyl]-1,2-benzisoxazole; 6-methoxy-3 [2-[1(phenylmethyl)-4-piperidinyl]ethyl]-1,2-benzisoxazole; 6-acetamide-3-[2-[1-(phenylmethyl)-4-piperidinyl]-ethyl]-1,2-benzisoxazole; 6-amino-3-[2-[1-(phenylmethyl)-4-piperidinyl]ethyl-1]-1,2-benzisoxazole; 6-(4-morpholinyl)-3-[2-[1-(phenylmethyl)-4-piperidinyl]ethyl]-1,2-benzisoxazole; 5,7-dihydro-3-[2-[1-(phenylmethyl)-4-piperidinyl]ethyl]-6H-pyrrolo[4,5-f]-1,2-benzisoxazol-6-one; 3-[2-[1-(phenylmethyl)-4-piperidinyl]ethyl]-1,2-benzisothiazole; 3-[2-[1-(phenylmethyl)-4-piperidinyl]ethenyl]-1, 2-benzisoxazole; 6-phenylamino-3-[2-[1-(phenylmethyl)-4-piperidinyl]ethyl]-1,2,-benzisoxazole; 6-(2-thiazoly)-3-[2-[1-(phenylmethyl)-4-piperidinyl]ethyl]-1,2-benzisoxazole; 6-(2-oxazolyl)-3-[2-[1-(phenylmethyl)-4-piperidinyl]ethyl]-1,2-benzisoxazole; 6-pyrrolidinyl-3-[2-[1-(phenylmethyl)-4-piperidinyl]ethyl]-1,-2-benzisoxazole; 5,7-dihydro-5,5-dimethyl-3-[2-[1-(phenylmethyl)-4-piperidinyl]ethyl]-6H-pyrrolo[4,5-f]-1,2-benzisoxazole-6-one; 6,8-dihydro-3-[2-[1-(phenylmethyl)-4-piperidinyl]ethyl]-7H-pyrrolo[5,4-g]-1,2-benzisoxazole-7-one; 3-[2-[1-(phenylmethyl)-4-piperidinyl]ethyl]-5,6,-8-trihydro-7H-isoxazolo[4,5-g]-quinolin-7-one; 1-benzyl-4-((5,6-dimethoxy-1-indanon)-2-yl)methylpiperidine, 1-benzyl-4-((5,6-dimethoxy-1-indanon)-2-ylidenyl)methylpiperidine, 1-benzyl-4-((5-methoxy-1-indanon)-2-yl)methylpiperidine, 1-benzyl-4-((5,6-diethoxy-1-indanon)-2-yl)methylpiperidine, 1-benzyl-4-((5,6-methylenedioxy-1-indanon)-2-yl)methylpiperidine, 1-(m-nitrobenzyl)-4-((5,6-dimethoxy-1-indanon)-2-yl)methylpiperidine, 1-cyclohexymethyl-4-((5,6-dimethoxy-1-indanon)-2-yl)methylpiperidine, 1-(m-fluorobenzyl)-4-((5,6-dimethoxy-1-indanon)-2-yl)methylpiperidine, 1-benzyl-4-((5,6-dimethoxy-1-indanon)-2-yl)propylpiperidine, and 1-benzyl-4-((5-isopropoxy-6-methoxy-1-indanon)-2-yl)methylpiperidine.

Exemplary calcium channel antagonists include diltiazem, omega-conotoxin GVIA, methoxyverapamil, amlodipine, felodipine, lacidipine, and mibefradil.

Exemplary GABA-A receptor modulators include clomethiazole; IDDB; gaboxadol (4,5,6,7-tetrahydroisoxazolo[5,4-c]pyridin-3-ol); ganaxolone (3.alpha.-hydroxy-3.beta.-methyl-5.alpha.-pregnan-20-one); fengabine (2-[(butylimino)-(2-chlorophenyl)methyl]-4-chlorophenol); 2-(4-methoxyphenyl)-2,5,6,7,8,9-hexahydro-pyrazolo[4,3-c]cinnolin-3-one; 7-cyclobutyl-6-(2-methyl-2H-1,2,4-triazol-3-ylmethoxy)-3-phenyl-1,2,4-triazolo[4,3-b]pyridazine; (3-fluoro-4-methylphenyl)-N-({-1-[(2-methylphenyl)methyl]-benzimidazol-2-yl}methyl)-N-pentylcarboxamide; and 3-(aminomethyl)-5-methylhexanoic acid.

Exemplary potassium channel openers include diazoxide, flupirtine, pinacidil, levcromakalim, rilmakalim, chromakalim, PCO-400 and SKP-450 (2-[2″(1″, 3″-dioxolone)-2-methyl]-4-(2′-oxo-1′-pyrrolidinyl)-6-nitro-2H-1-benzopyran).

Exemplary AMPA/kainate receptor antagonists include 6-cyano-7-nitroquinoxalin-2,3-di-one (CNQX); 6-nitro-7-sulphamoylbenzo[f]quinoxaline-2,3-dione (NBQX); 6,7-dinitroquinoxaline-2,3-dione (DNQX); 1-(4-aminophenyl)-4-methyl-7,8-m-ethylenedioxy-5H-2,3-benzodiazepine hydrochloride; and 2,3-dihydroxy-6-nitro-7-sulfamoylbenzo-[f]quinoxaline.

Exemplary sodium channel antagonists include ajmaline, procainamide, flecainide and riluzole.

Exemplary matrix-metalloprotease inhibitors include 4-[4-(4-fluorophenoxy)benzenesulfonylamino]tetrahydropyran-4-carboxylic acid hydroxyamide; 5-Methyl-5-(4-(4′-fluorophenoxy)-phenoxy)-pyrimidine-2,4,6-trione; 5-n-Butyl-5-(4-(4′-fluorophenoxy)-phenoxy)-pyrimidine-2,4,6-trione and prinomistat.

Poly(ADP ribose) polymerase (PARP) is an abundant nuclear enzyme which is activated by DNA strand single breaks to synthesize poly (ADP ribose) from NAD. Under normal conditions, PARP is involved in base excision repair caused by oxidative stress via the activation and recruitment of DNA repair enzymes in the nucleus. Thus, PARP plays a role in cell necrosis and DNA repair. PARP also participates in regulating cytokine expression that mediates inflammation. Under conditions where DNA damage is excessive (such as by acute excessive exposure to a pathological insult), PARP is over-activated, resulting in cell-based energetic failure characterized by NAD depletion and leading to ATP consumption, cellular necrosis, tissue injury, and organ damage/failure. PARP is thought to contribute to neurodegeneration by depleting nicotinamide adenine dinucleotide (NAD+) which then reduces adenosine triphosphate (ATP; Cosi and Marien, Ann. N.Y. Acad. Sci., 890:227, 1999) contributing to cell death which can be prevented by PARP inhibitors. Exemplory PARP inhibitors can be found in Southan and Szabo, Current Medicinal Chemistry, 10:321, 2003.

Exemplary inhibitors of p38 MAP kinase and c-jun-N-terminal kinases include pyridyl imidazoles, such as PD 169316, isomeric PD 169316, SB 203580, SB 202190, SB 220026, and RWJ 67657. Others are described in U.S. Pat. No. 6,288,089, and incorporated by reference herein.

In an exemplary embodiment, a combination therapy for treating or preventing MS comprises a therapeutically effective amount of one or more CLK-inhibiting compounds and one or more of Avonex® (interferon beta-1a), Tysabri® (natalizumab), or Fumaderm® (BG-12/Oral Fumarate).

In another embodiment, a combination therapy for treating or preventing diabetic neuropathy or conditions associated therewith comprises a therapeutically effective amount of one or more CLK-inhibiting compounds and one or more of tricyclic antidepressants (TCAs) (including, for example, imipramine, amytriptyline, desipramine and nortriptyline), serotonin reuptake inhibitors (SSRIs) (including, for example, fluoxetine, paroxetine, sertralene, and citalopram) and antiepileptic drugs (AEDs) (including, for example, gabapentin, carbamazepine, and topimirate).

In another embodiment, the invention provides a method for treating or preventing a polyglutamine disease using a combination comprising at least one CLK-inhibiting compound and at least one HDAC I/II inhibitor. Examples of HDAC I/II inhibitors include hydroxamic acids, cyclic peptides, benzamides, short-chain fatty acids, and depudecin.

Examples of hydroxamic acids and hydroxamic acid derivatives, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), valproic acid and pyroxamide. TSA was isolated as an antifungi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature: a polar hydroxamic terminal linked through a hydrophobic methylene spacer (e.g. 6 carbon at length) to another polar site which is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and depsipeptide. Trapoxin A is a cyclic tetrapeptide that contains a 2-amino-8-oxo-9,10-epoxy-decanoyl (AOE) moiety. Kijima et al. (1993) J. Biol. Chem. 268:22429-22435. Apicidin is a fungal metabolite that exhibits potent, broad-spectrum antiprotozoal activity and inhibits HDAC activity at nanomolar concentrations. Darkin-Rattray et al. (1996) Proc. Natl. Acad. Sci. USA. 93; 13143-13147. Depsipeptide is isolated from Chromobacterium violaceum, and has been shown to inhibit HDAC activity at micromolar concentrations.

Examples of benzamides include but are not limited to MS-27-275. Saito et al. (1990) Proc. Natl. Acad. Sci. USA. 96:4592-4597. Examples of short-chain fatty acids include but are not limited to butyrates (e.g., butyric acid, arginine butyrate and phenylbutyrate (PB)). Newmark et al. (1994) Cancer Lett. 78:1-5; and Carducci et al. (1997) Anticancer Res. 17:3972-3973. In addition, depudecin which has been shown to inhibit HDAC at micromolar concentrations (Kwon et al. (1998) Proc. Natl. Acad. Sci. USA. 95:3356-3361) also falls within the scope of histone deacetylase inhibitor as described herein.

v. Blood Coagulation Disorders

In other aspects, CLK-inhibiting compounds can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. After initiation of clotting, blood coagulation proceeds through the sequential activation of certain plasma proenzymes to their enzyme forms (see, for example, Coleman, R. W. et al. (eds.) Hemostasis and Thrombosis, Second Edition, (1987)). These plasma glycoproteins, including Factor XII, Factor XI, Factor IX, Factor X, Factor VII, and prothrombin, are zymogens of serine proteases. Most of these blood clotting enzymes are effective on a physiological scale only when assembled in complexes on membrane surfaces with protein cofactors such as Factor VIII and Factor V. Other blood factors modulate and localize clot formation, or dissolve blood clots. Activated protein C is a specific enzyme that inactivates procoagulant components. Calcium ions are involved in many of the component reactions. Blood coagulation follows either the intrinsic pathway, where all of the protein components are present in blood, or the extrinsic pathway, where the cell-membrane protein tissue factor plays a critical role. Clot formation occurs when fibrinogen is cleaved by thrombin to form fibrin. Blood clots are composed of activated platelets and fibrin.

Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place. It involves a cascade of complicated and regulated biochemical reactions between circulating blood proteins (coagulation factors), blood cells (in particular platelets), and elements of an injured vessel wall.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering a CLK-inhibiting compound. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. Examples of thrombotic disorders include, but are not limited to, thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, e.g., unstable angina, disseminated intravascular coagulation, thrombotic thrombocytopenic purpura, cancer metastasis, sickle cell disease, glomerular nephritis, and drug induced thrombocytopenia (including, for example, heparin induced thrombocytopenia). In addition, CLK-inhibiting compounds may be administered to prevent thrombotic events or to prevent re-occlusion during or after therapeutic clot lysis or procedures such as angioplasty or surgery.

In another embodiment, a combination drug regimen may include drugs or compounds for the treatment or prevention of blood coagulation disorders or secondary conditions associated with these conditions. Thus, a combination drug regimen may include one or more CLK-inhibiting compounds and one or more anti-coagulation or anti-thrombosis agents. For example, one or more CLK-inhibiting compounds can be combined with an effective amount of one or more of: aspirin, heparin, and oral Warfarin that inhibits Vit K-dependent factors, low molecular weight heparins that inhibit factors X and II, thrombin inhibitors, inhibitors of platelet GP IIbIIa receptors, inhibitors of tissue factor (TF), inhibitors of human von Willebrand factor, inhibitors of one or more factors involved in hemostasis (in particular in the coagulation cascade). In addition, CLK-inhibiting compounds can be combined with thrombolytic agents, such as t-PA, streptokinase, reptilase, TNK-t-PA, and staphylokinase.

vi. Weight Control

In another aspect, CLK-inhibiting compounds may be used for treating or preventing weight gain or obesity in a subject. For example, CLK-inhibiting compounds may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, CLK-inhibiting compounds may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Stunkard A J, Wadden T A. (Editors) Obesity: theory and therapy, Second Edition. New York: Raven Press, 1993. Finally, patients with AIDS can develop lipodystrophy or insulin resistance in response to combination therapies for AIDS.

In another embodiment, CLK-inhibiting compounds may be used for inhibiting adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used for treating or preventing obesity.

In other embodiments, CLK-inhibiting compounds may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

In other embodiments, a CLK-activating compound may be used to stimulate appetite and/or weight gain. A method may comprise administering to a subject, such as a subject in need thereof, a pharmaceutically effective amount of a CLK-activating compound that increases the level and/or activity of a CLK protein, such as CLK1, CLK2, CLK3 and/or CLK4. A subject in need of such a treatment may be a subject who has cachexia or may be likely to develop cachexia. A combination of agents may also be administered. A method may further comprise monitoring in the subject the state of the disease or activation of CLKs, for example, in adipose tissue.

Methods for stimulating fat accumulation in cells may be used in vitro, to establish cell models of weight gain, which may be used, e.g., for identifying other drugs that prevent weight gain.

Also provided are methods for modulating adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used to modulate obesity. A method for stimulating adipogenesis may comprise contacting a cell with a CLK-activating compound.

In another embodiment, the invention provides methods of decreasing fat or lipid metabolism in a subject by administering a CLK-activating compound. The method includes administering to a subject an amount of a CLK-activating compound, e.g., in an amount effective to decrease mobilization of fat to the blood from WAT cells and/or to decrease fat burning by BAT cells.

Methods for promoting appetite and/or weight gain may include, for example, prior identifying a subject as being in need of decreased fat or lipid metabolism, e.g., by weighing the subject, determining the BMI of the subject, or evaluating fat content of the subject or CLK activity in cells of the subject. The method may also include monitoring the subject, e.g., during and/or after administration of a CLK-activating compound. The administering can include one or more dosages, e.g., delivered in boluses or continuously. Monitoring can include evaluating a hormone or a metabolite. Exemplary hormones include leptin, adiponectin, resistin, and insulin. Exemplary metabolites include triglyercides, cholesterol, and fatty acids.

In one embodiment, a CLK-inhibiting compound may be used to modulate (e.g., decrease) the amount of subcutaneous fat in a tissue, e.g., in facial tissue or in other surface-associated tissue of the neck, hand, leg, or lips. The CLK-inhibiting compound may be used to increase the rigidity, water retention, or support properties of the tissue. For example, the CLK-inhibiting compound can be applied topically, e.g., in association with another agent, e.g., for surface-associated tissue treatment. The CLK-inhibiting compound may also be injected subcutaneously, e.g., within the region where an alteration in subcutaneous fat is desired.

A method for modulating weight may further comprise monitoring the weight of the subject and/or the level of modulation of CLKs, for example, in adipose tissue.

In an exemplary embodiment, CLK-inhibiting compounds may be administered as a combination therapy for treating or preventing weight gain or obesity. For example, one or more CLK-inhibiting compounds may be administered in combination with one or more anti-obesity agents. Exemplary anti-obesity agents include, for example, phenylpropanolamine, ephedrine, pseudoephedrine, phentermine, a cholecystokinin-A agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (leptin), a leptin analog, a leptin receptor agonist, a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin and ciliary neurotrophic factors such as Axokine.

In another embodiment, CLK-inhibiting compounds may be administered to reduce drug-induced weight gain. For example, a CLK-inhibiting compound may be administered as a combination therapy with medications that may stimulate appetite or cause weight gain, in particular, weight gain due to factors other than water retention. Examples of medications that may cause weight gain, include for example, diabetes treatments, including, for example, sulfonylureas (such as glipizide and glyburide), thiazolidinediones (such as pioglitazone and rosiglitazone), meglitinides, nateglinide, repaglinide, sulphonylurea medicines, and insulin; anti-depressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy; lithium carbonate; valproic acid; carbamazepine; chlorpromazine; thiothixene; beta blockers (such as propranolo); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, CLK-inhibiting compounds may be administered as part of a smoking cessation program to prevent weight gain or reduce weight already gained.

vii. Metabolic Disorders/Diabetes

In another aspect, CLK-inhibiting compounds may be used for treating or preventing a metabolic disorder, such as insulin-resistance, a pre-diabetic state, type II diabetes, and/or complications thereof. Administration of a CLK-inhibiting compound may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, CLK-inhibiting compounds may be administered as a combination therapy for treating or preventing a metabolic disorder. For example, one or more CLK-inhibiting compounds may be administered in combination with one or more anti-diabetic agents. Exemplary anti-diabetic agents include, for example, an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a peroxisome proliferator-activated receptor-γ (PPAR-γ) ligand such as troglitazone, rosaglitazone, pioglitazone or GW-1929, a sulfonylurea, glipazide, glyburide, or chlorpropamide wherein the amounts of the first and second compounds result in a therapeutic effect. Other anti-diabetic agents include a glucosidase inhibitor, a glucagon-like peptide-1 (GLP-1), insulin, a PPAR α/γ dual agonist, a meglitimide and an αP2 inhibitor. In an exemplary embodiment, an anti-diabetic agent may be a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrolidine) or MK-04301 from Merck (see e.g., Hughes et al., Biochemistry 38: 11597-603 (1999)).

viii. Inflammatory Diseases

In other aspects, CLK-inhibiting compounds can be used to treat or prevent a disease or disorder associated with inflammation. CLK-inhibiting compounds may be administered prior to the onset of, at, or after the initiation of inflammation. When used prophylactically, the compounds are preferably provided in advance of any inflammatory response or symptom. Administration of the compounds may prevent or attenuate inflammatory responses or symptoms.

Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, CLK-inhibiting compounds may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, CLK-inhibiting compounds may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more CLK-inhibiting compounds may be taken alone or in combination with other compounds useful for treating or preventing inflammation. Exemplary anti-inflammatory agents include, for example, steroids (e.g., cortisol, cortisone, fludrocortisone, prednisone, 6α-methylprednisone, triamcinolone, betamethasone or dexamethasone), nonsteroidal antiinflammatory drugs (NSAIDS (e.g., aspirin, acetaminophen, tolmetin, ibuprofen, mefenamic acid, piroxicam, nabumetone, rofecoxib, celecoxib, etodolac or nimesulide). In another embodiment, the other therapeutic agent is an antibiotic (e.g., vancomycin, penicillin, amoxicillin, ampicillin, cefotaxime, ceftriaxone, cefixime, rifampinmetronidazole, doxycycline or streptomycin). In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine). In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, α-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, carprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide, cyclazocine, deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocortolone, fluoresone, fluorometholone, fluperolone, flupirtine, fluprednidene, fluprednisolone, fluproquazone, flurandrenolide, flurbiprofen, fluticasone, formocortal, fosfosal, gentisic acid, glafenine, glucametacin, glycol salicylate, guaiazulene, halcinonide, halobetasol, halometasone, haloprednone, heroin, hydrocodone, hydrocortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate, myrophine, nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac.

In an exemplary embodiment, a CLK-inhibiting compound may be administered with a selective COX-2 inhibitor for treating or preventing inflammation. Exemplary selective COX-2 inhibitors include, for example, deracoxib, parecoxib, celecoxib, valdecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl)-1H -pyrazol-1-yl]benzenesulfonamide, tert-butyl 1 benzyl-4-[(4-oxopiperidin-1-yl}sulfonyl]piperidine-4-carboxylate, 4-[5-(phenyl)-3-(trifluoromethyl)-1H -pyrazol-1-yl]benzenesulfonamide, salts and prodrugs thereof.

ix. Flushing

In another aspect, CLK-inhibiting compounds may be used for reducing the incidence or severity of flushing and/or hot flashes which are symptoms of a disorder. For instance, the subject method includes the use of CLK-inhibiting compounds, alone or in combination with other agents, for reducing incidence or severity of flushing and/or hot flashes in cancer patients. In other embodiments, the method provides for the use of CLK-inhibiting compounds to reduce the incidence or severity of flushing and/or hot flashes in menopausal and post-menopausal woman.

In another aspect, CLK-inhibiting compounds may be used as a therapy for reducing the incidence or severity of flushing and/or hot flashes which are side-effects of another drug therapy, e.g., drug-induced flushing. In certain embodiments, a method for treating and/or preventing drug-induced flushing comprises administering to a patient in need thereof a formulation comprising at least one flushing inducing compound and at least one CLK-inhibiting compound. In other embodiments, a method for treating drug induced flushing comprises separately administering one or more compounds that induce flushing and one or more CLK-inhibiting compounds, e.g., wherein the CLK-inhibiting compound and flushing inducing agent have not been formulated in the same compositions. When using separate formulations, the CLK-inhibiting compound may be administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, CLK-inhibiting compounds may be used to reduce flushing side effects of a vasodilator or an antilipemic agent (including anticholesteremic agents and lipotropic agents). In an exemplary embodiment, a CLK-inhibiting compound may be used to reduce flushing associated with the administration of niacin.

Nicotinic acid, 3-pyridinecarboxylic acid or niacin, is an antilipidemic agent that is marketed under, for example, the trade names Nicolar®, SloNiacin®, Nicobid® and Time Release Niacin®. Nicotinic acid has been used for many years in the treatment of lipidemic disorders such as hyperlipidemia, hypercholesterolemia and atherosclerosis. This compound has long been known to exhibit the beneficial effects of reducing total cholesterol, low density lipoproteins or “LDL cholesterol,” triglycerides and apolipoprotein a (Lp(a)) in the human body, while increasing desirable high density lipoproteins or “HDL cholesterol”.

Typical doses range from about 1 gram to about 3 grams daily. Nicotinic acid is normally administered two to four times per day after meals, depending upon the dosage form selected. Nicotinic acid is currently commercially available in two dosage forms. One dosage form is an immediate or rapid release tablet which should be administered three or four times per day. Immediate release (“IR”) nicotinic acid formulations generally release nearly all of their nicotinic acid within about 30 to 60 minutes following ingestion. The other dosage form is a sustained release form which is suitable for administration two to four times per day. In contrast to IR formulations, sustained release (“SR”) nicotinic acid formulations are designed to release significant quantities of drug for absorption into the blood stream over specific timed intervals in order to maintain therapeutic levels of nicotinic acid over an extended period such as 12 or 24 hours after ingestion.

As used herein, the term “nicotinic acid” is meant to encompass nicotinic acid or a compound other than nicotinic acid itself which the body metabolizes into nicotinic acid, thus producing essentially the same effect as nicotinic acid. Exemplary compounds that produce an effect similar to that of nicotinic acid include, for example, nicotinyl alcohol tartrate, d-glucitol hexanicotinate, aluminum nicotinate, niceritrol and d, 1-alpha-tocopheryl nicotinate. Each such compound will be collectively referred to herein as “nicotinic acid.”

In another embodiment, the invention provides a method for treating and/or preventing hyperlipidemia with reduced flushing side effects. The method comprises the steps of administering to a subject in need thereof a therapeutically effective amount of nicotinic acid and a CLK-inhibiting compound in an amount sufficient to reduce flushing. In an exemplary embodiment, the nicotinic acid and/or CLK-inhibiting compound may be administered nocturnally.

In another representative embodiment, the method involves the use of CLK-inhibiting compounds to reduce flushing side effects of raloxifene. Raloxifene acts like estrogen in certain places in the body, but is not a hormone. It helps prevent osteoporosis in women who have reached menopause. Osteoporosis causes bones to gradually grow thin, fragile, and more likely to break. Evista slows down the loss of bone mass that occurs with menopause, lowering the risk of spine fractures due to osteoporosis. A common side effect of raloxifene is hot flashes (sweating and flushing). This can be uncomfortable for women who already have hot flashes due to menopause.

In another representative embodiment, the method involves the use of CLK-inhibiting compounds to reduce flushing side effects of antidepressants or anti-psychotic agent. For instance, CLK-inhibiting compounds can be used in conjunction (administered separately or together) with a serotonin reuptake inhibitor, a 5HT2 receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT1A receptor antagonist, a 5HT1D receptor antagonist, a CRF antagonist, a monoamine oxidase inhibitor, or a sedative-hypnotic drug.

In certain embodiments, CLK-inhibiting compounds may be used as part of a treatment with a serotonin reuptake inhibitor (SRI) to reduce flushing. In certain preferred embodiments, the SRI is a selective serotonin reuptake inhibitor (SSRI), such as a fluoxetinoid (fluoxetine, norfluoxetine) or a nefazodonoid (nefazodone, hydroxynefazodone, oxonefazodone). Other exemplary SSRI's include duloxetine, venlafaxine, milnacipran, citalopram, fluvoxamine, paroxetine and sertraline. The CLK-inhibiting compound can also be used as part of a treatment with sedative-hypnotic drug, such as selected from the group consisting of a benzodiazepine (such as alprazolam, chlordiazepoxide, clonazepam, chlorazepate, clobazam, diazepam, halazepam, lorazepam, oxazepam and prazepam), zolpidem, and barbiturates. In still other embodiments, a CLK-inhibiting compound may be used as part of a treatment with a 5-HT1A receptor partial agonist, such as selected from the group consisting of buspirone, flesinoxan, gepirone and ipsapirone. CLK-inhibiting compounds can also used as part of a treatment with a norepinephrine reuptake inhibitor, such as selected from tertiary amine tricyclics and secondary amine tricyclics. Exemplary tertiary amine tricyclic include amitriptyline, clomipramine, doxepin, imipramine and trimipramine. Exemplary secondary amine tricyclic include amoxapine, desipramine, maprotiline, nortriptyline and protriptyline. In certain embodiments, CLK-inhibiting compounds may be used as part of a treatment with a monoamine oxidase inhibitor, such as selected from the group consisting of isocarboxazid, phenelzine, tranylcypromine, selegiline and moclobemide.

In still another representative embodiment, CLK-inhibiting compounds may be used to reduce flushing side effects of chemotherapeutic agents, such as cyclophosphamide, tamoxifen.

In another embodiment, CLK-inhibiting compounds may be used to reduce flushing side effects of calcium channel blockers, such as amlodipine.

In another embodiment, CLK-inhibiting compounds may be used to reduce flushing side effects of antibiotics. For example, CLK-inhibiting compounds can be used in combination with levofloxacin. Levofloxacin is used to treat infections of the sinuses, skin, lungs, ears, airways, bones, and joints caused by susceptible bacteria. Levofloxacin also is frequently used to treat urinary infections, including those resistant to other antibiotics, as well as prostatitis. Levofloxacin is effective in treating infectious diarrheas caused by E. coli, campylobacter jejuni, and shigella bacteria. Levofloxacin also can be used to treat various obstetric infections, including mastitis.

x. Ocular Disorders

One aspect of the present invention is a method for inhibiting, reducing or otherwise treating vision impairment by administering to a patient a therapeutic dosage of a CLK-inhibiting compound, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

Glaucoma describes a group of disorders which are associated with a visual field defect, cupping of the optic disc, and optic nerve damage. These are commonly referred to as glaucomatous optic neuropathies. Most glaucomas are usually, but not always, associated with a rise in intraocular pressure. Exemplary forms of glaucoma include Glaucoma and Penetrating Keratoplasty, Acute Angle Closure, Chronic Angle Closure, Chronic Open Angle, Angle Recession, Aphakic and Pseudophakic, Drug-Induced, Hyphema, Intraocular Tumors, Juvenile, Lens-Particle, Low Tension, Malignant, Neovascular, Phacolytic, Phacomorphic, Pigmentary, Plateau Iris, Primary Congenital, Primary Open Angle, Pseudoexfoliation, Secondary Congenital, Adult Suspect, Unilateral, Uveitic, Ocular Hypertension, Ocular Hypotony, Posner-Schlossman Syndrome and Scleral Expansion Procedure in Ocular Hypertension & Primary Open-angle Glaucoma.

Intraocular pressure can also be increased by various surgical procedures, such as phacoemulsification (i.e., cataract surgery) and implanation of structures such as an artificial lens. In addition, spinal surgeries in particular, or any surgery in which the patient is prone for an extended period of time can lead to increased interoccular pressure.

Optic neuritis (ON) is inflammation of the optic nerve and causes acute loss of vision. It is highly associated with multiple sclerosis (MS) as 15-25% of MS patients initially present with ON, and 50-75% of ON patients are diagnosed with MS. ON is also associated with infection (e.g., viral infection, meningitis, syphilis), inflammation (e.g., from a vaccine), infiltration and ischemia.

Another condition leading to optic nerve damage is anterior ischemic optic neuropathy (AION). There are two types of AION. Arteritic AION is due to giant cell arteritis (vasculitis) and leads to acute vision loss. Non-arteritic AION encompasses all cases of ischemic optic neuropathy other than those due to giant cell arteritis. The pathophysiology of AION is unclear although it appears to incorporate both inflammatory and ischemic mechanisms.

Other damage to the optic nerve is typically associated with demyelination, inflammation, ischemia, toxins, or trauma to the optic nerve. Exemplary conditions where the optic nerve is damaged include Demyelinating Optic Neuropathy (Optic Neuritis, Retrobulbar Optic Neuritis), Optic Nerve Sheath Meningioma, Adult Optic Neuritis, Childhood Optic Neuritis, Anterior Ischemic Optic Neuropathy, Posterior Ischemic Optic Neuropathy, Compressive Optic Neuropathy, Papilledema, Pseudopapilledema and Toxic/Nutritional Optic Neuropathy.

Other neurological conditions associated with vision loss, albeit not directly associated with damage to the optic nerve, include Amblyopia, Bells Palsy, Chronic Progressive External Opthalmoplegia, Multiple Sclerosis, Pseudotumor Cerebri and Trigeminal Neuralgia.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disruption of the macula (e.g., exudative or non-exudative macular degeneration).

Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis. One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a CLK-inhibiting compound.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a CLK-inhibiting compound disclosed herein. Ocular surgeries include cataract, iridotomy and lens replacements.

Another aspect of the invention is the treatment, including inhibition and prophylactic treatment, of age related ocular diseases include cataracts, dry eye, retinal damage and the like, by administering to the subject in need of such treatment a therapeutic dosage of a CLK-inhibiting compound.

The formation of cataracts is associated with several biochemical changes in the lens of the eye, such as decreased levels of antioxidants ascorbic acid and glutathione, increased lipid, amino acid and protein oxidation, increased sodium and calcium, loss of amino acids and decreased lens metabolism. The lens, which lacks blood vessels, is suspended in extracellular fluids in the anterior part of the eye. Nutrients, such as ascorbic acid, glutathione, vitamin E, selenium, bioflavonoids and carotenoids are required to maintain the transparency of the lens. Low levels of selenium results in an increase of free radical-inducing hydrogen peroxide, which is neutralized by the selenium-dependent antioxidant enzyme glutathione peroxidase. Lens-protective glutathione peroxidase is also dependent on the amino acids methionine, cysteine, glycine and glutamic acid.

Cataracts can also develop due to an inability to properly metabolize galactose found in dairy products that contain lactose, a disaccharide composed of the monosaccharide galactose and glucose. Cataracts can be prevented, delayed, slowed and possibly even reversed if detected early and metabolically corrected.

Retinal damage is attributed, inter alia, to free radical initiated reactions in glaucoma, diabetic retinopathy and age-related macular degeneration (AMD). The eye is a part of the central nervous system and has limited regenerative capability. The retina is composed of numerous nerve cells which contain the highest concentration of polyunsaturated fatty acids (PFA) and subject to oxidation. Free radicals are generated by UV light entering the eye and mitochondria in the rods and cones, which generate the energy necessary to transform light into visual impulses. Free radicals cause peroxidation of the PFA by hydroxyl or superoxide radicals which in turn propagate additional free radicals. The free radicals cause temporary or permanent damage to retinal tissue.

Glaucoma is usually viewed as a disorder that causes an elevated intraocular pressure (IOP) that results in permanent damage to the retinal nerve fibers, but a sixth of all glaucoma cases do not develop an elevated IOP. This disorder is now perceived as one of reduced vascular perfusion and an increase in neurotoxic factors. Recent studies have implicated elevated levels of glutamate, nitric oxide and peroxynitrite in the eye as the causes of the death of retinal ganglion cells. Neuroprotective agents may be the future of glaucoma care. For example, nitric oxide synthase inhibitors block the formation of peroxynitrite from nitric oxide and superoxide. In a recent study, animals treated with aminoguanidine, a nitric oxide synthase inhibitor, had a reduction in the loss of retinal ganglion cells. It was concluded that nitric oxide in the eye caused cytotoxicity in many tissues and neurotoxicity in the central nervous system.

Diabetic retinopathy occurs when the underlying blood vessels develop microvascular abnormalities consisting primarily of microaneurysms and intraretinal hemorrhages. Oxidative metabolites are directly involved with the pathogenesis of diabetic retinopathy and free radicals augment the generation of growth factors that lead to enhanced proliferative activity. Nitric oxide produced by endothelial cells of the vessels may also cause smooth muscle cells to relax and result in vasodilation of segments of the vessel. Ischemia and hypoxia of the retina occur after thickening of the arterial basement membrane, endothelial proliferation and loss of pericytes. The inadequate oxygenation causes capillary obliteration or nonperfusion, arteriolar-venular shunts, sluggish blood flow and an impaired ability of RBCs to release oxygen. Lipid peroxidation of the retinal tissues also occurs as a result of free radical damage.

The macula is responsible for our acute central vision and composed of light-sensing cells (cones) while the underlying retinal pigment epithelium (RPE) and choroid nourish and help remove waste materials. The RPE nourishes the cones with the vitamin A substrate for the photosensitive pigments and digests the cones shed outer tips. RPE is exposed to high levels of UV radiation, and secretes factors that inhibit angiogenesis. The choroid contains a dense vascular network that provides nutrients and removes the waste materials.

In AMD, the shed cone tips become indigestible by the RPE, where the cells swell and die after collecting too much undigested material. Collections of undigested waste material, called drusen, form under the RPE. Photoxic damage also causes the accumulation of lipofuscin in RPE cells. The intracellular lipofuscin and accumulation of drusen in Bruch's membrane interferes with the transport of oxygen and nutrients to the retinal tissues, and ultimately leads to RPE and photoreceptor dysfunction. In exudative AMD, blood vessels grow from the choriocapillaris through defects in Bruch's membrane and may grow under the RPE, detaching it from the choroid, and leaking fluid or bleeding.

Macular pigment, one of the protective factors that prevent sunlight from damaging the retina, is formed by the accumulation of nutritionally derived carotenoids, such as lutein, the fatty yellow pigment that serves as a delivery vehicle for other important nutrients and zeaxanthin. Antioxidants such as vitamins C and E, beta-carotene and lutein, as well as zinc, selenium and copper, are all found in the healthy macula. In addition to providing nourishment, these antioxidants protect against free radical damage that initiates macular degeneration.

Another aspect of the invention is the prevention or treatment of damage to the eye caused by stress, chemical insult or radiation, by administering to the subject in need of such treatment a therapeutic dosage of a CLK modulator, and in particular a CLK-inhibiting compound, disclosed herein. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include one or more CLK inhibitors and one or more therapeutic agents for the treatment of an ocular disorder. For example, one or more CLK-inhibiting compounds can be combined with an effective amount of one or more of: an agent that reduces intraocular pressure, an agent for treating glaucoma, an agent for treating optic neuritis, an agent for treating CMV Retinopathy, an agent for treating multiple sclerosis, and/or an antibiotic, etc.

In one embodiment, a CLK-inhibiting compound can be administered in conjunction with a therapy for reducing intraocular pressure. One group of therapies involves blocking aqueous production. For example, topical beta-adrenergic antagonists (timolol and betaxolol) decrease aqueous production. Topical timolol causes IOP to fall in 30 minutes with peak effects in 1-2 hours. A reasonable regimen is Timoptic 0.5%, one drop every 30 minutes for 2 doses. The carbonic anhydrase inhibitor, acetazolamide, also decreases aqueous production and should be given in conjunction with topical beta-antagonists. An initial dose of 500 mg is administered followed by 250 mg every 6 hours. This medication may be given orally, intramuscularly, or intravenously. In addition, alpha 2-agonists (e.g., Apraclonidine) act by decreasing aqueous production. Their effects are additive to topically administered beta-blockers. They have been approved for use in controlling an acute rise in pressure following anterior chamber laser procedures, but has been reported effective in treating acute closed-angle glaucoma. A reasonable regimen is 1 drop every 30 minutes for 2 doses.

A second group of therapies for reducing intraocular pressure involve reducing vitreous volume. Hyperosmotic agents can be used to treat an acute attack. These agents draw water out of the globe by making the blood hyperosmolar. Oral glycerol in a dose of 1 mL/kg in a cold 50% solution (mixed with lemon juice to make it more palatable) often is used. Glycerol is converted to glucose in the liver; persons with diabetes may need additional insulin if they become hyperglycemic after receiving glycerol. Oral isosorbide is a metabolically inert alcohol that also can be used as an osmotic agent for patients with acute angle-closure glaucoma. Usual dose is 100 g taken p.o. (220 cc of a 45% solution). This inert alcohol should not be confused with isosorbide dinitrate, a nitrate-based cardiac medication used for angina and for congestive heart failure. Intravenous mannitol in a dose of 1.0-1.5 mg/kg also is effective and is well tolerated in patients with nausea and vomiting. These hyperosmotic agents should be used with caution in any patient with a history of congestive heart failure.

A third group of therapies involve facilitating aqueous outflow from the eye. Miotic agents pull the iris from the iridocorneal angle and may help to relieve the obstruction of the trabecular meshwork by the peripheral iris. Pilocarpine 2% (blue eyes)-4% (brown eyes) can be administered every 15 minutes for the first 1-2 hours. More frequent administration or higher doses may precipitate a systemic cholinergic crisis. NSAIDS are sometimes used to reduce inflammation.

Exemplary therapeutic agents for reducing intraocular pressure include ALPHAGAN® P (Allergan) (brimonidine tartrate ophthalmic solution), AZOPT® (Alcon) (brinzolamide ophthalmic suspension), BETAGAN® (Allergan) (levobunolol hydrochloride ophthalmic solution, USP), BETIMOL®) (Vistakon) (timolol ophthalmic solution), BETOPTIC S® (Alcon) (betaxolol HCl), BRIMONIDINE TARTRATE (Bausch & Lomb), CARTEOLOL HYDROCHLORIDE (Bausch & Lomb), COSOPT® (Merck) (dorzolamide hydrochloride-timolol maleate ophthalmic solution), LUMIGAN® (Allergan) (bimatoprost ophthalmic solution), OPTIPRANOLOL® (Bausch & Lomb) (metipranolol ophthalmic solution), TIMOLOL GFS (Falcon) (timolol maleate ophthalmic gel forming solution), TIMOPTIC® (Merck) (timolol maleate ophthalmic solution), TRAVATAN® (Alcon) (travoprost ophthalmic solution), TRUSOPT® (Merck) (dorzolamide hydrochloride ophthalmic solution) and XALATAN® (Pharmacia & Upjohn) (latanoprost ophthalmic solution).

In one embodiment, a CLK-inhibiting compound can be administered in conjunction with a therapy for treating and/or preventing glaucoma. An example of a glaucoma drug is DARANIDE® Tablets (Merck) (Dichlorphenamide).

In one embodiment, a CLK-inhibiting compound can be administered in conjunction with a therapy for treating and/or preventing optic neuritis. Examples of drugs for optic neuritis include DECADRON® Phosphate Injection (Merck) (Dexamethasone Sodium Phosphate), DEPO-MEDROL® (Pharmacia & Upjohn)(methylprednisolone acetate), HYDROCORTONE® Tablets (Merck) (Hydrocortisone), ORAPRED® (Biomarin) (prednisolone sodium phosphate oral solution) and PEDIAPRED® (Celltech) (prednisolone sodium phosphate, USP).

In one embodiment, a CLK-inhibiting compound can be administered in conjunction with a therapy for treating and/or preventing CMV Retinopathy. Treatments for CMV retinopathy include CYTOVENE® (ganciclovir capsules) and VALCYTE® (Roche Laboratories) (valganciclovir hydrochloride tablets).

In one embodiment, a CLK-inhibiting compound can be administered in conjunction with a therapy for treating and/or preventing multiple sclerosis. Examples of such drugs include DANTRIUM® (Procter & Gamble Pharmaceuticals) (dantrolene sodium), NOVANTRONE® (Serono) (mitoxantrone), AVONEX® (Biogen Idec) (Interferon beta-1a), BETASERON® (Berlex) (Interferon beta-1b), COPAXONE® (Teva Neuroscience) (glatiramer acetate injection) and REBIF® (Pfizer) (interferon beta-1a).

In addition, macrolide and/or mycophenolic acid, which has multiple activities, can be co-administered with a CLK-inhibiting compound. Macrolide antibiotics include tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide.

xi. Mitochondrial-Associated Diseases and Disorders

In certain embodiments, the invention provides methods for treating diseases or disorders that would benefit from increased mitochondrial activity. The methods involve administering to a subject in need thereof a therapeutically effective amount of a CLK-inhibiting compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analysis and are summarized in Cohen and Gold, Cleveland Clinic Journal of Medicine, 68: 625-642 (2001). One method for diagnosing a mitochondrial dysfunction is the Thor-Byrne-ier scale (see e.g., Cohen and Gold, supra; Collin S. et al., Eur Neurol. 36: 260-267 (1996)). Other methods for determining mitochondrial number and function include, for example, enzymatic assays (e.g., a mitochondrial enzyme or an ATP biosynthesis factor such as an ETC enzyme or a Krebs cycle enzyme), determination or mitochondrial mass, mitochondrial volume, and/or mitochondrial number, quantification of mitochondrial DNA, monitoring intracellular calcium homeostasis and/or cellular responses to perturbations of this homeostasis, evaluation of response to an apoptogenic stimulus, determination of free radical production. Such methods are known in the art and are described, for example, in U.S. Patent Publication No. 2002/0049176 and references cited therein.

Mitochondria are critical for the survival and proper function of almost all types of eukaryotic cells. Mitochondria in virtually any cell type can have congenital or acquired defects that affect their function. Thus, the clinically significant signs and symptoms of mitochondrial defects affecting respiratory chain function are heterogeneous and variable depending on the distribution of defective mitochondria among cells and the severity of their deficits, and upon physiological demands upon the affected cells. Nondividing tissues with high energy requirements, e.g. nervous tissue, skeletal muscle and cardiac muscle are particularly susceptible to mitochondrial respiratory chain dysfunction, but any organ system can be affected.

Diseases and disorders associated with mitochondrial dysfunction include diseases and disorders in which deficits in mitochondrial respiratory chain activity contribute to the development of pathophysiology of such diseases or disorders in a mammal. This includes 1) congenital genetic deficiencies in activity of one or more components of the mitochondrial respiratory chain; and 2) acquired deficiencies in the activity of one or more components of the mitochondrial respiratory chain, wherein such deficiencies are caused by a) oxidative damage during aging; b) elevated intracellular calcium; c) exposure of affected cells to nitric oxide; d) hypoxia or ischemia; e) microtubule-associated deficits in axonal transport of mitochondria, or f) expression of mitochondrial uncoupling proteins.

Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis. Exemplary diseases or disorders that would benefit from increased mitochondrial activity include, for example, AD (Alzheimer's Disease), ADPD (Alzheimer's Disease and Parkinsons's Disease), AMDF (Ataxia, Myoclonus and Deafness), auto-immune disease, cancer, CIPO (Chronic Intestinal Pseudoobstruction with myopathy and Opthalmoplegia), congenital muscular dystrophy, CPEO (Chronic Progressive External Opthalmoplegia), DEAF (Maternally inherited DEAFness oraminoglycoside-induced DEAFness), DEMCHO (Dementia and Chorea), diabetes mellitus (Type I or Type II), DIDMOAD (Diabetes Insipidus, Diabetes Mellitus, Optic Atrophy, Deafness), DMDF (Diabetes Mellitus and Deafness), dystonia, Exercise Intolerance, ESOC (Epilepsy, Strokes, Optic atrophy, and Cognitive decline), FBSN (Familial Bilateral Striatal Necrosis), FICP (Fatal Infantile Cardiomyopathy Plus, a MELAS-associated cardiomyopathy), GER (Gastrointestinal Reflux), HD (Huntington's Disease), KSS (Kearns Sayre Syndrome), “later-onset” myopathy, LDYT (Leber's hereditary optic neuropathy and DYsTonia), Leigh's Syndrome, LHON (Leber Hereditary Optic Neuropathy), LIMM (Lethal Infantile Mitochondrial Myopathy), MDM (Myopathy and Diabetes Mellitus), MELAS (Mitochondrial Encephalomyopathy, Lactic Acidosis, and Stroke-like episodes), MEPR (Myoclonic Epilepsy and Psychomotor Regression), MERME (MERRF/MELAS overlap disease), MERRF (Myoclonic Epilepsy and Ragged Red Muscle Fibers), MHCM (Maternally Inherited Hypertrophic CardioMyopathy), MICM (Maternally Inherited Cardiomyopathy), MILS (Maternally Inherited Leigh Syndrome), Mitochondrial Encephalocardiomyopathy, Mitochondrial Encephalomyopathy, MM (Mitochondrial Myopathy), MMC (Maternal Myopathy and Cardiomyopathy), MNGIE (Myopathy and external opthalmoplegia, Neuropathy, Gastro-Intestinal, Encephalopathy), Multisystem Mitochondrial Disorder (myopathy, encephalopathy, blindness, hearing loss, peripheral neuropathy), NARP (Neurogenic muscle weakness, Ataxia, and Retinitis Pigmentosa; alternate phenotype at this locus is reported as Leigh Disease), PD (Parkinson's Disease), Pearson's Syndrome, PEM (Progressive Encephalopathy), PEO (Progressive External Opthalmoplegia), PME (Progressive Myoclonus Epilepsy), PMPS (Pearson Marrow-Pancreas Syndrome), psoriasis, RTT (Rett Syndrome), schizophrenia, SIDS (Sudden Infant Death Syndrome), SNHL (Sensorineural Hearing Loss), Varied Familial Presentation (clinical manifestations range from spastic paraparesis to multisystem progressive disorder & fatal cardiomyopathy to truncal ataxia, dysarthria, severe hearing loss, mental regression, ptosis, opthalmoparesis, distal cyclones, and diabetes mellitus), or Wolfram syndrome.

Other diseases and disorders that would benefit from increased mitochondrial activity include, for example, Friedreich's ataxia and other ataxias, amyotrophic lateral sclerosis (ALS) and other motor neuron diseases, macular degeneration, epilepsy, Alpers syndrome, Multiple mitochondrial DNA deletion syndrome, MtDNA depletion syndrome, Complex I deficiency, Complex II (SDH) deficiency, Complex III deficiency, Cytochrome c oxidase (COX, Complex IV) deficiency, Complex V deficiency, Adenine Nucleotide Translocator (ANT) deficiency, Pyruvate dehydrogenase (PDH) deficiency, Ethylmalonic aciduria with lactic acidemia, 3-Methyl glutaconic aciduria with lactic acidemia, Refractory epilepsy with declines during infection, Asperger syndrome with declines during infection, Autism with declines during infection, Attention deficit hyperactivity disorder (ADHD), Cerebral palsy with declines during infection, Dyslexia with declines during infection, materially inherited thrombocytopenia and leukemia syndrome, MARIAHS syndrome (Mitrochondrial ataxia, recurrent infections, aphasia, hypouricemia/hypomyelination, seizures, and dicarboxylic aciduria), ND6 dystonia, Cyclic vomiting syndrome with declines during infection, 3-Hydroxy isobutryic aciduria with lactic acidemia, Diabetes mellitus with lactic acidemia, Uridine responsive neurologic syndrome (URNS), Dilated cardiomyopathy, Splenic Lymphoma, and Renal Tubular Acidosis/Diabetes/Ataxis syndrome.

In other embodiments, the invention provides methods for treating a subject suffering from mitochondrial disorders arising from, but not limited to, Post-traumatic head injury and cerebral edema, Stroke (invention methods useful for preventing or preventing reperfusion injury), Lewy body dementia, Hepatorenal syndrome, Acute liver failure, NASH (non-alcoholic steatohepatitis), Anti-metastasis/prodifferentiation therapy of cancer, Idiopathic congestive heart failure, Atrial fibrillation (non-valvular), Wolff-Parkinson-White Syndrome, Idiopathic heart block, Prevention of reperfusion injury in acute myocardial infarctions, Familial migraines, Irritable bowel syndrome, Secondary prevention of non-Q wave myocardial infarctions, Premenstrual syndrome, Prevention of renal failure in hepatorenal syndrome, Anti-phospholipid antibody syndrome, Eclampsia/pre-eclampsia, Oopause infertility, Ischemic heart disease/Angina, and Shy-Drager and unclassified dysautonomia syndromes.

In still another embodiment, there are provided methods for the treatment of mitochondrial disorders associated with pharmacological drug-related side effects. Types of pharmaceutical agents that are associated with mitochondrial disorders include reverse transcriptase inhibitors, protease inhibitors, inhibitors of DHOD, and the like. Examples of reverse transcriptase inhibitors include, for example, Azidothymidine (AZT), Stavudine (D4T), Zalcitabine (ddC), Didanosine (DDI), Fluoroiodoarauracil (FIAU), and the like. Examples of protease inhibitors include, for example, Ritonavir, Indinavir, Saquinavir, Nelfinavir and the like. Examples of inhibitors of dihydroorotate dehydrogenase (DHOD) include, for example, Leflunomide, Brequinar and the like.

Common symptoms of mitochondrial diseases include cardiomyopathy, muscle weakness and atrophy, developmental delays (involving motor, language, cognitive or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

In certain embodiments, the invention provides methods for treating a disease or disorder that would benefit from increased mitochondrial activity that involves administering to a subject in need thereof one or more CLK-inhibiting compounds in combination with another therapeutic agent such as, for example, an agent useful for treating mitochondrial dysfunction (such as antioxidants, vitamins, or respiratory chain cofactors), an agent useful for reducing a symptom associated with a disease or disorder involving mitochondrial dysfunction (such as, an anti-seizure agent, an agent useful for alleviating neuropathic pain, an agent for treating cardiac dysfunction), a cardiovascular agent (as described further below), a chemotherapeutic agent (as described further below), or an anti-neurodegeneration agent (as described further below). In an exemplary embodiment, the invention provides methods for treating a disease or disorder that would benefit from increased mitochondrial activity that involves administering to a subject in need thereof one or more CLK-inhibiting compounds in combination with one or more of the following: coenzyme Q₁₀, L-carnitine, thiamine, riboflavin, niacinamide, folate, vitamin E, selenium, lipoic acid, or prednisone. Compositions comprising such combinations are also provided herein.

In exemplary embodiments, the invention provides methods for treating diseases or disorders that would benefit from increased mitochondrial activity by administering to a subject a therapeutically effective amount of a CLK-inhibiting compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migraine, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation.

A gene defect underlying Friedreich's Ataxia (FA), the most common hereditary ataxia, was recently identified and is designated “frataxin”. In FA, after a period of normal development, deficits in coordination develop which progress to paralysis and death, typically between the ages of 30 and 40. The tissues affected most severely are the spinal cord, peripheral nerves, myocardium, and pancreas. Patients typically lose motor control and are confined to wheel chairs, and are commonly afflicted with heart failure and diabetes. The genetic basis for FA involves GAA trinucleotide repeats in an intron region of the gene encoding frataxin. The presence of these repeats results in reduced transcription and expression of the gene. Frataxin is involved in regulation of mitochondrial iron content. When cellular frataxin content is subnormal, excess iron accumulates in mitochondria, promoting oxidative damage and consequent mitochondrial degeneration and dysfunction. When intermediate numbers of GAA repeats are present in the frataxin gene intron, the severe clinical phenotype of ataxia may not develop. However, these intermediate-length trinucleotide extensions are found in 25 to 30% of patients with non-insulin dependent diabetes mellitus, compared to about 5% of the nondiabetic population. In certain embodiments, CLK-inhibiting compounds may be used for treating patients with disorders related to deficiencies or defects in frataxin, including Friedreich's Ataxia, myocardial dysfunction, diabetes mellitus and complications of diabetes like peripheral neuropathy.

Muscular dystrophy refers to a family of diseases involving deterioration of neuromuscular structure and function, often resulting in atrophy of skeletal muscle and myocardial dysfunction. In the case of Duchenne muscular dystrophy, mutations or deficits in a specific protein, dystrophin, are implicated in its etiology. Mice with their dystrophin genes inactivated display some characteristics of muscular dystrophy, and have an approximately 50% deficit in mitochondrial respiratory chain activity. A final common pathway for neuromuscular degeneration in most cases is calcium-mediated impairment of mitochondrial function. In certain embodiments, CLK-inhibiting compounds may be used for reducing the rate of decline in muscular functional capacities and for improving muscular functional status in patients with muscular dystrophy.

Multiple sclerosis (MS) is a neuromuscular disease characterized by focal inflammatory and autoimmune degeneration of cerebral white matter. Periodic exacerbations or attacks are significantly correlated with upper respiratory tract and other infections, both bacterial and viral, indicating that mitochondrial dysfunction plays a role in MS. Depression of neuronal mitochondrial respiratory chain activity caused by Nitric Oxide (produced by astrocytes and other cells involved in inflammation) is implicated as a molecular mechanism contributing to MS. In certain embodiments, CLK-inhibiting compounds may be used for treatment of patients with multiple sclerosis, both prophylactically and during episodes of disease exacerbation.

Epilepsy is often present in patients with mitochondrial cytopathies, involving a range of seizure severity and frequency, e.g. absence, tonic, atonic, myoclonic, and status epilepticus, occurring in isolated episodes or many times daily. In certain embodiments, CLK-inhibiting compounds may be used for treating patients with seizures secondary to mitochondrial dysfunction, including reducing frequency and severity of seizure activity.

Metabolic studies on patients with recurrent migraine headaches indicate that deficits in mitochondrial activity are commonly associated with this disorder, manifesting as impaired-oxidative phosphorylation and excess lactate production. Such deficits are not necessarily due to genetic defects in mitochondrial DNA. Migraineurs are hypersensitive to nitric oxide, an endogenous inhibitor of Cytochrome c Oxidase. In addition, patients with mitochondrial cytopathies, e.g. MELAS, often have recurrent migraines. In certain embodiments, CLK-inhibiting compounds may be used for treating patients with recurrent migraine headaches, including headaches refractory to ergot compounds or serotonin receptor antagonists.

Delays in neurological or neuropsychological development are often found in children with mitochondrial diseases. Development and remodeling of neural connections requires intensive biosynthetic activity, particularly involving synthesis of neuronal membranes and myelin, both of which require pyrimidine nucleotides as cofactors. Uridine nucleotides are involved inactivation and transfer of sugars to glycolipids and glycoproteins. Cytidine nucleotides are derived from uridine nucleotides, and are crucial for synthesis of major membrane phospholipid constituents like phosphatidylcholine, which receives its choline moiety from cytidine diphosphocholine. In the case of mitochondrial dysfunction (due to either mitochondrial DNA defects or any of the acquired or conditional deficits like exicitoxic or nitric oxide-mediated mitochondrial dysfunction) or other conditions resulting in impaired pyrimidine synthesis, cell proliferation and axonal extension is impaired at crucial stages in development of neuronal interconnections and circuits, resulting in delayed or arrested development of neuropsychological functions like language, motor, social, executive function, and cognitive skills. In autism for example, magnetic resonance spectroscopy measurements of cerebral phosphate compounds indicates that there is global undersynthesis of membranes and membrane precursors indicated by reduced levels of uridine diphospho-sugars, and cytidine nucleotide derivatives involved in membrane synthesis. Disorders characterized by developmental delay include Rett's Syndrome, pervasive developmental delay (or PDD-NOS “pervasive developmental delay not otherwise specified” to distinguish it from specific subcategories like autism), autism, Asperger's Syndrome, and Attention Deficit/Hyperactivity Disorder (ADHD), which is becoming recognized as a delay or lag in development of neural circuitry underlying executive functions. In certain embodiments, CLK-inhibiting compounds may be useful for treating patients with neurodevelopmental delays (e.g., involving motor, language, executive function, and cognitive skills), or other delays or arrests of neurological and neuropsychological development in the nervous system and somatic development in non-neural tissues like muscle and endocrine glands.

The two most significant severe neurodegenerative diseases associated with aging, Alzheimer's Disease (AD) and Parkinson's Disease (PD), both involve mitochondrial dysfunction in their pathogenesis. Complex I deficiencies in particular are frequently found not only in the nigrostriatal neurons that degenerate in Parkinson's disease, but also in peripheral tissues and cells like muscle and platelets of Parkinson's Disease patients. In Alzheimer's Disease, mitochondrial respiratory chain activity is often depressed, especially Complex IV (Cytochrome c Oxidase). Moreover, mitochondrial respiratory function altogether is depressed as a consequence of aging, further amplifying the deleterious sequelae of additional molecular lesions affecting respiratory chain function. Other factors in addition to primary mitochondrial dysfunction underlie neurodegeneration in AD, PD, and related disorders. Excitotoxic stimulation and nitric oxide are implicated in both diseases, factors which both exacerbate mitochondrial respiratory chain deficits and whose deleterious actions are exaggerated on a background of respiratory chain dysfunction. Huntington's Disease also involves mitochondrial dysfunction in affected brain regions, with cooperative interactions of excitotoxic stimulation and mitochondrial dysfunction contributing to neuronal degeneration. In certain embodiments, CLK-inhibiting compounds may be useful for treating and attenuating progression of age-related neurodegenerative disease including AD and PD.

One of the major genetic defects in patients with Amyotrophic Lateral Sclerosis (ALS or Lou Gehrig's Disease) is mutation or deficiency in Copper-Zinc Superoxide Dismutase (SOD 1), an antioxidant enzyme. Mitochondria both produce and are primary targets for reactive oxygen species. Inefficient transfer of electrons to oxygen in mitochondria is the most significant physiological source of free radicals in mammalian systems. Deficiencies in antioxidants or antioxidant enzymes can result in or exacerbate mitochondrial degeneration. Mice transgenic for mutated SOD1 develop symptoms and pathology similar to those in human ALS. The development of the disease in these animals has been shown to involve oxidative destruction of mitochondria followed by functional decline of motor neurons and onset of clinical symptoms. Skeletal muscle from ALS patients has low mitochondrial Complex I activity. In certain embodiments, CLK-inhibiting compounds may be useful for treating ALS, for reversing or slowing the progression of clinical symptoms.

Oxygen deficiency results in both direct inhibition of mitochondrial respiratory chain activity by depriving cells of a terminal electron acceptor for Cytochrome c reoxidation at Complex IV, and indirectly, especially in the nervous system, via secondary post-anoxic excitotoxicity and nitric oxide formation. In conditions like cerebral anoxia, angina or sickle cell anemia crises, tissues are relatively hypoxic. In such cases, compounds that increase mitochondrial activity provide protection of affected tissues from deleterious effects of hypoxia, attenuate secondary delayed cell death, and accelerate recovery from hypoxic tissue stress and injury. In certain embodiments, CLK-inhibiting compounds may be useful for preventing delayed cell death (apoptosis in regions like the hippocampus or cortex occurring about 2 to 5 days after an episode of cerebral ischemia) after ischemic or hypoxic insult to the brain.

Acidosis due to renal dysfunction is often observed in patients with mitochondrial disease, whether the underlying respiratory chain dysfunction is congenital or induced by ischemia or cytotoxic agents like cisplatin. Renal tubular acidosis often requires administration of exogenous sodium bicarbonate to maintain blood and tissue pH. In certain embodiments, CLK-inhibiting compounds may be useful for treating renal tubular acidosis and other forms of renal dysfunction caused by mitochondrial respiratory chain deficits.

During normal aging, there is a progressive decline in mitochondrial respiratory chain function. Beginning about age 40, there is an exponential rise in accumulation of mitochondrial DNA defects in humans, and a concurrent decline in nuclear-regulated elements of mitochondrial respiratory activity. Many mitochondrial DNA lesions have a selection advantage during mitochondrial turnover, especially in postmitotic cells. The proposed mechanism is that mitochondria with a defective respiratory chain produce less oxidative damage to themselves than do mitochondria with intact functional respiratory chains (mitochondrial respiration is the primary source of free radicals in the body). Therefore, normally-functioning mitochondria accumulate oxidative damage to membrane lipids more rapidly than do defective mitochondria, and are therefore “tagged” for degradation by lysosomes. Since mitochondria within cells have a half life of about 10 days, a selection advantage can result in rapid replacement of functional mitochondria with those with diminished respiratory activity, especially in slowly dividing cells. The net result is that once a mutation in a gene for a mitochondrial protein that reduces oxidative damage to mitochondria occurs, such defective mitochondria will rapidly populate the cell, diminishing or eliminating its respiratory capabilities. The accumulation of such cells results in aging or degenerative disease at the organismal level. This is consistent with the progressive mosaic appearance of cells with defective electron transport activity in muscle, with cells almost devoid of Cytochrome c Oxidase (COX) activity interspersed randomly amidst cells with normal activity, and a higher incidence of COX-negative cells in biopsies from older subjects. The organism, during aging, or in a variety of mitochondrial diseases, is thus faced with a situation in which irreplaceable postmitotic cells (e.g. neurons, skeletal and cardiac muscle) must be preserved and their function maintained to a significant degree, in the face of an inexorable progressive decline in mitochondrial respiratory chain function. Neurons with dysfunctional mitochondria become progressively more sensitive to insults like excitotoxic injury. Mitochondrial failure contributes to most degenerative diseases (especially neurodegeneration) that accompany aging. Congenital mitochondrial diseases often involve early-onset neurodegeneration similar in fundamental mechanism to disorders that occur during aging of people born with normal mitochondria. In certain embodiments, CLK-inhibiting compounds may be useful for treating or attenuating cognitive decline and other degenerative consequences of aging.

Mitochondrial DNA damage is more extensive and persists longer than nuclear DNA damage in cells subjected to oxidative stress or cancer chemotherapy agents like cisplatin due to both greater vulnerability and less efficient repair of mitochondrial DNA. Although mitochondrial DNA may be more sensitive to damage than nuclear DNA, it is relatively resistant, in some situations, to mutagenesis by chemical carcinogens. This is because mitochondria respond to some types of mitochondrial DNA damage by destroying their defective genomes rather than attempting to repair them. This results in global mitochondrial dysfunction for a period after cytotoxic chemotherapy. Clinical use of chemotherapy agents like cisplatin, mitomycin, and cytoxan is often accompanied by debilitating “chemotherapy fatigue”, prolonged periods of weakness and exercise intolerance which may persist even after recovery from hematologic and gastrointestinal toxicities of such agents. In certain embodiments, CLK-inhibiting compounds may be useful for treatment and prevention of side effects of cancer chemotherapy related to mitochondrial dysfunction.

A crucial function of the ovary is to maintain integrity of the mitochondrial genome in oocytes, since mitochondria passed onto a fetus are all derived from those present in oocytes at the time of conception. Deletions in mitochondrial DNA become detectable around the age of menopause, and are also associated with abnormal menstrual cycles. Since cells cannot directly detect and respond to defects in mitochondrial DNA, but can only detect secondary effects that affect the cytoplasm, like impaired respiration, redox status, or deficits in pyrimidine synthesis, such products of mitochondrial function participate as a signal for oocyte selection and follicular atresia, ultimately triggering menopause when maintenance of mitochondrial genomic fidelity and functional activity can no longer be guaranteed. This is analogous to apoptosis in cells with DNA damage, which undergo an active process of cellular suicide when genomic fidelity can no longer be achieved by repair processes. Women with mitochondrial cytopathies affecting the gonads often undergo premature menopause or display primary cycling abnormalities. Cytotoxic cancer chemotherapy often induces premature menopause, with a consequent increased risk of osteoporosis. Chemotherapy-induced amenorrhea is generally due to primary ovarian failure. The incidence of chemotherapy-induced amenorrhea increases as a function of age in premenopausal women receiving chemotherapy, pointing toward mitochondrial involvement. Inhibitors of mitochondrial respiration or protein synthesis inhibit hormone-induced ovulation, and furthermore inhibit production of ovarian steroid hormones in response to pituitary gonadotropins. Women with Downs syndrome typically undergo menopause prematurely, and also are subject to early onset of Alzheimer-like dementia. Low activity of cytochrome oxidase is consistently found in tissues of Downs patients and in late-onset Alzheimer's Disease. Appropriate support of mitochondrial function or compensation for mitochondrial dysfunction therefore is useful for protecting against age-related or chemotherapy-induced menopause or irregularities of menstrual cycling or ovulation. In certain embodiments, CLK-inhibiting compounds may be useful for treating and preventing amenorrhea, irregular ovulation, menopause, or secondary consequences of menopause.

In certain embodiments, CLK modulating compounds, and in particular a CLK-inhibiting compound, may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external opthalmoplegia, the Kearns-Sayre syndrome (with opthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal). Muscle biopsy specimens stained with modified Gomori's trichrome stain show ragged red fibers due to excessive accumulation of mitochondria. Biochemical defects in substrate transport and utilization, the Krebs cycle, oxidative phosphorylation, or the respiratory chain are detectable. Numerous mitochondrial DNA point mutations and deletions have been described, transmitted in a maternal, nonmendelian inheritance pattern. Mutations in nuclear-encoded mitochondrial enzymes occur.

In certain embodiments, CLK-inhibiting compounds may be useful for treating patients suffering from toxic damage to mitochondria, such as, toxic damage due to calcium accumulation, excitotoxicity, nitric oxide exposure, or hypoxia.

A fundamental mechanism of cell injury, especially in excitable tissues, involves excessive calcium entry into cells, as a result of either leakage through the plasma membrane or defects in intracellular calcium handling mechanisms. Mitochondria are major sites of calcium sequestration, and preferentially utilize energy from the respiratory chain for taking up calcium rather than for ATP synthesis, which results in a downward spiral of mitochondrial failure, since calcium uptake into mitochondria results in diminished capabilities for energy transduction.

Excessive stimulation of neurons with excitatory amino acids is a common mechanism of cell death or injury in the central nervous system. Activation of glutamate receptors, especially of the subtype designated NMDA receptors, results in mitochondrial dysfunction, in part through elevation of intracellular calcium during excitotoxic stimulation. Conversely, deficits in mitochondrial respiration and oxidative phosphorylation sensitizes cells to excitotoxic stimuli, resulting in cell death or injury during exposure to levels of excitotoxic neurotransmitters or toxins that would be innocuous to normal cells.

Nitric oxide (about 1 micromolar) inhibits cytochrome oxidase (Complex IV) and thereby inhibits mitochondrial respiration; moreover, prolonged exposure to nitric oxide (NO) irreversibly reduces Complex I activity. Physiological or pathophysiological concentrations of NO thereby inhibit pyrimidine biosynthesis. Nitric oxide is implicated in a variety of neurodegenerative disorders including inflammatory and autoimmune diseases of the central nervous system, and is involved in mediation of excitotoxic and post-hypoxic damage to neurons.

Oxygen is the terminal electron acceptor in the respiratory chain. Oxygen deficiency impairs electron transport chain activity, resulting in diminished pyrimidine synthesis as well as diminished ATP synthesis via oxidative phosphorylation. Human cells proliferate and retain viability under virtually anaerobic conditions if provided with uridine and pyruvate (or a similarly effective agent for oxidizing NADH to optimize glycolytic ATP production).

In certain embodiments, CLK-inhibiting compounds may be useful for treating diseases or disorders associated with mitochondrial deregulation.

Transcription of mitochondrial DNA encoding respiratory chain components requires nuclear factors. In neuronal axons, mitochondria must shuttle back and forth to the nucleus in order to maintain respiratory chain activity. If axonal transport is impaired by hypoxia or by drugs like taxol which affect microtubule stability, mitochondria distant from the nucleus undergo loss of cytochrome oxidase activity. Accordingly, treatment with a CLK-inhibiting compound may be useful for promoting nuclear-mitochondrial interactions.

Mitochondria are the primary source of free radicals and reactive oxygen species, due to spillover from the mitochondrial respiratory chain, especially when defects in one or more respiratory chain components impairs orderly transfer of electrons from metabolic intermediates to molecular oxygen. To reduce oxidative damage, cells can compensate by expressing mitochondrial uncoupling proteins (UCP), of which several have been identified. UCP-2 is transcribed in response to oxidative damage, inflammatory cytokines, or excess lipid loads, e.g. fatty liver and steatohepatitis. UCPs reduce spillover of reactive oxygen species from mitochondria by discharging proton gradients across the mitochondrial inner membrane, in effect wasting energy produced by metabolism and rendering cells vulnerable to energy stress as a trade-off for reduced oxidative injury.

xii. Muscle Performance

In other embodiments, the invention provides methods for enhancing muscle performance by administering a therapeutically effective amount of a CLK-inhibiting compound. For example, CLK-inhibiting compounds may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a CLK inhibiting compound that increase mitochondrial activity, increase mitochondrial biogenesis, increase mitochondrial mass, or a high dose of a CLK-inhibiting compound.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. An athlete may be hard training, that is, performs sports activities intensely more than three days a week or for competition. An athlete may also be a fitness enthusiast who seeks to improve general health and well-being, improve energy levels, who works out for about 1-2 hours about 3 times a week. Enhanced sports performance in manifested by the ability to overcome muscle fatigue, ability to maintain activity for longer periods of time, and have a more effective workout.

In the arena of athlete muscle performance, it is desirable to create conditions that permit competition or training at higher levels of resistance for a prolonged period of time. However, acute and intense anaerobic use of skeletal muscles often results in impaired athletic performance, with losses in force and work output, and increased onset of muscle fatigue, soreness, and dysfunction. It is now recognized that even a single exhaustive exercise session, or for that matter any acute trauma to the body such as muscle injury, resistance or exhaustive muscle exercise, or elective surgery, is characterized by perturbed metabolism that affects muscle performance in both short and long term phases. Both muscle metabolic/enzymatic activity and gene expression are affected. For example, disruption of skeletal muscle nitrogen metabolism as well as depletion of sources of metabolic energy occur during extensive muscle activity. Amino acids, including branched-chain amino acids, are released from muscles followed by their deamination to elevate serum ammonia and local oxidation as muscle fuel sources, which augments metabolic acidosis. In addition, there is a decline in catalytic efficiency of muscle contraction events, as well as an alteration of enzymatic activities of nitrogen and energy metabolism. Further, protein catabolism is initiated where rate of protein synthesis is decreased coupled with an increase in the degradation of non-contractible protein. These metabolic processes are also accompanied by free radical generation which further damages muscle cells.

Recovery from fatigue during acute and extended exercise requires reversal of metabolic and non-metabolic fatiguing factors. Known factors that participate in human muscle fatigue, such as lactate, ammonia, hydrogen ion, etc., provide an incomplete and unsatisfactory explanation of the fatigue/recovery process, and it is likely that additional unknown agents participate (Baker et al., J. Appl. Physiol. 74:2294-2300, 1993; Bazzarre et al., J. Am. Coll. Nutr. 11:505-511, 1992; Dohm et al., Fed. Proc. 44:348-352, 1985; Edwards In: Biochemistry of Exercise, Proceedings of the Fifth International Symposium on the Biochemistry of Exercise (Kutrgen, Vogel, Poormans, eds.), 1983; MacDougall et al., Acta Physiol. Scand. 146:403-404, 1992; Walser et al., Kidney Int. 32:123-128, 1987). Several studies have also analyzed the effects of nutritional supplements and herbal supplements in enhancing muscle performance.

Aside from muscle performance during endurance exercise, free radicals and oxidative stress parameters are affected in pathophysiological states. A substantial body of data now suggests that oxidative stress contributes to muscle wasting or atrophy in pathophysiological states (reviewed in Clarkson, P. M. Antioxidants and physical performance. Crit. Rev. Food Sci. Nutr. 35: 31-41; 1995; Powers, S. K.; Lennon, S. L. Analysis of cellular responses to free radicals: Focus on exercise and skeletal muscle. Proc. Nutr. Soc. 58: 1025-1033; 1999). For example, with respect to muscular disorders where both muscle endurance and function are compensated, the role of nitric oxide (NO), has been implicated. In muscular dystrophies, especially those due to defects in proteins that make up the dystrophin-glycoprotein complex (DGC), the enzyme that synthesizes NO, nitric oxide synthase (NOS), has been associated. Recent studies of dystrophies related to DGC defects suggest that one mechanism of cellular injury is functional ischemia related to alterations in cellular NOS and disruption of a normal protective action of NO. This protective action is the prevention of local ischemia during contraction-induced increases in sympathetic vasoconstriction. Rando (Microsc Res Tech 55 (4):223-35, 2001), has shown that oxidative injury precedes pathologic changes and that muscle cells with defects in the DGC have an increased susceptibility to oxidant challenges. Excessive lipid peroxidation due to free radicals has also been shown to be a factor in myopathic diseases such as McArdle's disease (Russo et al., Med Hypotheses. 39 (2):147-51, 1992). Furthermore, mitochondrial dysfunction is a well-known correlate of age-related muscle wasting (sarcopenia) and free radical damage has been suggested, though poorly investigated, as a contributing factor (reviewed in Navarro, A.; Lopez-Cepero, J. M.; Sanchez del Pino, M. L. Front. Biosci. 6: D26-44; 2001). Other indications include acute sarcopenia, for example muscle atrophy and/or cachexia associated with burns, bed rest, limb immobilization, or major thoracic, abdominal, and/or orthopedic surgery. It is contemplated that the methods of the present invention will also be effective in the treatment of muscle related pathological conditions.

In certain embodiments, the invention provides novel dietary compositions comprising CLK-inhibiting compounds, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

xiii. Other Uses

CLK-inhibiting compounds may be used for treating or preventing viral infections (such as infections by influenza, herpes or papilloma virus) or as antifungal agents. In certain embodiments, CLK-inhibiting compounds may be administered as part of a combination drug therapy with another therapeutic agent for the treatment of viral diseases, including, for example, acyclovir, ganciclovir and zidovudine. In another embodiment, CLK-inhibiting compounds may be administered as part of a combination drug therapy with another anti-fungal agent including, for example, topical anti-fungals such as ciclopirox, clotrimazole, econazole, miconazole, nystatin, oxiconazole, terconazole, and tolnaftate, or systemic anti-fungal such as fluconazole (Diflucan), itraconazole (Sporanox), ketoconazole (Nizoral), and miconazole (Monistat I.V.).

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, modulating compounds may be administered to farm animals to improve their ability to withstand farming conditions longer.

CLK-inhibiting compounds may also be used to increase lifespan, stress resistance, and resistance to apoptosis in plants. In one embodiment, a compound is applied to plants, e.g., on a periodic basis, or to fungi. In another embodiment, plants are genetically modified to produce a compound. In another embodiment, plants and fruits are treated with a compound prior to picking and shipping to increase resistance to damage during shipping. Plant seeds may also be contacted with compounds described herein, e.g., to preserve them.

In other embodiments, CLK-inhibiting compounds may be used for modulating lifespan in yeast cells. Situations in which it may be desirable to extend the lifespan of yeast cells include any process in which yeast is used, e.g., the making of beer, yogurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

CLK-inhibiting compounds may also be used to increase lifespan, stress resistance and resistance to apoptosis in insects. In this embodiment, compounds would be applied to useful insects, e.g., bees and other insects that are involved in pollination of plants. In a specific embodiment, a compound would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of CLK-inhibiting compounds may also be used as a pesticide by interfering with the regulation of silenced genes and the regulation of apoptosis during development. In this embodiment, a compound may be applied to plants using a method known in the art that ensures the compound is bio-available to insect larvae, and not to plants.

In other embodiments, CLK-inhibiting compounds can be applied to affect the reproduction of organisms such as insects, animals and microorganisms.

3. CLK-Modulating Compounds

In various embodiments, CLK-modulators useful for the methods described herein may be small molecules, polypeptides (including antibodies), or nucleic acids (including antisense nucleic acids, ribozymes, and small interfering RNAs or siRNAs). Examples small molecule CLK-inhibiting compounds are described in U.S. Pat. No. application 2005/0171026 (“Therapeutic composition of treating abnormal splicing caused by the excessive kinase induction”) or are illustrated in FIG. 14 herein.

In another embodiment, a CLK-modulator may be an antisense nucleic acid. By “antisense nucleic acid,” it is meant a non-enzymatic nucleic acid compound that binds to a target nucleic acid by means of RNA-RNA, RNA-DNA or RNA-PNA (protein nucleic acid) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can form a loop and binds to a substrate nucleic acid which forms a loop. Thus, an antisense molecule can be complementary to two (or more) non-contiguous substrate sequences, or two (or more) non-contiguous sequence portions of an antisense molecule can be complementary to a target sequence, or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49.

In other embodiments, the CLK-modulating compound may be an siRNA. The term “short interfering RNA,” “siRNA,” or “short interfering nucleic acid,” refers to any nucleic acid compound capable of mediating RNAi or gene silencing when processed appropriately be a cell. For example, the siRNA can be a double-stranded polynucleotide molecule comprising self-complementary sense and antisense regions, wherein the antisense region comprises complementarity to a target nucleic acid compound (e.g., a CLK protein). The siRNA can be a single-stranded hairpin polynucleotide having self-complementary sense and antisense regions, wherein the antisense region comprises complementarity to a target nucleic acid compound. The siRNA can be a circular single-stranded polynucleotide having two or more loop structures and a stem comprising self-complementary sense and antisense regions, wherein the antisense region comprises complementarity to a target nucleic acid compound, and wherein the circular polynucleotide can be processed either in vivo or in vitro to generate an active siRNA capable of mediating RNAi. The siRNA can also comprise a single stranded polynucleotide having complementarity to a target nucleic acid compound, wherein the single stranded polynucleotide can further comprise a terminal phosphate group, such as a 5′-phosphate (see for example Martinez et al., 2002, Cell., 110, 563-574), or 5′,3′-diphosphate.

As described herein, the subject siRNAs are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of the subject siRNAs can be carried out by chemical synthetic methods or by recombinant nucleic acid techniques. Endogenous RNA polymerase of the treated cell may mediate transcription in vivo, or cloned RNA polymerase can be used for transcription in vitro. As used herein, siRNA molecules of the disclosure need not be limited to those molecules containing only RNA, but further encompasses chemically-modified nucleotides and non-nucleotides. For example, the dsRNAs may include modifications to either the phosphate-sugar backbone or the nucleoside, e.g., to reduce susceptibility to cellular nucleases, improve bioavailability, improve formulation characteristics, and/or change other pharmacokinetic properties. To illustrate, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The dsRNAs may be produced enzymatically or by partial/total organic synthesis, any modified ribonucleotide can be introduced by in vitro enzymatic or organic synthesis. Methods of chemically modifying RNA molecules can be adapted for modifying dsRNAs (see, e.g., Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an dsRNA can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). In certain cases, the dsRNAs of the disclosure lack 2′-hydroxy(2′-OH) containing nucleotides.

In a specific embodiment, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In another specific embodiment, the subject dsRNA can also be in the form of a long double-stranded RNA. For example, the dsRNA is at least 25, 50, 100, 200, 300 or 400 bases. In some cases, the dsRNA is 400-800 bases in length. Optionally, the dsRNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In a further specific embodiment, the dsRNA is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present disclosure provides a recombinant vector having the following unique characteristics: it comprises a viral replicon having two overlapping transcription units arranged in an opposing orientation and flanking a transgene for a dsRNA of interest, wherein the two overlapping transcription units yield both sense and antisense RNA transcripts from the same transgene fragment in a host cell.

In another embodiment, a CLK-modulator may be an antibody that binds to a CLK protein. The term “antibody” as used herein is intended to include fragments thereof which are also specifically reactive with a polypeptide of the invention. Antibodies can be fragmented using conventional techniques and the fragments screened for utility in the same manner as is suitable for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further intended to include bispecific and chimeric molecules, as well as single chain (scFv) antibodies. Also included are trimeric antibodies, humanized antibodies, human antibodies, and single chain antibodies. All of these modified forms of antibodies as well as fragments of antibodies are intended to be included in the term “antibody”.

Antibodies may be elicited by methods known in the art. For example, a mammal such as a mouse, a hamster or rabbit may be immunized with an immunogenic form of a CLK protein (e.g., an antigenic fragment which is capable of eliciting an antibody response). Alternatively, immunization may occur by using a nucleic acid, which in vivo expresses a CLK protein giving rise to the immunogenic response observed. Techniques for conferring immunogenicity on a protein or peptide include conjugation to carriers or other techniques well known in the art. For instance, a peptidyl portion of a polypeptide of the invention may be administered in the presence of adjuvant. The progress of immunization may be monitored by detection of antibody titers in plasma or serum. Standard ELISA or other immunoassays may be used with the immunogen as antigen to assess the levels of antibodies.

Following immunization, antisera reactive with a polypeptide of the invention may be obtained and, if desired, polyclonal antibodies isolated from the serum. To produce monoclonal antibodies, antibody producing cells (lymphocytes) may be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the polypeptides of the invention and the monoclonal antibodies isolated.

4. Assays

Yet other methods contemplated herein include screening methods for identifying compounds or agents that modulate CLK proteins. An agent may be a nucleic acid, such as an aptamer. Assays may be conducted in a cell based or cell free format. For example, an assay may comprise incubating (or contacting) a CLK with a test agent under conditions in which a CLK can be modulated by an agent known to modulate the CLK, and monitoring or determining the level of modulation of the CLK in the presence of the test agent relative to the absence of the test agent. The level of modulation of a CLK can be determined by determining its ability to deacetylate a substrate.

Methods for identifying an agent that modulates, e.g., stimulates or inhibits, CLKs in vivo may comprise (i) contacting a cell with a test agent and a substrate that is capable of entering a cell under conditions appropriate for the CLK to phosphorylate the substrate in the absence of the test agent; and (ii) determining the level of phosphorylation of the substrate, wherein (i) a lower level of phosphorylation of the substrate in the presence of the test agent relative to the level of phosphorylation in the absence of the test agent indicates that the test agent inhibits phosphorylation by the CLK, or (ii) wherein a higher level of phosphorylation of the substrate in the presence of the test agent relative to the level of phosphorylation in the absence of the test agent indicates that the test agent activates phosphorylation by the CLK.

In yet other embodiments, provided are methods (e.g., assays such as screening assays or high throughput screens) for identifying agents, such as CLK modulating compounds, that are useful for modulating mitochondrial mass and/or mitochondrial function in cells of an animal or human subject. In certain embodiments, candidate agents are screened for their ability to increase mitochondrial mass and/or improve mitochondrial function. In an exemplary embodiment, the methods described herein may be used to identify an agent that increases mitochondrial mass and/or improves mitochondrial function in cells, such as, for example, a CLK-inhibiting compound.

In one embodiment, a method for identifying an agent that modulates mitochondrial mass and/or function comprises contacting a candidate agent with a sample comprising a cell containing a mitochondrion, and determining a level of at least one indicator of mitochondrial function, wherein the candidate agent that alters the level of the indicator of mitochondrial function relative to the level of said indicator in the absence of the agent is indicative of an agent that alters mitochondrial function.

In another embodiment, a method for identifying an agent that modulates mitochondrial mass and/or function comprises identifying a regulator of mitochondrial biogenesis. The method may comprise contacting a stimulus with a cell comprising a mitochondrion under conditions and for a time sufficient to induce mitochondrial biogenesis; and detecting an altered level of a candidate signaling molecule, wherein an altered level of the candidate signaling molecule in a cell that has been contacted with the stimulus that induces mitochondrial biogenesis relative to the level of the candidate signaling molecule in a cell that has not been contacted with the stimulus indicates that the candidate signaling molecule is a regulator of mitochondrial biogenesis. In a further embodiment the stimulus is selected cold stress, an electrical stimulus or an adrenergic stimulus. In certain other embodiments mitochondrial biogenesis is detected by determining an indicator of mitochondrial function that is oxygen consumption, amount of mitochondrial DNA, mitochondrial mass or an ATP biosynthesis factor. In certain other embodiments the candidate signaling molecule regulates activity of a gene that is a PGC gene or a NRF gene. In certain other embodiments the candidate signaling molecule is regulated by a gene that is a PGC gene or a NRF gene. In certain other embodiments the altered level of the candidate signaling molecule is a level of a nucleic acid, a level of a polypeptide and a level of phosphorylation of a protein.

In certain embodiments, the indicator of mitochondrial function may be a mitochondrial electron transport chain enzyme. The methods may involve measuring electron transport chain enzyme catalytic activity, determining enzyme activity per mitochondrion in the sample, determining enzyme activity per unit of protein in the sample, measuring electron transport chain enzyme quantity, determining enzyme quantity per mitochondrion in the sample, and/or determining enzyme quantity per unit of protein in the sample. In certain embodiments the mitochondrial electron transport chain enzyme comprises at least one subunit of mitochondrial complex 1, mitochondrial complex II, mitochondrial complex III, mitochondrial complex IV, and/or mitochondrial complex V. The mitochondrial complex IV subunit may be COX1, COX2 or COX4 and the mitochondrial complex V subunit may be an ATP synthase subunit 8 or ATP synthase subunit 6.

In other embodiments, the indicator of mitochondrial function may be a mitochondrial matrix component. a mitochondrial membrane component, and/or a mitochondrial inner membrane component. The mitochondrial membrane component may be an adenine nucleotide translocator (ANT), voltage dependent anion channel (VDAC), malate-aspartate shuttle, calcium uniporter, UCP-1, UCP-2, UCP-3 (e.g., Boss et al., 2000 Diabetes 49:143; Klingenberg 1999 J. Bioenergetics Biomembranes 31:419), a hexokinase, a peripheral benzodiazepine receptor, a mitochondrial intermembrane creatine kinase, cyclophilin D, a Bcl-2 gene family encoded polypeptide, tricarboxylate carrier or dicarboxylate carrier.

In certain embodiments the indicator of mitochondrial function is a Krebs cycle enzyme. The methods may involve measuring Krebs cycle enzyme catalytic activity, determining enzyme activity per mitochondrion in the sample, determining enzyme activity per unit of protein in the sample, measuring Krebs cycle enzyme quantity, determining enzyme quantity per mitochondrion in the sample, and/or determining enzyme quantity per unit of protein in the sample. The Krebs cycle enzyme may be citrate synthase, aconitase, isocitrate dehydrogenase, alpha-ketoglutarate dehydrogenase, succinyl-coenzyme A synthetase, succinate dehydrogenase, fumarase or malate dehydrogenase.

In other embodiments, the indicator of mitochondrial function may be mitochondrial mass per cell in the sample. Mitochondrial mass may be determined using a mitochondria selective agent (such as nonylacridine orange) or by morphometric analysis. In certain embodiments, the indicator of mitochondrial function may be the number of mitochondria per cell in the sample which may be determined using a mitochondrion selective reagent, such as a fluorescent reagent.

In other embodiments, the indicator of mitochondrial function may be the amount of mitochondrial DNA (“mtDNA”) per cell in the sample. The amount of mitochondrial DNA per cell may be measured and/or expressed in absolute (e.g., mass of mtDNA per cell) or relative (e.g., proportion of mtDNA relative to nuclear DNA) terms. In certain embodiments, mitochondrial DNA is measured by contacting a biological sample containing mitochondrial DNA with an oligonucleotide primer having a nucleotide sequence that is complementary to a sequence present in the mitochondrial DNA, under conditions and for a time sufficient to allow hybridization of the primer to the mitochondrial DNA; and detecting hybridization of the primer to the mitochondrial DNA, and therefrom quantifying the mitochondrial DNA. In certain embodiments the step of detecting comprises a technique that may be polymerase chain reaction, oligonucleotide primer extension assay, ligase chain reaction, or restriction fragment length polymorphism analysis. In certain embodiments, mitochondrial DNA is measured by contacting a sample containing amplified mitochondrial DNA with an oligonucleotide primer having a nucleotide sequence that is complementary to a sequence present in the amplified mitochondrial DNA, under conditions and for a time sufficient to allow hybridization of the primer to the mitochondrial DNA; and detecting hybridization of the primer to the mitochondrial DNA, and therefrom quantifying the mitochondrial DNA. In certain embodiments the step of detecting comprises a technique that may be polymerase chain reaction, oligonucleotide primer extension assay, ligase chain reaction, or restriction fragment length polymorphism analysis. In certain embodiments the mitochondrial DNA is amplified using a technique that may be polymerase chain reaction, transcriptional amplification systems or self-sustained sequence replication. In certain embodiments, mitochondrial DNA is measured by contacting a biological sample containing mitochondrial DNA with an oligonucleotide primer having a nucleotide sequence that is complementary to a sequence present in the mitochondrial DNA, under conditions and for a time sufficient to allow hybridization of the primer to the mitochondrial DNA; and detecting hybridization and extension of the primer to the mitochondrial DNA to produce a product, and therefrom quantifying the mitochondrial DNA. In certain embodiments the step of comparing comprises measuring mitochondrial DNA by contacting a sample containing amplified mitochondrial DNA with an oligonucleotide primer having a nucleotide sequence that is complementary to a sequence present in the amplified mitochondrial DNA, under conditions and for a time sufficient to allow hybridization of the primer to the mitochondrial DNA; and detecting hybridization and extension of the primer to the mitochondrial DNA to produce a product, and therefrom quantifying the mitochondrial DNA. In certain embodiments the mitochondrial DNA is amplified using a technique that may be the polymerase chain reaction (PCR), including quantitative and competitive PCR (Ahmed et al., BioTechniques 26:290-300, 1999), transcriptional amplification systems or self-sustained sequence replication. In certain embodiments, the amount of mitochondrial DNA in the sample is determined using an oligonucleotide primer extension assay. In other embodiments, the amount of mitochondrial DNA is determined by subjecting a sample to a cesium chloride gradient to separate it from nuclear DNA (see, e.g., Welter et al., Mol. Biol. Rep. 13:17-120, 1988) in the presence of a detectably labeled compound that binds to double-stranded nucleic acids (e.g., ethidium bromide) and comparing the relative and/or absolute signals corresponding to the mitochondrial and nuclear DNAs.

In other embodiments, the indicator of mitochondrial function is the amount of ATP per cell in the sample. The methods may comprise measuring the amount of ATP per mitochondrion in the sample, measuring the amount of ATP per unit protein in the sample, measuring the amount of ATP per unit mitochondrial mass in the sample, measuring the amount of ATP per unit mitochondrial protein in the sample. In certain embodiments, the indicator of mitochondrial function is the rate of ATP synthesis in the sample or an ATP biosynthesis factor. The methods may comprise measuring ATP biosynthesis factor catalytic activity, determining ATP biosynthesis factor activity per mitochondrion in the sample, determining ATP biosynthesis factor activity per unit mitochondrial mass in the sample, determining ATP biosynthesis factor activity per unit of protein in the sample, measuring ATP biosynthesis factor quantity, determining ATP biosynthesis factor quantity per mitochondrion in the sample, and/or determining ATP biosynthesis factor quantity per unit of protein in the sample.

In other embodiments, the indicator of mitochondrial function may be one or more of the following: free radical production, reactive oxygen species, protein nitrosylation, protein carbonyl modification, DNA oxidation, mtDNA oxidation, protein oxidation, protein carbonyl modification, malondialdehyde adducts of proteins, a glycoxidation product, a lipoxidation product, 8′-OH-guanosine adducts, BARS, cellular response to elevated intracellular calcium, and/or cellular response to at least one apoptogen. In certain embodiments the indicator of mitochondrial function is oxygen consumption, which may be determined according to any of a variety of known methodologies (e.g., Wu et al., 1999 Cell 98:115; Li et al. 1999 J. Biol. Chem. 274:17534).

Functional mitochondria contain gene products encoded by mitochondrial genes situated in mitochondrial DNA (mtDNA) and by extramitochondrial genes (e.g., nuclear genes) not situated in the circular mitochondrial genome. The 16.5 kb mtDNA encodes 22 tRNAs, two ribosomal RNAs (rRNA) and 13 enzymes of the electron transport chain (ETC), the elaborate multi-complex mitochondrial assembly where, for example, respiratory oxidative phosphorylation takes place. The overwhelming majority of mitochondrial structural and functional proteins are encoded by extramitochondrial, and in most cases presumably nuclear, genes. Accordingly, mitochondrial and extramitochondrial genes may interact directly, or indirectly via gene products and their downstream intermediates, including metabolites, catabolites, substrates, precursors, cofactors and the like. Alterations in mitochondrial function, for example impaired electron transport activity, defective oxidative phosphorylation or increased free radical production, may therefore arise as the result of defective mtDNA, defective extramitochondrial DNA, defective mitochondrial or extramitochondrial gene products, defective downstream intermediates or a combination of these and other factors.

In certain embodiments, an enzyme is the indicator of mitochondrial function as provided herein. The enzyme may be a mitochondrial enzyme, which may further be an ETC enzyme or a Krebs cycle enzyme. The enzyme may also be an ATP biosynthesis factor, which may include an ETC enzyme and/or a Krebs cycle enzyme, or other enzymes or cellular components related to ATP production as provided herein. A “non-enzyme” refers to an indicator of mitochondrial function that is not an enzyme (i.e., that is not a mitochondrial enzyme or an ATP biosynthesis factor as provided herein). In certain other embodiments, an enzyme is a co-indicator of mitochondrial function. The following enzymes may not be indicators of mitochondrial function according to the present invention, but may be co-indicators of mitochondrial function as provided herein: citrate synthase (EC 4.1.3.7), hexokinase II (EC 2.7.1.1), cytochrome c oxidase (EC 1.9.3.1), phosphofructokinase (EC 2.7.1.11), glyceraldehyde phosphate dehydrogenase (EC 1.2.1.12), glycogen phosphorylase (EC 2.4.1.1) creatine kinase (EC 2.7.3.2), NADH dehydrogenase (EC 1.6.5.3), glycerol 3-phosphate dehydrogenase (EC 1.1.1.8), triose phosphate dehydrogenase (EC 1.2.1.12) and malate dehydrogenase (EC 1.1.1.37).

In other embodiments, the indicator of mitochondrial function is any ATP biosynthesis factor, ATP production, mitochondrial mass or mitochondrial number, free radical production, a cellular response to elevated intracellular calcium and/or a cellular response to an apoptogen. In certain embodiments, mitochondrial DNA content may not be an indicator of mitochondrial function but may be a co-predictor of mitochondrial function or a co-indicator of mitochondrial function, as provided herein.

i. Indicators of Mitochondrial Function that are Enzymes

In certain embodiments, methods for identifying agents that modulate mitochondrial mass and/or function include the detection and/or absolute or relative measurement of at least one indicator of mitochondrial function in biological test samples, wherein the indicator of mitochondrial function is an enzyme. As provided herein, such an enzyme may be a mitochondrial enzyme or an ATP biosynthesis factor that is an enzyme, for example an ETC enzyme or a Krebs cycle enzyme.

Reference to “enzyme quantity”, “enzyme catalytic activity” or “enzyme expression level” in the context of the methods for identifying agents that modulate mitochondrial mass and/or function, is meant to include a reference to any of a mitochondrial enzyme quantity, activity or expression level or an ATP biosynthesis factor quantity, activity or expression level; either of which may further include, for example, an ETC enzyme quantity, activity or expression level or a Krebs cycle enzyme quantity, activity or expression level. In the most preferred embodiments of the invention, an enzyme is a natural or recombinant protein or polypeptide that has enzyme catalytic activity as provided herein. Such an enzyme may be, by way of non-limiting examples, an enzyme, a holoenzyme, an enzyme complex, an enzyme subunit, an enzyme fragment, derivative or analog or the like, including a truncated, processed or cleaved enzyme.

A mitochondrial enzyme that may be an indicator of mitochondrial function as provided herein refers to a mitochondrial molecular component that has enzyme catalytic activity and/or functions as an enzyme cofactor capable of influencing enzyme catalytic activity. As used herein, mitochondria are comprised of “mitochondrial molecular components”, which may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or any covalently or non-covalently complexed combination of these components, or any other biological molecule that is a stable or transient constituent of a mitochondrion.

A mitochondrial enzyme that may be an indicator of mitochondrial function or a co-indicator of mitochondrial function as provided herein, or an ATP biosynthesis factor that may be an indicator of mitochondrial function as provided herein, may comprise an ETC enzyme, which refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC is typically described as comprising complex I (NADH:ubiquinone reductase), complex II (succinate dehydrogenase), complex III (ubiquinone: cytochrome c oxidoreductase), complex IV (cytochrome c oxidase) and complex V (mitochondrial ATP synthetase), where each complex includes multiple polypeptides and cofactors (for review see, e.g., Walker et al., 1995 Meths. Enzymol. 260:14; Emster et al., 1981 J. Cell Biol. 91:227s-255s, and references cited therein).

A mitochondrial enzyme that may be an indicator of mitochondrial function as provided herein, or an ATP biosynthesis factor that may be an indicator of mitochondrial function as provided herein, may also comprise a Krebs cycle enzyme, which includes mitochondrial molecular components that mediate the series of biochemical/bioenergetic reactions also known as the citric acid cycle or the tricarboxylic acid cycle (see, e.g., Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.). Krebs cycle enzymes include subunits and cofactors of citrate synthase, aconitase, isocitrate dehydrogenase, the a-ketoglutarate dehydrogenase complex, succinyl CoA synthetase, succinate dehydrogenase, fumarase and malate dehydrogenase. Krebs cycle enzymes further include enzymes and cofactors that are functionally linked to the reactions of the Krebs cycle, such as, for example, nicotinamide adenine dinucleotide, coenzyme A, thiamine pyrophosphate, lipoamide, guanosine diphosphate, flavin adenine dinucloetide and nucleoside diphosphokinase.

The methods described herein also pertain in part to the correlation of type 2 diabetes with an indicator of mitochondrial function that may be an ATP biosynthesis factor, an altered amount of ATP or an altered amount of ATP production. For example, decreased mitochondrial ATP biosynthesis may be an indicator of mitochondrial function from which a risk for type 2 diabetes may be identified.

An “ATP biosynthesis factor” refers to any naturally occurring cellular component that contributes to the efficiency of ATP production in mitochondria. Such a cellular component may be a protein, polypeptide, peptide, amino acid, or derivative thereof, a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like. An ATP biosynthesis factor includes at least the components of the ETC and of the Krebs cycle (see, e.g., Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.) and any protein, enzyme or other cellular component that participates in ATP synthesis, regardless of whether such ATP biosynthesis factor is the product of a nuclear gene or of an extranuclear gene (e.g., a mitochondrial gene). Participation in ATP synthesis may include, but need not be limited to, catalysis of any reaction related to ATP synthesis, transmembrane import and/or export of ATP or of an enzyme cofactor, transcription of a gene encoding a mitochondrial enzyme and/or translation of such a gene transcript.

Compositions and methods for determining whether a cellular component is an ATP biosynthesis factor are well known in the art, and include methods for determining ATP production (including determination of the rate of ATP production in a sample) and methods for quantifying ATP itself. The contribution of an ATP biosynthesis factor to ATP production can be determined, for example, using an isolated ATP biosynthesis factor that is added to cells or to a cell-free system. The ATP biosynthesis factor may directly or indirectly mediate a step or steps in a biosynthetic pathway that influences ATP production. For example, an ATP biosynthesis factor may be an enzyme that catalyzes a particular chemical reaction leading to ATP production. As another example, an ATP biosynthesis factor may be a cofactor that enhances the efficiency of such an enzyme. As another example, an ATP biosynthesis factor may be an exogenous genetic element introduced into a cell or a cell-free system that directly or indirectly affects an ATP biosynthetic pathway. Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, .sup.32P; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of mitochondrial function. In one embodiment, for instance, ATP may be quantified by measuring luminescence of luciferase catalyzed oxidation of D-luciferin, an ATP dependent process.

“Enzyme catalytic activity” refers to any function performed by a particular enzyme or category of enzymes that is directed to one or more particular cellular function(s). For example, “ATP biosynthesis factor catalytic activity” refers to any function performed by an ATP biosynthesis factor as provided herein that contributes to the production of ATP. Typically, enzyme catalytic activity is manifested as facilitation of a chemical reaction by a particular enzyme, for instance an enzyme that is an ATP biosynthesis factor, wherein at least one enzyme substrate or reactant is covalently modified to form a product. For example, enzyme catalytic activity may result in a substrate or reactant being modified by formation or cleavage of a covalent chemical bond, but the invention need not be so limited. Various methods of measuring enzyme catalytic activity are known to those having ordinary skill in the art and depend on the particular activity to be determined.

For many enzymes, including mitochondrial enzymes or enzymes that are ATP biosynthesis factors as provided herein, quantitative criteria for enzyme catalytic activity are well established. These criteria include, for example, activity that may be defined by international units (IU), by enzyme turnover number, by catalytic rate constant (K_{cat}), by Michaelis-Menten constant (Kₘ), by specific activity or by any other enzymological method known in the art for measuring a level of at least one enzyme catalytic activity. Specific activity of a mitochondrial enzyme, such as an ATP biosynthesis factor, may be expressed as units of substrate detectably converted to product per unit time and, optionally, further per unit sample mass (e.g., per unit protein or per unit mitochondrial mass).

In certain embodiments, enzyme catalytic activity may be expressed as units of substrate detectably converted by an enzyme to a product per unit time per unit total protein in a sample, as units of substrate detectably converted by an enzyme to product per unit time per unit mitochondrial mass in a sample, or as units of substrate detectably converted by an enzyme to product per unit time per unit mitochondrial protein mass in a sample. Products of enzyme catalytic activity may be detected by suitable methods that will depend on the quantity and physicochemical properties of the particular product. Thus, detection may be, for example by way of illustration and not limitation, by radiometric, colorimetric, spectrophotometric, fluorimetric, immunometric or mass spectrometric procedures, or by other suitable means that will be readily apparent to a person having ordinary skill in the art.

In certain embodiments, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals—Sixth Ed., Molecular Probes, Eugene, Oreg.; Scopes, R. K., Protein Purification Principles and Practice, 1987, Springer-Verlag, New York; Hermanson, G. T. et al., Immobilized Affinity Ligand Techniques, 1992, Academic Press, Inc., New York; Luo et al., 1998 J. Biotechnol. 65:225 and references cited therein). Spectroscopic methods may be used to detect dyes (including, for example, calorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well known techniques.

As noted above, enzyme catalytic activity of an ATP biosynthesis factor may further include other functional activities that lead to ATP production, beyond those involving covalent alteration of a substrate or reactant. For example by way of illustration and not limitation, an ATP biosynthesis factor that is an enzyme may refer to a transmembrane transporter molecule that, through its enzyme catalytic activity, facilitates the movement of metabolites between cellular compartments. Such metabolites may be ATP or other cellular components involved in ATP synthesis, such as gene products and their downstream intermediates, including metabolites, catabolites, substrates, precursors, cofactors and the like. As another non-limiting example, an ATP biosynthesis factor that is an enzyme may, through its enzyme catalytic activity, transiently bind to a cellular component involved in ATP synthesis in a manner that promotes ATP synthesis. Such a binding event may, for instance, deliver the cellular component to another enzyme involved in ATP synthesis and/or may alter the conformation of the cellular component in a manner that promotes ATP synthesis. Further to this example, such conformational alteration may be part of a signal transduction pathway, an allosteric activation pathway, a transcriptional activation pathway or the like, where an interaction between cellular components leads to ATP production.

Thus, an ATP biosynthesis factor may include, for example, a mitochondrial membrane protein. Suitable mitochondrial membrane proteins include such mitochondrial components as the adenine nucleotide transporter (ANT; e.g., Fiore et al., 1998 Biochimie 80:137; Klingenberg 1985 Ann. New York Acad. Sci. 456:279), the voltage dependent anion channel (VDAC, also referred to as porin; e.g., Manella, 1997 J. Bioenergetics Biomembr. 29:525), the malate-aspartate shuttle, the mitochondrial calcium uniporter (e.g., Litsky et al., 1997 Biochem. 36:7071), uncoupling proteins (UCP-1, -2, -3; see e.g., Jezek et al., 1998 Int. J. Biochem. Cell Biol. 30:1163), a hexokinase, a peripheral benzodiazepine receptor, a mitochondrial intermembrane creatine kinase, cyclophilin D, a Bcl-2 gene family encoded polypeptide, the tricarboxylate carrier (e.g., Iocobazzi et al., 1996 Biochim. Biophys. Acta 1284:9; Bisaccia et al., 1990 Biochim. Biophys. Acta 1019:250) and the dicarboxylate carrier (e.g., Fiermonte et al., 1998 J. Biol. Chem. 273:24754; Indiveri et al., 1993 Biochim. Biophys. Acta 1143:310; for a general review of mitochondrial membrane transporters, see, e.g., Zonatti et al., 1994 J. Bioenergetics Biomembr. 26:543 and references cited therein).

Enzyme quantity as used herein with reference to the methods for identifying modulators of mitochondrial mass and/or function refers to an amount of an enzyme including mitochondrial enzymes or enzymes that are ATP biosynthesis factors as provided herein, or of another ATP biosynthesis factor, that is present, i.e., the physical presence of an enzyme or ATP biosynthesis factor selected as an indicator of mitochondrial function, irrespective of enzyme catalytic activity. Depending on the physicochemical properties of a particular enzyme or ATP biosynthesis factor, the preferred method for determining the enzyme quantity will vary. In the most highly preferred embodiments of the invention, determination of enzyme quantity will involve quantitative determination of the level of a protein or polypeptide using routine methods in protein chemistry with which those having skill in the art will be readily familiar, for example by way of illustration and not limitation, those described in greater detail below.

Accordingly, determination of enzyme quantity may be by any suitable method known in the art for quantifying a particular cellular component that is an enzyme or an ATP biosynthesis factor as provided herein, and that in preferred embodiments is a protein or polypeptide. Depending on the nature and physicochemical properties of the enzyme or ATP biosynthesis factor, determination of enzyme quantity may be by densitometric, mass spectrometric, spectrophotometric, fluorimetric, immunometric, chromatographic, electrochemical or any other means of quantitatively detecting a particular cellular component. Methods for determining enzyme quantity also include methods described above that are useful for detecting products of enzyme catalytic activity, including those measuring enzyme quantity directly and those measuring a detectable label or reporter moiety. In certain preferred embodiments of the invention, enzyme quantity is determined by immunometric measurement of an isolated enzyme or ATP biosynthesis factor. In certain preferred embodiments of the invention, these and other immunological and immunochemical techniques for quantitative determination of biomolecules such as an enzyme or ATP biosynthesis factor may be employed using a variety of assay formats known to those of ordinary skill in the art, including but not limited to enzyme linked immunosorbent assay (ELISA), radioimmunoassay (RIA), immunofluorimetry, immunoprecipitation, equilibrium dialysis, immunodiffusion and other techniques. (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston.) For example, the assay may be performed in a Western blot format, wherein a preparation comprising proteins from a biological sample is submitted to gel electrophoresis, transferred to a suitable membrane and allowed to react with an antibody specific for an enzyme or an ATP biosynthesis factor that is a protein or polypeptide. The presence of the antibody on the membrane may then be detected using a suitable detection reagent, as is well known in the art and described above.

In certain embodiments, an indicator (or co-indicator) of mitochondrial function including, for example, an enzyme as provided herein, may be present in an isolated form, e.g., removed from its original environment (e.g., the natural environment if it is naturally occurring). For example, a naturally occurring polypeptide present in a living animal is not isolated, but the same polypeptide, separated from some or all of the co-existing materials in the natural system, is isolated. Such polypeptides could be part of a composition, and still be isolated in that such composition is not part of its natural environment.

Affinity techniques are useful in the context of isolating an enzyme or an ATP biosynthesis factor protein or polypeptide for use according to the methods of the present invention, and may include any method that exploits a specific binding interaction involving an enzyme or an ATP biosynthesis factor to effect a separation. For example, because an enzyme or an ATP biosynthesis factor protein or polypeptide may contain covalently attached oligosaccharide moieties, an affinity technique such as binding of the enzyme (or ATP biosynthesis factor) to a suitable immobilized lectin under conditions that permit carbohydrate binding by the lectin may be a particularly useful affinity technique.

Other useful affinity techniques include immunological techniques for isolating and/or detecting a specific protein or polypeptide antigen (e.g., an enzyme or ATP biosynthesis factor), which techniques rely on specific binding interaction between antibody combining sites for antigen and antigenic determinants present on the factor. Binding of an antibody or other affinity reagent to an antigen is “specific” where the binding interaction involves a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹. Affinities of binding partners or antibodies can be readily determined using conventional techniques, for example those described by Scatchard et al., Ann. New York Acad. Sci. 51:660 (1949).

Immunological techniques include, but need not be limited to, immunoaffinity chromatography, immunoprecipitation, solid phase immunoadsorption or other immunoaffinity methods. For these and other useful affinity techniques, see, for example, Scopes, R. K., Protein Purification: Principles and Practice, 1987, Springer-Verlag, New York; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques.

As noted above, an indicator of mitochondrial function can be a protein or polypeptide, for example an enzyme or an ATP biosynthesis factor. The protein or polypeptide may be an unmodified polypeptide or may be a polypeptide that has been posttranslationally modified, for example by glycosylation, phosphorylation, fatty acylation including glycosylphosphatidylinositol anchor modification or the like, phospholipase cleavage such as phosphatidylinositol-specific phospholipase c mediated hydrolysis or the like, protease cleavage, dephosphorylation or any other type of protein posttranslational modification such as a modification involving formation or cleavage of a covalent chemical bond.

ii. Indicators of Mitochondrial Function that are Mitochondrial Mass, Mitochondrial Volume or Mitochondrial Number

In certain embodiments, methods for identifying agents that modulate mitochondrial mass and/or function include the detection and/or measurement of at least one indicator of mitochondrial function in biological test samples, wherein the indicator of mitochondrial function is absolute or relative mitochondrial mass, mitochondrial volume or mitochondrial number.

Methods for quantifying mitochondrial mass, volume and/or mitochondrial number are known in the art, and may include, for example, quantitative staining of a representative biological sample. Typically, quantitative staining of mitochondrial may be performed using organelle-selective probes or dyes, including but not limited to mitochondrion selective reagents such as fluorescent dyes that bind to mitochondrial molecular components (e.g., nonylacridine orange, MitoTrackers) or potentiometric dyes that accumulate in mitochondria as a function of mitochondrial inner membrane electrochemical potential (see, e.g., Haugland, 1996 Handbook of Fluorescent Probes and Research Chemicals, Sixth Ed., Molecular Probes, Eugene, Oreg.). As another example, mitochondrial mass, volume and/or number may be quantified by morphometric analysis (e.g., Cruz-Orive et al., 1990 Am. J. Physiol. 258:L148; Schwerzmann et al., 1986 J. Cell Biol. 102:97). These or any other means known in the art for quantifying mitochondrial mass, volume and/or mitochondrial number in a sample are within the contemplated scope of the invention. For example, the use of such quantitative determinations for purposes of calculating mitochondrial density is contemplated and is not intended to be limiting. In certain embodiments, mitochondrial protein mass in a sample is determined using well known procedures. For example, a person having ordinary skill in the art can readily prepare an isolated mitochondrial fraction from a biological sample using established cell fractionation techniques, and therefrom determine protein content using any of a number of protein quantification methodologies well known in the art.

iii. Indicators of Mitochondrial Function that Include Mitochondrial DNA Content

In other embodiments, methods for identifying modulators of mitochondrial mass and/or function include the detection and/or measurement of at least one indicator of mitochondrial function in biological test samples, wherein the indicator of mitochondrial function is the absolute or relative amount of mitochondrial DNA. Quantification of mitochondrial DNA (mtDNA) content may be accomplished by any of a variety of established techniques that are useful for this purpose, including but not limited to oligonucleotide probe hybridization or polymerase chain reaction (PCR) using oligonucleotide primers specific for mitochondrial DNA sequences (see, e.g., Miller et al., 1996 J. Neurochem. 67:1897; Fahy et al., 1997 Nucl. Ac. Res. 25:3102; U.S. patent application Ser. No. 09/098,079; Lee et al., 1998 Diabetes Res. Clin. Practice 42:161; Lee et al., 1997 Diabetes 46 (suppl. 1):175A). A particularly useful method is the primer extension assay disclosed by Fahy et al. (Nucl. Acids Res. 25:3102, 1997) and by Ghosh et al. (Am. J. Hum. Genet. 58:325, 1996). Suitable hybridization conditions may be found in the cited references or may be varied according to the particular nucleic acid target and oligonucleotide probe selected, using methodologies well known to those having ordinary skill in the art (see, e.g., Ausubel et al., Current Protocols in Molecular Biology, Greene Publishing, 1987; Sambrook et al., Molecular Cloning: A Laboratory Manual, Cold Spring Harbor Press, 1989).

Examples of other useful techniques for determining the amount of specific nucleic acid target sequences (e.g., mtDNA) present in a sample based on specific hybridization of a primer to the target sequence include specific amplification of target nucleic acid sequences and quantification of amplification products, including but not limited to polymerase chain reaction (PCR, Gibbs et al., Nucl. Ac. Res. 17:2437, 1989), transcriptional amplification systems (e.g., Kwoh et al., 1989 Proc. Nat. Acad. Sci. 86:1173); strand displacement amplification (e.g., Walker et al., Nucl. Ac. Res. 20:1691, 1992; Walker et al., Proc. Nat. Acad. Sci. 89:392, 1992) and self-sustained sequence replication (3SR, see, e.g., Ghosh et al, in Molecular Methods for Virus Detection, 1995 Academic Press, New York, pp. 287-314; Guatelli et al., Proc. Nat. Acad. Sci. 87:1874, 1990), the cited references for which are incorporated herein by reference in their entireties. Other useful amplification techniques include, for example, ligase chain reaction (e.g., Barany, Proc. Nat. Acad. Sci. 88:189, 1991), Q-beta replicase assay (Cahill et al., Clin. Chem. 37:1482, 1991; Lizardi et al., Biotechnol. 6:1197, 1988; Fox et al., J. Clin. Lab. Analysis 3:378, 1989) and cycled probe technology (e.g., Cloney et al., Clin. Chem. 40:656, 1994), as well as other suitable methods that will be known to those familiar with the art.

Sequence length or molecular mass of primer extension assay products may be determined using any known method for characterizing the size of nucleic acid sequences with which those skilled in the art are familiar. In one embodiment, primer extension products are characterized by gel electrophoresis. In another embodiment, primer extension products are characterized by mass spectrometry (MS), which may further include matrix assisted laser desorption ionization/time of flight (MALDI-TOF) analysis or other MS techniques known to those skilled in the art. See, for example, U.S. Pat. Nos. 5,622,824, 5,605,798 and 5,547,835. In another embodiment, primer extension products are characterized by liquid or gas chromatography, which may further include high performance liquid chromatography (HPLC), gas chromatography-mass spectrometry (GC-MS) or other well known chromatographic methodologies.

iv. Indicators of Mitochondrial Function that are Cellular Responses to Elevated Intracellular Calcium

Certain aspects of the present invention, as it relates detecting and/or measuring an indicator of mitochondrial function, involve monitoring intracellular calcium homeostasis and/or cellular responses to perturbations of this homeostasis, including physiological and pathophysiological calcium regulation. The range of cellular responses to elevated intracellular calcium is broad, as is the range of methods and reagents for the detection of such responses. Many specific cellular responses are known to those having ordinary skill in the art; these responses will depend on the particular cell types present in a selected biological sample. As non-limiting examples, cellular responses to elevated intracellular calcium include secretion of specific secretory products, exocytosis of particular pre-formed components, increased glycogen metabolism and cell proliferation (see, e.g., Clapham, 1995 Cell 80:259; Cooper, The Cell—A Molecular Approach, 1997 ASM Press, Washington, D.C.; Alberts, B., Bray, D., et al., Molecular Biology of the Cell, 1995 Garland Publishing, New York).

As a brief background, normal alterations of intramitochondrial calcium are associated with normal metabolic regulation (Dykens, 1998 in Mitochondria & Free Radicals in Neurodegenerative Diseases, Beal, Howell and Bodis-Wollner, Eds., Wiley-Liss, New York, pp. 29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1991, Am. J. Physio. 27: C755; Gunter et al., 1994, Am. J. Physiol. 267:313). For example, fluctuating levels of mitochondrial free Calcium may be responsible for regulating oxidative metabolism in response to increased ATP utilization, via allosteric regulation of enzymes (reviewed by Crompton et al., 1993 Basic Res. Cardiol. 88: 513-523); and the glycerophosphate shuttle (Gunter et al., 1994 J. Bioenerg. Biomembr. 26: 471).

Normal mitochondrial function includes regulation of cytosolic free calcium levels by sequestration of excess calcium within the mitochondrial matrix. Depending on cell type, cytosolic calcium concentration is typically 50-100 nM. In normally functioning cells, when calcium levels reach 200-300 nM, mitochondria begin to accumulate calcium as a function of the equilibrium between influx via a calcium uniporter in the inner mitochondrial membrane and calcium efflux via both sodium dependent and sodium independent calcium carriers. In certain instances, such perturbation of intracellular calcium homeostasis is a feature of diseases (such as type 2 diabetes) associated with mitochondrial function, regardless of whether the calcium regulatory dysfunction is causative of, or a consequence of, mitochondrial function.

Elevated mitochondrial calcium levels thus may accumulate in response to an initial elevation in cytosolic free calcium, as described above. Such elevated mitochondrial calcium concentrations in combination with reduced ATP or other conditions associated with mitochondrial pathology, can lead to collapse of mitochondrial inner membrane potential (see Gunter et al., 1998 Biochim. Biophys. Acta 1366:5; Rottenberg and Marbach, 1990, Biochim. Biophys. Acta 1016:87). The extramitochondrial (cytosolic) level of calcium in a biological sample that is greater than that present within mitochondria may be used as a risk factor for type 2 diabetes in an individual. In the case of type 2 diabetes, mitochondrial or cytosolic calcium levels may vary from the above ranges and may range from, e.g., about 1 nM to about 500 mM, more typically from about 10 nM to about 100 mM and usually from about 20 nM to about 1 mM, where “about” indicates +/−10%. A variety of calcium indicators are known in the art, including but not limited to, for example, fura-2 (McCormack et al., 1989 Biochim. Biophys. Acta 973:420); mag-fura-2; BTC (U.S. Pat. No. 5,501,980); fluo-3, fluo-4 and fluo-5N (U.S. Pat. No. 5,049,673); rhod-2; benzothiaza-1; and benzothiaza-2 (all of which are available from Molecular Probes, Eugene, Oreg.). These or any other means for monitoring intracellular calcium are contemplated according to the subject invention method for identifying a risk for type 2 diabetes.

For monitoring an indicator of mitochondrial function that is a cellular response to elevated intracellular calcium, compounds that induce increased cytoplasmic and mitochondrial concentrations of calcium, including calcium ionophores, are well known to those of ordinary skill in the art, as are methods for measuring intracellular calcium and intramitochondrial calcium (see, e.g., Gunter and Gunter, 1994 J. Bioenerg. Biomembr. 26: 471; Gunter et al., 1998 Biochim. Biophys. Acta 1366:5; McCormack et al., 1989 Biochim. Biophys. Acta 973:420; Orrenius and Nicotera, 1994 J. Neural. Transm. Suppl. 43:1; Leist and Nicotera, 1998 Rev. Physiol. Biochem. Pharmacol. 132:79; and Haugland, 1996 Handbook of Fluorescent Probes and Research Chemicals, Sixth Ed., Molecular Probes, Eugene, Oreg.). Accordingly, a person skilled in the art may readily select a suitable ionophore (or another compound that results in increased cytoplasmic and/or mitochondrial concentrations of calcium ions) and an appropriate means for detecting intracellular and/or intramitochondrial calcium for use in the present invention, according to the instant disclosure and to well known methods.

Calcium ion influx into mitochondria appears to be largely dependent, and may be completely dependent, upon the negative transmembrane electrochemical potential (DY) established at the inner mitochondrial membrane by electron transfer, and such influx fails to occur in the absence of DY even when an eight-fold Calcium concentration gradient is imposed (Kapus et al., 1991 FEBS Lett. 282:61). Accordingly, mitochondria may release Calcium when the membrane potential is dissipated, as occurs with uncouplers like 2,4-dinitrophenol and carbonyl cyamide p-trifluoro-methoxyphenylhydrazone (FCCP). Thus, according to certain embodiments of the present invention, collapse of DY may be potentiated by influxes of cytosolic free calcium into the mitochondria, as may occur under certain physiological conditions including those encountered by cells of a subject having type 2 DM. Detection of such collapse may be accomplished by a variety of means as provided herein.

Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Emster et al., 1981 J. Cell Biol. 91:227s and references cited; see also Haugland, 1996 Handbook of Fluorescent Probes and Research Chemicals, Sixth Ed., Molecular Probes, Eugene, Oreg., pp. 266-274 and 589-594). For example, by way of illustration and not limitation, the fluorescent probes 2-,4-dimethylaminostyryl-N-methylpyridinium (DASPMI) and tetramethylrhodamine esters (e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, on the world wide web at probes.com/handbook/toc.html). Other fluorescent detectable compounds that may be used include but are not limited to rhodamine 123, rhodamine B hexyl ester, DiOC.sub.6(3), JC-1 [5,5′,6,6′-Tetrachloro-1,1′,3,3′-Tetraethylbez-imidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Gottingen, Germany; see Sakanoue et al., 1997 J. Biochem. 121:29). Methods for monitoring mitochondrial membrane potential are also disclosed in U.S. patent application Ser. No. 09/161,172.

Mitochondrial membrane potential can also be measured by non-fluorescent means, for example by using TTP (tetraphenylphosphonium ion) and a TTP-sensitive electrode (Kamo et al., 1979 J. Membrane Biol. 49:105; Porter and Brand, 1995 Am. J. Physiol. 269:RI213). Those skilled in the art will be able to select appropriate detectable compounds or other appropriate means for measuring DYm. By way of example and not limitation, TMRM is somewhat preferable to TMRE because, following efflux from mitochondria, TMRE yields slightly more residual signal in the endoplasmic reticulicum and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein).

Exquisite sensitivity to extraordinary mitochondrial accumulations of calcium that result from elevation of intracellular calcium, as described above, may also characterize type 2 diabetes. Such mitochondrial sensitivity may provide an indicator of mitochondrial function according to the present invention. Additionally, a variety of physiologically pertinent agents, including hydroperoxide and free radicals, may synergize with calcium to induce collapse of DY (Novgorodov et al., 1991 Biochem. Biophys. Acta 1058: 242; Takeyama et al., 1993 Biochem. J. 294: 719; Guidox et al., 1993 Arch. Biochem. Biophys. 306:139).

v. Indicators of Mitochondrial Function that Include Responses to Apoptogenic Stimuli

In another embodiment, methods for identifying a modulator of mitochondrial mass and/or function may include the detection and/or measurement of an indicator of mitochondrial function, wherein the mitochondrial function involves programmed cell death or apoptosis. The range of responses to various known apoptogenic stimuli is broad, as is the range of methods and reagents for the detection of such responses.

Mitochondrial dysfunction is thought to be critical in the cascade of events leading to apoptosis in various cell types (Kroemer et al., FASEB J 9:1277-87, 1995). Mitochondrial physiology may be among the earliest events in programmed cell death (Zamzami et al., J. Exp. Med. 182:367-77, 1995; Zamzami et al., J. Exp. Med. 181:1661-72, 1995) and elevated reactive oxygen species (ROS) levels that result from such mitochondrial function may initiate the apoptotic cascade (Ausserer et al., Mol Cell Biol 14:5032-42, 1994). In several cell types, reduction in the mitochondrial membrane potential (DYm) precedes the nuclear DNA degradation that accompanies apoptosis. In cell-free systems, mitochondrial, but not nuclear, enriched fractions are capable of inducing nuclear apoptosis (Newmeyer et al., Cell 70:353-64, 1994). Perturbation of mitochondrial respiratory activity leading to altered cellular metabolic states, such as elevated intracellular ROS, may occur in type 2 diabetes and may further induce pathogenetic events via apoptotic mechanisms.

Oxidatively stressed mitochondria may release a pre-formed soluble factor that can induce chromosomal condensation, an event preceding apoptosis (Marchetti et al., Cancer Res. 56:2033-38, 1996). In addition, members of the Bcl-2 family of anti-apoptosis gene products are located within the outer mitochondrial membrane (Monaghan et al., J. Histochem. Cytochem. 40:1819-25, 1992) and these proteins appear to protect membranes from oxidative stress (Korsmeyer et al., Biochim. Biophys. Act. 1271:63, 1995). Localization of Bcl-2 to this membrane appears to be indispensable for modulation of apoptosis (Nguyen et al., J. Biol. Chem. 269:16521-24, 1994). Thus, changes in mitochondrial physiology may be important mediators of apoptosis.

Impaired mitochondrial function may therefore be reflected in a lower threshold for induction of apoptosis by one or more apoptogens. A variety of apoptogens are known to those familiar with the art (see, e.g., Green et al., 1998 Science 281:1309 and references cited therein) and may include by way of illustration and not limitation: tumor necrosis factor-alpha (TNF-a); Fas ligand; glutamate; N-methyl-D-aspartate (NMDA); interleukin-3 (IL-3); herbimycin A (Mancinit et al., 1997 J. Cell. Biol. 138:449-469); paraquat (Costantini et al., 1995 Toxicology 99:1-2); ethylene glycols; protein kinase inhibitors, e.g., staurosporine, calphostin C, caffeic acid phenethyl ester, chelerythrine chloride, genistein; 1-(5-isoquinolinesulfonyl)-2-methylpiperazine; KN-93; N-[2-((p-bromocinnamyl)amino)ethyl]-5-5-isoquinolinesulfonamide; d-erythrosphingosine derivatives; UV irradiation; ionophores, e.g., ionomycin and valinomycin; MAP kinase inducers, e.g., anisomycin, anandamine; cell cycle blockers, e.g., aphidicolin, colcemid, 5-fluorouracil, homoharringtonine; acetylcholinesterase inhibitors, e.g. berberine; anti-estrogens, e.g., tamoxifen; pro-oxidants, e.g., tert-butyl peroxide, hydrogen peroxide; free radicals, e.g., nitric oxide; inorganic metal ions, e.g., cadmium; DNA synthesis inhibitors, e.g., actinomycin D; DNA intercalators, e.g., doxorubicin, bleomycin sulfate, hydroxyurea, methotrexate, mitomycin C, camptothecin, daunorubicin; protein synthesis inhibitors, e.g., cycloheximide, puromycin, rapamycin; agents that affect microtubulin formation or stability, e.g., vinblastine, vincristine, colchicine, 4-hydroxyphenylretinamide, paclitaxel; Bad protein, Bid protein and Bax protein (see, e.g., Jurgenmeier et al., 1998 Proc. Nat. Acad. Sci. USA 95:4997-5002 and references cited therein); calcium and inorganic phosphate (Kroemer et al., 1998 Ann. Rev. Physiol 60:619).

In one embodiment, wherein the indicator of mitochondrial function is a cellular response to an apoptogen, cells in a biological sample that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by a person having ordinary skill in the art, for example by using light microscopy. As another example, cells undergoing apoptosis may exhibit fragmentation and disintegration of chromosomes, which may be apparent by microscopy and/or through the use of DNA-specific or chromatin-specific dyes that are known in the art, including fluorescent dyes. Such cells may also exhibit altered plasma membrane permeability properties as may be readily detected through the use of vital dyes (e.g., propidium iodide, trypan blue) or by the detection of lactate dehydrogenase leakage into the extracellular milieu. These and other means for detecting apoptotic cells by morphologic criteria, altered plasma membrane permeability and related changes will be apparent to those familiar with the art.

In another embodiment, wherein the indicator of mitochondrial function is a cellular response to an apoptogen, cells in a biological sample may be assayed for translocation of cell membrane phosphatidylserine (PS) from the inner to the outer leaflet of the plasma membrane, which may be detected, for example, by measuring outer leaflet binding by the PS-specific protein annexin. (Martin et al., J. Exp. Med. 182:1545, 1995; Fadok et al., J. Immunol. 148:2207, 1992.) In still another embodiment, a cellular/biochemical response to an apoptogen is determined by an assay for induction of specific protease activity in any member of a family of apoptosis-activated proteases known as the caspases (see, e.g., Green et al., 1998 Science 281:1309). Those having ordinary skill in the art will be readily familiar with methods for determining caspase activity, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., 1997 J. Neurosci. 17:6165). Synthetic peptide substrates have been defined (Kluck et al., 1997 Science 275:1132; Nicholson et al., 1995 Nature 376:37). Other non-limiting examples of substrates include nuclear proteins such as U1-70 kDa and DNA-PKcs (Rosen and Casciola-Rosen, 1997 J. Cell. Biochem. 64:50; Cohen, 1997 Biochem. J. 326:1).

As described above, the mitochondrial inner membrane may exhibit highly selective and regulated permeability for many small solutes, but is impermeable to large (less than around 10 kDa) molecules. (See, e.g., Quinn, 1976 The Molecular Biology of Cell Membranes, University Park Press, Baltimore, Md.). In cells undergoing apoptosis, however, collapse of mitochondrial membrane potential may be accompanied by increased permeability permitting macromolecule diffusion across the mitochondrial membrane. Thus, in another embodiment of the subject invention method wherein the indicator of mitochondrial function is a cellular response to an apoptogen, detection of a mitochondrial protein, for example cytochrome c that has escaped from mitochondria in apoptotic cells, may provide evidence of a response to an apoptogen that can be readily determined. (Liu et al., Cell 86:147, 1996) Such detection of cytochrome c may be performed spectrophotometrically, immunochemically or by other well established methods for determining the presence of a specific protein.

For instance, release of cytochrome c from cells challenged with apoptotic stimuli (e.g., ionomycin, a well known calcium ionophore) can be followed by a variety of immunological methods. Matrix-assisted laser desorption ionization time-of-flight (MALDI-TOF) mass spectrometry coupled with affinity capture is particularly suitable for such analysis since apo-cytochrome c and holo-cytochrome c can be distinguished on the basis of their unique molecular weights. For example, the Surface-Enhanced Laser Desorption/Ionization (SELDI) system (Ciphergen, Palo Alto, Calif.) may be utilized to detect cytochrome c release from mitochondria in apoptogen treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular mass of the protein is determined by its time of flight to the detector of the SELDI mass spectrometer.

A person having ordinary skill in the art will readily appreciate that there may be other suitable techniques for quantifying apoptosis, and such techniques for purposes of determining an indicator of mitochondrial function that is a cellular response to an apoptogenic stimulus are within the scope of the methods provided by the present invention.

vi. Free Radical Production as an Indicator of Mitochondrial Function

In certain embodiments methods for identifying modulators of mitochondrial mass and/or function involve detecting free radical production in a biological sample as an indicator of mitochondrial function. Although mitochondria are a primary source of free radicals in biological systems (see, e.g., Murphy et al., 1998 in Mitochondria and Free Radicals in Neurodegenerative Diseases, Beal, Howell and Bodis-Wollner, Eds., Wiley-Liss, New York, pp. 159-186 and references cited therein), the methods described herein should not be so limited and free radical production can be an indicator of mitochondrial function regardless of the particular subcellular source site. For example, numerous intracellular biochemical pathways that lead to the formation of radicals through production of metabolites such as hydrogen peroxide, nitric oxide or superoxide radical via reactions catalyzed by enzymes such as flavin-linked oxidases, superoxide dismutase or nitric oxide synthetase, are known in the art, as are methods for detecting such radicals (see, e.g., Kelver, 1993 Crit. Rev. Toxicol. 23:21; Halliwell B. and J. M. C. Gutteridge, Free Radicals in Biology and Medicine, 1989 Clarendon Press, Oxford, UK; Davies, K. J. A. and F. Ursini, The Oxygen Paradox, Cleup Univ. Press, Padova, IT). Mitochondrial function, such as failure at any step of the ETC, may also lead to the generation of highly reactive free radicals. As noted above, radicals resulting from mitochondrial function include reactive oxygen species (ROS), for example, superoxide, peroxynitrite and hydroxyl radicals, and potentially other reactive species that may be toxic to cells. Accordingly, in certain embodiments, an indicator of mitochondrial function may be a detectable free radical species present in a biological sample. In certain embodiments, the detectable free radical will be a ROS.

Methods for detecting a free radical that may be useful as an indicator of mitochondrial function are known in the art and will depend on the particular radical. Typically, a level of free radical production in a biological sample may be determined according to methods with which those skilled in the art will be readily familiar, including but not limited to detection and/or measurement of: glycoxidation products including pentosidine, carboxymethylysine and pyrroline; lipoxidation products including glyoxal, malondialdehyde and 4-hydroxynonenal; thiobarbituric acid reactive substances (TBARS; see, e.g., Steinbrecher et al., 1984 Proc. Nat. Acad. Sci. USA 81:3883; Wolff, 1993 Br. Med. Bull. 49:642) and/or other chemical detection means such as salicylate trapping of hydroxyl radicals (e.g., Ghiselli et al., 1998 Meths. Mol. Biol. 108:89; Halliwell et al., 1997 Free Radic. Res. 27:239) or specific adduct formation (see, e.g., Mecocci et al. 1993 Ann. Neurol. 34:609; Giulivi et al., 1994 Meths. Enzymol. 233:363) including malondialdehyde formation, protein nitrosylation, DNA oxidation including mitochondrial DNA oxidation, 8-OH-guanosine adducts (e.g., Beckman et al., 1999 Mutat. Res. 424:51), protein oxidation, protein carbonyl modification (e.g., Baynes et al., 1991 Diabetes 40:405; Baynes et al., 1999 Diabetes 48:1); electron spin resonance (ESR) probes; cyclic voltametry; fluorescent and/or chemiluminescent indicators (see also e.g., Greenwald, R. A. (ed.), Handbook of Methods for Oxygen Radical Research, 1985 CRC Press, Boca Raton, Fla.; Acworth and Bailey, (eds.), Handbook of Oxidative Metabolism, 1995 ESA, Inc., Chelmsford, Mass.; Yla-Herttuala et al., 1989 J. Clin. Invest. 84:1086; Velazques et al., 1991 Diabetic Medicine 8:752; Belch et al., 1995 Int. Angiol. 14:385; Sato et al., 1979 Biochem. Med. 21:104; Traverso et al., 1998 Diabetologia 41:265; Haugland, 1996 Handbook of Fluorescent Probes and Research Chemicals—Sixth Ed., Molecular Probes, Eugene, Oreg., pp. 483-502, and references cited therein). For example, by way of illustration and not limitation, oxidation of the fluorescent probes dichlorodihydrofluorescein diacetate and its carboxylated derivative carboxydichlorodihydrofluorescein diacetate (see, e.g., Haugland, 1996, supra) may be quantified following accumulation in cells, a process that is dependent on, and proportional to, the presence of reactive oxygen species (see also, e.g., Molecular Probes On-line Handbook of Fluorescent Probes and Research Chemicals, world wide web at probes.com/handbook/toc.html). Other fluorescent detectable compounds that may be used in the invention for detection of free radical production include but are not limited to dihydrorhodamine and dihydrorosamine derivatives, cis-parinaric acid, resorufin derivatives, lucigenin and any other suitable compound that may be known to those familiar with the art.

Thus, as also described above, free radical mediated damage may inactivate one or more of the myriad proteins of the ETC and in doing so, may uncouple the mitochondrial chemiosmotic mechanism responsible for oxidative phosphorylation and ATP production. Indicators of mitochondrial function that are ATP biosynthesis factors, including determination of ATP production, are described in greater detail herein. Free radical mediated damage to mitochondrial functional integrity is also just one example of multiple mechanisms associated with mitochondrial function that may result in collapse of the electrochemical potential maintained by the inner mitochondrial membrane.

In other embodiments, provided are methods for treating an individual that may benefit from increased mitochondrial mass and/or function. The methods may involve first identifying a patient suffering from a mitochondrial dysfunction. The methods described above for identifying an agent that modulates mitochondrial mass and/or function may also be used for identifying an individual that would benefit from increased mitochondrial mass and/or activity. For example, the methods described above may be used to measure mitochondrial mass and/or function in a biological sample from one individual as compared to an individual (e.g., an individual having normal mitochondrial mass and/or function), a control population, or standard predetermined values of mitochondrial mass and/or function.

5. Pharmaceutical Compositions

The CLK-modulating compounds described herein may be formulated in a conventional manner using one or more physiologically acceptable carriers or excipients. For example, CLK-modulating compounds and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a CLK-modulating compound may be administered locally, at the site where the target cells are present, i.e., in a specific tissue, organ, or fluid (e.g., blood, cerebrospinal fluid, etc.).

CLK-modulating compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), CLK-modulating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

CLK-modulating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

CLK-modulating compounds may also be formulated in rectal compositions such as suppositories or retention enemas, e.g., containing conventional suppository bases such as cocoa butter or other glycerides.

In addition to the formulations described previously, CLK-modulating compounds may also be formulated as a depot preparation. Such long acting formulations may be administered by implantation (for example subcutaneously or intramuscularly) or by intramuscular injection. Thus, for example, CLK-modulating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the blood-brain-barrier (BBB)) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

One possibility to achieve sustained release kinetics is embedding or encapsulating the active compound into nanoparticles. Nanoparticles can be administrated as powder, as a powder mixture with added excipients or as suspensions. Colloidal suspensions of nanoparticles can easily be administrated through a cannula with small diameter.

Nanoparticles are particles with a diameter from about 5 nm to up to about 1000 nm. The term “nanoparticles” as it is used hereinafter refers to particles formed by a polymeric matrix in which the active compound is dispersed, also known as “nanospheres”, and also refers to nanoparticles which are composed of a core containing the active compound which is surrounded by a polymeric membrane, also known as “nanocapsules”. In certain embodiments, nanoparticles are preferred having a diameter from about 50 nm to about 500 nm, in particular from about 100 nm to about 200 nm.

Nanoparticles can be prepared by in situ polymerization of dispersed monomers or by using preformed polymers. Since polymers prepared in situ are often not biodegradable and/or contain toxicological serious byproducts, nanoparticles from preformed polymers are preferred. Nanoparticles from preformed polymers can be prepared by different techniques, e.g., by emulsion evaporation, solvent displacement, salting-out, mechanical grinding, microprecipitation, and by emulsification diffusion.

With the methods described above, nanoparticles can be formed with various types of polymers. For use in the method of the present invention, nanoparticles made from biocompatible polymers are preferred. The term “biocompatible” refers to material that after introduction into a biological environment has no serious effects to the biological environment. From biocompatible polymers those polymers are especially preferred which are also biodegradable. The term “biodegradable” refers to material that after introduction into a biological environment is enzymatically or chemically degraded into smaller molecules, which can be eliminated subsequently. Examples are polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen and albumin.

Suitable surface modifiers can preferably be selected from known organic and inorganic pharmaceutical excipients. Such excipients include various polymers, low molecular weight oligomers, natural products and surfactants. Preferred surface modifiers include nonionic and ionic surfactants. Representative examples of surface modifiers include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Further description on preparing nanoparticles can be found, for example, in U.S. Pat. No. 6,264,922, the contents of which are incorporated herein by reference.

Liposomes are a further drug delivery system which is easily injectable. Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent, for example, a coating of hydrophilic polymer chains such as polyethylene glycol (PEG) to extend the blood circulation lifetime of the liposomes. Such surface-modified liposomes are commonly referred to as “long circulating” or “sterically stabilized” liposomes.

One surface modification to a liposome is the attachment of PEG chains, typically having a molecular weight from about 1000 daltons (Da) to about 5000 Da, and to about 5 mole percent (%) of the lipids making up the liposomes (see, for example, Stealth Liposomes, CRC Press, Lasic, D. and Martin, F., eds., Boca Raton, Fla., (1995)), and the cited references therein. The pharmacokinetics exhibited by such liposomes are characterized by a dose-independent reduction in uptake of liposomes by the liver and spleen via the mononuclear phagocyte system (MPS), and significantly prolonged blood circulation time, as compared to non-surface-modified liposomes, which tend to be rapidly removed from the blood and accumulated in the liver and spleen.

In certain embodiments, the complex is shielded to increase the circulatory half-life of the complex or shielded to increase the resistance of nucleic acid to degradation, for example degradation by nucleases.

As used herein, the term “shielding”, and its cognates such as “shielded”, refers to the ability of “shielding moieties” to reduce the non-specific interaction of the complexes described herein with serum complement or with other species present in serum in vitro or in vivo. Shielding moieties may decrease the complex interaction with or binding to these species through one or more mechanisms, including, for example, non-specific steric or non-specific electronic interactions. Examples of such interactions include non-specific electrostatic interactions, charge interactions, Van der Waals interactions, steric-hindrance and the like. For a moiety to act as a shielding moiety, the mechanism or mechanisms by which it may reduce interaction with, association with or binding to the serum complement or other species does not have to be identified. One can determine whether a moiety can act as a shielding moiety by determining whether or to what extent a complex binds serum species.

It should be noted that “shielding moieties” can be multifunctional. For example, a shielding moiety may also function as, for example, a targeting factor. A shielding moiety may also be referred to as multifunctional with respect to the mechanism(s) by which it shields the complex. While not wishing to be limited by proposed mechanism or theory, examples of such a multifunctional shielding moiety are pH sensitive endosomal membrane-disruptive synthetic polymers, such as PPAA or PEAA. Certain poly(alkylacrylic acids) have been shown to disrupt endosomal membranes while leaving the-outer cell surface membrane intact (Stayton et al. (2000) J. Controll. Release 65:203-220; Murthy et al. (1999) J. Controll. Release 61:137-143; WO 99/34831), thereby increasing cellular bioavailability and functioning as a targeting factor. However, PPAA reduces binding of serum complement to complexes in which it is incorporated, thus functioning as a shielding moiety.

Another way to produce a formulation, particularly a solution, of a CLK modulator, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

The cyclodextrin of the compositions according to the invention may be α-, β-, or γ-cyclodextrin. α-cyclodextrin contains six glucopyranose units; β-cyclodextrin contains seven glucopyranose units; and γ-cyclodextrin contains eight glucopyranose units. The molecule is believed to form a truncated cone having a core opening of 4.7-5.3 angstroms, 6.0-6.5 angstroms, and 7.5-8.3 angstroms in α-, β-, or γ-cyclodextrin respectively. The composition according to the invention may comprise a mixture of two or more of the α-, β-, or γ-cyclodextrins. Typically, however, the composition according to the invention will comprise only one of the α-, β-, or γ-cyclodextrins.

Most preferred cyclodextrins in the compositions according to the invention are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

As mentioned above, the compositions of matter of the invention comprise an aqueous preparation of preferably substituted amorphous cyclodextrin and one or more CLK modulators. The relative amounts of CLK modulators and cyclodextrin will vary depending upon the relative amount of each of the CLK modulators and the effect of the cyclodextrin on the compound. In general, the ratio of the weight of compound of the CLK modulators to the weight of cyclodextrin compound will be in a range between 1:1 and 1:100. A weight to weight ratio in a range of 1:5 to 1:50 and more preferably in a range of 1:10 to 1:20 of the compound selected from CLK modulators to cyclodextrin are believed to be the most effective for increased circulating availability of the CLK modulator.

Importantly, if the aqueous solution comprising the CLK modulators and a cyclodextrin is to be administered parenterally, especially via the intravenous route, a cyclodextrin will be substantially free of pyrogenic contaminants. Various forms of cyclodextrin, such as forms of amorphous cyclodextrin, may be purchased from a number of vendors including Sigma-Aldrich, Inc. (St. Louis, Mo., USA). A method for the production of hydroxypropyl-β-cyclodextrin is disclosed in Pitha et al., U.S. Pat. No. 4,727,064 which is incorporated herein by reference.

Additional description of the use of cyclodextrin for solubilizing compounds can be found in US 2005/0026849, the contents of which are incorporated herein by reference.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast melt solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms. See e.g., patents and publications by Cima Labs such as U.S. Pat. Nos. 5,607,697, 5,503,846, 5,223,264, 5,401,513, 5,219,574, and 5,178,878, WO 98/46215, WO 98/14179; patents to Fuisz Technologies, now part of BioVail, such as U.S. Pat. Nos. 5,871,781, 5,869,098, 5,866,163, 5,851,553, 5,622,719, 5,567,439, and 5,587,172; U.S. Pat. No. 5,464,632 to Prographarm; patents to R. P. Scherer such as U.S. Pat. Nos. 4,642,903, 5,188,825, 5,631,023 and 5,827,541; patents to Yamanouchi-Shaklee such as U.S. Pat. Nos. 5,576,014 and 5,446,464; patents to Janssen such as U.S. Pat. Nos. 5,807,576, 5,635,210, 5,595,761, 5,587,180 and 5,776,491; U.S. Pat. Nos. 5,639,475 and 5,709,886 to Eurand America, Inc.; U.S. Pat. Nos. 5,807,578 and 5,807,577 to L.A.B. Pharmaceutical Research; patents to Schering Corporation such as U.S. Pat. Nos. 5,112,616 and 5,073,374; U.S. Pat. No. 4,616,047 to Laboratoire L. LaFon; U.S. Pat. No. 5,501,861 to Takeda Chemicals Inc., Ltd.; and U.S. Pat. No. 6,316,029 to Elan.

In one example of fast melt tablet preparation, granules for fast melt tablets made by either the spray drying or pre-compacting processes are mixed with excipients and compressed into tablets using conventional tablet making machinery. The granules can be combined with a variety of carriers including low density, high moldability saccharides, low moldability saccharides, polyol combinations, and then directly compressed into a tablet that exhibits an improved dissolution and disintegration profile.

The tablets according to the present invention typically have a hardness of about 2 to about 6 Strong-Cobb units (scu). Tablets within this hardness range disintegrate or dissolve rapidly when chewed. Additionally, the tablets rapidly disintegrate in water. On average, a typical 1.1 to 1.5 gram tablet disintegrates in 1-3 minutes without stirring. This rapid disintegration facilitates delivery of the active material.

The granules used to make the tablets can be, for example, mixtures of low density alkali earth metal salts or carbohydrates. For example, a mixture of alkali earth metal salts includes a combination of calcium carbonate and magnesium hydroxide. Similarly, a fast melt tablet can be prepared according to the methods of the present invention that incorporates the use of A) spray dried extra light calcium carbonate/maltodextrin, B) magnesium hydroxide and C) a eutectic polyol combination including Sorbitol Instant, xylitol and mannitol. These materials have been combined to produce a low density tablet that dissolves very readily and promotes the fast disintegration of the active ingredient. Additionally, the pre-compacted and spray dried granules can be combined in the same tablet.

For fast melt tablet preparation, a CLK modulator useful in the present invention can be in a form such as solid, particulate, granular, crystalline, oily or solution. The CLK modulator for use in the present invention may be a spray dried product or an adsorbate that has been pre-compacted to a harder granular form that reduces the medicament taste. A pharmaceutical active ingredient for use in the present invention may be spray dried with a carrier that prevents the active ingredient from being easily extracted from the tablet when chewed.

In addition to being directly added to the tablets of the present invention, the medicament drug itself can be processed by the pre-compaction process to achieve an increased density prior to being incorporated into the formulation.

The pre-compaction process used in the present invention can be used to deliver poorly soluble pharmaceutical materials so as to improve the release of such pharmaceutical materials over traditional dosage forms. This could allow for the use of lower dosage levels to deliver equivalent bioavailable levels of drug and thereby lower toxicity levels of both currently marketed drug and new chemical entities. Poorly soluble pharmaceutical materials can be used in the form of nanoparticles, which are nanometer-sized particles.

In addition to the active ingredient and the granules prepared from low density alkali earth metal salts and/or water soluble carbohydrates, the fast melt tablets can be formulated using conventional carriers or excipients and well established pharmaceutical techniques. Conventional carriers or excipients include, but are not limited to, diluents, binders, adhesives (i.e., cellulose derivatives and acrylic derivatives), lubricants (i.e., magnesium or calcium stearate, vegetable oils, polyethylene glycols, talc, sodium lauryl sulphate, polyoxy ethylene monostearate), disintegrants, colorants, flavorings, preservatives, sweeteners and miscellaneous materials such as buffers and adsorbents.

Additional description of the preparation of fast melt tablets can be found, for example, in U.S. Pat. No. 5,939,091, the contents of which are incorporated herein by reference.

Pharmaceutical compositions (including cosmetic preparations) may comprise from about 0.00001 to 100% such as from 0.001 to 10% or from 0.1% to 5% by weight of one or more CLK-modulating compounds described herein.

In one embodiment, a CLK-modulating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

CLK-modulating compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's (supra) ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

CLK-modulating compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor™ from Beiersdorf, Inc. (Norwalk, Conn.).

CLK-modulating compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

CLK-modulating compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

CLK-modulating compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C₁₀ MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C₂-C₆ alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol®) and diethylene glycol monoethyl ether oleate (available commercially as Softcutol®); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol®); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises a modulating compound-containing microemulsion as described above, but may contain alternative pharmaceutically acceptable carriers, vehicles, additives, etc. particularly suited to oral or parenteral drug administration. Alternatively, a modulating compound-containing microemulsion may be administered orally or parenterally substantially as described above, without modification.

Conditions of the eye can be treated or prevented by, e.g., systemic, topical, intraocular injection of a CLK-modulating compound, or by insertion of a sustained release device that releases a CLK-modulating compound. A CLK-modulating compound that increases or decreases the level and/or activity of a CLK protein may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye.

CLK-modulating compounds described herein may be stored in oxygen free environment according to methods in the art.

Cells, e.g., treated ex vivo with a CLK-modulating compound, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of CLK-modulating compounds can be determined by standard pharmaceutical procedures in cell cultures or experimental animals. The LD50 is the dose lethal to 50% of the population. The ED₅₀ is the dose therapeutically effective in 50% of the population. The dose ratio between toxic and therapeutic effects (LD₅₀/ED₅₀) is the therapeutic index. CLK-modulating compounds that exhibit large therapeutic indexes are preferred. While CLK-modulating compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

6. Kits

Also provided herein are kits, e.g., kits for therapeutic purposes or kits for modulating the lifespan of cells or modulating apoptosis. A kit may comprise one or more CLK-modulating compounds, e.g., in premeasured doses. A kit may optionally comprise devices for contacting cells with the compounds and instructions for use. Devices include syringes, stents and other devices for introducing a CLK-modulating compound into a subject (e.g., the blood vessel of a subject) or applying it to the skin of a subject.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. 1. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Exemplification

The invention now being generally described, it will be more readily understood by reference to the following examples which are included merely for purposes of illustration of certain aspects and embodiments of the present invention, and are not intended to limit the invention in any way.

### EXAMPLE 1

### CLK Interacts With and Phosphorylates Sirtuins and PGC-1alpha Proteins

A variety of experiments were conducted to examine the interaction between CLK and sirtuins or PGC-1 alpha. The results of these experiments are illustrated in the Figures. The materials and methods used to conduct the experiments shown in the figures is described below.

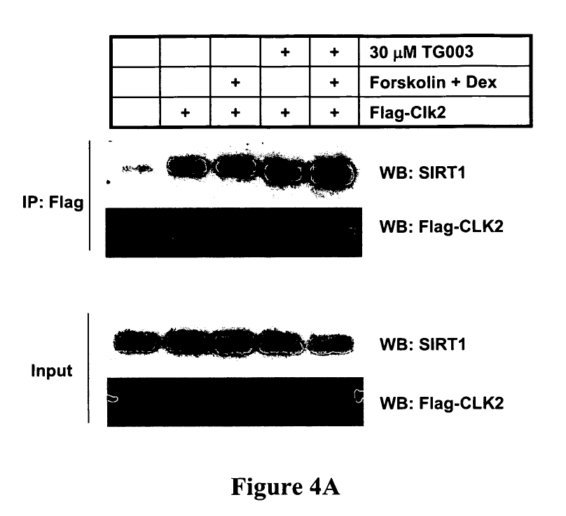

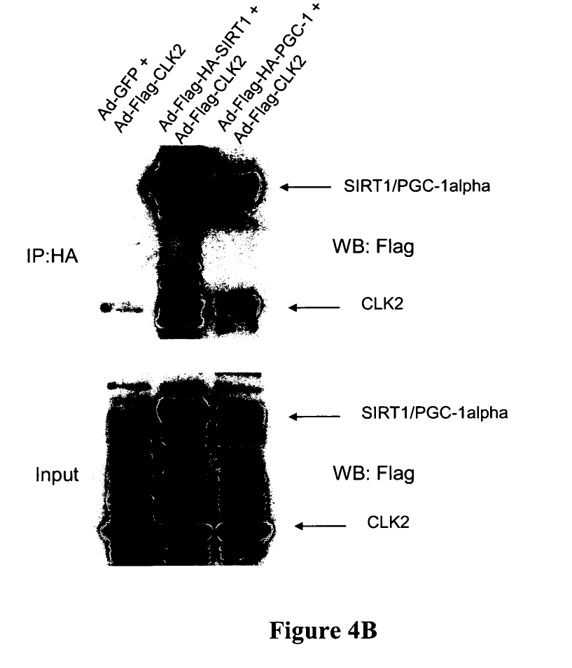

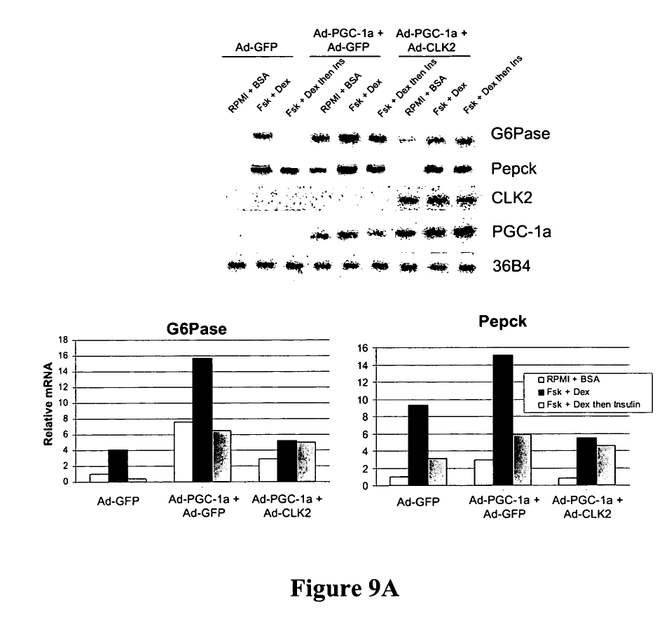

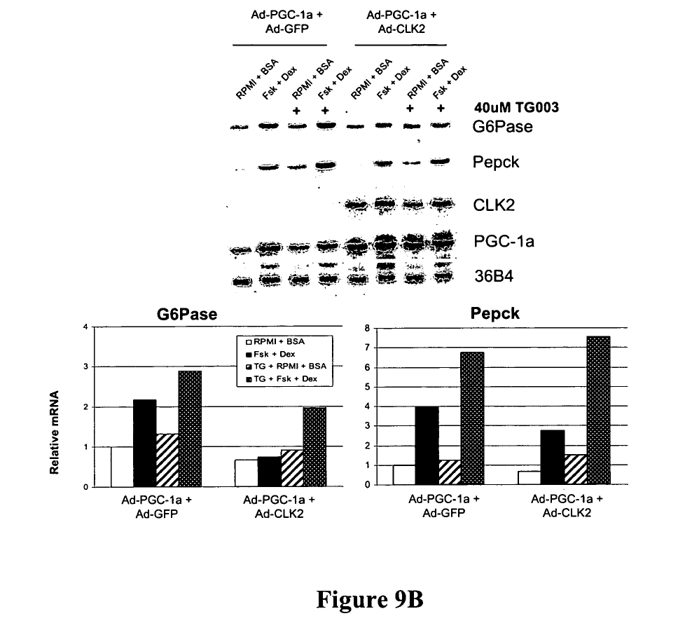

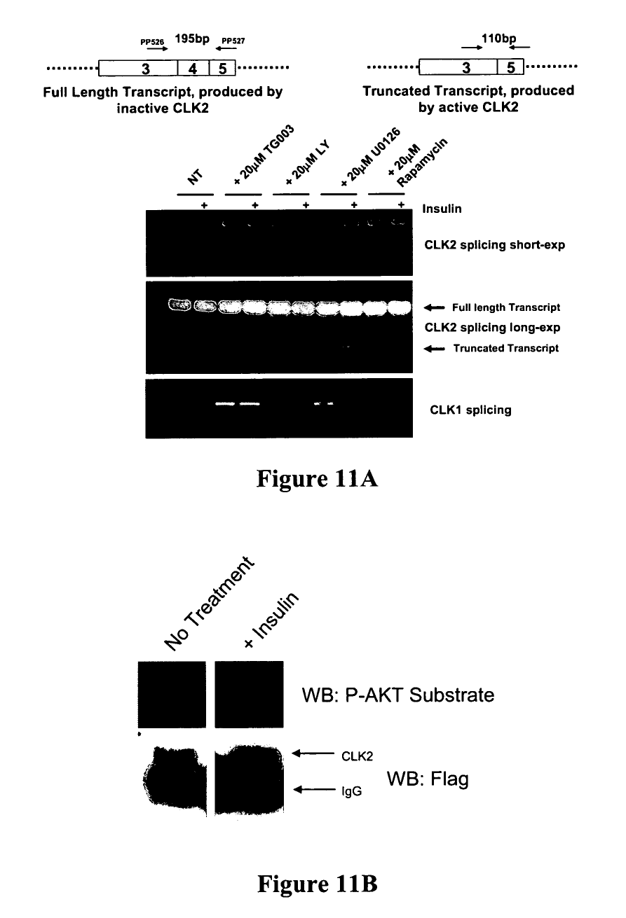

Cell Culture. HEK 293 Cells were cultured in DMEM+10% CCS. FAO Rat hepatocytes were grown in Hamm's F-12 media with 5% FBS. Prior to experiments or adenoviral infections media was switched to RPMI+0.5% BSA. H2.35 Mouse Hepatocytes were grown in DMEM (low glucose)+4% FBS and 2 μM Dexamethasome. Prior to experiments and infection, media was switched to DMEM (low glucose)+0.5% BSA. CLK inhibitor, TG003 (CalBiochem) was dissolved in DMSO. Treatments, as indicated in FIGS. 4A, 9A, 9B, 11A, and 11B were at final concentrations of: Insulin 200 nM, dexamethosome 1 μM, and forskolin 5 μM.

Plasmid and Adenovirus Construction. Mouse CLK2 (mCLK2) was cloned from mouse liver RNA using Superscript One-Step RT-PCR with Taq (Invitrogen) and cloned into pcDNA 3 with an N-terminal Flag tagged. CLK2 K192R mutation was created by site-directed mutagenesis. Adenovirus was constructed by cloning Flag-CLK2 into pAD-Track-CMV, full length adenovirus was made by recombination with pAd-Easy-1 in BJ5183-AD-1 bacteria (Stratagene). Oligos corresponding to Mouse, Rat and Human CLK2 (5′ cct tcg att tcc tca aag aca) (SEQ ID NO: 15) and control siRNA (5′ cct tcg att ccc tca aag aca) (SEQ ID NO: 16) were annealed into pLK0-puro. Adenovirus was constructed by cloning the U6 promoter and siRNA sequence into pAd-Track.

Transient Transfections. HEK 293 were transfected using PolyFect (Qiagen). 25 ng of reporter (gAF1-Luciferase) and 25 ng of pcDNA mouse HNF4-alpha were transfected with pcDNA mouse PGC-1 alpha and pcDNA Flag-CLK2. Equal amounts of total DNA were used for all transfections by adding appropriate amounts of empty vector pcDNA. TG003 (Calbiochem) was added 4 hours after transfection and cells were harvested the next morning. The data presented is the average of 3 replicates from a single experiment, all luciferase experiments were performed at least 3 times with similar results.

Immunoprecipitation and Co-Immunoprecipitation. Cells were washed once with PBS containing Phosphatase inhibitors (5 mM Glycerol-2-phosphate, 20 mM NaF, and 0.2 mM Na OrthoVanadate), scraped into tubes, spun down, and lysed by 2× freeze-thaw cycles in 0.4% Triton, 100 mM NaCl, 20 mM KHepes pH 7.9, 1 mM EDTA, Phosphatase inhibitors, 1 mM PMSF, and 1× Protease Inhibitors (Roche). Immunoprecipitations were performed with M2 anti-flag agarose (sigma) or Anti-HA agarose (Roche) for 2 hours rotating at 4 degrees C. followed by 3 washes in lysis buffer. Products were resolved by SDS-PAGE and transferred to PVDF membranes for Western blot analysis.

Metabolic Labeling. HEK 293 were transfected as described above. The next morning cells were switched to DMEM without PO₄+10% CCS for 30 minutes, cells were then treated with or without TG003 for 30 minutes then 200 μCi of ³²PO₄ was added for 2 hours. Cells were harvested and immunoprecipitation was performed as described above. Immunoprecipitates were resolved by SDS-PAGE and transferred to PVDF membrane, ³²PO₄ was detected by exposing membrane to Phospho-imaging screen and followed by Western-Blot using anti-flag antibodies (M2, Sigma).

FAO Hepatocytes were infected with adenovirus as indicated in the figures for 2 days in RPMI+0.5% BSA. Metabolic labeling was performed as described above, except cells were incubated in ³²PO₄ for 4 hours.

Northern Blot and RNA analysis. FAO hepatocytes were infected with indicated adenovirus overnight in RPMI+0.5% BSA. Cells were grown for 2 more days in RPMI+0.5% BSA. Cells were treated as indicated. Total RNA was isolated using Trizol (Invitrogen). Northern blots using indicated cDNAs were performed on 15 μg of total RNA. Quantitation of RNA was performed by exposing membranes to phosphor-imager screens and analyzed by a Bio-Rad Personal Imager FX and Quantity-One quantitation software (Bio-Rad).

Splicing Analysis. Total RNA was isolated from FAO hepatocytes pre-treated with indicated inhibitor then Insulin for 2 hours. RT-PCR was performed using SuperScript One-Step RT-PCR with taq (Invitrogen) on 1 ug of total RNA using primers flanking exon 4 on Clk2 and Clk1.

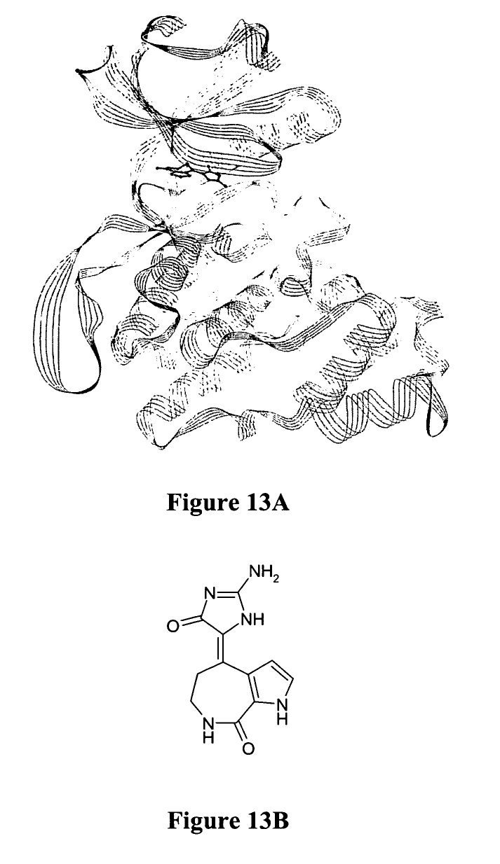

Expression and purification of CLK1 and 2. The expression protocol is partly based on the purification protocol for protein for human CLK1 and crystal structure in complex with 10Z-2 hymenialdisine at 1.7 Å as reported in the pdb data base (1Z57) and shown in FIGS. 13A and 13B.

Briefly, a T7 promoter based vector (Novagen) for expression of CLK1 and CLK2 is transformed into BL21 (DE3), BL21 (DE3) RIL, BL21 (DE3) RP or BL21 (DE3) pLys cells (Invitrogen) and plated onto an LB agar plate. One of the freshly grown colonies is picked and grown in a small culture (5 ml, 100 mg/mL ampicillin (AMP) of either LB, Terrific broth, Super broth (vendor all: RPI) or M9 media (vendor Tecknova) at 37° C. over night. The culture is 100-fold diluted into new media containing AMP (final concentration 1 mM) and grown at 37° C. to an OD₆₀₀ of 0.8. Cultures are iced to a temperature of 18° C. prior to induction with IPTG (final concentration 1 mM). Cultures are harvested after 12 hours of induction time at 18° C.

Cells are harvested at 8000×g for 6 minutes and resuspended in lysis buffer (50 mM HEPES pH 7.5, 500 mM NaCl, 5% Glycerol) and lyzed with lyzozyme (5 mg/g cell paste) for 30 minutes following sonication for 10 minutes. Cells are then centrifuged at 30,000×g for 45 minutes and the supernatant loaded onto a DE52 column (Whatman) for nucleic acid removal. The flow through is collected and loaded onto a Ni-chelating column for affinity chromatography. The column is washed with wash buffer (20 mM Imidazole, 300 mM NaCl, 50 mM KH₂PO₄, pH 8.0) to remove endogenous bound protein. The protein is cleaved from the column with either TEV or Pre scission protease (GE Healthcare) over night as well as dephosphorylated with GST-tagged Lambda phosphatase (New England Biolabs, Beverly, Mass.). The supernatant is collected and concentrated to 12 mg/mL for size exclusion chromatography. 2 mL fractions of the concentrated protein are loaded onto a S200 16/60 global sizing column (GE Healthcare) and protein peaks collected and analyzed for solubility by SDS and native polyacrylamide gels (Invitrogen). Additional purification with Ion exchange chromatography (GE Healthcare) is optional.

The protein is concentrated to 15 mg/mL, dialyzed against storage buffer (100 mM NaCl, 20% Glycerol, 20 mM Tris-HCl, pH 8.0) and stored in aliquots at −80° C.

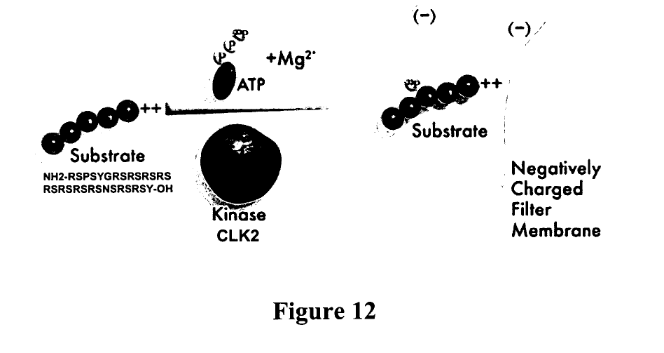

CLK In Vitro Kinase Assay. An exemplary kinase assay for determining activity of CLKs is shown schematically in FIG. 12. Briefly, CLKs are assayed in a reaction mixture containing 200 mM Tris-HCl (pH 7.5), 12.5 mM MgCl₂, 8 mM dithiothreitol, 4 mM EGTA, 1-20 μM ATP, 1 μCi of [gamma-³²P]ATP, 1 μg of synthetic peptide of SF2/ASF RS domain (NH2-RSPSYGRSRSRSRSRSRSRSRSNSRSRSY-OH) (SEQ ID NO: 9), and 0.1-1 μg of purified kinases in a final volume of 40 μL. The final concentration of DMSO is adjusted to 1% regardless of inhibitor concentration. The reaction mixture is incubated at 30 C for 10 min, and a half-portion is spotted on P81 phosphocellulose membrane (Whatman). The kinase assay conditions, including the incubation period and concentration of kinases and substrates, are optimized to maintain the linearity during incubation. The membrane is washed with 5% phosphoric acid solution for at least 15 min. The radioactivity is measured using a liquid scintillation counter. The net radioactivity is deduced by subtracting the background count from the reaction mixture without kinase, and the data are expressed as the percentage to the control sample containing the solvent.

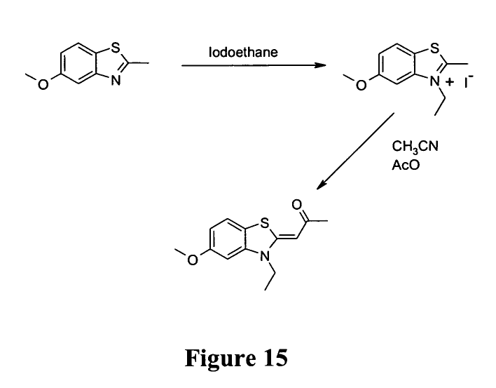

Examples of CLK inhibitors. FIG. 14 gives the structures of known CLK inhibitors as described in US patent application 2005/0171026. FIG. 15 describes the synthesis of one representative CLK inhibitor. Specifically, 5-methoxy-2-methylbenzothiazole (202 mg, 1.12 mmol) and ethyl iodide (2.70 ml, 33.7 mmol) was refluxed for 24.5 h. The precipitate was filtrated, washed with ethyl acetate (20 ml) on a funnel, and dried under reduced pressure to afford 3-ethyl-5-methoxy-2-methylbenzothiazolium iodide (270 mg, 0.805 mmol, 71.9%) as a pale green solid. To a suspension of 3-ethyl-5-methoxy-2-methylbenzothiazolium iodide (502 mg, 1.49 mmol) in acetonitrile (2.0 ml), acetic anhydride (330 μL, 3.49 mmol) and triethylamine (490 ul, 3.51 mmol) were successively added at room temperature. After refluxing for 2 hours, the mixture was cooled to room temperature and concentrated under reduced pressure. Water (50 ml) was added to the residue, and the mixture was extracted with ethyl acetate (three times with 15 ml). The combined organic extracts were washed with brine (30 ml), dried over sodium sulfate, filtered, and concentrated under reduced pressure. The residue was purified by silica gel column chromatography (18 g, CH₂Cl₂/ethyl acetate, 4:1) to afford (Z)-1-(3-ethyl-5-methoxy-2,3-dihydrobenzothiazol-2-ylidene) propan-2-one (201 mg, 0.806 mmol, 54.1%) as a pale yellow solid.

### EXAMPLE 2

### Sirtuin Expression and Activity Assays

A fluorescence polarization or mass spectrometry based assay may be used to measure the activity of sirtuins. The same assays may be used to measure changes in sirtuin enzymatic activity caused by post-translational modification, such as phosphorylation by CLK. The same assays can be used to look at the effects of post-translational modification and small molecules that modulate the activity of sirtuins. The fluorescence polarization assays may utilize a variety of peptide substrates comprising one of two different peptides based on a fragment of p53, a known sirtuin deacetylation target. The substrate may contain peptide 1 having 14 amino acid residues as follows: GQSTSSHSK(Ac)NleSTEG (SEQ ID NO: 11) wherein K(Ac) is an acetylated lysine residue and Nle is a norleucine, or peptide 2 having 20 amino acid residues as follows: EE-K(biotin)-GQSTSSHSK(Ac)NleSTEG-K(MR121)-EE-NH₂ (SEQ ID NO: 13) wherein K(biotin) is a biotinolated lysine residue, K(Ac) is an acetylated lysine residue, Nle is norleucine and K(MR121) is a lysine residue modified by an MR121 fluorophore. The peptide is labeled with the fluorophore MR121 (excitation 635 nm/emission 680 nm) at the C-terminus and biotin at the N-terminus. An alternative substrate contains a peptide having the same 20 amino acid residues as follows: Ac-Glu-Glu-Lys(Biotin)-Gly-Gln-Ser-Thr-Ser-Ser-His-Ser-Lys(Ac)-Nle-Ser-Thr-Glu-Gly-Lys(5TMR)-Glu-Glu-NH2 (SEQ ID NO: 14) wherein K(Ac) is an acetylated lysine residue and Nle is a norleucine and K(5TMR) is a lysine residue modified by an MR121 fluorophore. The peptide is labeled with the fluorophore 5TMR (excitation 540 nm/emission 580 nm) at the C-terminus. The sequences of both peptide substrates are based on p53 with several modifications. In particular, all arginine and leucine residues other than the acetylated lysine have replaced with serine so that the peptide is not susceptible to trypsin cleavage in the absence of deacetylation. In addition, the methionine residue naturally present in the sequence has been replaced with the norleucine because the methionine may be susceptible to oxidation during synthesis and purification.

The peptide substrate is exposed to a sirtuin protein, either before or after post translational modification, in the presence of NAD⁺ to allow deacetylation of the substrate and render it sensitive to cleavage by trypsin. Trypsin is then added and the reaction is carried to completion (i.e., the deacetylated substrate is cleaved) releasing the MR121 or 5TMR fragment. The uncleaved substrate (i.e., any remaining acetylated substrate) and the non-fluorescent portion of the cleaved peptide substrate (i.e., the biotin containing fragment) are removed from the reaction using streptavadin. The fluorescence polarization signal observed for the full length peptide substrate bound to streptavidin is higher than the fluorescence polarization signal observed for the released MR121 or 5TMR C-terminal fragment. In this way, the fluorescence polarization obtained is inversely proportional to the level of deacetylation (e.g., the signal is inversely proportional to the activity of the sirtuin protein). Results are read on a microplate fluorescence polarization reader (Molecular Devices Spectramax MD) with appropriate excitation and emission filters.

The fluorescence polarization assays using peptide 1 may be conducted as follows: 0.5 μM peptide substrate and 150 μM βNAD⁺ is incubated with 0.1 μg/mL of SIRT1 for 60 minutes at 37° C. in a reaction buffer (25 mM Tris-acetate pH8, 137 mM Na—Ac, 2.7 mM K—Ac, 1 mM Mg—Ac, 0.05% Tween-20, 0.1% Pluronic F127, 10 mM CaCl₂, 5 mM DTT, 0.025% BSA, 0.15 mM Nicotinamide). Fluorescence polarization assays using peptide 2 may be conducted as follows: 0.5 μM peptide substrate and 120 μM βNAD⁺ were incubated with 3 nM SIRT1 for 20 minutes at 25° C. in a reaction buffer (25 mM Tris-acetate pH8, 137 mM Na—Ac, 2.7 mM K—Ac, 1 mM Mg—Ac, 0.05% Tween-20, 0.1% Pluronic F127, 10 mM CaCl₂, 5 mM DTT, 0.025% BSA). The affect of test compounds can be looked at by addition of the test compounds to the reaction mixture following solubilization in DMSO. Test compounds may be added to the reaction at a variety of concentrations, for example, ranging from 0.7 μM to 300 μM. The SIRT1 protein used in the assays is overexpressed in E. coli as a His-tag fusion and was purified on a nickel chelate column using standard techniques. After the 60 minute incubation with SIRT1, nicotinamide is added to the reaction to a final concentration of 3 mM to stop the deacetylation reaction and 0.5 μg/mL of trypsin is added to cleave the deacetylated substrate. The reaction is incubated for 30 minutes at 37° C. in the presence of 1 mM streptavidin. Fluorescent polarization is determined at excitation (650 nm) and emissions (680 nm) wavelengths. The level of activity of the sirtuin protein in the presence of the various concentrations of test compound are then determined and may be compared to the level of activity of the sirtuin protein in the absence of the test compound, and/or the level of activity of the sirtuin proteins in the negative control (e.g., level of inhibition) and positive control (e.g., level of activation) described below.

For the Fluorescence Polarization assays, a control for inhibition of sirtuin activity is conducted by adding 1 μL of 500 mM nicotinamide as a negative control at the start of the reaction (e.g., permits determination of maximum sirtuin inhibition). A control for activation of sirtuin activity was conducted using 3 nM of sirtuin protein, with 1 μL of DMSO in place of compound, to reach maximum deacetylation of the substrate (e.g., to determine maximum sirtuin activation).

The mass spectrometry based assay utilizes a peptide having 20 amino acid residues as follows: Ac-Glu-Glu-Lys(Biotin)-Gly-Gln-Ser-Thr-Ser-Ser-His-Ser-Lys(Ac)-Nle-Ser-Thr-Glu-Gly-Lys(5TMR)-Glu-Glu-NH2 (SEQ ID NO: 14) wherein K(Ac) is an acetylated lysine residue and Nle is a norleucine. The peptide is labeled with the fluorophore 5TMR (excitation 540 nm/emission 580 nm) at the C-terminus. The sequence of the peptide substrate is based on p53 with several modifications. In addition, the methionine residue naturally present in the sequence was replaced with the norleucine because the methionine may be susceptible to oxidation during synthesis and purification.

The mass spectrometry assay is conducted as follows: 0.5 μM peptide substrate and 120 μM βNAD⁺ is incubated with 10 nM SIRT1 for 25 minutes at 25° C. in a reaction buffer (50 mM Tris-acetate pH 8, 137 mM NaCl, 2.7 mM KCl, 1 mM MgCl₂, 5 mM DTT, 0.05% BSA). Test compounds may be added to the reaction as described above. The SirT1 gene is cloned into a T7-promoter containing vector and transformed into BL21 (DE3). After the 25 minute incubation with SIRT1, 10 μL of 10% formic acid was added to stop the reaction. Reactions are sealed and frozen for later mass spec analysis. Determination of the mass of the substrate peptide allows for precise determination of the degree of acetylation (i.e. starting material) as compared to deacetylated peptide (product).

For the mass spectrometry based assay, a control for inhibition of sirtuin activity is conducted by adding 1 μL of 500 mM nicotinamide as a negative control at the start of the reaction (e.g., permits determination of maximum sirtuin inhibition). A control for activation of sirtuin activity is conducted using 10 nM of sirtuin protein, with 1 μL of DMSO in place of compound, to determine the amount of deacetylation of the substrate at a given timepoint within the linear range of the assay. This timepoint is the same as that used for test compounds and, within the linear range, the endpoint represents a change in velocity.

The SirT1 gene was cloned into a T7-promoter containing vector and transformed into BL21 (DE3). The protein was expressed by induction with 1 mM IPTG as an N-terminal His-tag fusion protein at 18° C. overnight and harvested at 30,000×g. Cells were lysed with lysozyme in lysis buffer (50 mM Tris-HCl, 2 mM Tris[2-carboxyethyl]phosphine (TCEP), 10 μM ZnCl₂, 200 mM NaCl) and further treated with sonication for 10 min for complete lysis. The protein was purified over a Ni-NTA column (Amersham) and fractions containing pure protein were pooled, concentrated and run over a sizing column (Sephadex S200 26/60 global). The peak containing soluble protein was collected and run on an Ion-exchange column (MonoQ). Gradient elution (200 mM-500 mM NaCl) yielded pure protein. This protein was concentrated and dialyzed against dialysis buffer (20 mM Tris-HCl, 2 mM TCEP) overnight. The protein was aliquoted and frozen at −80° C. until further use.

Post-translational modification of sirtuins, such as SIRT1, is accomplished by incubation of the recombinantly produced SIRT1 protein with recombinantly produced CLK enzyme produced as described above. CLK phosphorylation of sirtuins such as SIRT1 is done in conditions such as those described for the CLK in vitro assay described above. Conformation of the degree of phosphorylation and site of phosphorylation (Serine 172, 173 and/or 174 of SEQ ID NO: 10) is accomplished by standard mass spectrometer based peptide analysis.

### EXAMPLE 3

### CLK Splicing Assays

i. In vitro splicing assay. m⁷ GpppG-capped and ³²P-labeled pre-mRNA substrates are made by runoff transcription of linearized human beta-globin template DNA with SP6 RNA polymerase (Mayeda, A., and Kramer, A. R. (1992) Cell 68, 365). HeLa cell S100 extract and purified SF2/ASF are prepared as described (Mayeda, A., and Kramer, A. R. (1999) Methods Mol. Biol. 118, 309). In vitro splicing reaction mix containing the HeLa S100 extract, purified SF2/-ASF, and 20 fmol of ³²P-labeled pre-mRNA is incubated with/without CLK modulators at 30 degree C. for 3 to 4 h (Mayeda, A., and Krainer, A. R. (1999) Methods Mol. Biol. 118, 309). The RNA products are analyzed by electrophoresis on a 5.5% polyacrylamide, 7 M urea gel and autoradiography.

ii. In vivo splicing assay. COS-7 cells grown in a 60-mm dish are transfected with recombinant CLK expression vectors as described (Caceres, J. F., Stamm, S., Helfman, D. M., and Krainer, A. R. (1994) Science 265, 1706), using LipofectAMINE (Invitrogen) according to the manufacturer's instructions. Twenty four hours after transfection, either the total RNA is extracted using ISOGEN (Nippon Gene) or cells are lysed in SDS-gel loading buffer (0.1 M Tris-HCl (pH 6.8), 0.2 M dithiothreitol, 4% SDS, 20% glycerol) to prepare total cellular protein extract. Five micrograms of RNA is used for reverse transcription (RT), and then 1:50 was used for PCR amplification. PCR conditions, including the number of cycles and template concentrations, are optimized to maintain the linearity during amplification. PCR products are separated in agarose gel and stained with ethidium bromide. Total protein was separated in SDS-PAGE and transferred to PVDF membrane.

For splicing assay for endogenous genes, mouse embryonic fibroblasts (STO cells) are incubated in the presence or absence of CLK modulators for 4 h, and total RNA is extracted using TRIzol (Invitrogen) before RT-PCR using primers for SC35 and Clk1/Sty designed as per Pilch et al. (Pilch, B., Allemand, E., Facompre, M., Bailly, C., Riou, J. F., Soret, J., and Tazi, J. (2001) Cancer Res. 61, 6876).

iii. Western assays. SR proteins (SRp75, SRp70, SRp55, SRp40, SRp30 and SRp20) are serine-arginine rich proteins and have a conserved phosphorylation site in the RS domain that is a target for CLK kinases. The monoclonal antibody mAB 104 (ATCC® Number: CRL-2067™) recognizes this phopho-epitope and can be used to monitor the efficiency of phosphorylation by CLK kinase in a western assay. To perform compound inhibition assays by western blotting, either Hela nuclear extracts or S100 extracts would be incubated with recombinant CLK kinase in splicing assay buffer (12 mM HEPES-KOH (pH 7.9), 20 mM creatine phosphate, 0 to 42 mM (NH)₄SO₄, 20 to 60 mM KCl, 2.1 to 3.2 mM MgCl₂, 0.12 mM EDTA, 0.5 mM dithiothreitol, 2.6% polyvinal alcohol, 2 U of RNasin, and 6 to 10% glycerol). The data could be simultaneously normalized with the use of anti-SR protein (ATCC® Number: CRL-2383). At the end of the reactions (90 minutes), proteins in the splicing assays would be diluted 10-fold with water and precipitated with 10% trichloroacetic acid for 60 min on ice. Pellets will be washed with acetone before resuspending in sodium dodecyl sulfate (SDS) gel sample buffer. Proteins would then be fractionated by SDS-10% PAGE and then transferred to PVDF membrane before revealing with monoclonal antibody (MAb) 104 as described above.

iv. Reporter assays. HIV tat pre-mRNA has a weak 3′ splice site and several purine-rich sequences in tat pre-mRNA exons resemble the ASF/SF2 recognition sequence. ClK family of kinases directly act on ASF/SF2 protein and control their splicing ability by inhibiting it. Skipping of exon 10 of Tau protein is another CLK kinase dependent phenomenon. In the presence of CLK2 kinase, exon 10 skipping of tau is increased from 30% to 70%. By utilizing the splice sites of the above two proteins with luciferase or GFP reporters integrated in the genome, a novel assay system can be generated that will be responsive to CLK2 kinase activity. Depending on the CLK2 activity modulated by compounds, GFP or luciferase gene could be spliced out in vivo and a readout can be generated for activity of CLK2.

### EXAMPLE 4

### CLK Cell-Based Assays

i. Fat mobilization assay. 3T3 L1 cells are plated with 2 ml of 30,000 cells/ml in Dulbecco's Modified Eagle Medium (DMEM)/10% newborn calf serum in 24-well plates. Individual wells are then allowed to differentiate by addition of 100 nM Rosiglitazone. Undifferentiated control cells are maintained in fresh DMEM/10% newborn calf serum throughout the duration of the assay. At 48 hours (2 days), adipogenesis is initiated by addition of DMEM/10% fetal calf serum/0.5 mM 3-isobutyl-1-methylxanthine (IBMX)/1 μM dexamethasone. At 96 hours (4 days), adipogenesis is allowed to progress by removal of the media and adding 2 ml of DMEM/10% fetal calf serum to each well along with either 10 μg/mL insulin or 100 nM Rosiglitazone. At 144 hours (6 days) and 192 hours (8 days), all wells are changed to DMEM/10% fetal calf serum.

At 240 hours (10 days from the original cell plating), test compounds at a range of concentrations are added to individual wells in triplicate along with 100 nM Rosiglitazone. Three wells of undifferentiated cells are maintained in DMEM/10% newborn calf serum and three wells of differentiated control cells are maintained in fresh DMEM/10% newborn calf serum with 100 nM Rosiglitazone. As a positive control for fat mobilization, resveratrol (a SIRT1 activator) is used at concentrations ranging in three fold dilutions from 100 μM to 0.4 μM.

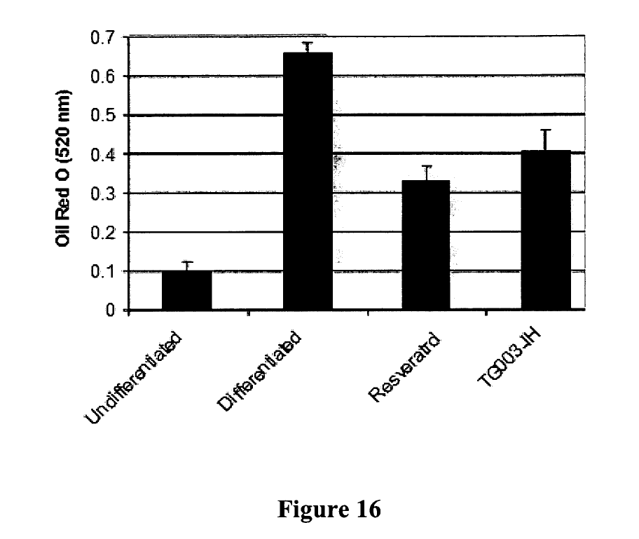

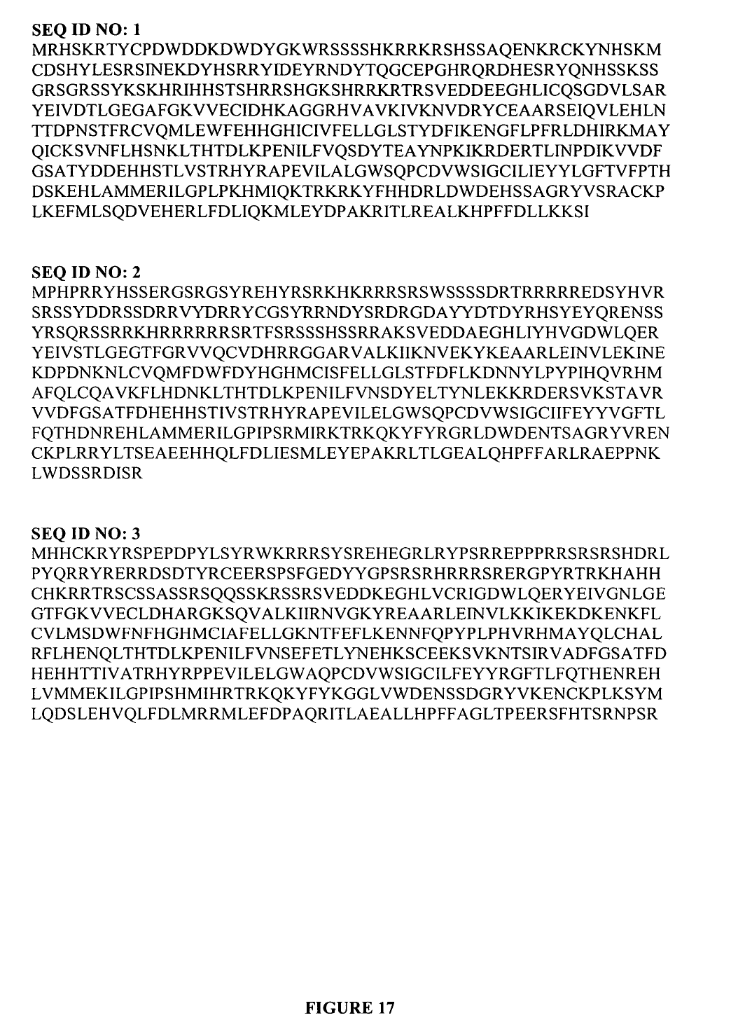

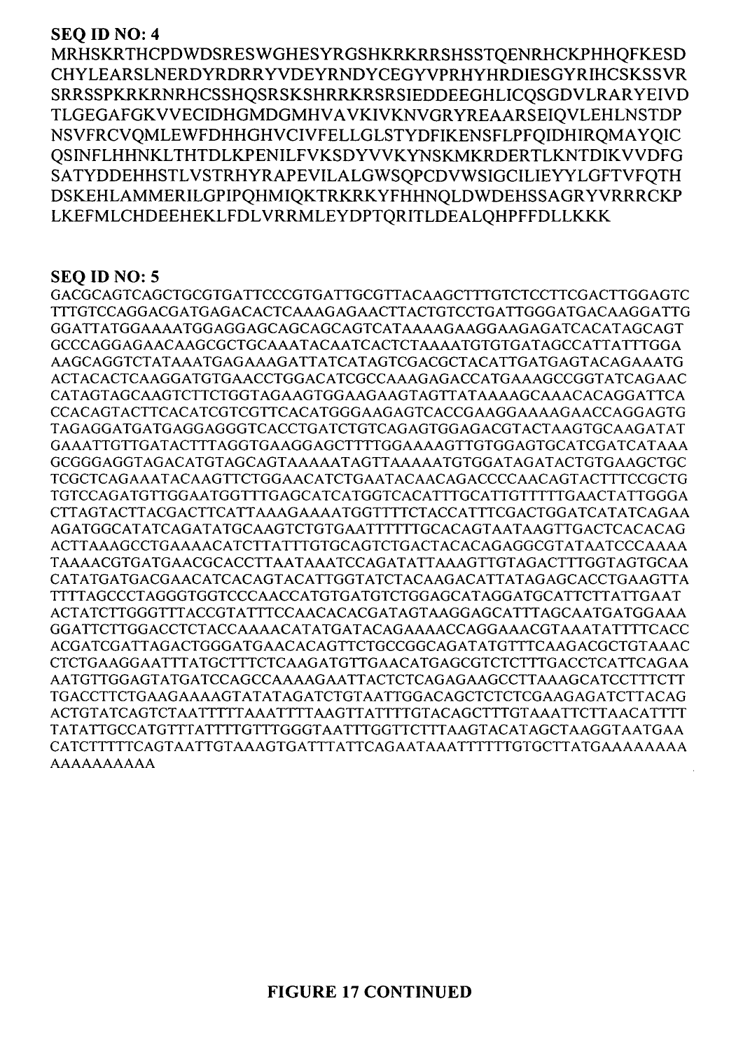

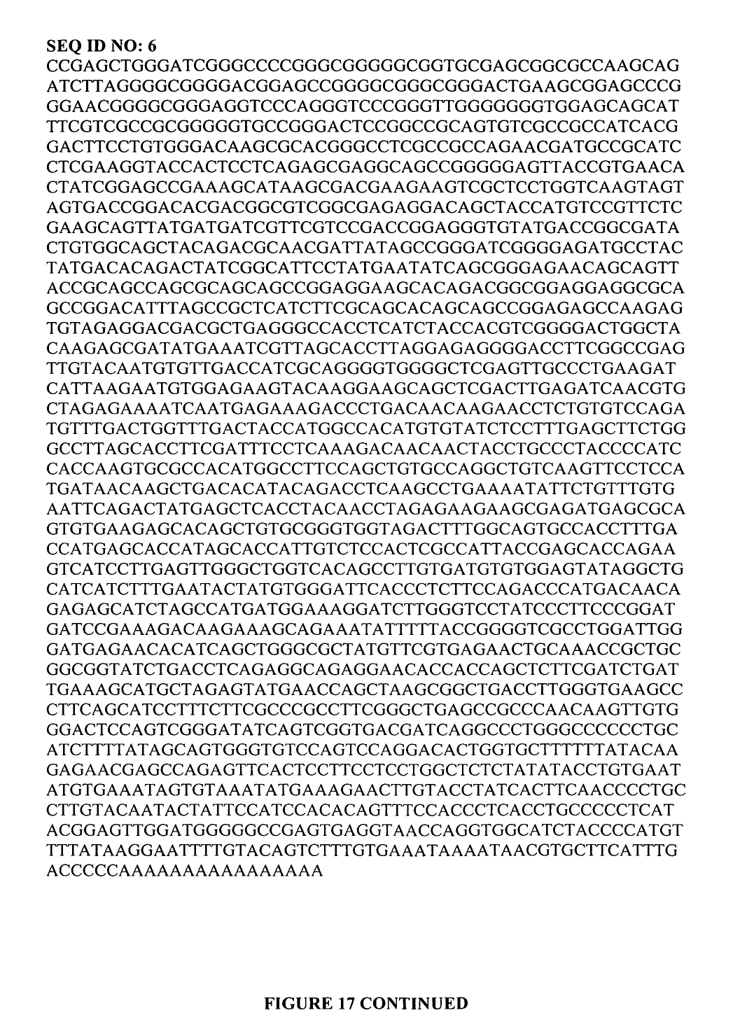

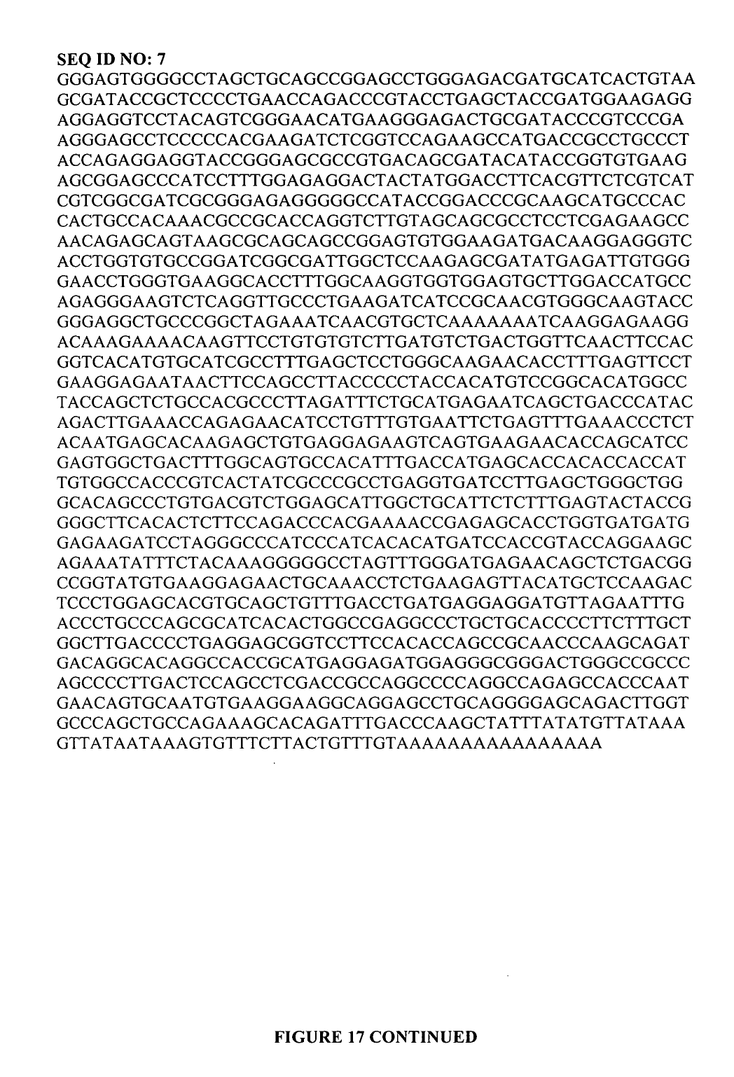

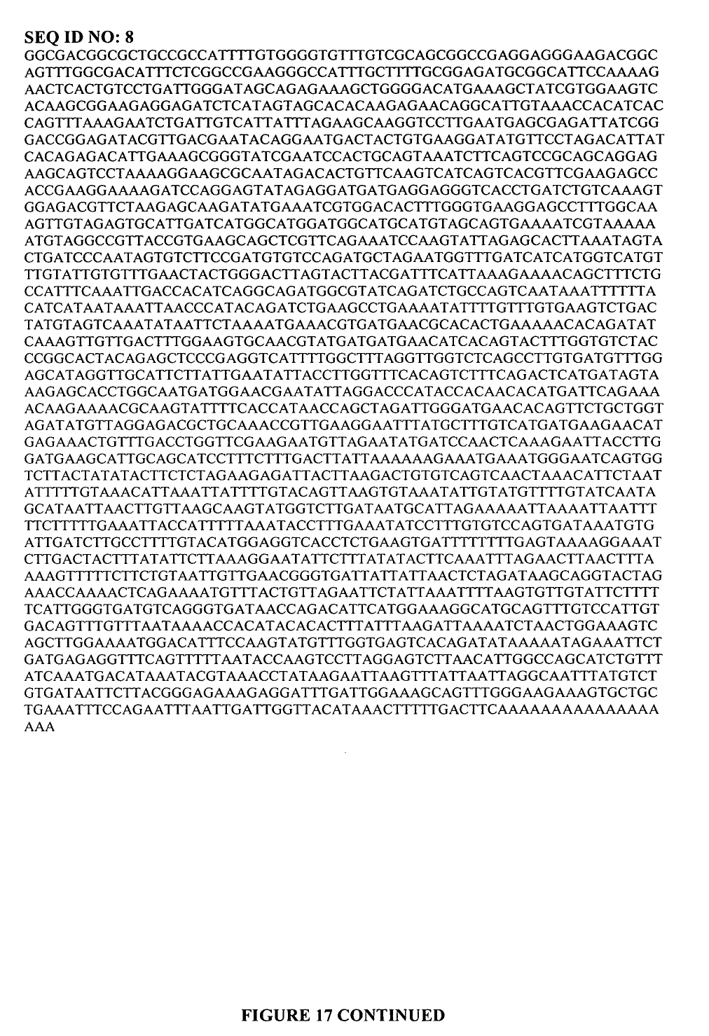

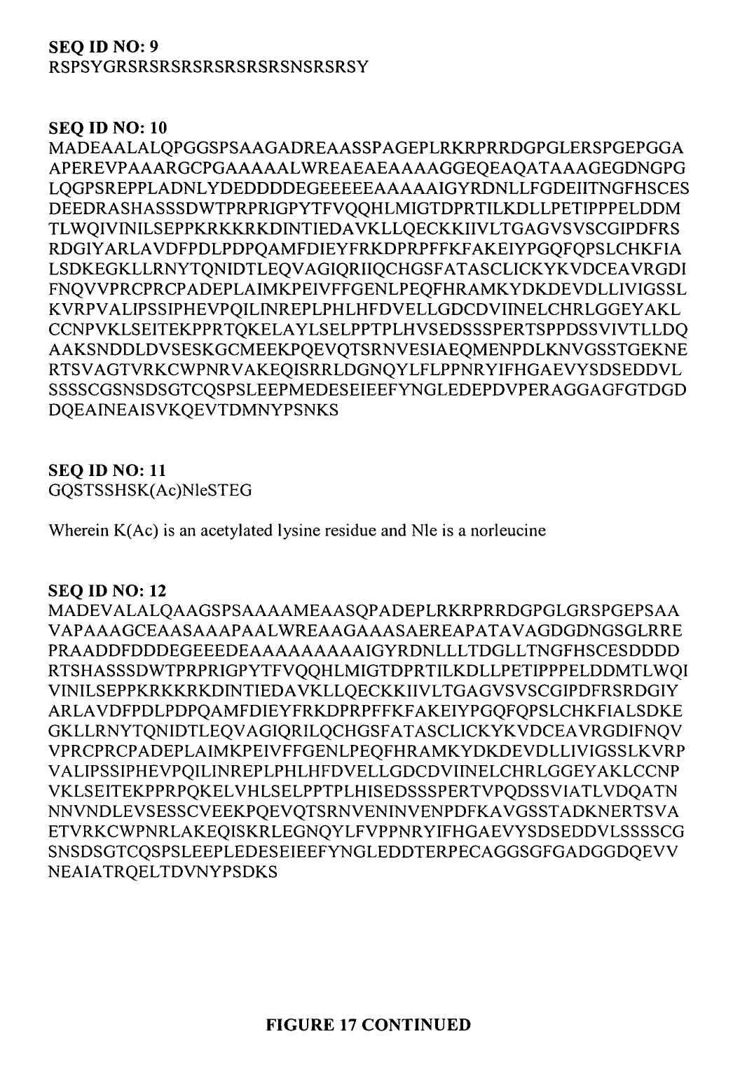

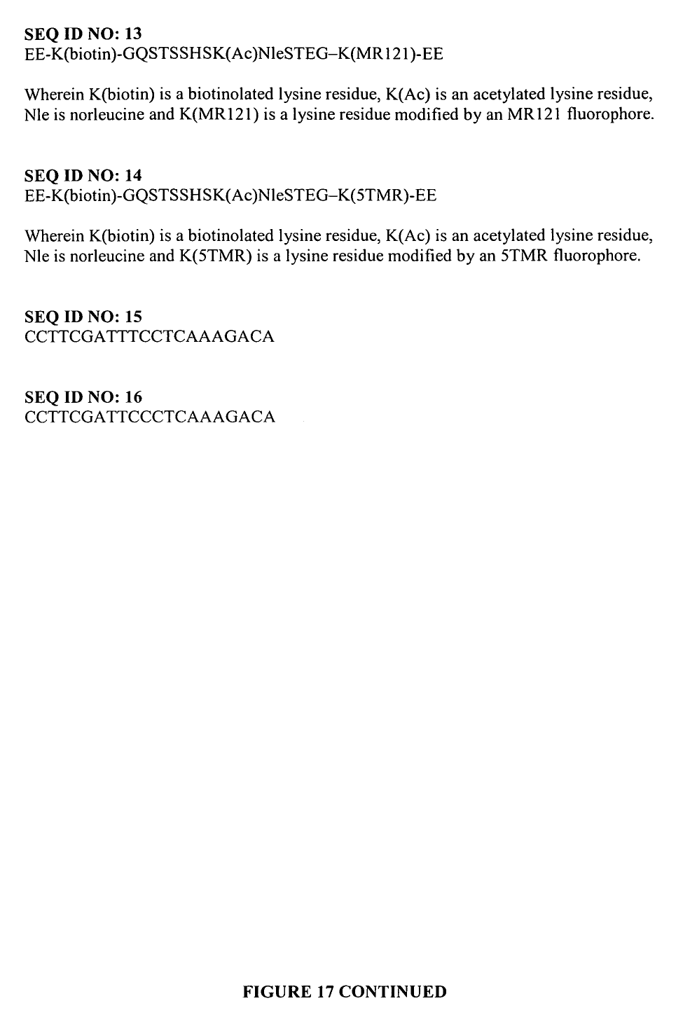

At 312 hours (13 days), the media is removed and cells are washed twice with PBS. 0.5 mL of Oil Red O solution (supplied in Adipogenesis Assay Kit, Cat.# ECM950, Chemicon International, Temecula, Calif.) is added per well, including wells that have no cells as background control. Plates are incubated for 15 minutes at room temperature, and then the Oil Red O staining solution is removed and the wells are washed 3 times with 1 mL wash solution (Adipogenesis Assay Kit). After the last wash is removed, stained plates are visualized, scanned or photographed. Dye is extracted (Adipogenesis Assay Kit) and quantified in a plate reader at 520 nM. Quantitative results are shown in FIG. 16.

II. Primary dorsal root ganglion (DRG) cell protection assay. CLK modulators are tested in an axon protection assay as described (Araki et al. (2004) Science 305 (5686):1010-3). Briefly, mouse DRG explants from E12.5 embryos are cultured in the presence of 1 nM nerve growth factor. Non-neuronal cells are removed from the cultures by adding 5-fluorouracil to the culture medium. CLK modulators are added 12 to 24 hours prior to axon transections. Transection of neurites was performed at 10-20 days in vitro (DIV) using an 18-gauge needle to remove the neuronal cell bodies.

### EXAMPLE 5

### Effects of CLK Modulators in Normal Mice

C57BL6 mice (male, 6 weeks old) are allowed to acclimatise for 48 hours. Mice are divided in to 4 groups (n=10) and receive a single, daily intraperitoneal injection of a CLK modulator (10, 30 or 100 mg/kg) or vehicle for 7 days. Daily body weights and visual observations are taken. At the end of the dosing period, mice are sacrificed by CO2 asphyxiation and blood, brain, a leg muscle and the liver collected. Blood is processed for collection of plasma, white and red blood cells. All tissues are snap frozen for storage prior to assay.

### EXAMPLE 6

### Treatment of Amyotrophic Lateral Sclerosis (ALS) (Murine Model) using CLK Modulators

ALS is a rapidly progressive motor neuron disease that invariably leads to death. In the United States alone, as many as 20,000 people are affected, and an estimated additional 5,000 people are diagnosed with the disease each year. ALS most commonly affects people between 40 and 60 years of age. In the vast majority of patients, ALS is sporadic and occurs apparently at random with no clearly associated risk factors. A particularly devastating effect of ALS is that a person's mind, personality, intelligence or memory is not affected, but their ability to react, communicate, and to control voluntary and involuntary muscles is lost.

CNS Penetration and Distribution of Radiolabeled Compound. For a compound to exhibit efficacy in an animal model of ALS, it must achieve therapeutic concentrations within the CNS and reach the sites within the CNS that are relevant to the degeneration observed. In the mouse models of ALS, the primary site of neuronal loss is the lumbar spinal cord that innervates the hind limbs and tail. To confirm that the compound of interest reaches the CNS, brain and spinal cord penetration and distribution are studied. The compound of interest is radiolabeled and administered to mice. Distribution of the compound within the CNS is determined by autoradiography and extraction.

Briefly, male Swiss Webster mice weighing 20-25 g at the time of the experiment are maintained under a light-dark cycle of 12 h-12 h at a room temperature of 21±2° C., with 50±15% humidity. The mice have free access to commercial mouse food and tap water.

The ¹⁴C-labeled CLK modulator is administered as intraperitoneal (i.p.) injections to mice every 12 h for 2 days. The amount of ¹⁴C-labeled compound injected is determined based on its specific activity and in vitro activity.

Following administration, animals are sacrificed at 30 minutes, 3 hours and 6 hours. The brains and spinal cords are rapidly removed and frozen in 2-methylbutane at −20° C., then kept below −70° C. until sectioning or solid phase extraction.

Frozen brains are mounted on cryostat chucks and cut into 20 μm thick coronal sections at −20° C. in a Microm HM 500 O microtome cryostat. Sections are thaw-mounted near the edge of slides and dried overnight under a gentle stream of air. The slides are exposed to ¹⁴C-sensitive film (Hyperfilm MP, Amersham Biosciences) at 5° C. for 3 days. Images are analyzed using an HP Scanjet 8200C scanner and analyzed using an image analysis software package (Image, NIH software). ¹⁴C standards (¹⁴C-microscales) (30-860 nCi/g) are used for quantifying the autoradiograms. Density readings for standards of known radioactivity are taken for comparison of optical density to isotope levels on each sheet of film. Standard curves for converting optical density to nCi/g values are best-fit by linear transformation. Background readings of optical density are used in determining the relative amount of drug bound to each section. Different regions of the brain selected are examined for labeling with ¹⁴C-labeled compound. Regions are identified using an atlas of the brain (Paxinos G., Franklin K. B. J., The mouse brain in stereotaxic coordinates Academic Press, New-York, 2003). The amount of ¹⁴C-labeled compound bound to each area is expressed as the mean for each slide (3 sections per slide). Data taken from areas found in both the left and right hemispheres are pooled from each section to determine the overall mean for that region of brain.

To determine compound exposure to the spinal cord, the spinal cord is homogenized and centrifuged to remove any solids from the sample. An aliquot of the sample is combined with 1% phosphoric acid with water in a 96-well plate and mixed. The sample is added to a Phenomenex StrataX extraction plate that has been equilibrated with methanol and water. Following washing, the sample is eluted with 100% acetonitrile into a clean 96-well plate. The samples are evaporated under a stream of N₂ and the residue reconstituted in solvent. The quantity of compound is assessed by mass spectrometry (LC-MS/MS).

Data are analyzed for statistical significance by ANOVA and Dunnett's t-test using the software Statview (BrainPower, Calabasas, Calif., U.S.A.). Statistical significance is taken as p<0.05.

Compound Efficacy in an Animal Model of Progressive Motor Neuron Disease (pmn/pmn). The pmn mouse model is a widely used genetic animal model for studying degeneration of motor neurons. The mice carry a spontaneous autosomal recessive mutation that leads to progressive motor neuronopathy (Schmalbruch, H., et al. J Neuropathol Exp Neurol, 1991. 50 (3): p. 192-204). pmn homozygous mice develop weakness in the hind limbs during the third week of life and die at approximately 6 weeks of age. At this latter age, the animals show a severe muscle wasting particularly in those muscles of the thoracic and pelvic regions. Heterozygous pmn mice are phenotypically normal. Histological studies have revealed that the sciatic and phrenic nerves of pmn animals are severely affected (Schmalbruch, H., et al., supra; Sagot, Y., et al. Eur J Neurosci, 1995. 7 (6): p. 1313-22; Sagot, Y., et al. J Neurosci, 1995. 15 (11): p. 7727-33; and Sagot, Y., et al. J Neurosci, 1996. 16 (7): p. 2335-41) and that 30% of the facial nucleus motor neurons degenerate (Sendtner, M., et al. Nature, 1992. 358 (6386): p. 502-4). The pmn mouse model of motor neuron disease is used to examine the potential neuroprotective properties of CLK modulators. The effects of CLK modulators on disease onset, motor function, motor neuron loss, and survival of the pmn/pmn mouse are determined.

Heterozygous pmn mice are obtained from the laboratory of Dr. Ann Kato from the Centre Medical Universitaire (Geneva, Switzerland). A large colony of pmn mice is generated; pmn/pmn homozygotes are infertile and are obtained from double heterozygous crosses at the Mendelian ratio of 25%. Starting at 12 days of age, the mice are examined for grasp activity of the hind limb paws. The first clinical signs of weakness usually appear between days 14 and 16. Animals are divided into groups at two weeks of age. Controls and treated pmn mice have access to commercial food and tap water ad libitum throughout the study. When it is determined by examiners that the mice are unable to reach dry food and/or water, a water-based nutrient gel will be placed on the bottom of the cage, and a longer spout will be attached to the water bottle.

The mice are divided into four test groups: Group A: negative-control animals (heterozygote and wild type mice) treated with vehicle; group B: positive-control animals (pmn/pmn homozygotes) treated with vehicle; group C: pmn/pmn homozygotes treated with CLK modulator (dose 1); and group D: pmn/pmn homozygotes treated with CLK modulator (dose 2).

Briefly, Group A serves as negative-control animals that do not exhibit motor neuron loss (heterozygote and wild type mice). Group A is treated with vehicle daily throughout the study. Group B is the positive-control animals and is dosed with vehicle daily throughout the study. Groups C and D are treated with the CLK modulating compound at 2 different doses. The dose is determined based on compound activity in vitro and CNS penetration determined using radiolabeled compound as described above. For these studies, test compounds or vehicle is administered i.p. twice a day with 10 to 12 hours between injections. The treatment is administered from two weeks of age throughout the study. Animals from each group are used for histological evaluation. These mice are sacrificed at a late disease stage (35 days) to assess the extent of motor neuron loss and the extent of gliosis.

The parameters followed for this study are body weight, behavior, motor neuron loss, gliosis, and life span. Throughout the study, body weight is determined daily by weighing the animals at the same time each morning prior to the administration of the CLK modulator or vehicle. The body weight evolution is expressed as the cumulative sum of the variation in the percentage of the initial body weight.

For the behavioural assessment, the mice are tested for their ability to execute the following behavioural tests: back leg grasping, bar crossing, inclined plane test and grip test.

Back leg grasping. This test measures the ability of pmn mice to hold onto the side of their cage with their hind limbs. The mice, held head-down by the tail, will be allowed to grasp the cage and remain suspended. As early as day 15, pmn homozygous animals can be diagnosed by their inability to grasp onto the side of the cage. The mice are tested every 2 days.

Bar crossing. In this test, the time to cross a 25 cm long cylindrical bar is measured. If the mice fall from the bar, the test is considered unsuccessful and is repeated three times. The mice are tested every 2 days.

Inclined plane test. The mice are tested 1 time per week for their ability to stay on an inclined plane within a maximum of 5 seconds. The slope that each animal remains on the plane is recorded.

Grip test. The mice are tested 1 time per week for their ability to hold a horizontal bar two times, within a maximum of 30 seconds. The time each animal remains on the bar is recorded.

For histological and stereological analysis, mice are perfused with phosphate buffered saline followed by paraformaldehyde. The spinal cords are dissected and the lumbar segments identified. Tissues are postfixed and blocks will be cryoprotected. To quantify motor neurons numbers, high-precision stereological analysis are performed. Serial coronal sections are cut through the lumbar (L1 to L4) spinal cord. The sections are mounted onto slides and stained for Niss1 substance using cresyl violet. A separate set of sections are collected as free-floating sections and processed for immunohistochemistry, which is aimed at determining the extent of gliosis or astrocyte and microglial involvement. The sections are immunostained with CD40 (microglial marker) and GFAP (astrocyte marker) antibodies using double label immunofluorescence.

Life span is determined for each test group. In order to reduce animal suffering, new guidelines have been established to determine endpoint (survival); animals are euthanized when they are unable to do any of the following: right themselves within 15 seconds when placed on their sides, groom their faces (as determined by infection in one or both eyes), or move around the cage, even by use of front limbs, to reach food placed at the bottom of the cage. Negative control animals are euthanized at the end of the study by CO₂ inhalation.

For statistical evaluation of the data, the life span results are submitted to a Kaplan-Meier test. Two different tests of measuring statistical significance are used; the Log-Rank test and the Wilcoxon test. Data related to quantitative behavioral assessments are analyzed with Kruskal-Wallis followed by non parametric Mann-Whitney U-test. Significance is considered as p<0.05.

Compound Efficacy in an Animal Model of ALS Disease (SOD1^{G93A}). The SOD1^{G93A} mice are obtained from the Jackson Laboratories (Gurney, M. E., et al. Science, 1994. 264 (5166): p. 1772-5). The mice express high levels of human SOD1 containing a substitution of glycine to alanine at position 93. This mutation is found mutated in 20% of familial ALS patients and thus represents a useful and relevant model for studying the efficacy of CLK modulators. The effects of the CLK modulator across standard experimental parameters are examined: disease onset, motor function, motor neuron loss, gliosis, and survival of the SOD^{G93A} mouse.

The specific mouse strain, designated G1H, is maintained as a heterozygous hybrid line which is a cross between C57B6/J and SJL mice. Transgenic males are crossed with nontransgenic B6SJLF1 females. Animals are genotyped at weaning, approximately 21-30 days of age by PCR amplification from DNA extracted from tail biopsies while the animals are temporarily anesthetized by inhalation of isoflurane. For the DNA extraction, a QIAamp Tissue Kit from Qiagen is used. PCR amplification is performed using a primer pair specific for exon 4 of the human SOD1 gene. At 30 days of age, the mice are randomized into three different treatment arms. All animals have access to commercial food and tap water ad libitum throughout the study. When it is determined by examiners that the mice are unable to reach dry food and/or water, a water-based nutrient gel will be placed on the bottom of the cage and a longer spout will be attached to the water bottle.

The following three test groups are studied: Group A: SOD1^{G93A} mice treated with vehicle serve as the positive control group; Group B: SOD1^{G93A} mice treated with the CLK modulator (dose 1); and Group C: SOD1^{G93A} mice treated with the CLK modulator (dose 2).

Briefly, Group A serves as positive-control animals that exhibit motor neuron loss. Group A is treated with vehicle daily throughout the study. Groups B and C are treated with the CLK modulating compound at 2 different doses. The dose is determined based on compound activity in vitro and CNS penetration. For these studies, test compounds or vehicle are administered i.p. twice daily with 10 to 12 hours between injections. The treatment is initiated on day 30 and continues throughout the study. Animals from each group will be used for histological evaluation. These mice are sacrificed at a late stage in the disease (120 days) to assess the extent of motor neurons loss and the extent of gliosis.

The parameters followed for this study are body weight, disease onset, gait, life span, motor neuron loss, and gliosis. Throughout the study body weight is determined daily by weighing the animals at the same time each morning prior to the administration of the test compound or vehicle. The body weight evolution is expressed as the cumulative sum of the variation in the percentage of the initial body weight.

The mice are examined twice weekly to determine disease onset. Onset is defined as the day of the first appearance of limb tremor when the animals are held suspended briefly by their tails. This usually begins unilaterally, followed by bilateral tremulousness and weakness in the affected limb(s). Following initial diagnosis, animals are examined daily for early stages of hind-limb paralysis.

Gait analysis is performed to assess motor functioning of the test groups. Briefly, footprint patterns are studied using mouse fore- and hindpaws dipped in blue and red non-toxic, water based paint, respectively. The mice are placed in a clear Perspex runway that has a black goal box fixed to one of the distal ends. White paper is used to line the runway floor. Mice are permitted to walk to the goal box from the opposite end of the runway thus allowing their footprints to leave patterns on the paper. Five separate parameters are measured; stride length, hind- and forepaw base width, overlap between fore and hindpaws, and latency to travel the runway.

Life span determination, histological analysis, stereological analysis and statistical evaluation are carried out as described above.

### EXAMPLE 7

### Treatment of Multiple Sclerosis (MS) (Murine Modulator) using CLK Modulators

Multiple Sclerosis (MS) is the most common cause of non-traumatic neurological disability affecting young adults. An estimated 2.5 million people have MS worldwide and approximately 400,000 in the U.S (source: NINDS). MS is an inflammatory disease of the central nervous system (CNS) in which demyelination and axonal injury result in a permanent neurological disability. The disease can present in different forms, such as primary progressive (accumulation of disability without remission) or relapsing remitting (acute attacks followed by periods of recovery). About 40% of patients enter a secondary progressive stage (attacks with incomplete recovery that lead to progressive disability between exacerbations). There is no cure for MS. Recently approved drugs focus on the inflammatory autoimmune components of the disease, and they appear to control relapses and may be effective in slowing progression from relapsing-remitting to secondary progressive. However, these immunomodulatory interventions do not address the underlying axonal injuries, and therefore do not impact the neurological damage resulting from acute demyelinating events, acute axonal transection and axonal loss.

Experimental autoimmune encephalomyelitis (EAE) is an animal model of MS induced by immunization with proteolipid protein (PLP). Animals mount an immune response resulting in inflammation, demyelination, and neuronal damage in the brain, spinal cord, and optic nerve, similar to MS patients. Assessment of clinical/neurological symptoms, and histological analysis of demyelination and axonal damage in the thoracic spinal cord are examined.

Chronic relapsing EAE is induced in 8-12 week old female SJL mice by subcutaneous (s.c.) injection with an emulsion containing PLP 139-151 peptide and complete Freund's adjuvant containing 150 μg of peptide and 200 μg of Mycobacterium tuberculosis in a total volume of 0.2 ml. In addition, mice are injected intraperitoneally (i.p.) with 200 ng pertussis toxin (List Biological, Campbell, Calif.) in 0.1 ml PBS on day 0 (day of immunization) and again on day 2. The animals are housed in standard conditions: constant temperature (22±1° C.), humidity (relative, 25%) and a 12-h light/12-h dark cycle, and are allowed free access to food and water. Animals are assessed daily for weight and clinical signs of EAE, beginning 11 days after immunization. Clinical assessment is on a scale from 0-5 (with “5” being moribund, “4” being quadriplegic through to “0” which is an apparently healthy animal). Assessment continues until day 40 after the initial inoculation. During this time animals undergo an initial phase of EAE, followed by recovery. A relapse of EAE typically occurs 20-30 days post-immunization. Mice are considered to have had a relapse if they have an increase by 1 on the clinical scale for two or more days after a period of five or more days of stable or improved appearance.

In order to assess the effect of CLK modulators on neurodegeneration, it is critical not to interfere with the lymphoid development of effector cells early in the disease process. Therefore, the CLK modulator is administered at the onset of clinical EAE. At the onset of clinical EAE, mice are divided randomly into groups and treated with CLK modulator (50, 100, and 200 mg/kg) or vehicle. All treatments are given by daily i.p. injection until the termination of the study.

At day 40 post-immunization, mice from each group are sacrificed with an overdose of ketamine/xylazine. Spinal cords are dissected, fixed in 10% buffered formalin, and embedded in paraffin. Five micron thick sections are stained with Hematoxylin and Eosin (H&E) and Luxol Fast Blue (LFB) to assess myelin loss. Bielshowesky's silver impregnation is used to evaluate axonal integrity. To assess the amount of axonal loss, paraffin sections are exposed to monoclonal antibodies against mouse non-phosphorylated neurofilament H (Clone SMI-32, Sternberger Monoclonals, Baltimore, USA) and monoclonal antibodies against APP (Clone 22C11, Chemicon). SMI-32 is detected with a Cy3-labeled antibody and visualized by fluorescence microscopy. Anti-APP antibodies are detected by incubation with ColonoPAP, and APP-positive axons are visualized with 3,3′-diaminobenzidine (DAB).

To evaluate the extent of axonal loss, images of slides are captured and the areas stained by immunohistochemistry are quantified blinded to treatment status. Axonal integrity and demyelination are assessed qualitatively.

Even though immunosuppression is responsible for reducing the clinical severity of the initial phase of EAE, a recent study suggests that a combination of immunosuppression and neuroprotection may be critical to effectively inhibit relapses, demyelination and axonal injury, and that chronic immunosuppression in the absence of effective neuroprotection may worsen the clinical outcome in EAE and, perhaps, MS. This issue is addressed by evaluating the effect of immunosuppression (by Copaxone (glatiramer acetate)) in combination with neuroprotection (by CLK modulators) in the PLP-induced EAE mouse model.

Chronic relapsing EAE is induced as described above. Mice are divided into three treatment groups: Group 1: vehicle control, daily i.p. injections of cyclodextrin (days 12-39); Group 2: Copaxone treatment, daily s.c. injection (days 0-9); and Group 3: Copaxone (days 0-9) and CLK modulator (days 12-39). As described above, disease progression is monitored, and mice from each group are sacrificed, the spinal cords harvested and analyzed for demyelination, axonal integrity and axonal damage.

### EXAMPLE 8

### Treatment of Huntington's Disease (Murine Model) using CLK Modulators

The R6/2 mutant mouse model of Huntington's disease (HD) is used to test the efficacy of CLK modulating compounds to attenuate HD disease-related symptoms.

R6/2 mice are treated with a CLK modulating compound for at least 12 weeks. The mice are evaluated at 4, 6, 8 and 12 weeks of age (except for Grip Strength which will only be tested 12 weeks of age) using the Rotarod, grip strength, rearing/climbing, open field, and body weight/survival test.

During the course of the study, 12/12 light/dark cycles are maintained. The room temperature is maintained between 20 and 23° C. with a relative humidity maintained around 50%. Chow and water are provided ad libitum for the duration of the study. Each mouse is randomly assigned across the dose groups and balanced by cage numbers. The test is performed during the animal's light cycle phase unless otherwise specified.

Rotarod. Motor coordination and exercise capacity are assessed by rotarod at 4, 6, 8 and 12 weeks of age. Tests are performed on three separate days, with four trials per day. Animals are loaded on the continuous rotating rod (Accuscan, Columbus, Ohio) 8 animals at a time. They are given a 5-min training period at a slow speed of 4 rpm. If an animal falls off the rod it is placed back on the rod for the duration of the 5-min training period. Animals are then placed back into the home or test cage for at least one hour prior to actual testing. The mice are then placed on the rotarod and the speed is gradually and uniformly increased to a speed of 40 rpm by 300 s. The time that each mouse remains on the rotating rod before falling 20 cm onto a foam pad is recorded. Any abnormal behavior is also noted, i.e., looping behavior recording the number of rotation times per session trial, walking forward against the rod direction, and number of fecal boli. After rotarod testing animals are placed back into the test or home cage

Grip-strength test. Grip strength is used to assess muscular strength in limb muscles and mice are tested at 12 weeks of age. Mice are held by the tail and lowered towards the mesh grip piece on the push-pull gauge (San Diego Instruments, San Diego, Calif.) until the animal grabs with both front paws. The animal is lowered toward the platform and gently pulled backwards with consistent force by the experimenter until it releases its grip. The forelimb grip force is recorded on the strain gauge. The experimenter continues to pull the animal backwards along the platform until the animal's hind paws grab the mesh grip piece on the push-pull gauge. The animal is gently pulled backwards with consistent force by the experimenter until it releases its grip. The hind limb grip force is recorded on the strain gauge. After testing animals are placed back into the test or home cage.

Rearing-Climbing. Rearing-climbing behavior is used to assess motor movement and coordination. The mouse is placed on a flat surface and a closed-top wire mesh cylinder 15 cm×20 cm tall is placed over the mouse. The animal's behavior is videotaped. The following parameters are then measured over a 5 min period: number of free rears, the number of times the animal rears in contact with the wall, number of times the animal lifts either 1, 2 or 3 paws from the floor, the number of climbing episodes (lifting 4 paws), the number of hanging episodes (from the mesh), and the time spent hanging and climbing. After the 5-min session animals are placed back into the home cage.

Open field—locomotor activity. Mice are acclimated to the test room at least 1 hour prior to the commencing the test. The open field test (OF) is used to assess both anxiety-like behavior and motor activity. The open field chambers are plexi-glass square chambers (27.3×27.3×20.3 cm; Med Associates Incs., St Albans, Vt.) surrounded by infrared photobeam sources (16×16×16). The enclosure is configured to split the open field into a center and periphery zone and the photocell beams are set to measure activity in the center and in the periphery of the OF chambers. Animals having higher levels of anxiety or lower levels of activity tend to stay in the corners of the OF enclosures. On the other hand, mice that have high levels of activity and low levels of anxiety tend to spend more time in the center of the enclosure. Horizontal activity (distance traveled) and vertical activity (rearing) are measured from consecutive beam breaks. Animals will be placed in the OF chambers for 30 minutes. Ambulatory distance in center and periphery; rearing in center and periphery; the number of zone entries and average velocity are measured.

Body Weight and Survival. Body weights are measured daily. The survival times of the mice tested as described above are determined. Fatalities are evaluated in the context of the other parameters measured. In our previous studies in R6/2 Huntington's disease model mice, we found no differences between survival times in experimental versus non-experimental groups.

Statistical Analysis. Data are analyzed by a one-way or two-way analysis of variance (ANOVA) followed by post-hoc comparisons. An effect is considered significant if p<0.05. Data are represented as the mean and standard error to the mean (s.e.m.). Animals are removed from the group if the data is two standard deviations away from the mean.

### EXAMPLE 9

### Treatment of Chemotherapeutic-Induced Neuropathy (Rodent Model) using CLK Modulators

The oncology drug Taxol (paclitaxel) is an effective treatment of ovarian, lung, breast and other cancers but its anti-microtubule activity can induce peripheral neuropathies. Taxol administration, either in a single large dose or several smaller doses, has been demonstrated to produce both sensory-motor deficits and histologically identified axonal abnormalities in rodent models. These models are thought to be predictive of those neuropathies often seen in patients given Taxol for chemotherapy for various forms of cancer. Both sensory-motor behavioral testing and histological evaluation of nerve tissue in animals treated with Taxol and concomitantly treated with either vehicle or a CLK modulator are used to evaluate the effectiveness of CLK modulating compounds to attenuate the effects of Taxol on the peripheral nervous system.

Male Sprague-Dawley rats (Harlan Sprague Dawley Inc., Indianapolis, Ind., USA) are injected intra-peritoneally with Taxol at 20 mL/kg i.p. (32 mg/kg total dose) on Day 0 using a syringe and sterile needle. A first set of rats are treated with Normal Saline vehicle. The rats are dosed on Day 0 in combination with Taxol and are injected subcutaneously using a syringe and sterile needle. This dosing procedure is repeated at 24 and 48 hours post-Taxol injection. The volume of vehicle administered is 1 ml/kg bodyweight. A second set of rats are treated with a CLK modulating compound. The rats are treated with a CLK modulating compound commence on Day 0 in combination with Taxol.

Behavioral tests. Behavioral tests will include thermal paw stimulation for pain assessment test and the open field test for activity.

Thermal paw stimulation is a commonly-used method to assess hyper- and hypoalgesia in rodents. Using a thermal paw stimulator (UCSD), the latency for the rat to lift its paw is recorded in response to a heat source placed beneath the hindpaw. The rat is placed on a glass surface maintained at a constant temperature (30±1° C.) and then habituated to the apparatus for approximately 15 min prior to testing. Two measurements of paw lift latency are averaged for each animal if they are within 2 sec. of each other. If not, additional testing is performed until this criterion is met. Baseline testing is performed on Day −3. Further tests will be conducted on Days 4 and 7.

Necropsy. On day 14 animals are euthanized by CO₂ asphyxiation and cervical dislocation. Following euthanasia the dorsal ganglia of the lumbar vertebra, sciatic nerve and hind paw dermis are harvested and fixed overnight in 10% neutral buffered formalin.

Histology. The harvested tissue is blocked, embedded in paraffin, sectioned and stained with H&E. The tissue is examined using light microscopy and scored by an evaluator blind to the treatment regimen. The tissue is ranked on a scale of 0 to 3 based on the degree and amount of axonal disruption observed in the section, with 0 being a normal appearance of the axon, 1 to 2 being a mild to moderate disruption of the axons and a 3 being a complete disruption and Wallerian degeneration of the axons.

Statistics. A two-way repeated measures ANOVA is performed on the thermal paw stimulation and open field measurements (group×time) to assess the effects of time and treatment on the behavioral performance in these rats. If there are any overall significant differences, a factorial ANOVA is performed at specific time points to determine where the difference occurred. The neuroanatomical evaluation is assessed for statistical significance using a non-parametric analysis of the rating scores for axonal disruption.

### EXAMPLE 10

### Metabolic Activities of CLK Inhibitors in a Diet Induced Obesity (DIO) Mouse Model

In order to define whether CLK inhibitors protect against the development of obesity and associated insulino-resistance, a CLK inhibitor is chronically administered (such as via food admix) to male C57BL6J mice that are subjected during 16 weeks to a high fat diet. The mice undergo an extensive phenotypic and molecular analysis to define the regulatory pathways affected by CLK inhibition.

In this long-term study, 50 male C57BL6J mice (5 weeks of age) are analyzed during a period of 18 weeks. Five groups of 10 animals are assigned as follows:
1: chow diet
2: chow diet+CLK inhibitor (200 mg/kg/day)
3: high fat diet
4: high fat diet+CLK inhibitor (200 mg/kg/day)
5: high fat diet+CLK inhibitor (400 mg/kg/day)

During the entire study, body weight and food intake are monitored twice weekly.

During week 1, body composition is analyzed, for all groups, by dual energy X-ray absorptiometry (dexascan).

During week 2, serum levels of glucose, triglycerides, cholesterol, HDL-C, LDL-C and insulin are measured in all groups after a fasting period of 12 h and mice are then placed on the diets as indicated (Day 0).

During week 10, glucose tolerance is determined by subjecting all the animals to an intraperitoneal glucose tolerance test (IPGTT). Animals are fasted for 12 h prior to this test.

Nocturnal energy expenditure of groups 1, 3 and 5 (chow diet, high fat diet and high fat diet 400 mg) is measured by indirect calorimetry.

During week 12, body weight composition is again analysed by dexascan for all groups.

During week 13, circadian activity of groups 3, 4 and 5 (high fat diet fed mice) is studied during a period of 30 h.

During week 14, measurement of blood pressure and heart rate is performed on groups 3, 4 and 5.

During week 15, rectal temperature of all animals is measured at room temperature at 10:00 am.

A circadian activity measurement is performed on groups 1, 2 and 3.

During week 16, glucose tolerance is analysed by performing an oral glucose tolerance test (OGTT) on a subset of animals (n=5) of groups 3, 4 and 5, and an intraperitoneal insulin sensitivity test (IPIST) on another subset of animals (n=5). During these experiments, blood is also collected to analyze insulin levels. Animals are fasted 12 h prior these tests.

Feces are collected in all groups over a 24 h time period and fecal lipids content are measured.

During week 17, serum levels are measured on a subset of mice (n=5) at 7:00 am which corresponds to the beginning of the light cycle and on another subset of mice (n=5) three hours later (10:00 am). Moreover, thyroid hormone T3 levels are measured in the blood collected at 7:00 am and plasma lipoproteins levels are measured in the blood collected at 10:00 am.

During week 18, a cold test is performed on all animals by measuring body temperature of animals exposed to 4° C.

Three days later, animals are sacrificed.

At sacrifice, blood is collected and analyzed for: plasma lipids (TC, TG, HDL-C, FFAs); liver functions (ALAT, ASAT, alkaline Pase, γ-GT); and glucose and insulin lipoprotein profiles of selected groups of plasma (size-exclusion chromatography).

Liver, small intestine, adipose tissues (WAT and BAT), pancreas, heart and muscle are collected and weighed. These can be analyzed by standard histology (HE staining, succinate dehydrogenase staining, oil-red-O staining and cell morphology); for tissue lipid content; and by electron microscopy on BAT and muscle to analyze mitochondria. RNA isolation can be conducted for expression studies of selected genes involved in metabolism and energy homeostasis by quantitative RT-PCR. Microarray experiments can also be performed on selected tissues. In addition, protein extraction can be performed for the study of changes in protein level and post-translational modifications such as acetylation of proteins of interest (e.g. PGC-1α).

Methods

Animal housing and handling. Mice are group housed (5 animals/cage) in specific pathogen-free conditions with a 12 h:12 h (on at 7:00) light-dark cycle, in a temperature (20-22° C.) and humidity controlled vivarium, according to the European Community specifications. Animals are allowed free access to water and food.

Drinking water. Chemical composition of the tap water is regularly analyzed to verify the absence of potential toxic substances at the Institut d'Hydrologie, ULP, Strasbourg. Drinking water is treated with HCl and HClO₄ to maintain pH between 5 and 5.5 and chlorin concentration between 5 and 6 ppm.

Diet. The standard rodent chow diet is obtained from UAR and the high fat diet is obtained from Research Diet. Mice are fed, either with chow diet (16% protein, 3% fat, 5% fiber, 5% ash) or with high fat diet (26.2% protein, 26.3% carbohydrate, 34.9% fat). A CLK modulators is mixed with either powdered chow diet or powdered high fat diet and pellets are reconstituted. Control groups receive pellets as provided by the company. In case of the chow, which is harder to reconstitute, a minimal amount of water is added to the powder to reconstitute pellets, which are then air-dried. New batches of food are prepared weekly.

Blood collection. Blood is collected either from the retro-orbital sinus or from the tail vein.

Anesthesia. For the dexa scanning experiment, animals are anesthetized with a mixture of ketamine (200 mg/kg)/Xylasine (10 mg/kg) administered by intra-peritoneal injection.

Analysis of lipids and lipoproteins. Serum triglycerides, total and HDL cholesterol are determined by enzymatic assays. Serum HDL cholesterol content is determined after precipitation of apo B-containing lipoproteins with phosphotungstic acid/Mg (Roche Diagnostics, Mannheim, Germany). Free fatty acids level is determined with a kit from Wako (Neuss, Germany) as specified by the provider.

Metabolic and endocrine exploration. Blood glucose concentration is measured by a Precision Q.I.D analyzer (Medisense system), using Medisense Precis electrodes (Abbot Laboratories, Medisense products, Bedford, USA). This method has been validated, by comparing Precision Q.I.D analyzer values with classical glucose measurements. The Precision Q.I.D method was chosen since it requires a minimal amount of blood and can hence be employed for multiple measurements such as during an IPGTT. Plasma insulin (Crystal Chem, Chicago, Ill.) is determined by ELISA according to the manufacturer's specifications. Plasma level of T3 is determined by standard radio-immunoassays (RIA) according to the protocol specified by the providers.

Lipoprotein profiles. Lipoprotein profiles are obtained by fast protein liquid chromatography, allowing separation of the three major lipoprotein classes VLDL, LDL, and HDL.

Intraperitoneal glucose tolerance test—Oral glucose tolerance test. IPGTT and OGTT are performed in mice which are fasted overnight (12 h). Mice are either injected intraperitoneally (IPGTT) or orally gavaged (OGTT) with a solution of 20% glucose in sterile saline (0.9% NaCl) at a dose of 2 g glucose/kg body weight. Blood is collected from the tail vein, for glucose and insulin monitoring, prior to and at 15, 30, 45, 75, 90, 120, 150, 180 min after administration of the glucose solution. The incremental area of the glucose curve is calculated as a measure of insulin sensitivity, whereas the corresponding insulin levels indicate insulin secretory reserves.

Intraperitoneal insulin sensitivity test. Fasted animals are submitted to an IP injection of regular porcine insulin (0.5-1.0 IU/kg; Lilly, Indianapolis, Ind.). Blood is collected at 0, 15, 30, 45, 60, and 90 min after injection and glucose analyzed as described above. Insulin sensitivity is measured as the slope of the fall in glucose over time after injection of insulin.

Energy expenditure. Energy expenditure is evaluated through indirect calorimetry by measuring oxygen consumption with the Oxymax apparatus (Columbus Instruments, Columbus, Ohio) during 12 h. This system consists of an open circuit with air coming in and out of plastic cages (one mouse per cage). Animals are allowed free access to food and water. A very precise CO₂ and O₂ sensor measures the difference in O₂ and CO₂ concentrations in both air volumes, which gives the amount of oxygen consumed in a period of time given that the air flow of air coming in the cage is constant. The data coming out of the apparatus are processed in a connected computer, analyzed, and shown in an exportable Excel file. The values are expressed as ml·kg⁻¹·h⁻¹, which is commonly known as the VO₂.

Determination of body fat content by Dexa scanning. The Dexa analyses are performed by the ultra high resolution PIXIMUS Series Densitometer (0.18×0.18 mm pixels, GE Medical Systems, Madison, Wis., USA). Bone mineral density (BMD in g/cm²) and body composition are determined by using the PIXIMUS software (version 1.4×, GE Medical Systems).

Non-invasive Blood Pressure and heart Rate measurements. The Visitech BP-2000 Blood Pressure Analysis System is a computer-automated tail cuff system that is used for taking multiple measurements on 4 awake mice simultaneously without operator intervention. The mice are contained in individual dark chambers on a heated platform with their tails threaded through a tail cuff. The system measures blood pressure by determining the cuff pressure at which the blood flow to the tail is eliminated. A photoelectric sensor detects the specimen's pulse. The system generates results that Applicants have shown correspond closely with the mean intra-arterial pressure measured simultaneously in the carotid artery. This allows obtaining reproducible values of systolic blood pressure and heart beat rate. This requires training of the animals for one week in the system.

Circadian Activity. Spontaneous locomotor activity is measured using individual boxes, each composed with a sliding floor, a detachable cage, and equipped with infra-red captors allowing measurement of ambulatory locomotor activity and rears. Boxes are linked to a computer using an electronic interface (Imetronic, Pessac, France). Mice are tested for 32 h in order to measure habituation to the apparatus as well as nocturnal and diurnal activities. The quantity of water consumed is measured during the test period using an automated lickometer.

### EXAMPLE 11

### CLK2 Mediated Events in Hepatocytes

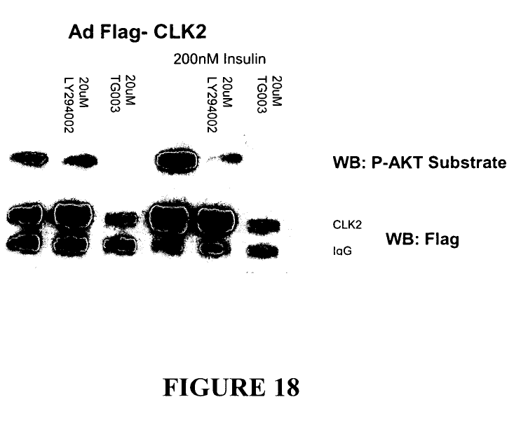

This experiment demonstrates that CLK2 phosphorylation is stimulated by insulin. H2.35 hepatocytes were infected for 2 days with Adenovirus Flag-Clk2 in low glucose DMEM 4% FBS. Cells were then serum starved in low glucose DMEM 0.5% BSA for 24 hours. Cells were pretreated with inhibitors for 30 minutes before stimulation with 200 nM insulin for 40 minutes. Cells were lysed and CLK2 was immunopurified using anti-flag agarose (Sigma-Aldrich, Cat. #A4596), immunoprecipitates where analyzed by SDS-PAGE and western blotting using anti-phospho-akt substrate antibodies (Cell Signaling Technology, Cat. #9614) as shown in FIG. 18.

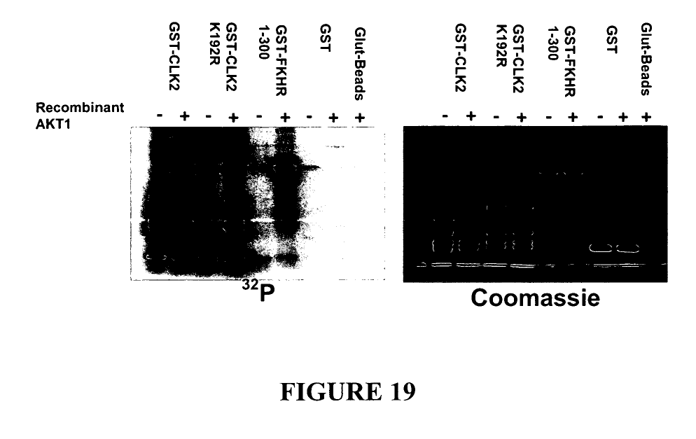

The next experiment demonstrated that AKT phosphorylates CLK2 in vitro. Purified recombinant GST-CLK2, GST-Clk2 K192R (catalytic mutant), GST-FKHR AAs1-300 (a control GST fusion protein), or GST alone were incubated overnight with recombinant Akt1 (Cell Signaling Technology, Cat. #7500) with 10 uCi of ³²P ATP and 50 uM cold ATP. Reactions were stopped by boiling in sample buffer then analyzed by SDS-PAGE. The gel was coomassie stained to show loading and then dried and exposed to film for detection of ³²P as shown in FIG. 19.

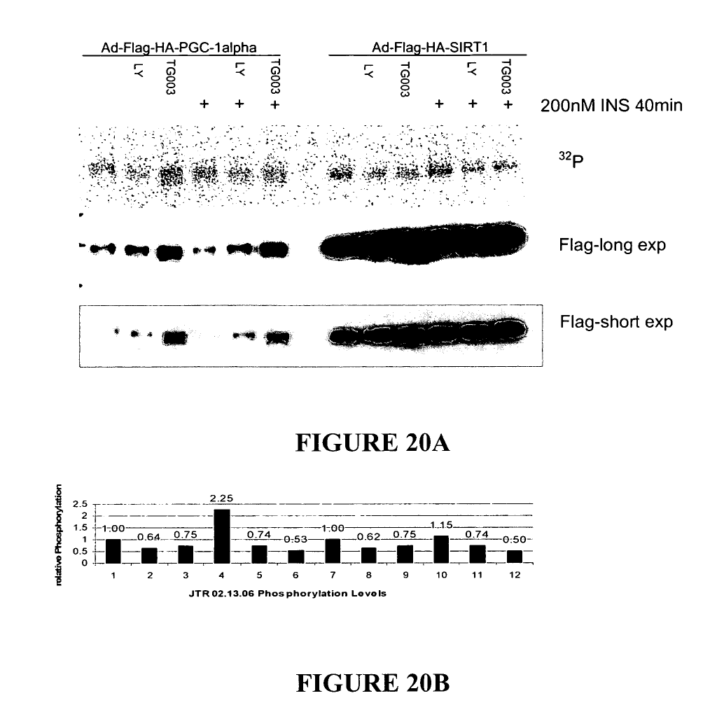

PGC-1alpha and SIRT1 phosphorylation in vivo is stimulated by insulin and blocked by LY and TG003 as determined by metabolic ³²P PO₄ labeling of H2.35 cells infected with adenovirus Flag-Ha-PGC-1alpha or Flag-HA-SIRT1. Cells were infected for 24 hours low glucose DMEM 4% FBS then serum starved for 24 hours in low glucose DMEM 0.5% BSA. Media was then replaced with PO₄ free DMEM 0.5% BSA for 30 minutes, and cells were then pretreated with LY or TG003 for 30 minutes before addition of 100 uCi ³²P PO₄. Cells were incubated in ³²P PO₄ for 20 minutes before a 40 minute insulin stimulation. Cells were then washed with ice-cold PBS, harvested and lysed. Flag-HA-PCC-1alpha and Flag-HA-SIRT1 were immunoprecipitated used anti-flag agarose (Sigma-Aldrich, Cat. #A4596). Immunoprecipitates were washed extensively and analyzed by SDS-PAGE, transferred to PVDF membrane where ³²P signal was analyzed by a Bio-Rad Phosphor-Imager (Bio-Rad Laboratories) (FIG. 20A) and normalized to protein quantitated by western blot using anti-flag antibodies by a Versa-Doc camera system and quantitation software (Bio-Rad Laboratories) (FIG. 20B).

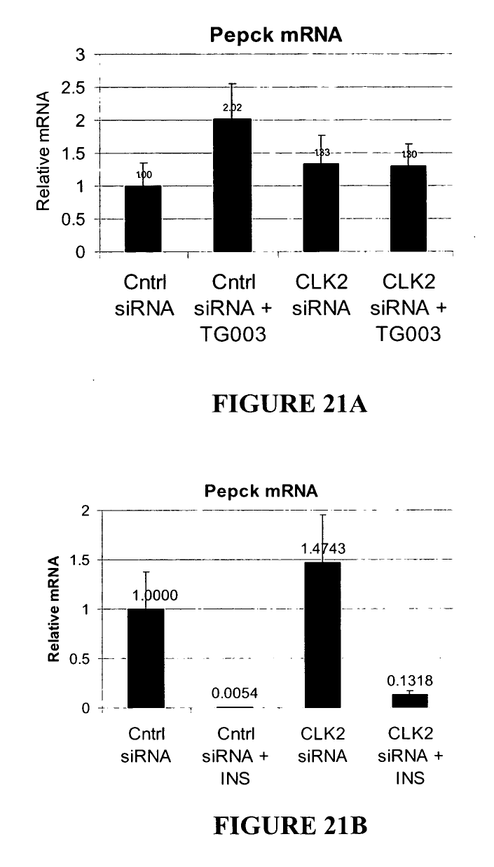

CLK2 mediates TG003 induction of PEPCK as demonstrated in FAO hepatocytes infected with adenovirus encoding CLK2 siRNA or Control siRNA. Cells were grown in F-12+5% FBS then switched to 10% FBS and treated with or without 20 uM TG003 for 7 hours. Total RNA was isolated using Trizol (Invitrogen, Cat. # 15596-026) and reverse transcribed using oligo dT and super script II (Inivtrogen, Cat. # 18064-014). Pepck mRNA was measured by Q-RT-PCR relative to 36B4 control mRNA. Each bar is average+/−stdev N=3. Results are shown in FIG. 21A.

CLK2 knock-down causes partial insulin resistance as demonstrated in FAO hepatocytes infected with Adenovirus encoding CLK2 siRNA or Control siRNA. Cells were serum starved O/N and then treated with or without 200 nM Insulin for 2 hours. Total RNA was isolated using Trizol (Invitrogen, Cat. # 15596-026) and reverse transcribed using oligo dT and super script II (Inivtrogen, Cat. # 18064-014). Pepck mRNA was measured by Q-RT-PCR relative to 36B4 control mRNA. Each bar is average+/−stdev N=3. Results are shown in FIG. 21B.

### EXAMPLE 12

### Effect of CLK2 Modulation In Vivo

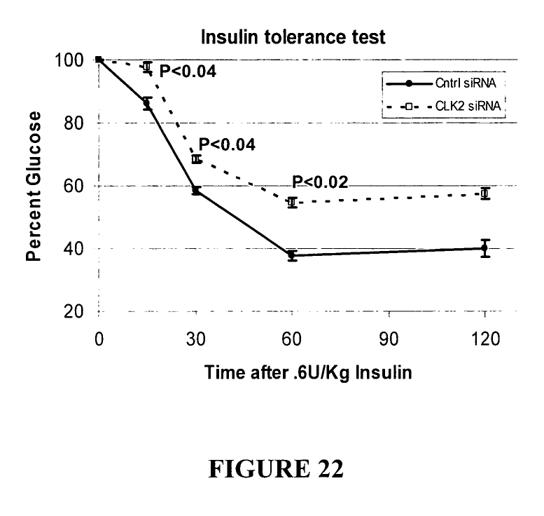

Hepatic CLK2 knock-down causes partial insulin resistance in whole animals. Six 8 week old male Balb/c albino mice were infected with 5×10⁹ infectious particles/animal of control siRNA or Clk2 siRNA adenovirus for 5 days. Mice were fasted for 12 hours before injection of 0.6 U/kg Insulin in PBS. Blood glucose levels were measured by tail bleed using an Ascencia Elite Glucometer (Bayer) at the time points indicated in FIG. 22. Graph is average+/−SEM (N=8). Significance was determined by two-tailed unpaired students T-Test.

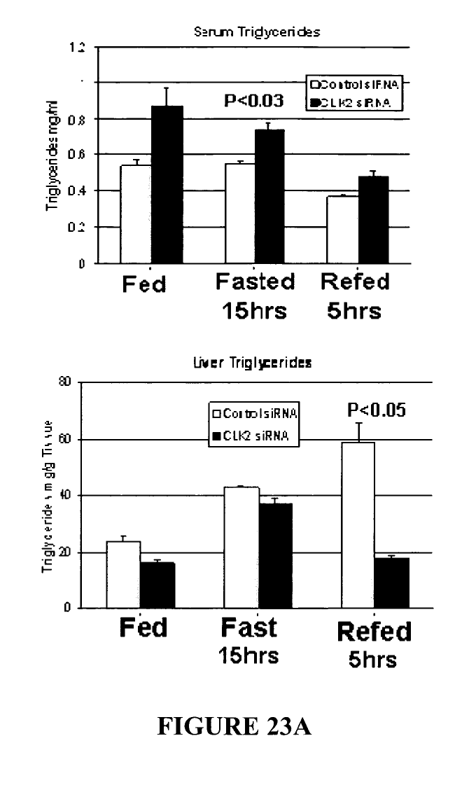

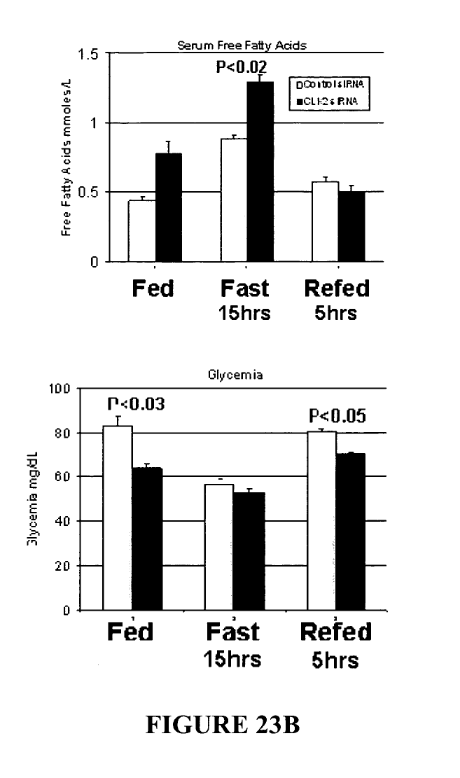

Hepatic CLK2 knock-down affects serum and liver triglycerides. 6-8 week old male Balb/c albino mice were infected with 5×10⁹ infectious particles/animal of control siRNA or Clk2 siRNA adenovirus for 8 days. At sacrifice mice were either fed, fasted for 15 hours, or fasted for 15 hours followed by 5 hours of refeeding. Triglycerides were measured using Triglyceride Reagent (Sigma) and Free Glycerol reagent (Sigma) from serum samples taken at sacrifice or from liver tissue normalized to tissue weight. Results are shown in FIG. 23A. Each bar is average+/−SEM N=4. Significance was determined by two-tailed unpaired students T-Test.

Hepatic CLK2 knock-down affects serum free fatty acids and glycemia. 6-8 week old male Balb/c albino mice were infected with 5×10⁹ infectious particles/animal of control siRNA or Clk2 siRNA adenovirus for 8 days. At sacrifice mice were either: fed, fasted for 15 hours, or fasted for 15 hours followed by 5 hours of refeeding. Serum Free fatty acids was measured using a NEFA-C kit (Wako Diagnostics) and glycemia was measured using an Ascencia Elite Glucometer (Bayer) at sacrifice. Results are shown in FIG. 23B. Each bar is average+/−SEM N=4. Significance was determined by two-tailed unpaired students T-Test.

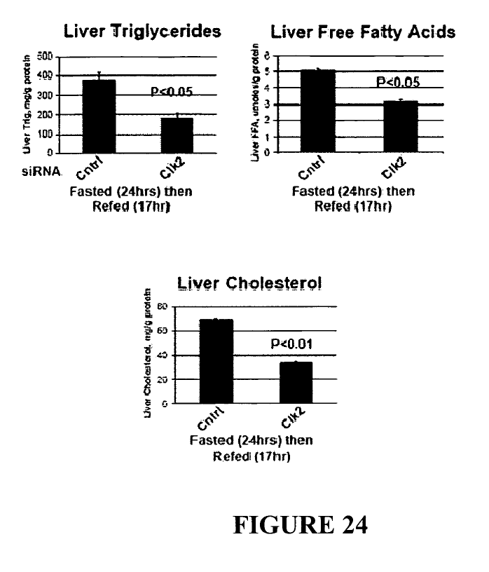

Hepatic CLK2 knock-down decreases liver lipids. 6-8 week old male Balb/c albino mice were infected with 1×10⁹ infectious particles/animal of control or Clk2 siRNA adenovirus for 8 days. Mice were sacrificed after 17 hours of refeeding following a 24 hour fast. Triglycerides were measured using Triglyceride Reagent (Sigma-Aldrich, Cat. #T2449) and Free Glycerol reagent (Sigma-Aldrich, Cat. #F6428), free fatty acids were measured using NEFA-C kit (Wako Diagnostics) and cholesterol was measured using Cholesterol Reagent (Pointe Scientific, Cat. #C7510). All measurements were normalized to protein content of liver extract. Results are shown in FIG. 24. Each bar is average+/−SEM N=4. Significance was determined by two-tailed unpaired students T-Test.

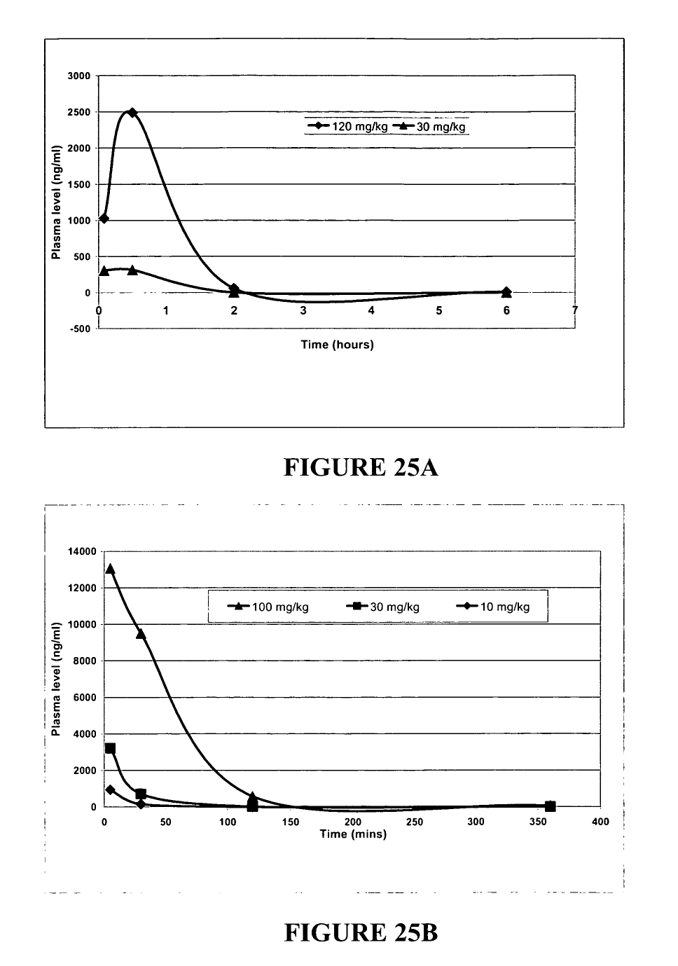

The results observed with liver only inhibition of CLK2 as exemplified in Example 12 with adenoviral delivery of CLK2 siRNA are consistent with previous experiments involving modulation of Sirt1 and/or PGC1 alpha in the liver or hepatocytes (Rodgers et al. Nature, 434, 113-118; Rhee et al. JBC, 281 (21) 14683-14690). This finding supports a role for CLK2 Inhibition that is analogous to activation or overexpression of either Sirt1 or its downstream target, PGC1alpha. Although CLK inhibition which is limited to the liver leads to an increase in insulin resistance and a reduction in liver triglycerides, liver free fatty acids and cholesterol, the effects of CLK inhibition observed upon systemic delivery support a beneficial effect of CLK inhibition, similar to SIRT1 activation, for the treatment of disorders such as diabetes, insulin resistance, metabolic disorders, weight loss, and other diseases or disorders. In particular, Example 14 and FIGS. 25-29 show that systemic intraperitoneal delivery of a CLK inhibitor leads to a decrease in body weight, a decrease in blood insulin levels, and a decrease in blood glucose levels, all of which would be beneficial for the treatment of diabetes or other metabolic diseases and disorders. Not wishing to be bound by theory, the net positive effect of the particular CLK2 inhibitor used herein (TG003) following systemic exposure may arise either because the compound is quickly metabolized in the liver limiting its effects in this organ; or that on balance the effects of inhibition of CLK2 in non-liver tissue far outweigh the effects of CLK2 inhibition in the liver.

### EXAMPLE 13

### TG003 Dosing PO and IP in Mice

Five week old C57BL/6 mice (male, 18-22 grams, Charles River Labs, Willmington, Mass.) were dosed with TG003 suspended in 2% HPMC+0.2% DOSS via IP injection at 10, 30 and 100 mg/kg (total volume of injection 0.2 ml, 12 mice per dose). Alternatively, mice were dosed with TG003 suspended in 2% HPMC+0.2% DOSS via oral gavage at 30 and 120 mg/kg (total volume of gavage 0.2 ml, 12 mice per dose).

Mice are dosed one at a time every 2 minutes. Mice are sacrificed at proper time points (5, 30, 120 and 360 minutes post dosing) using CO₂ overdose (place in CO₂ chamber 40 seconds before time point). Three mice are sacrificed per time point per dosing level. Approximately 0.5 ml blood is immediately taken via cardiac stick with a 25 G1 ml syringe. The needle is removed and the sample is added to a BD microtainer tube with Lit.Heparin and placed on ice until ready to spin. Three samples are spun every 15 minutes. The plasma is transferred to a snap tube and frozen on dry ice. TG003 plasma concentration is determined by GC/Mass Spec analysis.

Results

The plasma levels of TG003 following oral or IP dosing at the indicated doses are shown in FIGS. 25A and 25B respectively.

### EXAMPLE 14

### IP Dosing of TG003 in DIO Mouse Model

Obesity and type II diabetes are being intensively studied in animal models, particularly the mouse. One such model is commonly referred to as the diet-induced obese (DIO) model. Typically, C57BL/6 males are fed a high fat diet for 8 to 12 weeks and, as a result, become obese, mildly to moderately hyperglycemic, and glucose intolerant. These mice are then used to study the genetic and physiological mechanisms of obesity and type II diabetes.

Specifically, 5 week old C57BL/6 mice (male 18-22 grams, Charles River Labs, Willmington, Mass.) are placed either on a high fat diet (Research Diets Inc., New Brunswick, N.J., 60% kcal fat Rodent Diet Cat#D12492) or regular chow. Mice are weighed once a week for 5 weeks, test baseline fed glucose, lactate, triglycerides and insulin. At approximately 6 weeks or when mean weight of the DIO groups reach 40 grams, dosing is initiated. TG003 was dosed via IP injection at either 30 or 100 mg/kg (TG003 suspended in 2% HPMC+0.2% DOSS as in previous example). Control DIO and chow fed animals were dosed with IP injection of vehicle alone. Once dosing starts data collections was as follows: Week 1 time point includes a fasted blood glucose measurement, Week 2 blood collection, Week 3 an IPGTT and Week 4 is an endpoint blood and tissue collection.

Mice are dosed at the same time daily. Body weights are taken 2 times a week once dosing starts. Baseline measurements of fed glucose, lactate, triglycerides and insulin are taken at time 0 (commencement of dosing).

Results

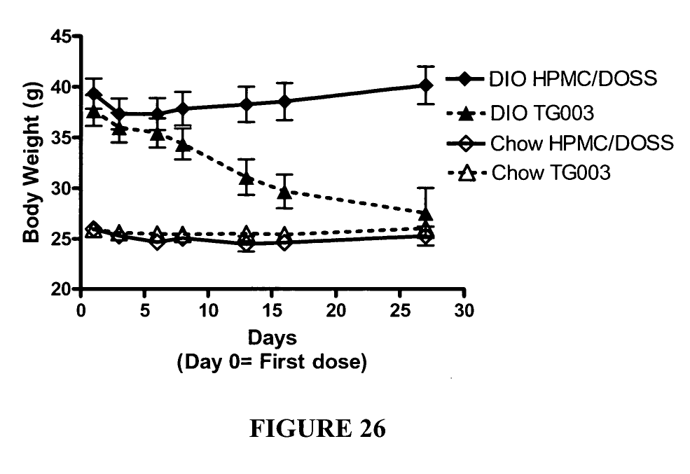

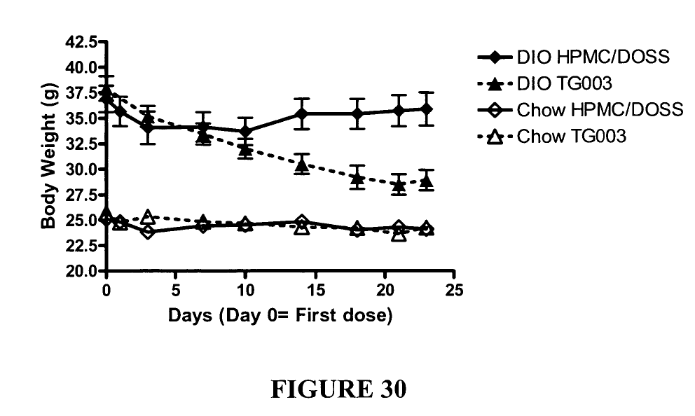

Body weights: Change in body weight of mice in each group upon commencement of dosing is shown in FIG. 26 for 100 mg/kg TG003 study and in FIG. 30 for 30 mg/kg TG003 study.

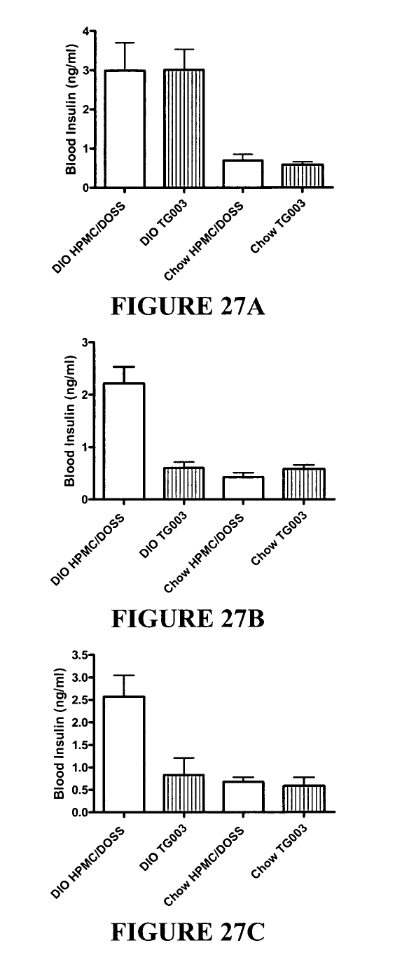

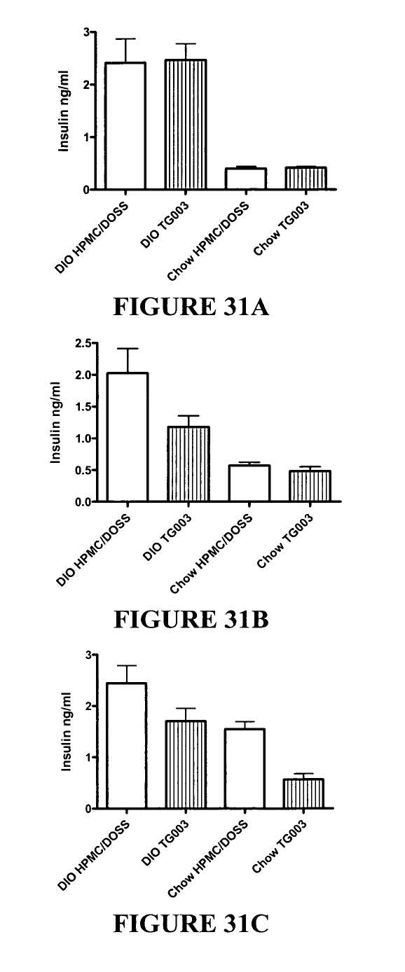

Insulin Assay: Mouse insulin levels were measured using the Linco Rat/Mouse Insulin ELISA kit (Cat. #EZRMI-13K). 0 week, 2 week and 4 week blood insulin levels are shown in FIGS. 27A, 27B and 27C respectively for 100 mg/kg TG003 study. 0 week, 2 week and 4 week blood insulin levels are shown in FIGS. 31A, 31B and 31C respectively for 30 mg/kg TG003 study.

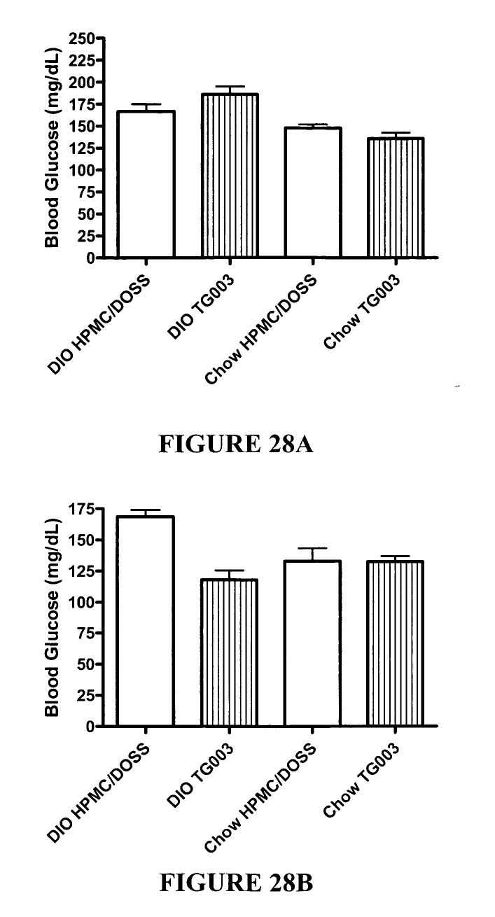

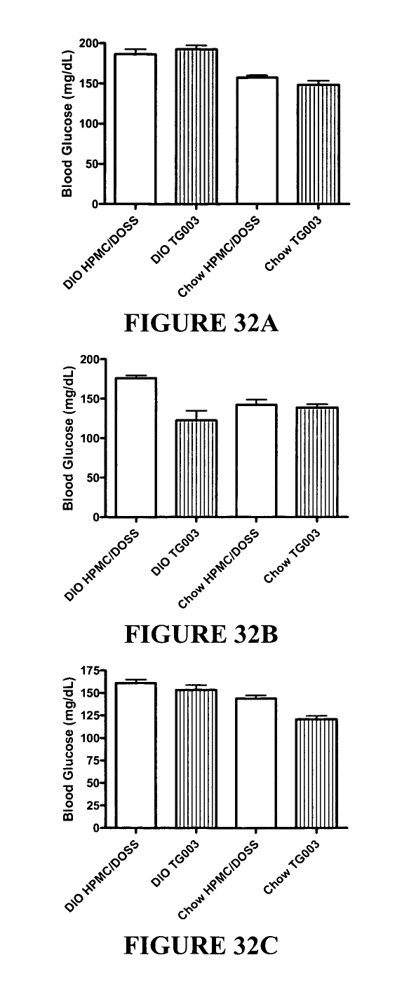

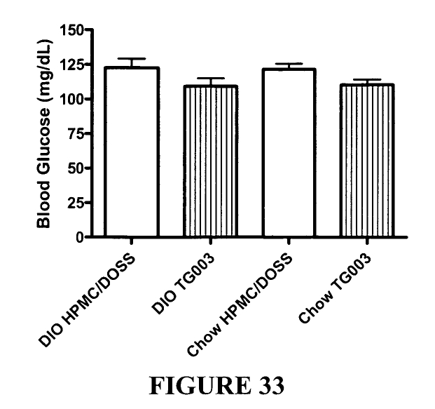

Fed blood glucose: Mouse fed blood glucose levels were measured at 0 and 2 weeks of the 100 mg/kg TG003 study (FIGS. 28A and 28B) and at 0, 2 and 4 weeks of the 30 mg/kg TG003 study (FIGS. 32A, 32B and 32C). In addition, a fasted blood glucose at 3 weeks is shown in FIG. 33 for the 30 mg/kg TG003 study.

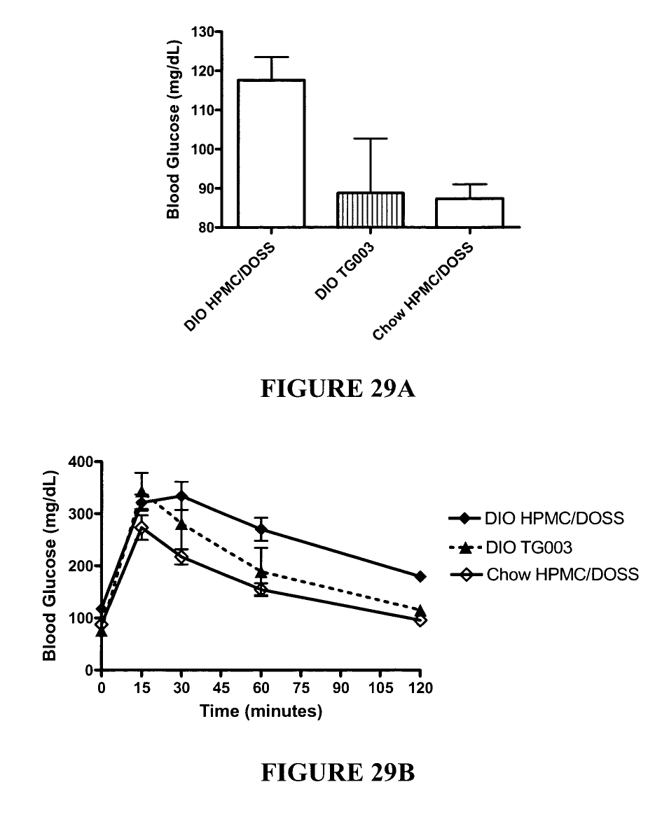

IPGTT: Mice are fasted for a minimum of 16 hrs. A glucose reading is taken at time Zero using a glucose meter. Mice are injected with 2 g/kg D-Glucose at one minute time points. A glucose reading is taken at 15, 30, 60, and 120 minutes (Medisense Precision Extra, Blood Glucose Meter, Abbott Cat# 70297-01). Initial fasted blood glucose at 3 weeks is shown in FIG. 29A and IPGTT curves are shown in FIG. 29B.

### EXAMPLE 15

### Oral Dosing of TG003 in DIO Mouse Model

Twenty 9 week old C57BL/6 mice were placed on 60% kcal % fat diet (high fat diet with 60% of calories from fat; Research Diets, Inc., New Brunswick, N.J. Cat. No. D12492) and 15 mice were placed on regular chow. Mice were weighed once a week for 5 weeks, baseline fed glucose, triglycerides and insulin was measured. At approximately 6 weeks on the high fat diet, or when the average body weight of the DIO mice becomes 40 grams, dosing began at various doses and preparations. Mice were dosed daily with either vehicle (2% HPMC+0.2% DOSS) or with TG003 suspended in vehicle and dosed via oral gavage at 100 mg/kg (10 animals each in DIO group; 9 animals in vehicle chow group and 6 animals in TG003 chow group). The study was divided into DIO groups with mean average body weight/cage. Chow fed groups were also sorted by mean average body weight/cage. Body weights were measured once weekly to adjust dosing concentrations for body weight.

Baseline blood on all mice for glucose, triglycerides, insulin, etc. was taken following a one hour food withdrawal. Once dosing started, data collections were as follows: Week 1 time point included fasted blood glucose and body temps for select groups, Week 2 was a fed blood collection, Week 3 fasted blood glucose, Week 4 body temps on select groups and endpoint blood and tissue collection. Body temperature of select groups post dosing on week 1 and week 4 was also taken. Concentration of TG003 compound is adjusted to proper dose according to the mean weight for each group weekly. Final blood collection of all groups was taken 1 hour after dosing in order to determine levels of drug in blood compared to original PK. Mice were not dosed on the day of week 2 blood collections. Mice are typically dosed in the a.m. and only dosed in the p.m. on a day following a 16 hr fast. A test for Free Fatty Acids is done with the plasma final blood collection and with final plasma collection. Assays were done as described above for TG003 IP dosing example.

Results

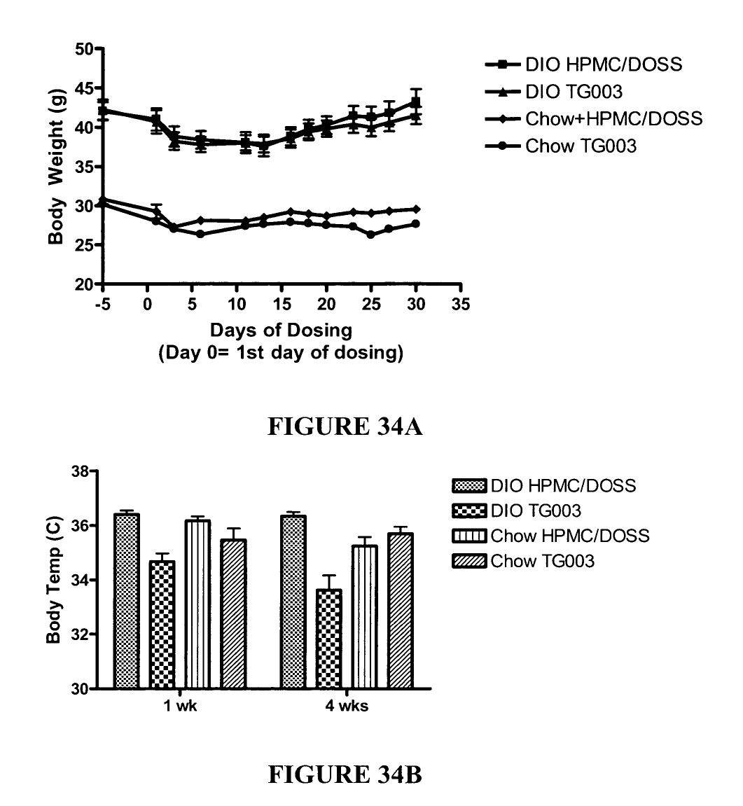

Change in body weight of mice in each group upon commencement of dosing is shown in FIG. 34A for oral dosing at 100 mg/kg TG003 versus vehicle in both the DIO and chow fed groups. Body temperature for all four groups was measured at 1 week and at 4 weeks post dosing. As can be seen in FIG. 34B, there was a significant drop in body temperature in the DIO animals dosed with TG003 following 1 week of dosing and more than a 2 degree drop following 4 weeks of dosing. TG003 had no significant effect on body temperature in the chow fed group.

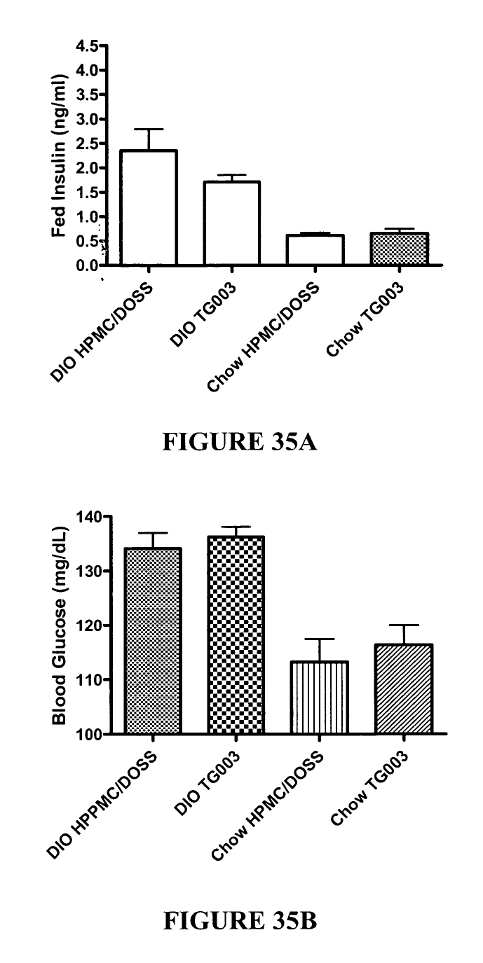

2 week fed insulin and 4 week fed blood glucose results are shown in FIGS. 35A and 35B respectively for oral dosing at 100 mg/kg TG003 versus vehicle in both the DIO and chow fed groups. In general there was not much of an effect upon oral dosing of TG003 as compared to vehicle for either insulin or blood glucose levels in either the DIO or chow groups.

While the initial PK comparison of IP versus oral dosing of TG003 would have suggested similar overall drug exposure for the 30 mg/kg IP dosing as compared to the 100 mg/kg oral dosing, the in vivo effects on body weight, blood glucose and insulin did not repeat with oral dosing. One explanation for this is that for the IP dosing, drug was observed in the intraperitoneal cavity upon sacrifice. This could have served as a depot, allowing for a very different overall drug exposure as compared to oral dosing, especially after multiple injections. The overall effect of TG003 observed after IP dosing may be due to a more continuous drug exposure than could be achieved from oral dosing. Future experiments will address this possibility, including implantable minipumps for continuous release of TG003 in the DIO mouse model.

### EQUIVALENTS

The present invention provides among other things CLK-modulating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

We claim:

1. A method for treating or preventing insulin resistance, a metabolic syndrome, diabetes, or complications thereof, or for increasing insulin sensitivity in a subject, comprising administering to a subject in need thereof a therapeutically effective amount of at least one CLK-inhibiting compound, or a pharmaceutically acceptable salt or prodrug thereof.

2. The method of claim 1, further comprising administering to the subject at least one sirtuin-activating compound.

3. The method of claim 2, wherein the sirtuin-activating compound is selected from the group consisting of: resveratrol, butein, fisetin, piceatannol, quercetin, and nicotinamide riboside.

4. The method of claim 1, wherein said CLK-inhibiting compound is TG003.

5. The method of claim 1, wherein said CLK-inhibiting compound decreases CLK associated phosphorylation of a sirtuin protein and/or PGC-1 alpha.

6. The method of claim 1, wherein the CLK-inhibiting compound is an siRNA, an antisense oligonucleotide, a ribozyme, an aptamer, or an antibody.

7. The method of claim 1, wherein the CLK-inhibiting compound is an inhibitor of at least one human CLK protein.

8. The method of claim 7, wherein the human CLK protein is one or more of hCLK1, hCLK2, hCLK3, and/or hCLK4.

9. The method of claim 8, wherein the human CLK protein is hCLK2.

10. A method for reducing the weight of a subject, or preventing weight gain in a subject, comprising administering to a subject in need thereof a therapeutically effective amount of at least one CLK-inhibiting compound, or a pharmaceutically acceptable salt or prodrug thereof.

11. The method of claim 10, wherein said subject does not reduce calorie consumption, increase activity or a combination thereof to an extent sufficient to cause weight loss in the absence of a CLK-inhibiting compound.

12. A method for treating a disease or disorder in a subject that would benefit from increased mitochondrial activity, comprising administering to a subject in need thereof a therapeutically effective amount of at least one CLK-inhibiting compound, or a pharmaceutically acceptable salt or prodrug thereof.

13. The method of claim 12, further comprising administering to the subject one or more of the following: a vitamin, cofactor or antioxidant.

14. The method of claim 12, further comprising administering to the subject one or more of the following: coenzyme Q₁₀, L-carnitine, thiamine, riboflavin, niacinamide, folate, vitamin E, selenium, lipoic acid, or prednisone.

15. The method of claim 12, further comprising administering to the subject one or more agents that alleviate a symptom of the disease or disorder.

16. The method of claim 15, wherein the agent alleviates seizures, neuropathic pain or cardiac dysfunction.

17. The method of claim 12, wherein the disorder is associated with administration of a pharmaceutical agent that decreases mitochondrial activity.

18. The method of claim 17, wherein the pharmaceutical agent is a reverse transcriptase inhibitor, a protease inhibitor, or an inhibitor or dihydroorotate dehydrogenase (DHOD).

19. A method for (i) promoting survival of a eukaryotic cell, or (ii) preventing the differentiation of a pre-adipocyte, comprising contacting the cell with at least one CLK-inhibiting compound, or a pharmaceutically acceptable salt or prodrug thereof.

20. A method for (i) treating or preventing a disease or disorder associated with cell death or aging in a subject, (ii) treating or preventing a neurodegenerative disorder in a subject, (iii) treating or preventing a blood coagulation disorder in a subject, (iv) treating or preventing an ocular disease or disorder, (v) treating or preventing chemotherapeutic induced neuropathy, (vi) treating or preventing neuropathy associated with an ischemic event or disease, (v) treating or preventing a polyglutamine disease, (vi) treating or preventing a condition wherein motor performance or muscle endurance is reduced, (vii) treating or preventing muscle tissue damage associated with hypoxia or ischemia, (viii) enhancing motor performance or muscle endurance, decreasing fatigue, or increasing recovery from fatigue, or (ix) increasing muscle ATP levels in a subject, comprising administering to a subject in need thereof a therapeutically effective amount of at least one CLK-inhibiting compound, or a pharmaceutically acceptable salt or prodrug thereof.

21. A method for prolonging the lifespan of a subject comprising administering to a subject a therapeutically effective amount of at least one CLK-inhibiting compound, or a pharmaceutically acceptable salt or prodrug thereof.

22. A method for (i) treating or preventing cancer in a subject, or (ii) stimulating weight gain in a subject, comprising administering to a subject in need thereof (a) a therapeutically effective amount of at least one CLK-activating compound, or a pharmaceutically acceptable salt or prodrug thereof, or (b) a polynucleotide that promotes overexpression of a CLK protein.

23. A method for increasing the radiosensitivty or chemosensitivity of a cell comprising (i) contacting the cell with at least one CLK-activating compound, or a pharmaceutically acceptable salt or prodrug thereof, or (ii) introducing into the cell a polynucleotide that promotes overexpression of a CLK protein.

24. A composition comprising at least one CLK-inhibiting compound and at least one sirtuin-activating compound.

25. A composition comprising at least one CLK-activating compound and at least one sirtuin-inhibiting compound.


SEQUENCE LISTING (16 sequences)

SEQ ID NO: 1 - protein, 484 aa, Homo sapiens
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRACKPLKEFMLSQDVEHERLFDLIQKMLEYDPAKRITLREALKHPFFDLLKKSI

SEQ ID NO: 2 - protein, 498 aa, Homo sapiens
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 3 - protein, 490 aa, Homo sapiens
MHHCKRYRSPEPDPYLSYRWKRRRSYSREHEGRLRYPSRREPPPRRSRSRSHDRLPYQRRYRERRDSDTYRCEERSPSFGEDYYGPSRSRHRRRSRERGPYRTRKHAHHCHKRRTRSCSSASSRSQQSSKRSSRSVEDDKEGHLVCRIGDWLQERYEIVGNLGEGTFGKVVECLDHARGKSQVALKIIRNVGKYREAARLEINVLKKIKEKDKENKFLCVLMSDWFNFHGHMCIAFELLGKNTFEFLKENNFQPYPLPHVRHMAYQLCHALRFLHENQLTHTDLKPENILFVNSEFETLYNEHKSCEEKSVKNTSIRVADFGSATFDHEHHTTIVATRHYRPPEVILELGWAQPCDVWSIGCILFEYYRGFTLFQTHENREHLVMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHTSRNPSR

SEQ ID NO: 4 - protein, 481 aa, Homo sapiens
MRHSKRTHCPDWDSRESWGHESYRGSHKRKRRSHSSTQENRHCKPHHQFKESDCHYLEARSLNERDYRDRRYVDEYRNDYCEGYVPRHYHRDIESGYRIHCSKSSVRSRRSSPKRKRNRHCSSHQSRSKSHRRKRSRSIEDDEEGHLICQSGDVLRARYEIVDTLGEGAFGKVVECIDHGMDGMHVAVKIVKNVGRYREAARSEIQVLEHLNSTDPNSVFRCVQMLEWFDHHGHVCIVFELLGLSTYDFIKENSFLPFQIDHIRQMAYQICQSINFLHHNKLTHTDLKPENILFVKSDYVVKYNSKMKRDERTLKNTDIKVVDFGSATYDDEHHSTLVSTRHYRAPEVILALGWSQPCDVWSIGCILIEYYLGFTVFQTHDSKEHLAMMERILGPIPQHMIQKTRKRKYFHHNQLDWDEHSSAGRYVRRRCKPLKEFMLCHDEEHEKLFDLVRRMLEYDPTQRITLDEALQHPFFDLLKKK

SEQ ID NO: 5 - DNA, 1773 bp, Homo sapiens
gacgcagtcagctgcgtgattcccgtgattgcgttacaagctttgtctccttcgacttggagtctttgtccaggacgatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttctttaagtacatagctaaggtaatgaacatctttttcagtaattgtaaagtgatttattcagaataaattttttgtgcttatgaaaaaaaaaaaaaaaaaa

SEQ ID NO: 6 - DNA, 2110 bp, Homo sapiens
ccgagctgggatcgggccccgggcgggggcggtgcgagcggcgccaagcagatcttaggggcggggacggagccggggcgggcgggactgaagcggagcccgggaacggggcgggaggtcccagggtcccgggttgggggggtggagcagcatttcgtcgccgcgggggtgccgggactccggccgcagtgtcgccgccatcacggacttcctgtgggacaagcgcacgggcctcgccgccagaacgatgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcagcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtgacgatcaggccctgggcccccctgcatcttttatagcagtgggtgtccagtccaggacactggtgcttttttatacaagagaacgagccagagttcactccttcctcctggctctctatatacctgtgaatatgtgaaatagtgtaaatatgaaagaacttgtacctatcacttcaacccctgccttgtacaatactattccatccacacagtttccaccctcacctgccccctcatacggagttggatgggggccgagtgaggtaaccaggtggcatctaccccatgttttataaggaattttgtacagtctttgtgaaataaaataacgtgcttcatttgacccccaaaaaaaaaaaaaaaa

SEQ ID NO: 7 - DNA, 1760 bp, Homo sapiens
gggagtggggcctagctgcagccggagcctgggagacgatgcatcactgtaagcgataccgctcccctgaaccagacccgtacctgagctaccgatggaagaggaggaggtcctacagtcgggaacatgaagggagactgcgatacccgtcccgaagggagcctcccccacgaagatctcggtccagaagccatgaccgcctgccctaccagaggaggtaccgggagcgccgtgacagcgatacataccggtgtgaagagcggagcccatcctttggagaggactactatggaccttcacgttctcgtcatcgtcggcgatcgcgggagagggggccataccggacccgcaagcatgcccaccactgccacaaacgccgcaccaggtcttgtagcagcgcctcctcgagaagccaacagagcagtaagcgcagcagccggagtgtggaagatgacaaggagggtcacctggtgtgccggatcggcgattggctccaagagcgatatgagattgtggggaacctgggtgaaggcacctttggcaaggtggtggagtgcttggaccatgccagagggaagtctcaggttgccctgaagatcatccgcaacgtgggcaagtaccgggaggctgcccggctagaaatcaacgtgctcaaaaaaatcaaggagaaggacaaagaaaacaagttcctgtgtgtcttgatgtctgactggttcaacttccacggtcacatgtgcatcgcctttgagctcctgggcaagaacacctttgagttcctgaaggagaataacttccagccttaccccctaccacatgtccggcacatggcctaccagctctgccacgcccttagatttctgcatgagaatcagctgacccatacagacttgaaaccagagaacatcctgtttgtgaattctgagtttgaaaccctctacaatgagcacaagagctgtgaggagaagtcagtgaagaacaccagcatccgagtggctgactttggcagtgccacatttgaccatgagcaccacaccaccattgtggccacccgtcactatcgcccgcctgaggtgatccttgagctgggctgggcacagccctgtgacgtctggagcattggctgcattctctttgagtactaccggggcttcacactcttccagacccacgaaaaccgagagcacctggtgatgatggagaagatcctagggcccatcccatcacacatgatccaccgtaccaggaagcagaaatatttctacaaagggggcctagtttgggatgagaacagctctgacggccggtatgtgaaggagaactgcaaacctctgaagagttacatgctccaagactccctggagcacgtgcagctgtttgacctgatgaggaggatgttagaatttgaccctgcccagcgcatcacactggccgaggccctgctgcaccccttctttgctggcttgacccctgaggagcggtccttccacaccagccgcaacccaagcagatgacaggcacaggccaccgcatgaggagatggagggcgggactgggccgcccagccccttgactccagcctcgaccgccaggccccaggccagagccacccaatgaacagtgcaatgtgaaggaaggcaggagcctgcaggggagcagacttggtgcccagctgccagaaagcacagatttgacccaagctatttatatgttataaagttataataaagtgtttcttactgtttgtaaaaaaaaaaaaaaaa

SEQ ID NO: 8 - DNA, 2524 bp, Homo sapiens
ggcgacggcgctgccgccattttgtggggtgtttgtcgcagcggccgaggagggaagacggcagtttggcgacatttctcggccgaagggccatttgcttttgcggagatgcggcattccaaaagaactcactgtcctgattgggatagcagagaaagctggggacatgaaagctatcgtggaagtcacaagcggaagaggagatctcatagtagcacacaagagaacaggcattgtaaaccacatcaccagtttaaagaatctgattgtcattatttagaagcaaggtccttgaatgagcgagattatcgggaccggagatacgttgacgaatacaggaatgactactgtgaaggatatgttcctagacattatcacagagacattgaaagcgggtatcgaatccactgcagtaaatcttcagtccgcagcaggagaagcagtcctaaaaggaagcgcaatagacactgttcaagtcatcagtcacgttcgaagagccaccgaaggaaaagatccaggagtatagaggatgatgaggagggtcacctgatctgtcaaagtggagacgttctaagagcaagatatgaaatcgtggacactttgggtgaaggagcctttggcaaagttgtagagtgcattgatcatggcatggatggcatgcatgtagcagtgaaaatcgtaaaaaatgtaggccgttaccgtgaagcagctcgttcagaaatccaagtattagagcacttaaatagtactgatcccaatagtgtcttccgatgtgtccagatgctagaatggtttgatcatcatggtcatgtttgtattgtgtttgaactactgggacttagtacttacgatttcattaaagaaaacagctttctgccatttcaaattgaccacatcaggcagatggcgtatcagatctgccagtcaataaattttttacatcataataaattaacccatacagatctgaagcctgaaaatattttgtttgtgaagtctgactatgtagtcaaatataattctaaaatgaaacgtgatgaacgcacactgaaaaacacagatatcaaagttgttgactttggaagtgcaacgtatgatgatgaacatcacagtactttggtgtctacccggcactacagagctcccgaggtcattttggctttaggttggtctcagccttgtgatgtttggagcataggttgcattcttattgaatattaccttggtttcacagtctttcagactcatgatagtaaagagcacctggcaatgatggaacgaatattaggacccataccacaacacatgattcagaaaacaagaaaacgcaagtattttcaccataaccagctagattgggatgaacacagttctgctggtagatatgttaggagacgctgcaaaccgttgaaggaatttatgctttgtcatgatgaagaacatgagaaactgtttgacctggttcgaagaatgttagaatatgatccaactcaaagaattaccttggatgaagcattgcagcatcctttctttgacttattaaaaaagaaatgaaatgggaatcagtggtcttactatatacttctctagaagagattacttaagactgtgtcagtcaactaaacattctaatatttttgtaaacattaaattattttgtacagttaagtgtaaatattgtatgttttgtatcaatagcataattaacttgttaagcaagtatggtcttgataatgcattagaaaaattaaaattaatttttctttttgaaattaccatttttaaatacctttgaaatatcctttgtgtccagtgataaatgtgattgatcttgccttttgtacatggaggtcacctctgaagtgattttttttgagtaaaaggaaatcttgactactttatattcttaaaggaatattctttatatacttcaaatttagaacttaactttaaaagtttttcttctgtaattgttgaacgggtgattattattaactctagataagcaggtactagaaaccaaaactcagaaaatgtttactgttagaattctattaaattttaagtgttgtattctttttcattgggtgatgtcagggtgataaccagacattcatggaaaggcatgcagtttgtccattgtgacagtttgtttaataaaaccacatacacactttatttaagattaaaatctaactggaaagtcagcttggaaaatggacatttccaagtatgtttggtgagtcacagatataaaaatagaaattctgatgagaggtttcagtttttaataccaagtccttaggagtcttaacattggccagcatctgtttatcaaatgacataaatacgtaaacctataagaattaagtttattaattaggcaatttatgtctgtgataattcttacgggagaaagaggatttgattggaaagcagtttgggaagaaagtgctgctgaaatttccagaatttaattgattggttacataaactttttgacttcaaaaaaaaaaaaaaaaaa

SEQ ID NO: 9 - protein, 29 aa, Artificial Sequence
  synthetic peptide of SF2/ASF RS domain
RSPSYGRSRSRSRSRSRSRSRSNSRSRSY

SEQ ID NO: 10 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 11 - protein, 14 aa, Artificial Sequence
  acetylated peptide derived from p53
  VARIANT 9: Xaa = acetylated lysine residue
  VARIANT 10: Xaa = norleucine
GQSTSSHSXXSTEG

SEQ ID NO: 12 - protein, 737 aa, Mus musculus
MADEVALALQAAGSPSAAAAMEAASQPADEPLRKRPRRDGPGLGRSPGEPSAAVAPAAAGCEAASAAAPAALWREAAGAAASAEREAPATAVAGDGDNGSGLRREPRAADDFDDDEGEEEDEAAAAAAAAAIGYRDNLLLTDGLLTNGFHSCESDDDDRTSHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRILQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRPQKELVHLSELPPTPLHISEDSSSPERTVPQDSSVIATLVDQATNNNVNDLEVSESSCVEEKPQEVQTSRNVENINVENPDFKAVGSSTADKNERTSVAETVRKCWPNRLAKEQISKRLEGNQYLFVPPNRYIFHGAEVYSDSEDDVLSSSSCGSNSDSGTCQSPSLEEPLEDESEIEEFYNGLEDDTERPECAGGSGFGADGGDQEVVNEAIATRQELTDVNYPSDKS

SEQ ID NO: 13 - protein, 20 aa, Artificial Sequence
  acetylated and fluorescently labeled peptide derived from p53
  VARIANT 3: Xaa = biotinolated lysine residue
  VARIANT 12: Xaa = acetylated lysine residue
  VARIANT 13: Xaa = norleucine
  VARIANT 18: Xaa = lysine residue modified by an MR121 fluorophore
EEXGQSTSSHSXXSTEGXEE

SEQ ID NO: 14 - protein, 20 aa, Artificial Sequence
  acetylated and fluorescently labeled peptide derived from p53
  VARIANT 3: Xaa = biotinolated lysine residue
  VARIANT 12: Xaa = acetylated lysine residue
  VARIANT 13: Xaa = norleucine
  VARIANT 18: Xaa = lysine residue modified by an 5TMR fluorophore
EEXGQSTSSHSXXSTEGXEE

SEQ ID NO: 15 - DNA, 21 bp, Artificial Sequence
  oligonucleotide corresponding to mouse, rat and human CLK2
ccttcgatttcctcaaagaca

SEQ ID NO: 16 - DNA, 21 bp, Artificial Sequence
  oligonucleotide corresponding to a control siRNA
ccttcgattccctcaaagaca